(19) United States
(12) Patent Application Publication
(10) Pub. No.: US 2025/0145608 A1
(43) Pub. Date: May 8, 2025

(54) KRAS DEGRADING COMPOUNDS COMPRISING ANNULATED 2-AMINO-3-CYANO THIOPHENES

(71) Applicants: Boehringer Ingelheim Int'l GmbH Corp., Ingelheim am Rhein (DE); Vanderbilt University, Nashville, TN (US)

(72) Inventors: Joachim BROEKER, Moedling (AT); Jason ABBOTT, Libertyville, IL (US); Alessio CIULLI, Dundee (GB); Jianwen CUI, Franklin, TN (US); Emelyne DIERS, MoIndal (SE); William FARNABY, St.Andrews,Fife (GB); Stephen W. FESIK, Nashville, TN (US); Andreas GOLLNER, Vienna (AT); Jake GOODWIN-TINDALL, Cambridge (GB); Tim HODGES, Chicago, IL (US); Christiane KOFINK, Perchtoldsdorf (AT); Andrew LITTLE, Sudbury, MA (US); Andreas MANTOULIDIS, Vienna (AT); Ross MCLENNAN, Dundee (GB); Johannes POPOW, Vienna (AT); Dhruba SARKAR, Bangalore (IN); Martin SCHMIEDEL, Vienna (AT); Christian Alan Paul SMETHURST; Nicole TRAINOR, Melbourne (AU); Alex WATERSON, Murfreesboro, TN (US); Harald WEINSTABL, Vienna (AT); Siying ZHONG, Cambridge (GB); David ZOLLMAN, Dundee (GB)

(21) Appl. No.: 18/715,145

(22) PCT Filed: Nov. 30, 2022
(86) PCT No.: PCT/EP2022/083950

§ 371 Date: May 31, 2024

Related U.S. Application Data
(60) Provisional application No. 63/284,751, filed on Dec. 1, 2021.

(51) Int. Cl. C07D 417/14; A61K 31/551; A61P 35/00; C07D 413/14

(52) U.S. Cl. CPC C07D 417/14; A61K 31/551; A61P 35/00; C07D 413/14

(57) ABSTRACT

The present invention relates to compounds and derivatives of formula (I) which target KRAS and can be useful as agents for treatment and/or prevention of oncological diseases.

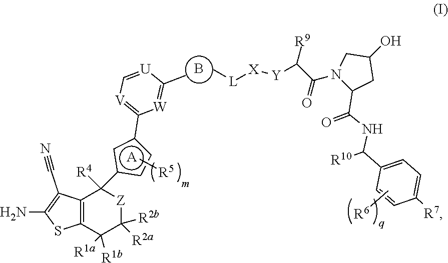

### FIELD OF THE INVENTION

The present invention relates to compounds and derivatives of formula (I):

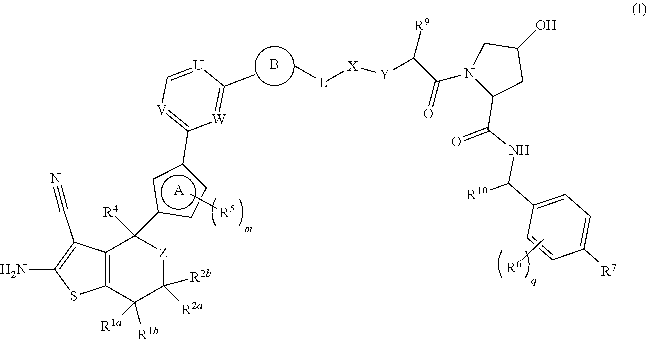

wherein R^{1a}, R^{1b}, R^{2a}, R^{2b}, Z, R⁴, ring A, R⁵, m, U, V, W, ring B, L, X, Y, R⁹, R¹⁰, q, R⁵ and R⁷ have the meanings given in the claims and specification, their use as degraders of KRAS, pharmaceutical compositions comprising the same and their medical uses, especially as agents for treatment and/or prevention of oncological diseases.

### BACKGROUND OF THE INVENTION

V-Ki-ras2 Kirsten rat sarcoma viral oncogene homolog (KRAS) is a small GTPase of the Ras family of proteins that exists in cells in either GTP-bound or GDP-bound states (McCormick et al., J. Mol. Med. (Berl)., 2016, 94(3):253-8; Nimnual et al., Sci. STKE., 2002, 2002(145):pe36). Binding of GTPase activating proteins (GAPs) such as NF1 increases the GTPase activity of Ras family proteins. The binding of guanine nucleotide exchange factors (GEFs) such as SOS1 (Son of Sevenless 1) promotes release of GDP from Ras family proteins, enabling GTP binding (Chardin et al., Science, 1993, 260(5112):1338-43). When in the GTP-bound state, Ras family proteins are active and engage effector proteins including C-RAF and phosphoinositide 3-kinase (PI3K) to promote the RAF/mitogen or extracellular signal-regulated kinases (MEK/ERK) pathway, PI3K/AKT/mammalian target of rapamycin (mTOR) pathway and RaIGDS (Ral guanine nucleotide dissociation stimulator) pathway (McCormick et al., J. Mol. Med. (Berl)., 2016, 94(3):253-8; Rodriguez-Viciana et al., Cancer Cell. 2005, 7(3):205-6). These pathways affect diverse cellular processes such as proliferation, survival, metabolism, motility, angiogenesis, immunity and growth (Young et al., Adv. Cancer Res., 2009, 102:1-17; Rodriguez-Viciana et al., Cancer Cell. 2005, 7(3):205-6).

Cancer-associated mutations in Ras family proteins suppress their intrinsic and GAP-induced GTPase activity leading to an increased population of GTP-bound/active mutant Ras family proteins (McCormick et al., Expert Opin. Ther. Targets., 2015, 19(4):451-4; Hunter et al., Mol. Cancer Res., 2015, 13(9):1325-35). This in turn leads to persistent activation of effector pathways (e.g. RAF/MEK/ERK, PI3K/AKT/mTOR, RaIGDS pathways) downstream of mutant Ras family proteins.

KRAS mutations (e.g. amino acids G12, G13, Q61, A146) are found in a variety of human cancers including lung cancer, colorectal cancer and pancreatic cancer (Cox et al., Nat. Rev. Drug Discov., 2014, 13(11):828-51). Alterations (e.g. mutation, over-expression, gene amplification) in Ras family proteins/Ras genes have also been described as a resistance mechanism against cancer drugs such as the EGFR antibodies cetuximab and panitumumab (Leto et al., J. Mol. Med. (Berl). 2014 July; 92(7):709-22) and the EGFR tyrosine kinase inhibitor osimertinib/AZD9291 (Ortiz-Cuaran et al., Clin. Cancer Res., 2016, 22(19):4837-47; Eberlein et al., Cancer Res., 2015, 7 5(12):2489-500).

In a subset of tumor indications such as gastric cancer, gastroesophageal junction cancer and esophageal cancer prominent amplification of the wildtype (WT) KRAS proto-oncogene acts as a driver alteration and renders tumor models bearing this genotype addicted to KRAS in vitro and in vivo (Wong et al. Nat Med., 2018, 24(7):968-977). In contrast, non-amplified KRAS WT cell lines are KRAS independent, unless they carry secondary alterations in genes indirectly causing activation of KRAS (Meyers et al., Nat Genet., 2017, 49:1779-1784). Based on these data, a therapeutic window is expected for a KRAS targeting agent with a KRAS WT targeting activity.

Genetic alterations affecting e.g. codon 12 of KRAS substitute the glycine residue naturally occurring at this position for different amino acids such as aspartic acid (the G12D mutation or KRAS G12D), cysteine (the G12C mutation or KRAS G12C), valine (the G12V mutation or KRAS G12V) among others. Similarly, mutations within codons 13, 61 and 146 of KRAS are commonly found in the KRAS gene. Altogether KRAS mutations are detectable in 35% of lung, 45% of colorectal, and up to 90% of pancreatic cancers (Herdeis et al., Curr Opin Struct Biol., 2021, 71:136-147).

Proteolysis targeting chimeras (PROTACs) bind to proteins causing their degradation by inducing their ubiquitination. PROTACs are tripartite or heterobifunctional molecules consisting of a part binding to the protein that is to be degraded, a second part that binds to and can artificially recruit an E3 ubiquitin ligase, and a linker that connects the two parts. Whenever a trimeric complex consisting of the target protein, the PROTAC, and the ligase is formed, the close proximity of the ligase to the target results in target protein ubiquitination. Ubiquitination acts as a posttranslational modification of proteins causing, among others, their recruitment to the proteasome resulting in proteolytic degradation. The multi-ubiquitin chain on the target protein is then recognized by the proteasome and the target protein is degraded (Collins, I. et al., Biochem J., 2017, 474, 1127-1147; Hughes, S. J. and Ciulli, A, Essays Biochem., 2017, 61, 505-516; Toure, M. and Crews, C. M., Angew. Chem. Int. Ed. Engl., 2016, 55, 1966-1973).

In contrast to classical small molecule drugs, PROTAC driven degradation functions in a sub-stoichiometric nature thus requiring lower systemic exposures to achieve efficacy (Bondeson, D. P. et al., Nat. Chem. Bio., 2015, 11, 611; Winter, G. E. et al., Science, 2015, 348, 1376-1381). PROTACs have been shown to display higher degrees of selectivity for protein degradation than the target ligand itself due to complementarity differences in the protein-protein-interaction interfaces of the formed ternary complexes (Bondeson, D. P. et al., Cell Chemical Biology, 2018, 25, 78-87.e75; Gadd, M. S. et al. (2017), Nature Chemical Biology, 13, 514-521; Nowak, R. P. et al. (2018), Nat. Chem. Bio., 14(7):706-714; Zengerle, M. et al., ACS Chemical Biology, 2015, 10, 1770-1777). In addition, PROTACs promise to expand the druggable proteome as degradation is not limited to the protein domain functionally responsible for the disease. In the case of challenging multidomain proteins, traditionally viewed as undruggable targets, the most ligandable domain can be targeted for degradation independent of its functionality or vulnerability to small molecule blockade (Gechijian, L. N. et al., Nat. Chem. Bio., 2018, 14, 405-412).

Irreversible in nature, induced degradation of KRAS by recruitment of an E3 ubiquitin ligase is expected to induce comparable cellular effects as irreversible inhibition. Moreover, since mutant KRAS still is expected to be subject to GEF/GAP induced cycling between the GTP-bound active and GDP-bound inactive states, induced degradation of mutant KRAS by PROTACs engaging the GDP-bound state may lead to gradual degradation of a large fraction of the entire cellular KRAS pool. Hence, degradation of oncogenic KRAS mutants may inhibit downstream signaling in tumors delivering anticancer efficacy as described for KRAS inhibition. Upon irreversible target degradation recovery of downstream signaling activity not only depends on elimination of the drug from the treated subject (e.g. by clearance) but is further limited by de novo resynthesis of the target protein by the ribosome. Irreversible inhibition is so far restricted to the KRAS G12C protein, which only constitutes a fraction of the whole complement of KRAS mutant tumors. In contrast, induced degradation of KRAS has the potential to enable irreversible inhibition of KRAS signaling for most remaining KRAS mutations/alterations driving tumor growth provided they can be bound by a heterobifunctional degrader molecule.

In summary, degraders of wild-type (e.g. amplified or overexpressed) or mutated KRAS (e.g. G12C, G12D, G12V, G13D) are expected to deliver anti-cancer efficacy. Therefore, there remains the need for new compounds capable of degrading KRAS, in its wild-type or mutated forms, said compounds ideally being effective across a panel of protein forms. Such compounds would be useful in the treatment of a cancer dependent on or mediated by KRAS, especially KRAS mutated in position 12 or 13 and/or in wild-type amplified KRAS mediated cancer, which also possess desirable pharmacological properties, including but not limited to: metabolic stability, plasma protein binding, solubility and permeability.

### DETAILED DESCRIPTION OF THE INVENTION

### Compounds

It has now been found that, surprisingly, compounds of the present invention have additional advantages. In particular, compounds of formula (I) as herein defined can act as degraders of KRAS. Surprisingly, the compounds described herein have been found to possess anti-tumour activity. Advantageously, the compounds of the invention are effective against wild-type (e.g. amplified or overexpressed) as well as mutant KRAS, e.g. G12C, G12D, G12V, G13D. In particular, they can be effective against a panel of KRAS mutated forms. Thus, the compounds of the invention may be used for example for the treatment of diseases mediated by KRAS and/or characterised by excessive or abnormal cell proliferation.

In addition, the compounds of the invention advantageously possess desirable pharmacological properties, including but not limited to: metabolic stability, plasma protein binding, solubility and permeability.

It is therefore an object of the present invention a compound of formula (I):

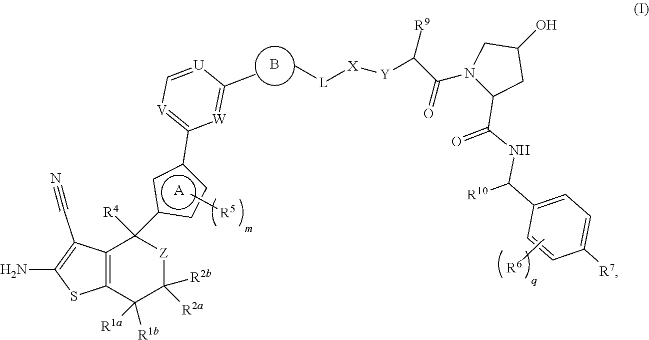

wherein:
R^{1a} and R^{1b} are both independently selected from the group consisting of hydrogen, C₁₋₄alkyl, C₁₋₄haloalkyl, C₁₋₄alkoxy, C₁₋₄haloalkoxy, halogen, —NH₂, —NH(C₁₋₄alkyl), —N(C₁₋₄alkyl)₂, C₃₋₅cycloalkyl and 3-5 membered heterocycloalkyl;
R^{2a} and R^{2b} are both independently selected from the group consisting of hydrogen, C₁₋₄alkyl, C₁₋₄haloalkyl, C₁₋₄alkoxy, C₁₋₄haloalkoxy, halogen, —NH₂, —NH(C₁₋₄alkyl), —N(C₁₋₄alkyl)₂, C₃₋₅cycloalkyl and 3-5 membered heterocycloalkyl;
and/or, optionally, one of R^{1a} or R^{1b} and one of R^{2a} or R^{2b} together with the carbon atoms they are attached to form a cyclopropane ring;
Z is —(CR^{3a}R^{3b})ₙ—;
each R^{3a} and R^{3b} is independently selected from the group consisting of hydrogen, C₁₋₄alkyl, C₁₋₄haloalkyl, C₁₋₄alkoxy, C₁₋₄haloalkoxy, halogen, —NH₂, —NH(C₁₋₄alkyl), —N(C₁₋₄alkyl)₂, C₃₋₅cycloalkyl and 3-5 membered heterocycloalkyl;
or R^{3a} and R^{3b} together with the carbon atom they are attached to form a cyclopropane ring; n is selected from the group consisting of 0, 1 and 2;
R⁴ is selected from the group consisting of hydrogen, C₁₋₆-alkyl, C₁₋₆-haloalkyl, C₁₋₆-alkoxy, C₁₋₆-haloalkoxy, cyano-C₁₋₆-alkyl, halogen, —OH, —NH₂, —NH(C₁₋₄alkyl), —N(C₁₋₄alkyl)₂, —CN, C₃₋₅cycloalkyl and 3-5 membered heterocycloalkyl;
ring A is a 5 membered heteroarylene;
each R⁵, if present, is independently selected from the group consisting of C₁₋₆-alkyl, C₁₋₆ haloalkyl, C₁₋₆alkoxy, C₁₋₆haloalkoxy, cyano-C₁₋₆alkyl, halogen, —OH, —NH₂, —NH(C₁₋₄alkyl), —N(C₁₋₄alkyl)₂, —CN, C₃₋₅cycloalkyl and 3-5 membered heterocycloalkyl;
m is selected from the group consisting of 0, 1, 2 and 3;
W is nitrogen (—N═) or —CH═;
V is nitrogen (—N═) or —CH═;
U is nitrogen (—N═) or —C(R¹¹)═;
R¹¹ is selected from hydrogen, halogen and C₁₋₄alkoxy;
ring B is a 3-11 membered heterocycloalkylene optionally substituted with one or more identical or different C₁₋₆-alkyl, C₁₋₆-alkoxy or a 5-6 membered heterocycloalkyl, wherein the C₁₋₆-alkyl is optionally substituted with cyclopropyl;
L is selected from the group consisting of a bond, C₁₋₈alkylene, C₂₋₈alkenylene, C₂₋₈alkynylene and C₁₋₈alkoxylene;
X is —(CH₂)— or —O—;
Y is a 5 membered heteroarylene or —C(O)(NR¹²)—, wherein said 5 membered heteroarylene comprises at least one nitrogen atom and wherein said —C(O)(NR¹²)— is linked to X via the C atom;
R⁹ is C₁₋₄ alkyl;
R¹⁰ is selected from the group consisting of hydrogen, C₁₋₆alkyl, C₁₋₆alkoxy, —C(O)R¹² and —C(O)OR¹², wherein said C₁₋₆alkyl is optionally substituted by —OH or —OP(O)(OH)₂; each R¹² is independently hydrogen or C₁₋₄alkyl;
q is selected from the group consisting of 0, 1 and 2;
each R⁵, if present, is independently at each occurrence halogen or C₁₋₃alkyl;
R⁷ is selected from the group consisting of halogen, C₁₋₃alkyl, —CN and 5 membered heteroaryl, wherein said 5 membered heteroaryl comprises at least one nitrogen atom and is optionally substituted with R⁸;
R⁸ is C₁₋₃alkyl or C₁₋₃hydroxyalkyl;
or a salt thereof.

In one aspect, the compound or salt of formula (I) is of formula (I*):

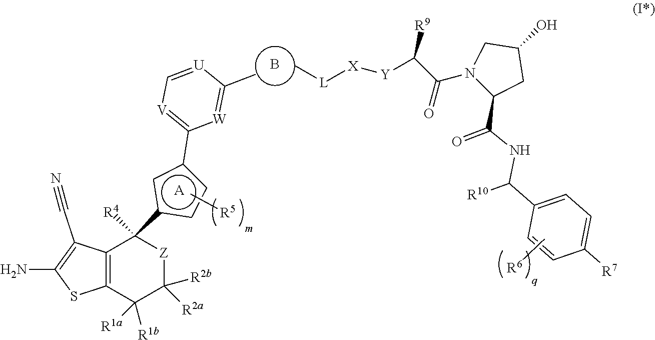

wherein R^{1a}, R^{1b}, R^{2a}, R^{2b}, Z, R⁴, ring A, R⁵, m, U, V, W, ring B, L, X, Y, R⁹, R¹⁰, q, R⁵ and R⁷ are as herein defined.

In another aspect, the compound or salt of formula (I) is of formula (I**):

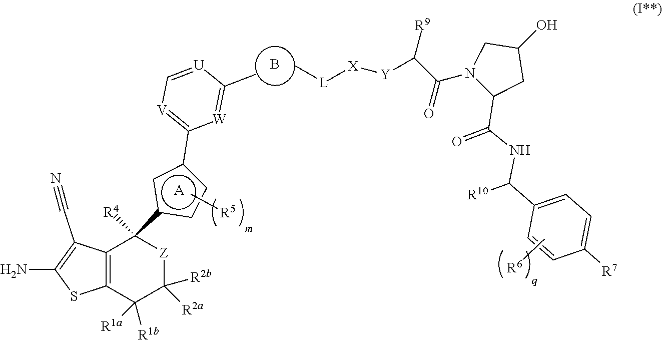

wherein R^{1a}, R^{1b}, R^{2a}, R^{2b}, Z, R⁴, ring A, R⁵, m, U, V, W, ring B, L, X, Y, R⁹, R¹⁰, q, R⁵ and R⁷ are as herein defined.

In another aspect, the compound or salt of formula (I) is of formula (I***):

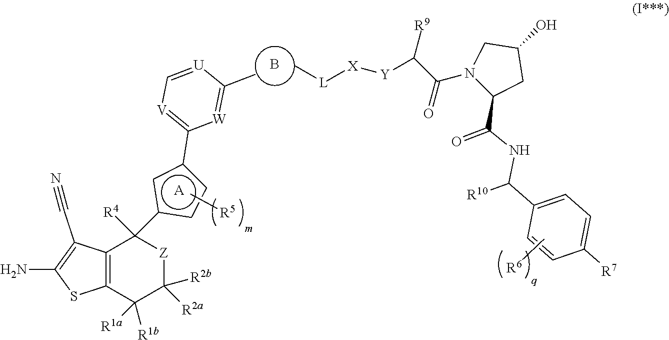

wherein R^{1a}, R^{1b}, R^{2a}, R^{2b}, Z, R⁴, ring A, R⁵, m, U, V, W, ring B, L, X, Y, R⁹, R¹⁰, q, R⁵ and R⁷ are as herein defined.

In another aspect, the compound of formula (I) is of formula (I****):

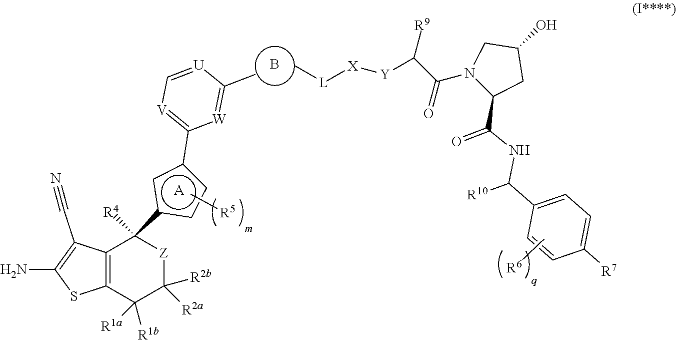

wherein R^{1a}, R^{1b}, R^{2a}, R^{2b}, Z, R⁴, ring A, R⁵, m, U, V, W, ring B, L, X, Y, R⁹, R¹⁰, q, R⁵ and R⁷ are as herein defined.

It is to be understood that the configuration at the asymmetric carbon atoms depicted in formulas (I*), (I**), (I***) and (I****) can be applied to any one or more of the aspects and/or preferred embodiments defined below.

In another aspect, the compound of formula (I) is of formula (Ia)

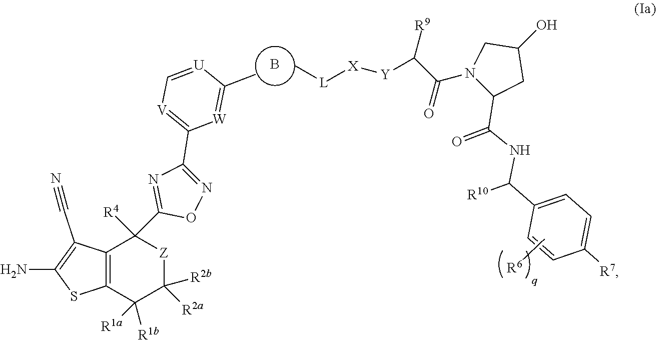

wherein R^{1a}, R^{1b}, R^{2a}, R^{2b}, Z, R⁴, U, V, W, ring B, L, X, Y, R⁹, R¹⁰, q, R⁶, R⁷ and the stereochemistry are as herein defined. Preferably, the stereochemistry at the chiral centres in formula (Ia) is as defined in any one of formulas (I*), (I**), (I***) or (I****).

In another aspect, the compound of formula (I) is of formula (Ib):

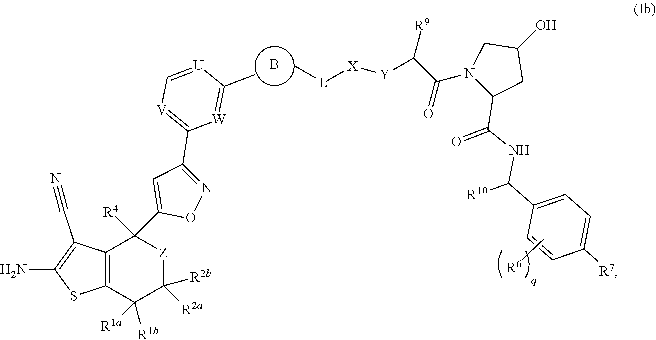

wherein R^{1a}, R^{1b}, R^{2a}, R^{2b}, Z, R⁴, U, V, W, ring B, L, X, Y, R⁹, R¹⁰, q, R⁶, R⁷ and the stereochemistry are as herein defined. Preferably, the stereochemistry at the chiral centres in formula (Ib) is as defined in any one of formulas (I*), (I**), (I***) or (I****).

In another aspect, the compound of formula (I) is of formula (Ic):

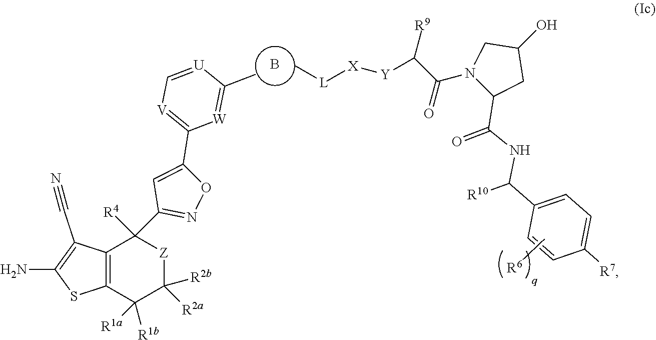

wherein R^{1a}, R^{1b}, R^{2a}, R^{2b}, Z, R⁴, U, V, W, ring B, L, X, Y, R⁹, R¹⁰, q, R⁶, R⁷ and the stereochemistry are as herein defined. Preferably, the stereochemistry at the chiral centres in formula (Ic) is as defined in any one of formulas (I*), (I**), (I***) or (I****).

In another aspect, the compound of formula (I) is of formula (Id):

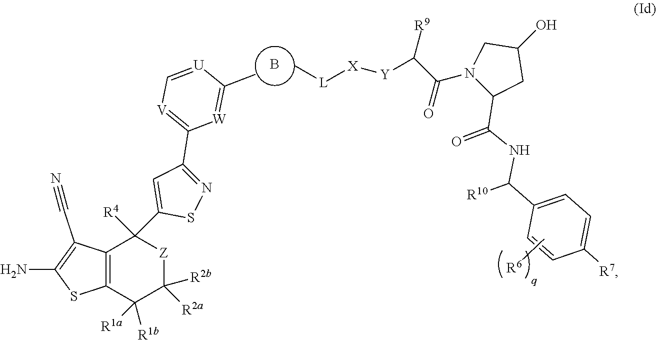

wherein R^{1a}, R^{1b}, R^{2a}, R^{2b}, Z, R⁴, U, V, W, ring B, L, X, Y, R⁹, R¹⁰, q, R⁶, R⁷ and the stereochemistry are as herein defined. Preferably, the stereochemistry at the chiral centres in formula (Id) is as defined in any one of formulas (I*), (I**), (I***) or (I****).

In another aspect, the compound of formula (I) is of formula (Ie):

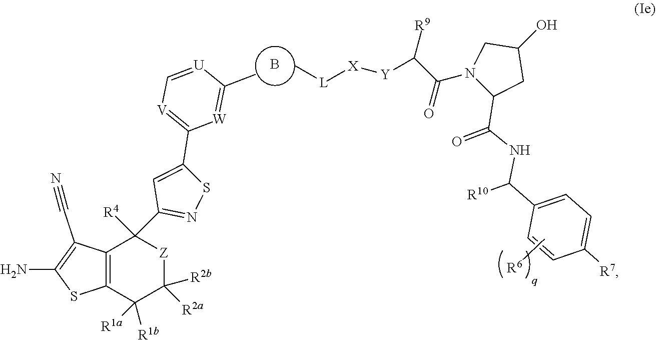

wherein R^{1a}, R^{1b}, R^{2a}, R^{2b}, Z, R⁴, U, V, W, ring B, L, X, Y, R⁹, R¹⁰, q, R⁶, R⁷ and the stereochemistry are as herein defined. Preferably, the stereochemistry at the chiral centres in formula (Ie) is as defined in any one of formulas (I*), (I**), (I***) or (I****).

In another aspect, the compound of formula (I) is of formula (If):

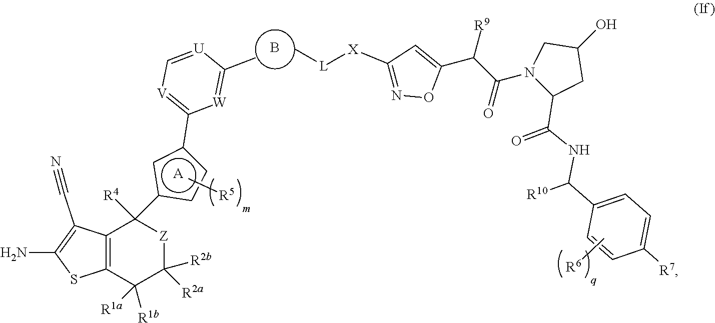

wherein R^{1a}, R^{1b}, R^{2a}, R^{2b}, Z, R⁴, ring A, R⁵, m, U, V, W, ring B, L, X, R⁹, R¹⁰, q, R⁵, R⁷ and the stereochemistry are as herein defined. Preferably, the stereochemistry at the chiral centres in formula (If) is as defined in any one of formulas (I*), (I**), (I***) or (I****).

In another aspect, the compound of formula (I) is of formula (Ig):

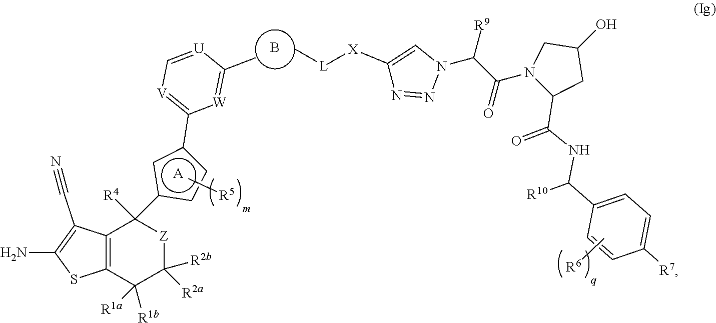

wherein R^{1a}, R^{1b}, R^{2a}, R^{2b}, Z, R⁴, ring A, R⁵, m, U, V, W, ring B, L, X, R⁹, R¹⁰, q, R⁵, R⁷ and the stereochemistry are as herein defined. Preferably, the stereochemistry at the chiral centres in formula (Ig) is as defined in any one of formulas (I*), (I**), (I***) or (I****).

In another aspect, the compound of formula (I) is of formula (Ih):

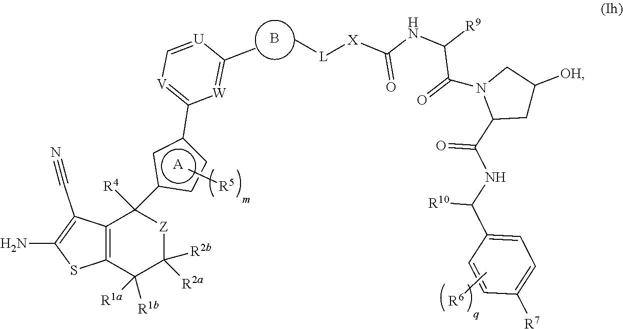

wherein R^{1a}, R^{1b}, R^{2a}, R^{2b}, Z, R⁴, ring A, R⁵, m, U, V, W, ring B, L, X, R⁹, R¹⁰, q, R⁶, R⁷ and the stereochemistry are as herein defined. Preferably, the stereochemistry at the chiral centres in formula (Ih) is as defined in any one of formulas (I*), (I**), (I***) or (I****).

It is to be understood that compounds of formula (I*), (I**), (I***), (I****), (Ia), (Ib), (Ic), (Id), (Ie), (If), (Ig), and (Ih) each are a subset of compounds of formula (I). Any reference to compounds of formula (I) is meant to also refer to and include compounds of each of formulas (I*), (I**), (I***), (I****), (Ia), (Ib), (Ic), (Id), (Ie), (If), (Ig), and (Ih) unless stated otherwise. Formulas (I*), (I**), (I***), (I****), (Ia), (Ib), (Ic), (Id), (Ie), (If), (Ig), and (Ih) alone or together can be referred to as “subformula” or “subformulas” of formula (I).

In one aspect of the compound of formula (I), (I*), (I**), (I***), (I****), (Ia), (Ib), (Ic), (Id), (Ie), (If), (Ig), or (Ih), one of R^{1a} or R^{1b} is hydrogen.

In another aspect of the compound of formula (I), (I*), (I**), (I***), (I****), (Ia), (Ib), (Ic), (Id), (Ie), (If), (Ig), or (Ih), R^{1a} and R^{1b} are hydrogen.

In another aspect of the compound of formula (I), (I*), (I**), (I***), (I****), (Ia), (Ib), (Ic), (Id), (Ie), (If), (Ig), or (Ih), one of R^{2a} or R^{2b} is hydrogen.

In another aspect of the compound of formula (I), (I*), (I**), (I***), (I****), (Ia), (Ib), (Ic), (Id), (Ie), (If), (Ig), or (Ih), R^{2a} and R^{2b} are hydrogen.

In another aspect of the compound of formula (I), (I*), (I**), (I***), (I****), (Ia), (Ib), (Ic), (Id), (Ie), (If), (Ig), or (Ih), R^{1a}, R^{1b}, R^{2a} and R^{2b} are hydrogen.

In another aspect of the compound of formula (I), (I*), (I**), (I***), (I****), (Ia), (Ib), (Ic), (Id), (Ie), (If), (Ig), or (Ih), one of R^{3a} or R^{3b} is hydrogen.

In another aspect of the compound of formula (I), (I*), (I**), (I***), (I****), (Ia), (Ib), (Ic), (Id), (Ie), (If), (Ig), or (Ih), R^{3a} and R^{3b} are hydrogen.

In another aspect of the compound of formula (I), (I*), (I**), (I***), (I****), (Ia), (Ib), (Ic), (Id), (Ie), (If), (Ig), or (Ih), R^{1a}, R^{1b}, R^{2a}, R^{2b}, R^{3a} and R^{3b} are hydrogen.

In another aspect of the compound of formula (I), (I*), (I**), (I***), (I****), (Ia), (Ib), (Ic), (Id), (Ie), (If), (Ig), or (Ih), n is 1.

In another aspect of the compound of formula (I), (I*), (I**), (I***), (I****), (Ia), (Ib), (Ic), (Id), (Ie), (If), (Ig), or (Ih), Z is —CH₂—.

In another aspect of the compound of formula (I), (I*), (I**), (I***), (I****), (Ia), (Ib), (Ic), (Id), (Ie), (If), (Ig), or (Ih), R^{1a}, R^{1b}, R^{2a} and R^{2b} are hydrogen and Z is —CH₂—.

In another aspect of the compound of formula (I), (I*), (I**), (I***), (I****), (Ia), (Ib), (Ic), (Id), (Ie), (If), (Ig), or (Ih), the carbon atom to which R⁴ and ring A are attached is in the (S) configuration.

In another aspect of the compound of formula (I), (I*), (I**), (I***), (I****), (Ia), (Ib), (Ic), (Id), (Ie), (If), (Ig), or (Ih), R⁴ is selected from the group consisting of hydrogen, C₁₋₃alkyl, C₁₋₃haloalkyl, C₁₋₃alkoxy, C₁₋₃haloalkoxy, cyano-C₁₋₃alkyl, halogen, —OH, —NH₂, —NH(C₁₋₃alkyl), —N(C₁₋₃alkyl)₂, —CN, C₃₋₄cycloalkyl and 3-4 membered heterocycloalkyl.

In another aspect of the compound of formula (I), (I*), (I**), (I***), (I****), (Ia), (Ib), (Ic), (Id), (Ie), (If), (Ig), or (Ih), R⁴ is C₁₋₆alkyl.

In another aspect of the compound of formula (I), (I*), (I**), (I***), (I****), (Ia), (Ib), (Ic), (Id), (Ie), (If), (Ig), or (Ih), R⁴ is C₁₋₃alkyl.

In another aspect of the compound of formula (I), (I*), (I**), (I***), (I****), (Ia), (Ib), (Ic), (Id), (Ie), (If), (Ig), or (Ih), R⁴ is methyl.

In another aspect of the compound of formula (I), (I*), (I**), (I***), (I****), (Ia), (Ib), (Ic), (Id), (Ie), (If), (Ig), or (Ih), R^{1a}, R^{1b}, R^{2a} and R^{2b} are hydrogen, Z is —CH₂— and R⁴ is methyl.

In another aspect of the compound of formula ((I), (I*), (I**), (I***), (I****), (Ia), (Ib), (Ic), (Id), (Ie), (If), (Ig), or (Ih), R^{1a}, R^{1b}, R^{2a} and R^{2b} are hydrogen, Z is —CH₂—, R⁴ is methyl and the carbon atom to which R⁴ and ring A are attached is in the (S) configuration.

In another aspect of the compound of formula (I), (I*), (I**), (I***), (I****), (If), (Ig), or (Ih), ring A is selected from the group consisting of pyrrole, furan, thiophene, imidazole, pyrazole, oxazole, isoxazole, thiazole, isothiazole, oxadiazole, thiadiazole and triazole.

In another aspect of the compound of formula (I), (I*), (I**), (I***), (I****), (If), (Ig), or (Ih), ring A is selected from the group consisting of oxazole, isoxazole, thiazole, isothiazole, oxadiazole and thiadiazole.

In another aspect of the compound of formula (I), (I*), (I**), (I***), (I****), (If), (Ig), or (Ih), ring A is selected from the group consisting of isoxazole, isothiazole and oxadiazole.

In another aspect of the compound of formula (I), (I*), (I**), (I***), (I****), (If), (Ig), or (Ih), ring A is selected from the group consisting of:

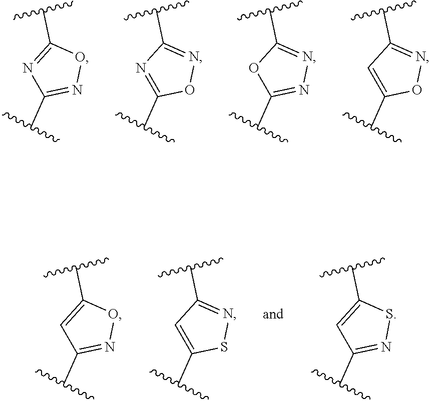

In another aspect of the compound of formula (I), (I*), (I**), (I***), (I****), (If), (Ig), or (Ih), ring A is selected from the group consisting of:

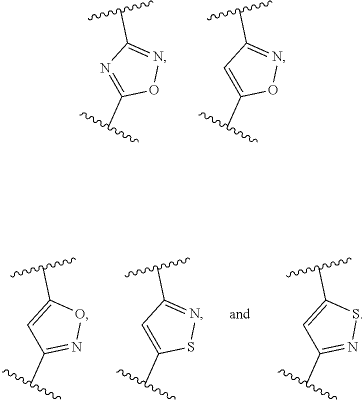 [SMILES: *c1noc(*)n1 ; *c1cc(*)on1 ; *c1cc(*)on1 ; *c1cc(*)sn1 ; "and" ; *c1cc(*)sn1]

In another aspect of the compound of formula (I), (I*), (I**), (I***), (I****), (Ia), (Ib), (Ic), (Id), (Ie), (If), (Ig), or (Ih), at any occurrence oxadiaziole is selected from the group consisting of

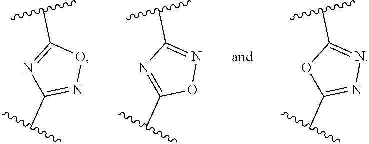 [SMILES: *c1noc(*)n1 ; *c1noc(*)n1 ; "and" ; *c1nnc(*)o1]

In another aspect of the compound of formula (I), (I*), (I**), (I***), (I****), (Ia), (Ib), (Ic), (Id), (Ie), (If), (Ig), or (Ih), at any occurrence oxadiaziole is

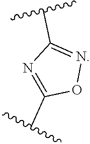 [SMILES: *c1noc(*)n1]

In another aspect of the compound of formula (I), (I*), (I**), (I***), (I****), (Ia), (Ib), (Ic), (Id), (Ie), (If), (Ig), or (Ih), at any occurrence isoxazole is:

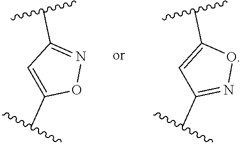 [SMILES: *c1cc(*)on1 ; "or" ; *c1cc(*)on1]

In another aspect of the compound of formula (I), (I*), (I**), (I***), (I****), (Ia), (Ib), (Ic), (Id), (Ie), (If), (Ig), or (Ih), at any occurrence isothiazole is:

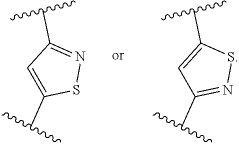 [SMILES: *c1cc(*)sn1 ; "or" ; *c1cc(*)sn1]

In another aspect of the compound of formula (I), (I*), (I**), (I***), (I****), (If), (Ig), or (Ih), m is 0.

In another aspect of the compound of formula (I), (I*), (I**), (I***), (I****), (Ia), (Ib), (Ic), (Id), (Ie), (If), (Ig), or (Ih), R¹¹ is hydrogen

In another aspect of the compound of formula (I), (I*), (I**), (I***), (I****), (Ia), (Ib), (Ic), (Id), (Ie), (If), (Ig), or (Ih), U, V and W are each independently ═N— or ═C(H)—.

In another aspect of the compound of formula (I), (I*), (I**), (I***), (I****), (Ia), (Ib), (Ic), (Id), (Ie), (If), (Ig), or (Ih), at least one of V or W is ═N—.

In another aspect of the compound of formula (I), (I*), (I**), (I***), (I****), (Ia), (Ib), (Ic), (Id), (Ie), (If), (Ig), or (Ih), one of V or W is ═N—.

In another aspect of the compound of formula (I), (I*), (I**), (I***), (I****), (Ia), (Ib), (Ic), (Id), (Ie), (If), (Ig), or (Ih), both V and W are ═N—.

In another aspect of the compound of formula (I), (I*), (I**), (I***), (I****), (Ia), (Ib), (Ic), (Id), (Ie), (If), (Ig), or (Ih), W is ═N—.

In another aspect of the compound of formula (I), (I*), (I**), (I***), (I****), (Ia), (Ib), (Ic), (Id), (Ie), (If), (Ig), or (Ih), U is ═N—, V is ═C(H)— and W is ═N—.

In another aspect of the compound of formula (I), (I*), (I**), (I***), (I****), (Ia), (Ib), (Ic), (Id), (Ie), (If), (Ig), or (Ih), U is ═C(H)—, V is ═N— and W is ═N—.

In another aspect of the compound of formula (I), (I*), (I**), (I***), (I****), (Ia), (Ib), (Ic), (Id), (Ie), (If), (Ig), or (Ih), R^{1a}, R^{1b}, R^{2a} and R^{2b} are hydrogen, Z is —CH₂—, R⁴ is methyl and W is ═N—.

In another aspect of the compound of formula (I), (I*), (I**), (I***), (I****), (Ia), (Ib), (Ic), (Id), (Ie), (If), (Ig), or (Ih), R^{1a}, R^{1b}, R^{2a} and R^{2b} are hydrogen, Z is —CH₂—, R⁴ is methyl, W is ═N and the carbon atom to which R⁴ and ring A are attached is in the (S) configuration.

In another aspect of the compound of formula (I), (I*), (I**), (I***), (I****), (Ia), (Ib), (Ic), (Id), (Ie), (If), (Ig), or (Ih), R^{1a}, R^{1b}, R^{2a} and R^{2b} are hydrogen, Z is —CH₂—, R⁴ is methyl, U is ═N—, V is ═C(H)— and W is ═N—.

In another aspect of the compound of formula (I), (I*), (I**), (I***), (I****), (Ia), (Ib), (Ic), (Id), (Ie), (If), (Ig), or (Ih), R^{1a}, R^{1b}, R^{2a} and R^{2b} are hydrogen, Z is —CH₂—, R⁴ is methyl, U is ═C(H)—, V is ═N— and W is ═N—.

In another aspect of the compound of formula (I), (I*), (I**), (I***), (I****), (Ia), (Ib), (Ic), (Id), (Ie), (If), (Ig), or (Ih), R^{1a}, R^{1b}, R^{2a} and R^{2b} are hydrogen, Z is —CH₂—, R⁴ is methyl, U is ═N—, V is ═C(H)—, W is ═N— and the carbon atom to which R⁴ and ring A are attached is in the (S) configuration.

In another aspect of the compound of formula (I), (I*), (I**), (I***), (I****), (Ia), (Ib), (Ic), (Id), (Ie), (If), (Ig), or (Ih), R^{1a}, R^{1b}, R^{2a} and R^{2b} are hydrogen, Z is —CH₂—, R⁴ is methyl, U is ═C(H)—, V is ═N—, W is ═N— and the carbon atom to which R⁴ and ring A are attached is in the (S) configuration.

In another aspect of the compound of formula ((I), (I*), (I**), (I***), (I****), (Ia), (Ib), (Ic), (Id), (Ie), (If), (Ig), or (Ih), ring B comprises at least one nitrogen atom.

In another aspect of the compound of formula (I), (I*), (I**), (I***), (I****), (Ia), (Ib), (Ic), (Id), (Ie), (If), (Ig), or (Ih), ring B comprises two nitrogen atoms.

In another aspect of the compound of (I), (I*), (I**), (I***), (I****), (Ia), (Ib), (Ic), (Id), (Ie), (If), (Ig), or (Ih), ring B is a 3-11 membered heterocycloalkylene substituted with one C₁₋₆alkyl and optionally further substituted with one or more identical or different C₁₋₆alkyl, C₁₋₆alkoxy or a 5-6 membered heterocycoalklyl, wherein any of the C₁₋₆alkyl is optionally and independently substituted with cyclopropyl, preferably wherein said 3-11 membered heterocycloalkylene comprises at least one nitrogen atom or wherein said 3-11 membered heterocycloalkylene comprises two nitrogen atoms.

In another aspect of the compound of formula (I), (I*), (I**), (I***), (I****), (Ia), (Ib), (Ic), (Id), (Ie), (If), (Ig), or (Ih), ring B is a 5-8 membered heterocycloalkylene optionally substituted with one or more identical or different C₁₋₃alkyl, C₁₋₃alkoxy or a 5-6 membered heterocycloalkyl, wherein the C₁₋₃alkyl is optionally substituted with cyclopropyl, preferably wherein said 5-8 membered heterocycloalkylene comprises at least one nitrogen atom or wherein said 5-8 membered heterocycloalkylene comprises two nitrogen atoms.

In another aspect of the compound of formula (I), (I*), (I**), (I***), (I****), (Ia), (Ib), (Ic), (Id), (Ie), (If), (Ig), or (Ih), ring B is a 5-8 membered heterocycloalkylene substituted with one C₁₋₃ alkyl and optionally further substituted with one or more identical or different C₁₋₃alkyl, C₁₋₃ alkoxy or a 5-6 membered heterocycloalkyl, preferably wherein said 5-8 membered heterocycloalkylene comprises at least one nitrogen atom or wherein said 5-8 membered heterocycloalkylene comprises two nitrogen atoms.

In another aspect of the compound of formula (I), (I*), (I**), (I***), (I****), (Ia), (Ib), (Ic), (Id), (Ie), (If), (Ig), or (Ih), ring B is

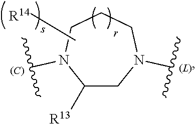

wherein r is 0, 1 or 2, s is 0, 1, 2, 3 or 4, R¹³ is C₁₋₆alkyl, each R¹⁴ is independently at each occurrence C₁₋₆alkyl, C₁₋₆alkoxy or a 5-6 membered heterocycloalkyl and any of the C₁₋₆alkyl is optionally substituted with cyclopropyl.

In another aspect of the compound of formula (I), (I*), (I**), (I***), (I****), (Ia), (Ib), (Ic), (Id), (Ie), (If), (Ig), or (Ih), ring B is

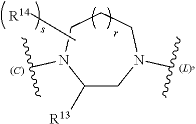

wherein r is 0, 1 or 2, s is 0, 1, 2, 3 or 4, R¹³ is C₁₋₃alkyl, each R¹⁴ is independently at each occurrence C₁₋₃alkyl, C₁₋₃alkoxy or a 5-6 membered heterocycloalkyl.

In another aspect of the compound of formula (I), (I*), (I**), (I***), (I****), (Ia), (Ib), (Ic), (Id), (Ie), (If), (Ig), or (Ih), ring B is

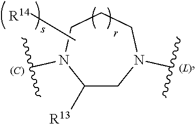

wherein r is 0, 1 or 2, s is 0, 1 or 2, R¹³ is C₁₋₃alkyl and each R¹⁴ is independently at each occurrence the same or different C₁₋₃alkyl.

Preferably, the carbon atom to which R¹³ is attached is in the (S) configuration.

Preferably, r is 0 or 1. Preferably, r is 0. Preferably, r is 1.

Preferably, s is 0, 1 or 2. Preferably, s is 0. Preferably, s is 1. Preferably, s is 2.

Preferably, R¹³ is methyl.

Preferably, when present, R¹⁴ is methyl.

In another aspect of the compound of formula (I), (I*), (I**), (I***), (I****), (Ia), (Ib), (Ic), (Id), (Ie), (If), (Ig), or (Ih), ring B is

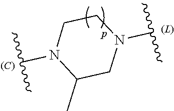

wherein p is selected from the group consisting of 0, 1, 2 and 3.

In another aspect of the compound of formula (I), (I*), (I**), (I***), (I****), (Ia), (Ib), (Ic), (Id), (Ie), (If), (Ig), or (Ih), ring B is

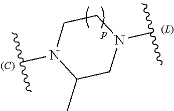

wherein p is 1 or 2 and the chiral carbon atom is in the (S) configuration.

In another aspect of the compound of formula (I), (I*), (I**), (I***), (I****), (Ia), (Ib), (Ic), (Id), (Ie), (If), (Ig), or (Ih), ring B is selected from the group consisting of:

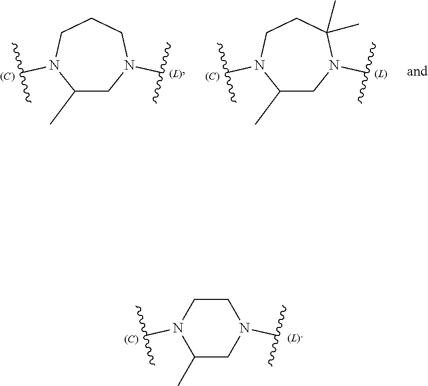

In another aspect of the compound of formula (I), (I*), (I**), (I***), (I****), (Ia), (Ib), (Ic), (Id), (Ie), (If), (Ig), or (Ih), ring B is selected from the group consisting of:

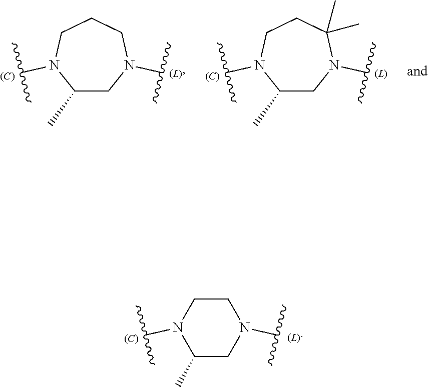

It is to be understood that “(C)” and “(L)” indicate the atom or substituent of formula (I) to which ring B is attached.

In another aspect of the compound of formula (I), (I*), (I**), (I***), (I****), (Ia), (Ib), (Ic), (Id), (Ie), (If), (Ig), or (Ih), L is selected from the group consisting of C₁₋₈alkylene, C₂₋₈alkenylene and C₁₋₈alkoxylene.

In another aspect of the compound of formula (I), (I*), (I**), (I***), (I****), (Ia), (Ib), (Ic), (Id), (Ie), (If), (Ig), or (Ih), L is selected from the group consisting of C₁₋₆alkylene, C₂₋₆alkenylene and C₁₋₆alkoxylene.

In another aspect of the compound of formula (I), (I*), (I**), (I***), (I****), (Ia), (Ib), (Ic), (Id), (Ie), (If), (Ig), or (Ih), L is selected from the group consisting of:
—(CH₂)₂—, —(CH₂)₃—, —(CH₂)₄—, —(CH₂)₆—,

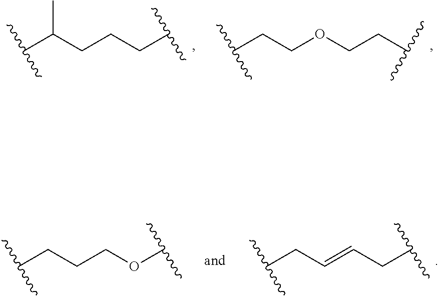

In another aspect of the compound of formula (I), (I*), (I**), (I***), (I****), (Ia), (Ib), (Ic), (Id), (Ie), (If), (Ig), or (Ih), L is selected from the group consisting of:
—(CH₂)₂—, —(CH₂)₃—, —(CH₂)₄—, —(CH₂)₆—,

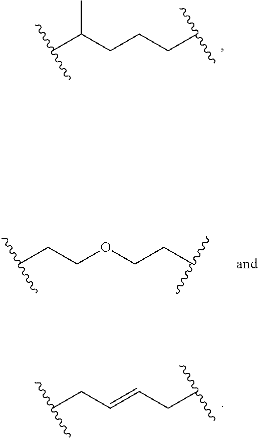

In another aspect of the compound of formula (I), (I*), (I**), (I***), (I****), (Ia), (Ib), (Ic), (Id), (Ie), (If), (Ig), or (Ih), L is selected from the group consisting of:
—(CH₂)₂—, —(CH₂)₃—, —(CH₂)₄—, —(CH₂)₆—,

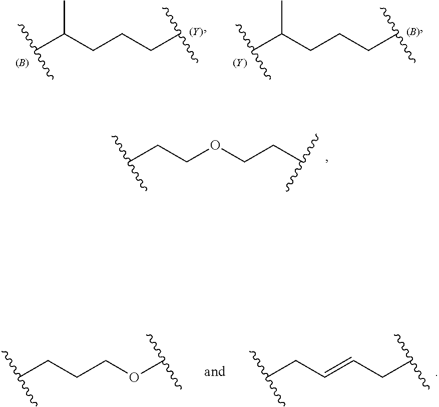

In another aspect of the compound of formula (I), (I*), (I**), (I***), (I****), (Ia), (Ib), (Ic), (Id), (Ie), (If), (Ig), or (Ih), L is selected from the group consisting of:
—(CH₂)₂—, —(CH₂)₃—, —(CH₂)₄—, —(CH₂)₆—,

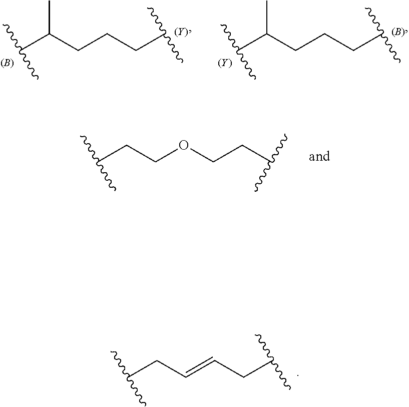

It is to be understood that “(B)” and “(Y)” indicate the substituents of formula (I) to which L is attached.

In another aspect of the compound of formula (I), (I*), (I**), (I***), (I****), (Ia), (Ib), (Ic), (Id), (Ie), (If), (Ig), or (Ih), X is —(CH₂)—.

In another aspect of the compound of formula (I), (I*), (I**), (I***), (I****), (Ia), (Ib), (Ic), (Id), (Ie), (If), (Ig), or (Ih), X is —O—.

In another aspect of the compound of formula (I), (I*), (I**), (I***), (I****), (Ia), (Ib), (Ic), (Id), or (Ie), Y is a 5 membered heteroarylene or —C(O)(NH)—, wherein said 5 membered heteroarylene comprises at least one nitrogen atom and wherein said —C(O)(NH)— is linked to X via the C atom.

In another aspect of the compound of formula (I), (I*), (I**), (I***), (I****), (Ia), (Ib), (Ic), (Id), or (Ie), Y is selected from the group consisting of pyrrole, imidazole, pyrazole, oxazole, isoxazole, thiazole, isothiazole, oxadiazole, thiadiazole, triazole and —C(O)(NR¹²)—, wherein said —C(O)(NR¹²)— is linked to X via the C atom, preferably wherein R¹² is hydrogen.

In another aspect of the compound of formula (I), (I*), (I**), (I***), (I****), (Ia), (Ib), (Ic), (Id), or (Ie), Y is selected from the group consisting of pyrrole, imidazole, pyrazole, oxazole, isoxazole, thiazole, isothiazole, oxadiazole, thiadiazole and triazole.

In another aspect of the compound of formula (I), (I*), (I**), (I***), (I****), (Ia), (Ib), (Ic), (Id), or (Ie), Y is isoxazole, triazole or —C(O)(NR¹²)—, wherein said —C(O)(NR¹²)— is linked to X via the C atom.

In another aspect of the compound of formula (I), (I*), (I**), (I***), (I****), (Ia), (Ib), (Ic), (Id), or (Ie), Y is isoxazole, triazole or —C(O)(NH)—, wherein said —C(O)(NH)— is linked to X via the C atom.

In another aspect of the compound of formula (I), (I*), (I**), (I***), (I****), (Ia), (Ib), (Ic), (Id), or (Ie), Y is —C(O)(NH)—, wherein said —C(O)(NH)— is linked to X via the C atom.

In another aspect of the compound of formula (I), (I*), (I**), (I***), (I****), (Ia), (Ib), (Ic), (Id), or (Ie), Y is isoxazole or triazole.

In another aspect of the compound of formula (I), (I*), (I**), (I***), (I****), (Ia), (Ib), (Ic), (Id), or (Ie), Y is isoxazole.

In another aspect of the compound of formula (I), (I*), (I**), (I***), (I****), (Ia), (Ib), (Ic), (Id), or (Ie), Y is triazole.

In another aspect of the compound of formula (I), (I*), (I**), (I***), (I****), (Ia), (Ib), (Ic), (Id), or (Ie), Y is selected from the group consisting of:

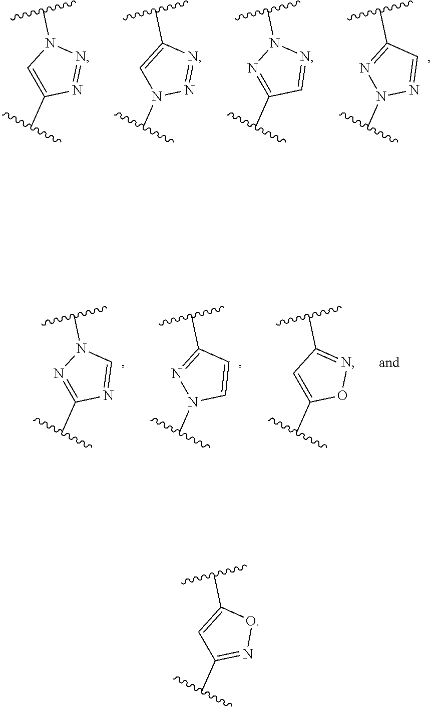

In another aspect of the compound of formula (I), (I*), (I**), (I***), (I****), (Ia), (Ib), (Ic), (Id), or (Ie), Y is selected from the group consisting of:

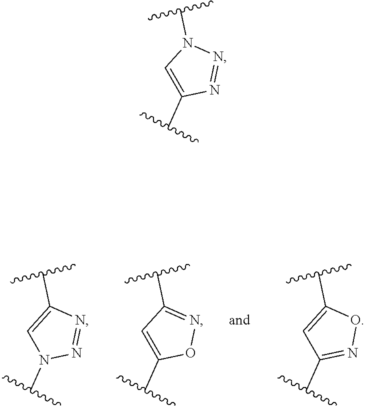 [SMILES: *c1cn(*)nn1 ; *c1cn(*)nn1 ; *c1cc(*)on1 ; "and" ; *c1cc(*)on1]

In another aspect of the compound of formula (I), (I*), (I**), (I***), (I****), (Ia), (Ib), (Ic), (Id), or (Ie), Y is selected from the group consisting of:

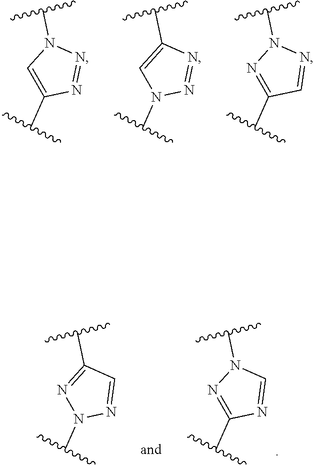 [SMILES: *c1cn(*)nn1 ; *c1cn(*)nn1 ; *c1cnn(*)n1 ; *c1cnn(*)n1 ; "and" ; *c1ncn(*)n1]

In another aspect of the compound of formula (I), (I*), (I**), (I***), (I****), (Ia), (Ib), (Ic), (Id), or (Ie), Y is:

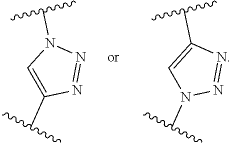 [SMILES: *c1cn(*)nn1 ; *c1cn(*)nn1]

In another aspect of the compound of formula (I), (I*), (I**), (I***), (I****), (Ia), (Ib), (Ic), (Id), or (Ie), Y is:

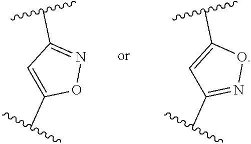 [SMILES: *c1cc(*)on1 ; "or" ; *c1cc(*)on1]

In another aspect of the compound of formula (I), (I*), (I**), (I***), (I****), (Ia), (Ib), (Ic), (Id), (Ie), (If), (Ig), or (Ih), at any occurrence triazole is selected from the group consisting of:

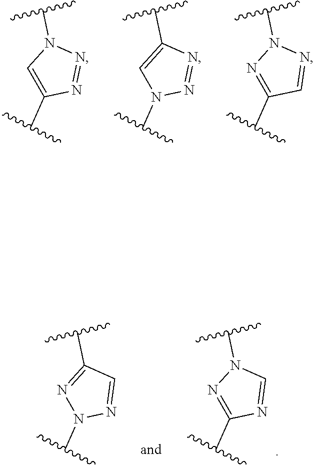 [SMILES: *c1cn(*)nn1 ; *c1cn(*)nn1 ; *c1cnn(*)n1 ; *c1cnn(*)n1 ; "and" ; *c1ncn(*)n1]

In another aspect of the compound of formula (I), (I*), (I**), (I***), (I****), (Ia), (Ib), (Ic), (Id), (Ie), (If), (Ig), or (Ih), when Y is —C(O)(NR¹²)—, X is —(CH₂)—, wherein said —C(O)(NR¹²)— is linked to X via the C atom.

In another aspect of the compound of formula (I), (I*), (I**), (I***), (I****), (Ia), (Ib), (Ic), (Id), or (Ie), X is —(CH₂)— and Y is —C(O)(NR¹²)—, or X is —(CH₂)— or —O— and Y is a 5 membered heteroarylene comprising at least one nitrogen atom, wherein said —C(O)(NR¹²)— is linked to X via the C atom.

In another aspect of the compound of formula (I), (I*), (I**), (I***), (I****), (Ia), (Ib), (Ic), (Id), or (Ie), X is —(CH₂)— and Y is —C(O)(NR¹²)—, wherein said —C(O)(NR¹²)— is linked to X via the C atom.

In another aspect of the compound of formula (I), (I*), (I**), (I***), (I****), (Ia), (Ib), (Ic), (Id), or (Ie), X is —(CH₂)—, Y is —C(O)(NR¹²)—, wherein said —C(O)(NR¹²)— is linked to X via the C atom and L is C₁₋₈alkylene or a C₁₋₈alkoxylene.

In another aspect of the compound of formula (I), (I*), (I**), (I***), (I****), (Ia), (Ib), (Ic), (Id), or (Ie), X is —(CH₂)—, Y is —C(O)(NR¹²)—, wherein said —C(O)(NR¹²)— is linked to X via the C atom and L is C₁₋₆alkylene or a C₁₋₄alkoxylene.

In another aspect of the compound of formula (I), (I*), (I**), (I***), (I****), (Ia), (Ib), (Ic), (Id), or (Ie), X is —(CH₂)— or —O— and Y is a 5 membered heteroarylene comprising at least one nitrogen atom.

In another aspect of the compound of formula (I), (I*), (I**), (I***), (I****), (Ia), (Ib), (Ic), (Id), or (Ie), X is —(CH₂)— and Y is a 5 membered heteroarylene comprising at least one nitrogen atom.

In another aspect of the compound of formula (I), (I*), (I**), (I***), (I****), (Ia), (Ib), (Ic), (Id), or (Ie), X is —(CH₂)—, Y is a 5 membered heteroarylene comprising at least one nitrogen atom and L is C₁₋₈alkylene.

In another aspect of the compound of formula (I), (I*), (I**), (I***), (I****), (Ia), (Ib), (Ic), (Id), or (Ie), X is —O— and Y is a 5 membered heteroarylene comprising at least one nitrogen atom.

In another aspect of the compound of formula (I), (I*), (I**), (I***), (I****), (Ia), (Ib), (Ic), (Id), or (Ie), X is —(CH₂)— and Y is selected from the group consisting of pyrrole, imidazole, pyrazole, oxazole, isoxazole, thiazole, isothiazole, oxadiazole, thiadiazole, triazole.

In another aspect of the compound of formula (I), (I*), (I**), (I***), (I****), (Ia), (Ib), (Ic), (Id), or (Ie), X is —(CH₂)—, Y is selected from the group consisting of pyrrole, imidazole, pyrazole, oxazole, isoxazole, thiazole, isothiazole, oxadiazole, thiadiazole, triazole and L is C₁₋₈alkylene.

In another aspect of the compound of formula (I), (I*), (I**), (I***), (I****), (Ia), (Ib), (Ic), (Id), or (Ie), X is —O— and Y is selected from the group consisting of pyrrole, imidazole, pyrazole, oxazole, isoxazole, thiazole, isothiazole, oxadiazole, thiadiazole, triazole.

In another aspect of the compound of formula (I), (I*), (I**), (I***), (I****), (Ia), (Ib), (Ic), (Id), or (Ie), X is —(CH₂)— and Y is isoxazole or triazole.

In another aspect of the compound of formula (I), (I*), (I**), (I***), (I****), (Ia), (Ib), (Ic), (Id), or (Ie), X is —(CH₂)—, Y is isoxazole or triazole and L is C₁₋₈alkylene.

In another aspect of the compound of formula (I), (I*), (I**), (I***), (I****), (Ia), (Ib), (Ic), (Id), or (Ie), X is —(CH₂)— and Y is triazole.

In another aspect of the compound of formula (I), (I*), (I**), (I***), (I****), (Ia), (Ib), (Ic), (Id), or (Ie), X is —(CH₂)—, Y is triazole and L is C₁₋₈alkylene.

In another aspect of the compound of formula (I), (I*), (I**), (I***), (I****), (Ia), (Ib), (Ic), (Id), or (Ie), X is —(CH₂)—, Y is triazole and L is C₁₋₆-alkylene.

In another aspect of the compound of formula (I), (I*), (I**), (I***), (I****), (Ia), (Ib), (Ic), (Id), or (Ie), X is —O— and Y is isoxazole or triazole.

In another aspect of the compound of formula (I), (I*), (I**), (I***), (I****), (Ia), (Ib), (Ic), (Id), or (Ie), X is —O— and Y is isoxazole.

In another aspect of the compound of formula (I), (I*), (I**), (I***), (I****), (Ia), (Ib), (Ic), (Id), or (Ie), X is —O— and Y is triazole.

In another aspect of the compound of formula (I), (I*), (I**), (I***), (I****), (Ia), (Ib), (Ic), (Id), or (Ie), X is —(CH₂)— and Y is selected from the group consisting of:

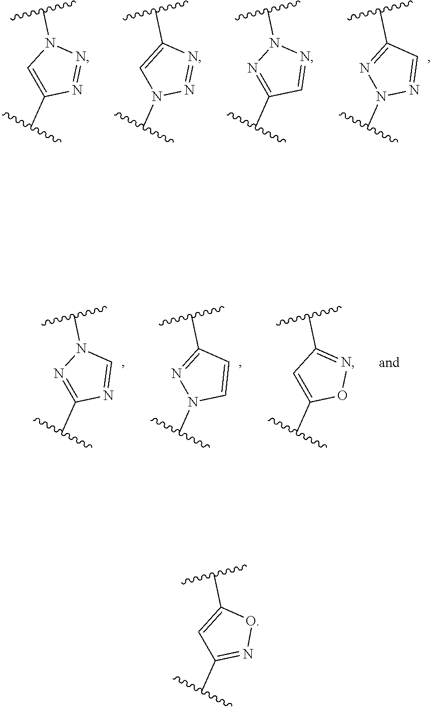

In another aspect of the compound of formula (I), (I*), (I**), (I***), (I****), (Ia), (Ib), (Ic), (Id), or (Ie), X is —(CH₂)—, Y is selected from the group consisting of:

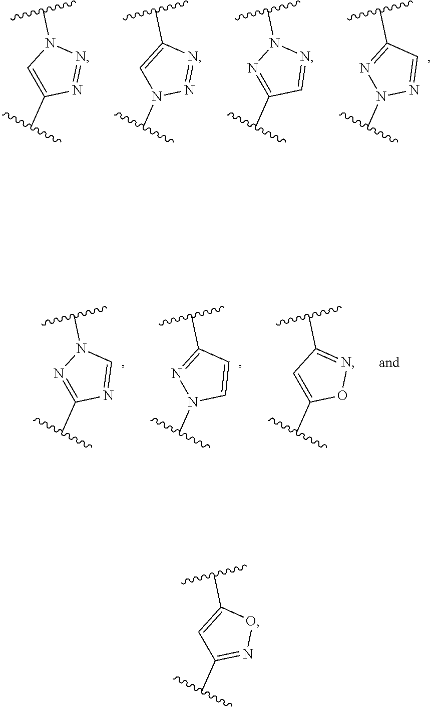

### and L is C₁₋₈alkylene.

In another aspect of the compound of formula (I), (I*), (I**), (I***), (I****), (Ia), (Ib), (Ic), (Id), or (Ie), X is —O— and Y is selected from the group consisting of:

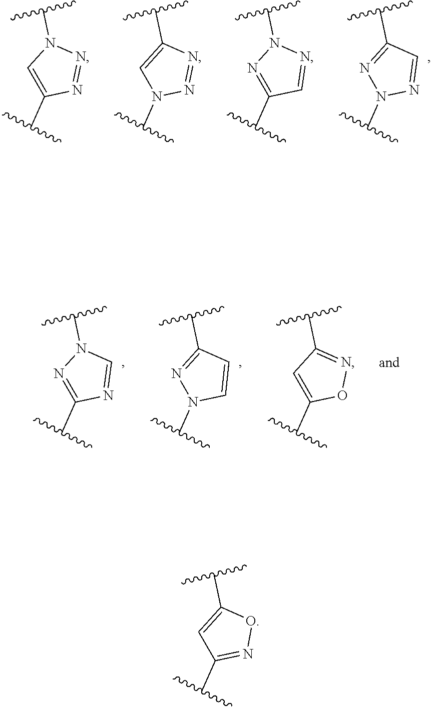

In another aspect of the compound of formula (I), (I*), (I**), (I***), (I****), (Ia), (Ib), (Ic), (Id), or (Ie), X is —(CH₂)— and Y is selected from the group consisting of:

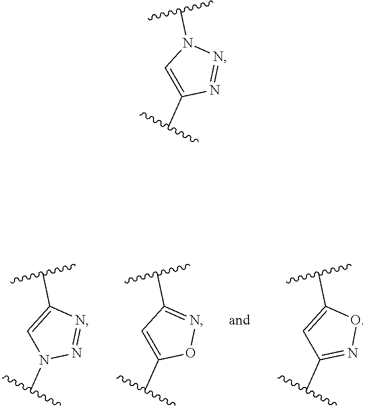 [SMILES: *c1cn(*)nn1 ; *c1cn(*)nn1 ; *c1cc(*)on1 ; "and" ; *c1cc(*)on1]

In another aspect of the compound of formula (I), (I*), (I**), (I***), (I****), (Ia), (Ib), (Ic), (Id), or (Ie), X is —(CH₂)— and Y is selected from the group consisting of:

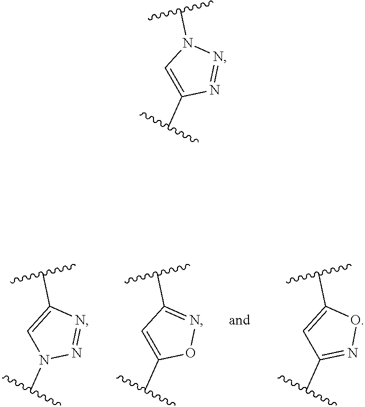 [SMILES: *c1cn(*)nn1 ; *c1cn(*)nn1 ; *c1cc(*)on1 ; "and" ; *c1cc(*)on1]

### and L is C₁₋₈alkylene.

In another aspect of the compound of formula (I), (I*), (I**), (I***), (I****), (Ia), (Ib), (Ic), (Id), or (Ie), X is —O— and Y is selected from the group consisting of:

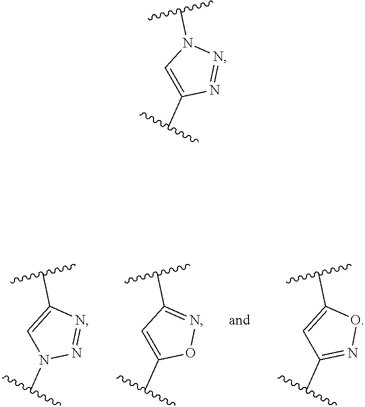 [SMILES: *c1cn(*)nn1 ; *c1cn(*)nn1 ; *c1cc(*)on1 ; "and" ; *c1cc(*)on1]

In another aspect of the compound of formula (I), (I*), (I**), (I***), (I****), (Ia), (Ib), (Ic), (Id), or (Ie), X is —(CH₂)— and Y is a 5 membered heteroarylene or —C(O)(NR¹²)—, wherein said 5 membered heteroarylene comprises at least one nitrogen atom and wherein said —C(O)(NR¹²)— is linked to X via the C atom, or X is —O— and Y is a 5 membered heteroarylene comprising at least one nitrogen atom.

In another aspect of the compound of formula (I), (I*), (I**), (I***), (I****), (Ia), (Ib), (Ic), (Id), or (Ie), X is —(CH₂)— and Y is a 5 membered heteroarylene comprising at least one nitrogen atom or —C(O)(NR¹²)—, wherein said —C(O)(NR¹²)— is linked to X via the C atom.

In another aspect of the compound of formula (I), (I*), (I**), (I***), (I****), (Ia), (Ib), (Ic), (Id), or (Ie), X is —(CH₂)—, Y is a 5 membered heteroarylene comprising at least one nitrogen atom or —C(O)(NR¹²)—, wherein said —C(O)(NR¹²)— is linked to X via the C atom and L is C₁₋₈alkylene.

In another aspect of the compound of formula (I), (I*), (I**), (I***), (I****), (Ia), (Ib), (Ic), (Id), or (Ie), X is —(CH₂)— and Y is selected from the group consisting of pyrrole, imidazole, pyrazole, oxazole, isoxazole, thiazole, isothiazole, oxadiazole, thiadiazole, triazole and —C(O)(NR¹²)—, or X is —O— and Y is selected from the group consisting of pyrrole, imidazole, pyrazole, oxazole, isoxazole, thiazole, isothiazole, oxadiazole, thiadiazole, triazole, wherein said —C(O)(NR¹²)— is linked to X via the C atom.

In another aspect of the compound of formula (I), (I*), (I**), (I***), (I****), (Ia), (Ib), (Ic), (Id), or (Ie), X is —(CH₂)— and Y is selected from the group consisting of pyrrole, imidazole, pyrazole, oxazole, isoxazole, thiazole, isothiazole, oxadiazole, thiadiazole, triazole and —C(O)(NR¹²)—, wherein said —C(O)(NR¹²)— is linked to X via the C atom.

In another aspect of the compound of formula (I), (I*), (I**), (I***), (I****), (Ia), (Ib), (Ic), (Id), or (Ie), X is —(CH₂)— and Y is triazole or —C(O)(NR¹²)—, or X is —O— and Y is triazole or isoxazole, wherein said —C(O)(NR¹²)— is linked to X via the C atom.

In another aspect of the compound of formula (I), (I*), (I**), (I***), (I****), (Ia), (Ib), (Ic), (Id), or (Ie), X is —(CH₂)— and Y is triazole or —C(O)(NR¹²)—, wherein said —C(O)(NR¹²)— is linked to X via the C atom.

In another aspect of the compound of formula (I), (I*), (I**), (I***), (I****), (Ia), (Ib), (Ic), (Id), or (Ie), X is —(CH₂)—, Y is triazole or —C(O)(NR¹²)—, wherein said —C(O)(NR¹²)— is linked to X via the C atom and L is C₁₋₈alkylene.

In another aspect of the compound of formula (I), (I*), (I**), (I***), (I****), (Ia), (Ib), (Ic), (Id), or (Ie), (B)-L-X-Y-(C) is selected from the group consisting of:

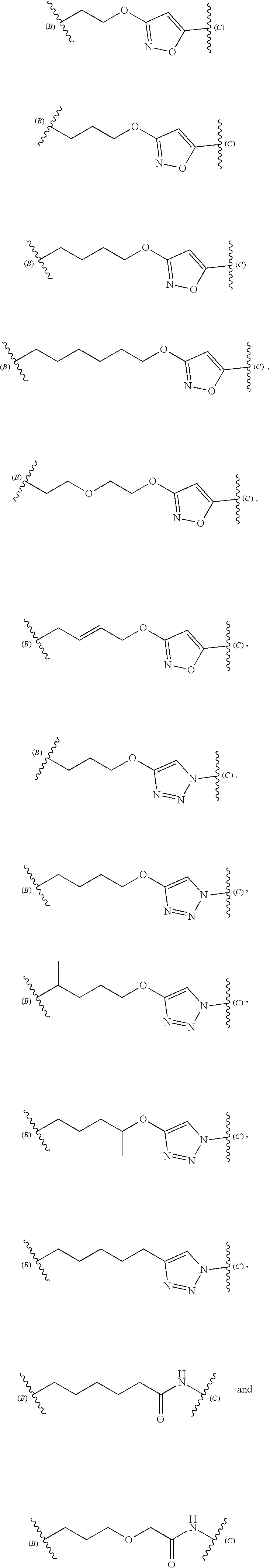

In another aspect of the compound of formula (I), (I*), (I**), (I***), (I****), (Ia), (Ib), (Ic), (Id), or (Ie), (B)-L-X-Y-(C) is selected from the group consisting of:

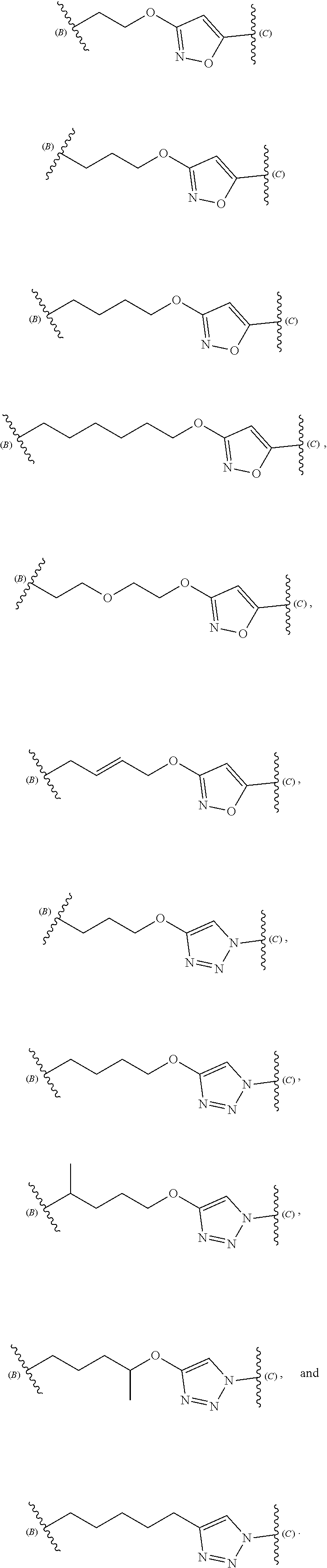

In another aspect of the compound of formula (I), (I*), (I**), (I***), (I****), (Ia), (Ib), (Ic), (Id), (Ie), (If), (Ig), or (Ih), R⁹ is a branched C₁₋₄ alkyl.

In another aspect of the compound of formula (I), (I*), (I**), (I***), (I****), (Ia), (Ib), (Ic), (Id), (Ie), (If), (Ig), or (Ih), R⁹ is iso-propyl or tert-butyl.

In another aspect of the compound of formula (I), (I*), (I**), (I***), (I****), (Ia), (Ib), (Ic), (Id), (Ie), (If), (Ig), or (Ih), R⁹ is iso-propyl.

In another aspect of the compound of formula (I), (I*), (I**), (I***), (I****), (Ia), (Ib), (Ic), (Id), or (Ie), R⁹ is iso-propyl and Y is a 5 membered heteroarylene comprising at least one nitrogen atom.

In another aspect of the compound of formula (I), (I*), (I**), (I***), (I****), (Ia), (Ib), (Ic), (Id), or (Ie), R⁹ is iso-propyl and Y is selected from the group consisting of pyrrole, imidazole, pyrazole, oxazole, isoxazole, thiazole, isothiazole, oxadiazole, thiadiazole, triazole and —C(O)(NR¹²)—, wherein said —C(O)(NR¹²)— is linked to X via the C atom, preferably wherein R¹² is hydrogen.

In another aspect of the compound of formula (I), (I*), (I**), (I***), (I****), (Ia), (Ib), (Ic), (Id), or (Ie), R⁹ is iso-propyl and Y is isoxazole or triazole.

In another aspect of the compound of formula (I), (I*), (I**), (I***), (I****), (Ia), (Ib), (Ic), (Id), or (Ie), R⁹ is iso-propyl and Y is selected from the group consisting of:

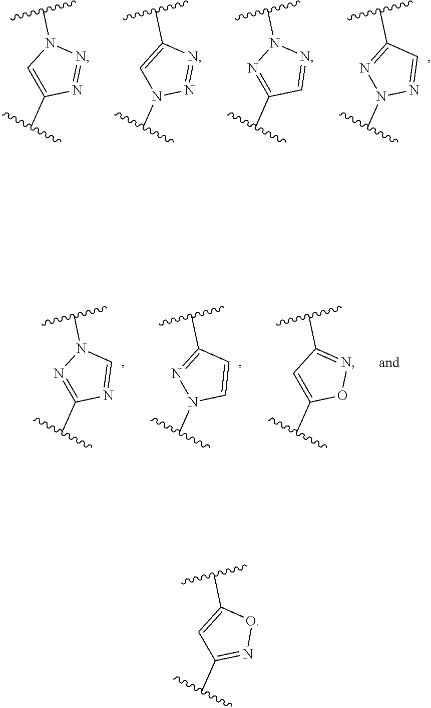

In another aspect of the compound of formula (I), (I*), (I**), (I***), (I****), (Ia), (Ib), (Ic), (Id), or (Ie), R⁹ is iso-propyl and Y is selected from the group consisting of:

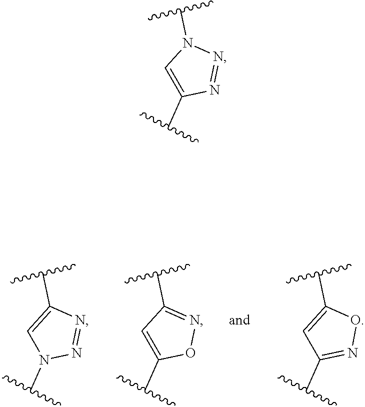 [SMILES: *c1cn(*)nn1 ; *c1cn(*)nn1 ; *c1cc(*)on1 ; "and" ; *c1cc(*)on1]

In another aspect of the compound of formula (I), (I*), (I**), (I***), (I****), (Ia), (Ib), (Ic), (Id), or (Ie), R⁹ is tert-butyl.

In another aspect of the compound of formula (I), (I*), (I**), (I***), (I****), (Ia), (Ib), (Ic), (Id), or (Ie), R⁹ is tert-butyl and Y is —C(O)(NR¹²)—, wherein said —C(O)(NR¹²)— is linked to X via the C atom.

In another aspect of the compound of formula ((I), (I*), (I**), (I***), (I****), (Ia), (Ib), (Ic), (Id), or (Ie), R⁹ is tert-butyl and Y is —C(O)(NH)—, wherein said —C(O)(NH)— is linked to X via the C atom.

In another aspect of the compound of formula (I), (I**), (I***), (I****), (Ia), (Ib), (Ic), (Id), (Ie), (If), (Ig), or (Ih), the carbon atom to which R⁹ is attached is in the (S) configuration.

In another aspect of the compound of formula (I), (I**), (I***), (I****), (Ia), (Ib), (Ic), (Id), (Ie), (If), (Ig), or (Ih), the carbon atom to which R⁹ is attached is in the (R) configuration.

In another aspect of the compound of formula (I), (I**), (I***), (I****), (Ia), (Ib), (Ic), (Id), (Ie), (If), (Ig), or (Ih), R⁹ is iso-propyl and the carbon atom to which R⁹ is attached is in the (S) configuration.

In another aspect of the compound of formula (I), (I**), (I***), (I****), (Ia), (Ib), (Ic), (Id), (Ie), (If), (Ig), or (Ih), R⁹ is iso-propyl and the carbon atom to which R⁹ is attached is in the (R) configuration.

In another aspect of the compound of formula (I), (I*), (I**), (I***), (I****), (Ia), (Ib), (Ic), (Id), (Ie), (If), (Ig), or (Ih), R^{1a}, R^{1b}, R^{2a} and R^{2b} are hydrogen, Z is —CH₂—, R⁴ is methyl, W is ═N— and R⁹ is iso-propyl.

In another aspect of the compound of formula (I), (I*), (I**), (I***), (I****), (Ia), (Ib), (Ic), (Id), (Ie), (If), (Ig), or (Ih), R^{1a}, R^{1b}, R^{2a} and R^{2b} are hydrogen, Z is —CH₂—, R⁴ is methyl, W is ═N, R⁹ is iso-propyl, the carbon atom to which R⁴ and ring A are attached is in the (S) configuration and the carbon atom to which R⁹ is attached is in the (S) configuration.

In another aspect of the compound of formula (I), (I*), (I**), (I***), (I****), (Ia), (Ib), (Ic), (Id), (Ie), (If), (Ig), or (Ih), R¹⁰ is selected from the group consisting of hydrogen, C₁₋₆alkyl, and —C(O)OR¹², wherein said C₁₋₆alkyl is optionally substituted by —OH or —OP(O)(OH)₂.

In another aspect of the compound of formula (I), (I*), (I**), (I***), (I****), (Ia), (Ib), (Ic), (Id), (Ie), (If), (Ig), or (Ih), R¹⁰ is hydrogen.

In another aspect of the compound of formula (I), (I*), (I**), (I***), (I****), (Ia), (Ib), (Ic), (Id), (Ie), (If), (Ig), or (Ih), R¹⁰ is selected from the group consisting of C₁₋₆alkyl, and —C(O)OR¹², wherein said C₁₋₆alkyl is optionally substituted by —OH or —OP(O)(OH)₂.

In another aspect of the compound of formula (I), (I*), (I**), (I***), (I****), (Ia), (Ib), (Ic), (Id), (Ie), (If), (Ig), or (Ih), R¹⁰ is selected from the group consisting of hydrogen, C₁₋₄alkyl, and —C(O)OR¹², wherein said C₁₋₄alkyl is optionally substituted by —OH or —OP(O)(OH)₂.

In another aspect of the compound of formula (I), (I*), (I**), (I***), (I****), (Ia), (Ib), (Ic), (Id), (Ie), (If), (Ig), or (Ih), R¹⁰ is selected from the group consisting of hydrogen, C₁₋₄alkyl, and —C(O)OR¹², wherein said C₁₋₄alkyl is optionally substituted by —OH.

In another aspect of the compound of formula (I), (I*), (I**), (I***), (I****), (Ia), (Ib), (Ic), (Id), (Ie), (If), (Ig), or (Ih), R¹⁰ is not hydrogen and the carbon atom to which R¹⁰ is attached is in the (S) configuration.

In another aspect of the compound of formula (I), (I*), (I**), (I***), (I****), (Ia), (Ib), (Ic), (Id), (Ie), (If), (Ig), or (Ih), R¹⁰ is not hydrogen and the carbon atom to which R¹⁰ is attached is in the (R) configuration.

In another aspect of the compound of formula (I), (I*), (I**), (I***), (I****), (Ia), (Ib), (Ic), (Id), (Ie), (If), (Ig), or (Ih), R¹⁰ is selected from the group consisting of hydrogen, methyl, —CH₂OH, —CH₂CH₂OH,

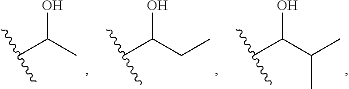

—CH₂CH₂OP(O)(OH)₂, —C(O)OCH₃ and —C(O)OH.

In another aspect of the compound of formula (I), (I*), (I**), (I***), (I****), (Ia), (Ib), (Ic), (Id), (Ie), (If), (Ig), or (Ih), R¹² is hydrogen or methyl.

In another aspect of the compound of formula (I), (I*), (I**), (I***), (I****), (Ia), (Ib), (Ic), (Id), (Ie), (If), (Ig), or (Ih), R¹² is hydrogen.

In another aspect of the compound of formula (I), (I*), (I**), (I***), (I****), (Ia), (Ib), (Ic), (Id), (Ie), (If), (Ig), or (Ih), R¹² is methyl.

In another aspect of the compound of formula (I), (I*), (I**), (I***), (I****), (Ia), (Ib), (Ic), (Id), (Ie), (If), (Ig), or (Ih), q is 0. It is to be understood that when q is 0, R⁶ is hydrogen.

In another aspect of the compound of formula (I), (I*), (I**), (I***), (I****), (Ia), (Ib), (Ic), (Id), (Ie), (If), (Ig), or (Ih), q is 1. When q is 1, R⁶ may be meta- or ortho- to R⁷. When R⁶ is meta- to R⁷, it is labelled as Rea. When R⁶ is ortho- to R⁷, it is labelled as R^{6b}.

When q is 1, R^{6a} and R^{6b} are each independently selected from the group consisting of: hydrogen, halogen and C₁₋₃alkyl. It is to be understood that when q is 1, one of R^{6a} or R^{6b} is hydrogen.

In another aspect of the compound of formula formula (I), (I*), (I**), (I***), (I****), (Ia), (Ib), (Ic), (Id), (Ie), (If), (Ig), or (Ih), q is 2. When q is 2, there are two R⁶'s. The R⁶ that is meta- to R⁷ is labelled as Rea. The R⁶ that is ortho- to R⁷ is labelled as R^{6b}.

When q is 2, R^{6a} and R^{6b} are each independently halogen or C₁₋₃alkyl.

In another aspect of the compound of formula formula (I), (I*), (I**), (I***), (I****), (Ia), (Ib), (Ic), (Id), (Ie), (If), (Ig), or (Ih), q is 0 or 1.

Preferably, R^{6a} is hydrogen or halogen.

Preferably, R^{6a} is hydrogen or fluorine.

Preferably, R^{6a} is hydrogen.

Preferably, R^{6a} is fluorine.

Preferably, R^{6b} is selected from the group consisting of hydrogen, fluorine, chlorine and methyl.

Preferably, R^{6a} is hydrogen or halogen and R^{6b} is hydrogen, halogen or C₁₋₃alkyl.

Preferably, R^{6a} is hydrogen and R^{6b} is hydrogen, halogen and C₁₋₃alkyl.

Preferably, R^{6a} and R^{6b} are each independently selected from the group consisting of hydrogen, fluorine, chlorine and methyl.

Preferably, R^{6a} is hydrogen or fluorine and R^{6b} is selected from the group consisting of hydrogen, fluorine, chlorine and methyl.

Preferably, R^{6a} is hydrogen and R^{6b} is selected from the group consisting of fluorine, chlorine and methyl.

Preferably, R^{6a} and R^{6b} are hydrogen.

In another aspect of the compound of formula (I), (I*), (I**), (I***), (I****), (Ia), (Ib), (Ic), (Id), (Ie), (If), (Ig), or (Ih), R⁷ is selected from the group consisting of: chlorine, bromine, iso-propyl, —CN, thiazole, triazole and imidazole, wherein said thiazole, triazole or imidazole is optionally substituted with R⁸.

In another aspect of the compound of formula (I), (I*), (I**), (I***), (I****), (Ia), (Ib), (Ic), (Id), (Ie), (If), (Ig), or (Ih), R⁷ is selected from the group consisting of: halogen, C₁₋₃alkyl, —CN, thiazole, triazole and imidazole, wherein said thiazole is substituted with R⁸.

In another aspect of the compound of formula (I), (I*), (I**), (I***), (I****), (Ia), (Ib), (Ic), (Id), (Ie), (If), (Ig), or (Ih), R⁷ is selected from the group consisting of: halogen, C₁₋₃alkyl, —CN, thiazole, triazole and imidazole, wherein said thiazole is substituted with methyl or —CH₂OH.

In another aspect of the compound of formula (I), (I*), (I**), (I***), (I****), (Ia), (Ib), (Ic), (Id), (Ie), (If), (Ig), or (Ih), R⁷ is selected from the group consisting of: chlorine, bromine, iso-propyl, —CN, thiazole, triazole and imidazole, wherein said thiazole is substituted with methyl or —CH₂OH.

In another aspect of the compound of formula (I), (I*), (I**), (I***), (I****), (Ia), (Ib), (Ic), (Id), (Ie), (If), (Ig), or (Ih), R⁷ is selected from the group consisting of: chlorine, bromine, iso-propyl, —CN,

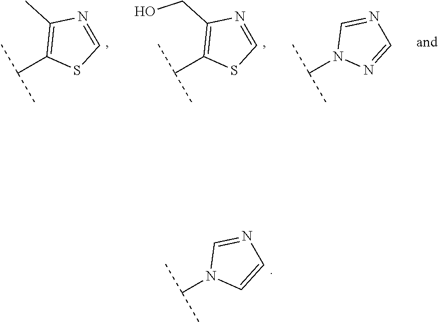

In another aspect of the compound of formula (I), (I*), (I**), (I***), (I****), (Ia), (Ib), (Ic), (Id), (Ie), (If), (Ig), or (Ih), R⁸ is methyl or —CH₂OH.

In another aspect of the compound of formula (I), (I*), (I**), (I***), (I****), (Ia), (Ib), (Ic), (Id), (Ie), (If), (Ig), or (Ih), R⁸ is methyl.

Preferred embodiments of compound of formula (I) are represented by compounds of formulas I-1 to I-48, as defined hereinbelow, including any stereoisomer thereof.

It is to be understood that any two or more aspects and/or preferred embodiments of formula (I)—or subformulas thereof—may be combined in any way leading to a chemically stable structure to obtain further aspects and/or preferred embodiments of formula (I)—or subformulas thereof.

The present invention further relates to hydrates, solvates, polymorphs, metabolites, derivatives, stereoisomers and prodrugs of a compound of formula (I), (I*), (I**), (I***), (I****), (Ia), (Ib), (Ic), (Id), (Ie), (If), (Ig), or (Ih), (including all aspects and preferred embodiments thereof).

Compounds of formula (I), (I*), (I**), (I***), (I****), (Ia), (Ib), (Ic), (Id), (Ie), (If), (Ig), or (Ih), (including all aspects and preferred embodiments thereof) which e.g. bear ester groups are potential prodrugs the ester being cleaved under physiological conditions and are also part of the invention.

The present invention further relates to a pharmaceutically acceptable salt of a compound of formula (I), (I*), (I**), (I***), (I****), (Ia), (Ib), (Ic), (Id), (Ie), (If), (Ig), or (Ih), (including all aspects and preferred embodiments thereof).

It is a further object of the present invention a compound of formula (II):

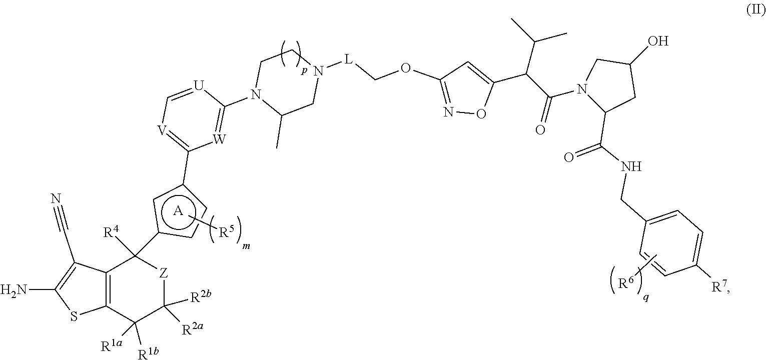

wherein:
R^{1a} and R^{1b} are both independently selected from the group consisting of hydrogen, C₁₋₄alkyl, C₁₋₄haloalkyl, C₁₋₄alkoxy, C₁₋₄haloalkoxy, halogen, —NH₂, —NH(C₁₋₄alkyl), —N(C₁₋₄alkyl)₂, C₃₋₅cycloalkyl and 3-5 membered heterocycloalkyl;
R^{2a} and R^{2b} are both independently selected from the group consisting of hydrogen, C₁₋₄alkyl, C₁₋₄haloalkyl, C₁₋₄alkoxy, C₁₋₄haloalkoxy, halogen, —NH₂, —NH(C₁₋₄alkyl), —N(C₁₋₄alkyl)₂, C₃₋₅cycloalkyl and 3-5 membered heterocycloalkyl;
and/or, optionally, one of R^{1a} or R^{1b} and one of R^{2a} or R^{2b} together with the carbon atoms they are attached to form a cyclopropane ring;
Z is —(CR^{3a}R^{3b})ₙ—;
each R^{3a} and R^{3b} is independently selected from the group consisting of hydrogen, C₁₋₄alkyl, C₁₋₄haloalkyl, C₁₋₄alkoxy, C₁₋₄haloalkoxy, halogen, —NH₂, —NH(C₁₋₄alkyl), —N(C₁₋₄alkyl)₂, C₃₋₅cycloalkyl and 3-5 membered heterocycloalkyl;
or R^{3a} and R^{3b} together with the carbon atom they are attached to form a cyclopropane ring; n is selected from the group consisting of 0, 1 and 2;
R⁴ is selected from the group consisting of hydrogen, C₁₋₆-alkyl, C₁₋₆-haloalkyl, C₁₋₆-alkoxy, C₁₋₆-haloalkoxy, cyano-C₁₋₆-alkyl, halogen, —OH, —NH₂, —NH(C₁₋₄alkyl), —N(C₁₋₄alkyl)₂, —CN, C₃₋₅cycloalkyl and 3-5 membered heterocycloalkyl;
ring A is selected from the group consisting of pyrrole, furan, thiophene, imidazole, pyrazole, oxazole, isoxazole, thiazole, isothiazole, oxadiazole, thiadiazole and triazole;
each R⁵, if present, is independently selected from the group consisting of C₁₋₆-alkyl, C₁₋₆ haloalkyl, C₁₋₆alkoxy, C₁₋₆haloalkoxy, cyano-C₁₋₆alkyl, halogen, —OH, —NH₂, —NH(C₁₋₄alkyl), —N(C₁₋₄alkyl)₂, —CN, C₃₋₅cycloalkyl and 3-5 membered heterocycloalkyl;
m is selected from the group consisting of 0, 1, 2 and 3;
U, V and W are each independently ═N— or ═C(H)—;
p is selected from the group consisting of 0, 1, 2 and 3;
L is selected from the group consisting of a bond, C₁₋₆alkylene, C₂₋₆alkenylene and C₁₋₆alkoxylene;
q is selected from the group consisting of 0, 1 and 2;
each R⁵, if present, is independently at each occurrence halogen or C₁₋₃alkyl;
R⁷ is selected from the group consisting of halogen, C₁₋₃alkyl, —CN and 5 membered heteroaryl, wherein said 5 membered heteroaryl comprises at least one nitrogen atom and is optionally substituted with R⁸;
R⁸ is C₁₋₃alkyl or C₁₋₃hydroxyalkyl;
or a salt thereof.

In one aspect, the compound or salt of formula (II) is of formula (II*):

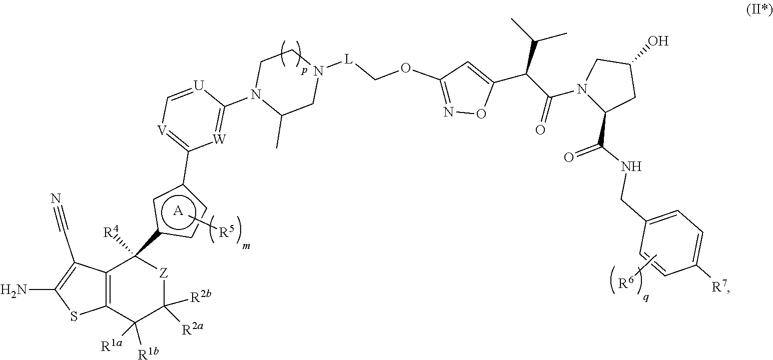

wherein R^{1a}, R^{1b}, R^{2a}, R^{2b}, Z, R⁴, ring A, R⁵, m, U, V, W, p, L, q, R⁵ and R⁷ are as herein defined.

In another aspect, the compound or salt of formula (II) is of formula (II**):

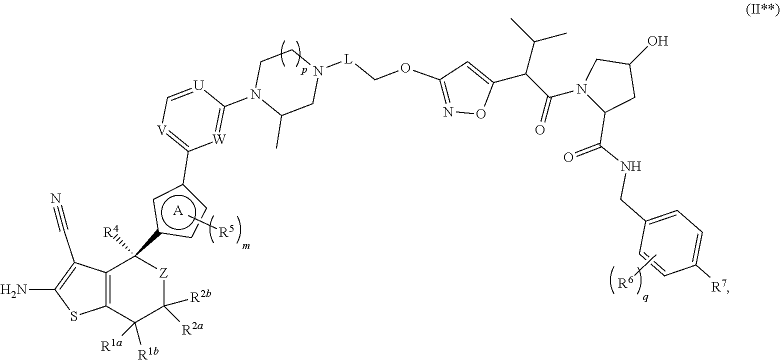

wherein R^{1a}, R^{1b}, R^{2a}, R^{2b}, Z, R⁴, ring A, R⁵, m, U, V, W, p, L, q, R⁵ and R⁷ are as herein defined.

In another aspect, the compound or salt of formula (II) is of formula (II***):

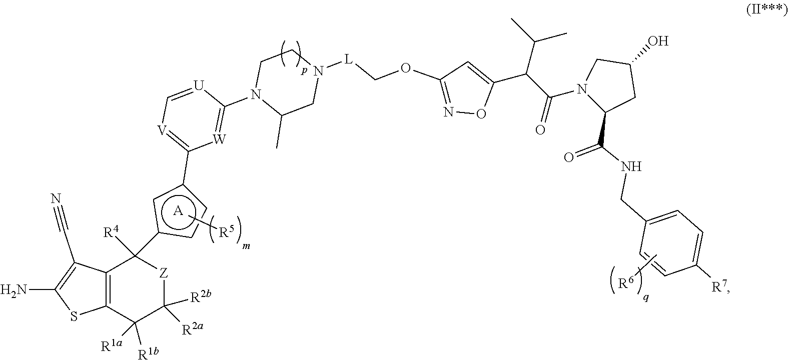

wherein R^{1a}, R^{1b}, R^{2a}, R^{2b}, Z, R⁴, ring A, R⁵, m, U, V, W, p, L, q, R⁵ and R⁷ are as herein defined.

In another aspect, the compound of formula (II) is of formula (II****):

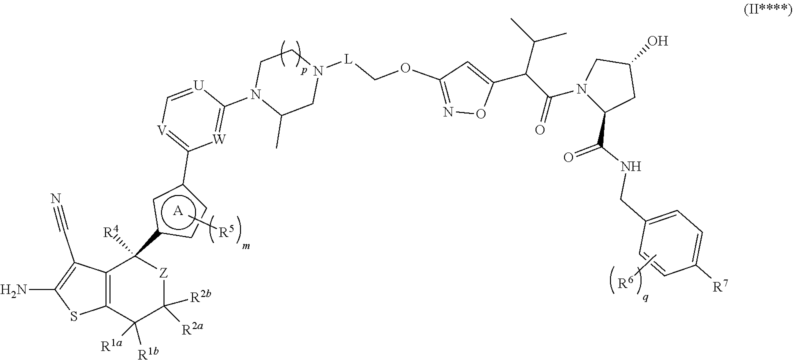

wherein R^{1a}, R^{1b}, R^{2a}, R^{2b}, Z, R⁴, ring A, R⁵, m, U, V, W, p, L, q, R⁵ and R⁷ are as herein defined.

It is to be understood that the configuration at the asymmetric carbon atoms depicted in formulas (II*), (II**), (II***) and (II****) can be applied to any one or more of the aspects and/or preferred embodiments defined below.

In one aspect, the compound of formula (II) is of formula (IIa):

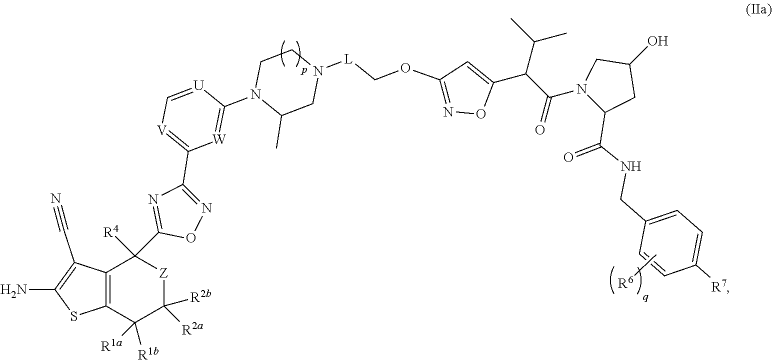

wherein R^{1a}, R^{1b}, R^{2a}, R^{2b}, Z, R⁴, U, V, W, p, L, q, R⁶ and R⁷ are as herein defined.

Preferably, it is of formula (II*a):

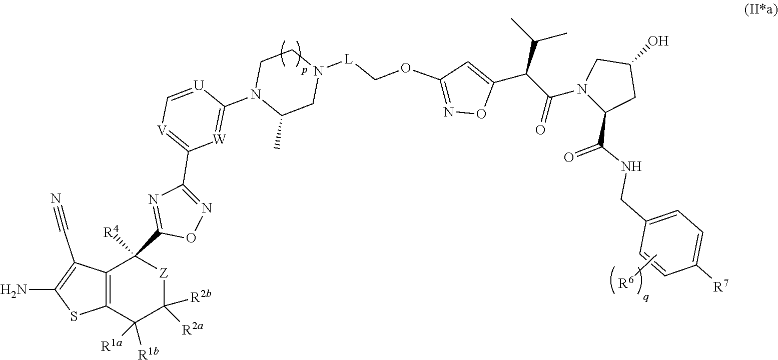

wherein R^{1a}, R^{1b}, R^{2a}, R^{2b}, Z, R⁴, U, V, W, p, L, q, R⁶ and R⁷ are as herein defined.

In another aspect, the compound of formula (II) is of formula (IIb):

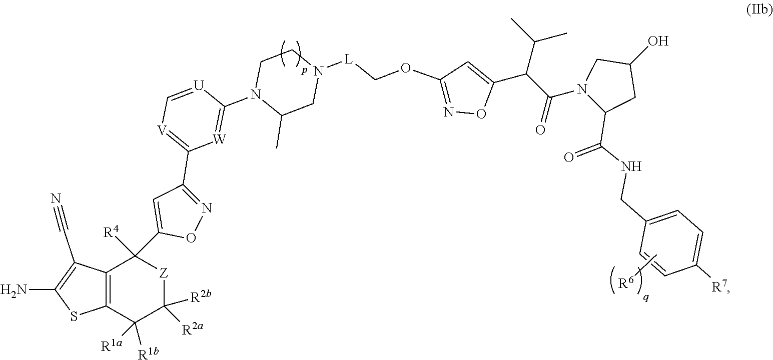

wherein R^{1a}, R^{1b}, R^{2a}, R^{2b}, Z, R⁴, U, V, W, p, L, q, R⁶ and R⁷ are as herein defined.

Preferably, it is of formula (II*b):

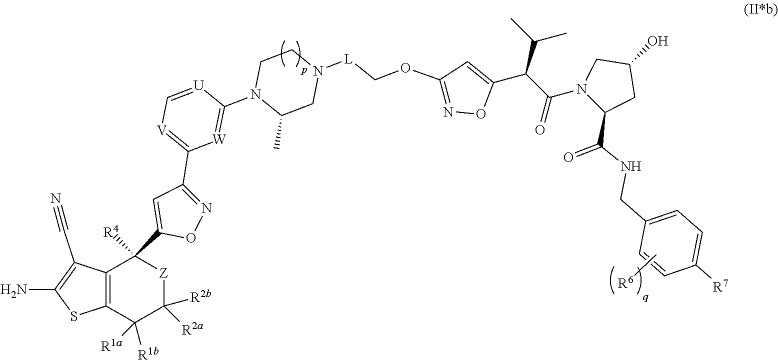

wherein R^{1a}, R^{1b}, R^{2a}, R^{2b}, Z, R⁴, U, V, W, p, L, q, R⁶ and R⁷ are as herein defined.

In another aspect, the compound of formula (II) is of formula (IIc):

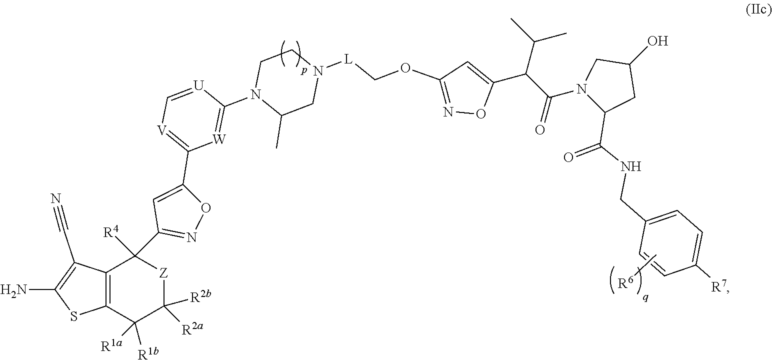

wherein R^{1a}, R^{1b}, R^{2a}, R^{2b}, Z, R⁴, U, V, W, p, L, q, R⁶ and R⁷ are as herein defined.

Preferably, it is of formula (II*c):

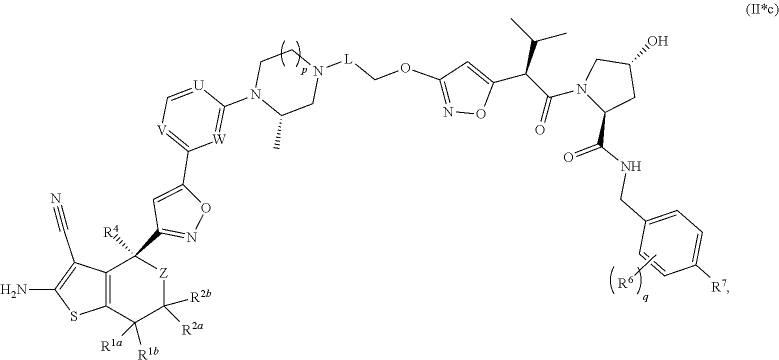

wherein R^{1a}, R^{1b}, R^{2a}, R^{2b}, Z, R⁴, U, V, W, p, L, q, R⁶ and R⁷ are as herein defined.

In another aspect, the compound of formula (II) is of formula (IId):

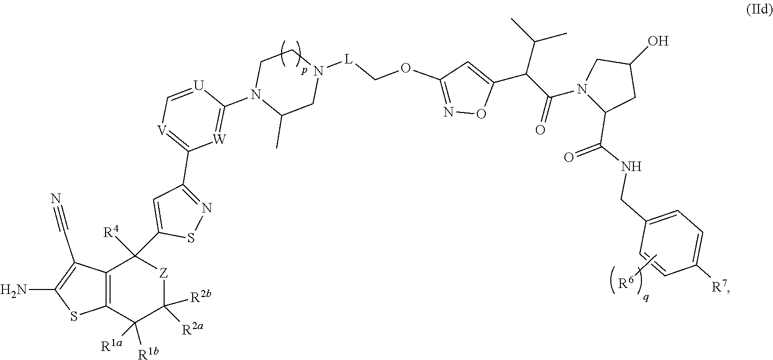

wherein R^{1a}, R^{1b}, R^{2a}, R^{2b}, Z, R⁴, U, V, W, p, L, q, R⁶ and R⁷ are as herein defined.

Preferably, it is of formula (II*d):

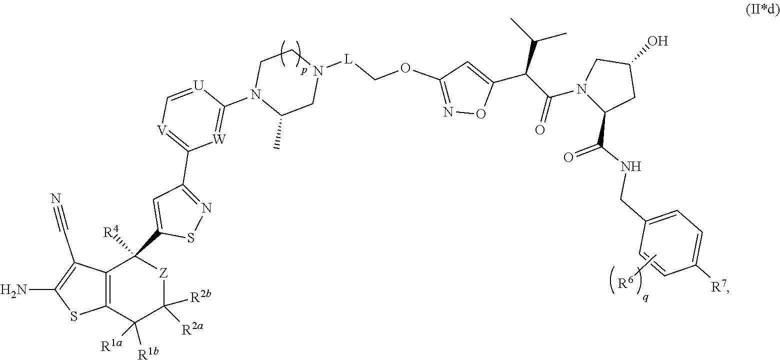

wherein R^{1a}, R^{1b}, R^{2a}, R^{2b}, Z, R⁴, U, V, W, p, L, q, R⁶ and R⁷ are as herein defined.

In another aspect, the compound of formula (II) is of formula (IIe):

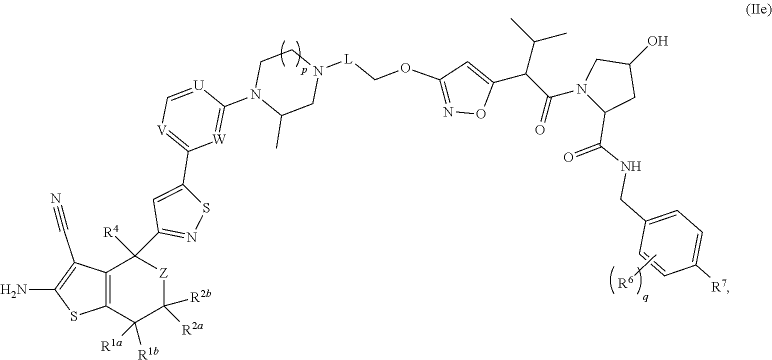

wherein R^{1a}, R^{1b}, R^{2a}, R^{2b}, Z, R⁴, U, V, W, p, L, q, R⁶ and R⁷ are as herein defined.

Preferably, it is of formula (II*e):

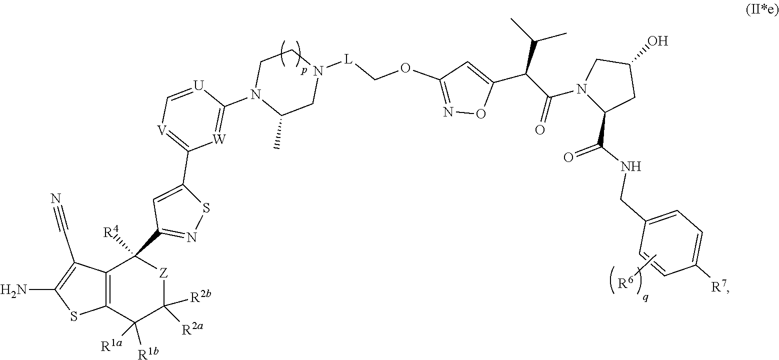

wherein R^{1a}, R^{1b}, R^{2a}, R^{2b}, Z, R⁴, U, V, W, p, L, q, R⁶ and R⁷ are as herein defined.

It is to be understood that compounds of formula (II*), (II**), (II***), (II****), (IIa), (II*a), (IIb), (II*b), (IIc), (II*c), (IId), (II*d), (IIe), (II*e), and (IIf) each are a subset of compounds of formula (II) and that whenever it is referred to compounds of formula (II) this is meant to also refer to and include compounds (II*), (II**), (II***), (II****), (IIa), (II*a), (IIb), (II*b), (IIc), (II*c), (IId), (II*d), (IIe), (II*e), and (IIf) unless stated otherwise. Formulas (II*), (II**), (II***), (II****), (IIa), (II*a), (IIb), (II*b), (IIc), (II*c), (IId), (II*d), (IIe), (II*e), and (IIf) can be referred to as “subformulas” of formula (II).

In one aspect of the compound of formula (II), (II*), (II**), (II***), (II****), (IIa), (II*a), (IIb), (II*b), (IIc), (II*c), (IId), (II*d), (IIe), (II*e), or (IIf), one of R^{1a} or R^{1b} is hydrogen.

In another aspect of the compound of formula (II), (II*), (II**), (II***), (II****), (IIa), (II*a), (IIb), (II*b), (IIc), (II*c), (IId), (II*d), (IIe), (II*e), or (IIf), R^{1a} and R^{1b} are hydrogen.

In another aspect of the compound of formula (II), (II*), (II**), (II***), (II****), (IIa), (II*a), (IIb), (II*b), (IIc), (II*c), (IId), (II*d), (IIe), (II*e), or (IIf), one of R^{2a} or R^{2b} is hydrogen.

In another aspect of the compound of formula (II), (II*), (II**), (II***), (II****), (IIa), (II*a), (IIb), (II*b), (IIc), (II*c), (IId), (II*d), (IIe), (II*e), or (IIf), R^{2a} and R^{2b} are hydrogen.

In another aspect of the compound of formula (II), (II*), (II**), (II***), (II****), (IIa), (II*a), (IIb), (II*b), (IIc), (II*c), (IId), (II*d), (IIe), (II*e), or (IIf), R^{1a}, R^{1b}, R^{2a}, R^{2b} are hydrogen.

In another aspect of the compound of formula (II), (II*), (II**), (II***), (II****), (IIa), (II*a), (IIb), (II*b), (IIc), (II*c), (IId), (II*d), (IIe), (II*e), or (IIf), one of R^{3a} or R^{3b} is hydrogen.

In another aspect of the compound of formula (II), (II*), (II**), (II***), (II****), (IIa), (II*a), (IIb), (II*b), (IIc), (II*c), (IId), (II*d), (IIe), (II*e), or (IIf), R^{3a} and R^{3b} are hydrogen.

In another aspect of the compound of formula (II), (II*), (II**), (II***), (II****), (IIa), (II*a), (IIb), (II*b), (IIc), (II*c), (IId), (II*d), (IIe), (II*e), or (IIf), R^{1a}, R^{1b}, R^{2a}, R^{2b}, R^{3a} and R^{3b} are hydrogen.

In another aspect of the compound of formula (II), (II*), (II**), (II***), (II****), (IIa), (II*a), (IIb), (II*b), (IIc), (II*c), (IId), (II*d), (IIe), (II*e), or (IIf), n is 1.

In another aspect of the compound of formula (II), (II*), (II**), (II***), (II****), (IIa), (II*a), (IIb), (II*b), (IIc), (II*c), (IId), (II*d), (IIe), (II*e), or (IIf), Z is —CH₂—.

In another aspect of the compound of formula (II), (II*), (II**), (II***), (II****), (IIa), (II*a), (IIb), (II*b), (IIc), (II*c), (IId), (II*d), (IIe), (II*e), or (IIf), R^{1a}, R^{1b}, R^{2a} and R^{2b} are hydrogen and Z is —CH₂—.

In another aspect of the compound of formula (II), (II***), (IIa), (II*a), (IIb), (II*b), (IIc), (II*c), (IId), (II*d), (IIe), (II*e), or (IIf), the carbon atom to which R⁴ and ring A are attached is in the (S) configuration.

In another aspect of the compound of formula (II), (II*), (II**), (II***), (II****), (IIa), (II*a), (IIb), (II*b), (IIc), (II*c), (IId), (II*d), (IIe), (II*e), or (IIf), R⁴ is selected from the group consisting of hydrogen, C₁₋₃alkyl, C₁₋₃haloalkyl, C₁₋₃alkoxy, C₁₋₃haloalkoxy, cyano-C₁₋₃alkyl, halogen, —OH, —NH₂, —NH(C₁₋₃alkyl), —N(C₁₋₃alkyl)₂, —CN, C₃₋₄cycloalkyl and 3-4 membered heterocycloalkyl.

In another aspect of the compound of formula (II), (II*), (II**), (II***), (II****), (IIa), (II*a), (IIb), (II*b), (IIc), (II*c), (IId), (II*d), (IIe), (II*e), or (IIf), R⁴ is C₁₋₆alkyl.

In another aspect of the compound of formula (II), (II*), (II**), (II***), (II****), (IIa), (II*a), (IIb), (II*b), (IIc), (II*c), (IId), (II*d), (IIe), (II*e), or (IIf), R⁴ is C₁₋₃alkyl.

In another aspect of the compound of formula (II), (II*), (II**), (II***), (II****), (IIa), (II*a), (IIb), (II*b), (IIc), (II*c), (IId), (II*d), (IIe), (II*e), or (IIf), R⁴ is methyl.

In another aspect of the compound of formula (II), (II*), (II**), (II***), (II****), (IIa), (II*a), (IIb), (II*b), (IIc), (II*c), (IId), (II*d), (IIe), (II*e), or (IIf), R^{1a}, R^{1b}, R^{2a} and R^{2b} are hydrogen, Z is —CH₂— and R⁴ is methyl.

In another aspect of the compound of formula (II), (II*), (II**), (II***), (II****), (IIa), (II*a), (IIb), (II*b), (IIc), (II*c), (IId), (II*d), (IIe), (II*e), or (IIf), R^{1a}, R^{1b}, R^{2a} and R^{2b} are hydrogen, Z is —CH₂—, R⁴ is methyl and the carbon atom to which R⁴ and ring A are attached is in the (S) configuration.

In another aspect of the compound of formula (II), (II*), (II**), (II***), (II****), (IIa), (II*a), (IIb), (II*b), (IIc), (II*c), (IId), (II*d), (IIe), (II*e), or (IIf), ring A is selected from the group consisting of oxazole, isoxazole, thiazole, isothiazole, oxadiazole and thiadiazole.

In another aspect of the compound of formula (II), (II*), (II**), (II***), (II****), (IIa), (II*a), (IIb), (II*b), (IIc), (II*c), (IId), (II*d), (IIe), (II*e), or (IIf), ring A is selected from the group consisting of isoxazole, isothiazole and oxadiazole.

In another aspect of the compound of formula (II), (II*), (II**), (II***), (II****), (IIa), (II*a), (IIb), (II*b), (IIc), (II*c), (IId), (II*d), (IIe), (II*e), or (IIf), ring A is selected from the group consisting of:

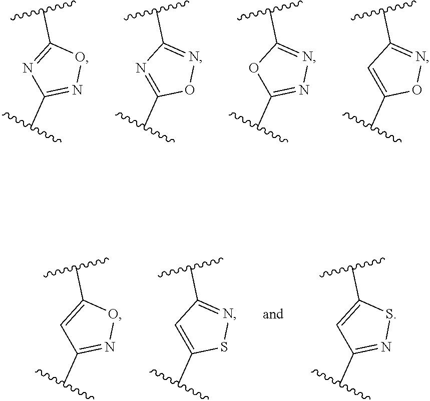

In another aspect of the compound of formula (II), (II*), (II**), (II***), (II****), (IIa), (II*a), (IIb), (II*b), (IIc), (II*c), (IId), (II*d), (IIe), (II*e), or (IIf), A is selected from the group consisting of:

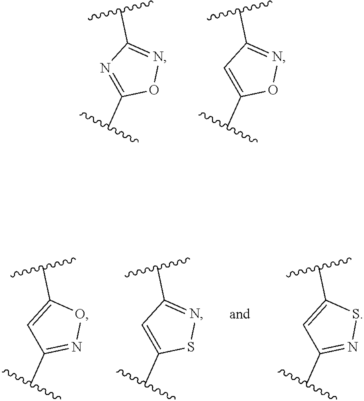 [SMILES: *c1noc(*)n1 ; *c1cc(*)on1 ; *c1cc(*)on1 ; *c1cc(*)sn1 ; "and" ; *c1cc(*)sn1]

In another aspect of the compound of formula (II), (II*), (II**), (II***), (II****), (IIa), (II*a), (IIb), (II*b), (IIc), (II*c), (IId), (II*d), (IIe), (II*e), or (IIf), oxadiaziole is selected from the group consisting of

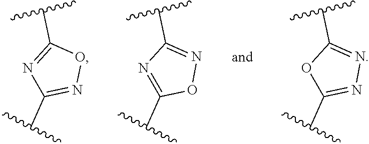 [SMILES: *c1noc(*)n1 ; *c1noc(*)n1 ; "and" ; *c1nnc(*)o1]

In another aspect of the compound of formula (II), (II*), (II**), (II***), (II****), (IIa), (II*a), (IIb), (II*b), (IIc), (II*c), (IId), (II*d), (IIe), (II*e), or (IIf), oxadiaziole is

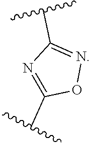 [SMILES: *c1noc(*)n1]

In another aspect of the compound of formula (II), (II*), (II**), (II***), (II****), (IIa), (II*a), (IIb), (II*b), (IIc), (II*c), (IId), (II*d), (IIe), (II*e), or (IIf), isoxazole is:

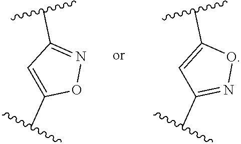 [SMILES: *c1cc(*)on1 ; "or" ; *c1cc(*)on1]

In another aspect of the compound of formula (II), (II*), (II**), (II***), (II****), (IIa), (II*a), (IIb), (II*b), (IIc), (II*c), (IId), (II*d), (IIe), (II*e), or (IIf), isothiazole is:

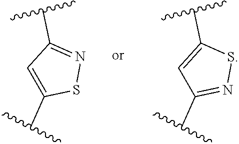 [SMILES: *c1cc(*)sn1 ; "or" ; *c1cc(*)sn1]

In another aspect of the compound of formula (II), (II*), (II**), (II***), (II****), (IIa), (II*a), (IIb), (II*b), (IIc), (II*c), (IId), (II*d), (IIe), (II*e), or (IIf), m is 0.

In another aspect of the compound of formula (II), (II*), (II**), (II***), (II****), (IIa), (II*a), (IIb), (II*b), (IIc), (II*c), (IId), (II*d), (IIe), (II*e), or (IIf), at least one of V or W is ═N—.

In another aspect of the compound of formula (II), (II*), (II**), (II***), (II****), (IIa), (II*a), (IIb), (II*b), (IIc), (II*c), (IId), (II*d), (IIe), (II*e), or (IIf), one of V or W is ═N—.

In another aspect of the compound of formula (II), (II*), (II**), (II***), (II****), (IIa), (II*a), (IIb), (II*b), (IIc), (II*c), (IId), (II*d), (IIe), (II*e), or (IIf), both V and W are ═N—.

In another aspect of the compound of formula (II), (II*), (II**), (II***), (II****), (IIa), (II*a), (IIb), (II*b), (IIc), (II*c), (IId), (II*d), (IIe), (II*e), or (IIf), W is ═N—.

In another aspect of the compound of formula (II), (II*), (II**), (II***), (II****), (IIa), (II*a), (IIb), (II*b), (IIc), (II*c), (IId), (II*d), (IIe), (II*e), or (IIf), U is ═N—, V is ═C(H)— and W is ═N—.

In another aspect of the compound of formula (II), (II*), (II**), (II***), (II****), (IIa), (II*a), (IIb), (II*b), (IIc), (II*c), (IId), (II*d), (IIe), (II*e), or (IIf), U is ═C(H)—, V is ═N— and W is ═N—.

In another aspect of the compound of formula (II), (II*), (II**), (II***), (II****), (IIa), (II*a), (IIb), (II*b), (IIc), (II*c), (IId), (II*d), (IIe), (II*e), or (IIf), R^{1a}, R^{1b}, R^{2a} and R^{2b} are hydrogen, Z is —CH₂—, R⁴ is methyl and W is ═N—.

In another aspect of the compound of formula (II), (II*), (II**), (II***), (II****), (IIa), (II*a), (IIb), (II*b), (IIc), (II*c), (IId), (II*d), (IIe), (II*e), or (IIf), p is 1 or 2.

In another aspect of the compound of formula (II), (II*), (II**), (II***), (II****), (IIa), (II*a), (IIb), (II*b), (IIc), (II*c), (IId), (II*d), (IIe), (II*e), or (IIf), p is 1.

In another aspect of the compound of formula (II), (II*), (II**), (II***), (II****), (IIa), (II*a), (IIb), (II*b), (IIc), (II*c), (IId), (II*d), (IIe), (II*e), or (IIf), p is 2.

In another aspect of the compound of formula (II), (II*), (II**), (II***), (II****), (IIa), (II*a), (IIb), (II*b), (IIc), (II*c), (IId), (II*d), (IIe), (II*e), or (IIf), L is selected from the group consisting of C₁₋₆alkylene, C₂₋₆alkenylene and C₁₋₆alkoxylene. Preferably, said C₁₋₆alkylene, C₂₋₆alkenylene and C₁₋₆alkoxylene are linear.

In another aspect of the compound of formula (II), (II*), (II**), (II***), (II****), (IIa), (II*a), (IIb), (II*b), (IIc), (II*c), (IId), (II*d), (IIe), (II*e), or (IIf), L is selected from the group consisting of a bond, C₁₋₄alkylene, C₂₋₄alkenylene and C₁₋₄alkoxylene. Preferably, said C₁₋₄alkylene, C₂₋₄alkenylene and C₁₋₄alkoxylene are linear.

In another aspect of the compound of formula (II), (II*), (II**), (II***), (II****), (IIa), (II*a), (IIb), (II*b), (IIc), (II*c), (IId), (II*d), (IIe), (II*e), or (IIf), L is selected from the group consisting of C₁₋₄alkylene, C₂₋₄alkenylene and C₁₋₄alkoxylene.

In another aspect of the compound of formula (II), (II*), (II**), (II***), (II****), (IIa), (II*a), (IIb), (II*b), (IIc), (II*c), (IId), (II*d), (IIe), (II*e), or (IIf), L is selected from the group consisting of: C₁₋₅alkylene, C₃alkenylene and C₃₋₄alkoxylene.

In another aspect of the compound of formula (II), (II*), (II**), (II***), (II****), (IIa), (II*a), (IIb), (II*b), (IIc), (II*c), (IId), (II*d), (IIe), (II*e), or (IIf), L is selected from the group consisting of:
—CH₂—, —(CH₂)₂—, —(CH₂)₃—, —(CH₂)₅—,

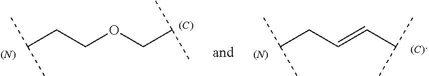

It is to be understood that “(N)” and “(C)” indicate the atom of formula (I) to which L is attached.

In another aspect of the compound of formula (II), (II*), (II**), (II***), (II****), (IIa), (II*a), (IIb), (II*b), (IIc), (II*c), (IId), (II*d), (IIe), (II*e), or (IIf), each R⁶, if present, is independently at each occurrence halogen or C₁₋₃alkyl.

In another aspect of the compound of formula (II), (II*), (II**), (II***), (II****), (IIa), (II*a), (IIb), (II*b), (IIc), (II*c), (IId), (II*d), (IIe), (II*e), or (IIf), q is 0. It is to be understood that when q is 0, R⁶ is hydrogen.

In another aspect of the compound of formula (II), (II*), (II**), (II***), (II****), (IIa), (II*a), (IIb), (II*b), (IIc), (II*c), (IId), (II*d), (IIe), (II*e), or (IIf), q is 1. When q is 1, R⁶ may be meta- or ortho- to R⁷. When R⁶ is meta- to R⁷, it is labelled as Rea. When R⁶ is ortho- to R⁷, it is labelled as R^{6b}.

In another aspect of the compound of formula (II), (II*), (II**), (II***), (II****), (IIa), (II*a), (IIb), (II*b), (IIc), (II*c), (IId), (II*d), (IIe), (II*e), or (IIf), when q is 1, R^{6a} and R^{6b} are each independently selected from the group consisting of: hydrogen, halogen and C₁₋₃alkyl. It is to be understood that when q is 1, one of R^{6a} or R^{6b} is hydrogen.

In another aspect of the compound of formula (II), (II*), (II**), (II***), (II****), (IIa), (II*a), (IIb), (II*b), (IIc), (II*c), (IId), (II*d), (IIe), (II*e), or (IIf), q is 2. When q is 2, there are two R⁶'s. The R⁶ that is meta- to R⁷ is labelled as Rea. The R⁶ that is ortho- to R⁷ is labelled as R^{6b}.

In another aspect of the compound of formula (II), (II*), (II**), (II***), (II****), (IIa), (II*a), (IIb), (II*b), (IIc), (II*c), (IId), (II*d), (IIe), (II*e), or (IIf), when q is 2, R^{6a} and R^{6b} are each independently halogen or C₁₋₃alkyl.

In another aspect of the compound of formula (II), (II*), (II**), (II***), (II****), (IIa), (II*a), (IIb), (II*b), (IIc), (II*c), (IId), (II*d), (IIe), (II*e), or (IIf), q is 0 or 1.

In another aspect of the compound of formula (II), (II*), (II**), (II***), (II****), (IIa), (II*a), (IIb), (II*b), (IIc), (II*c), (IId), (II*d), (IIe), (II*e), or (IIf), p is 1 or 2 and q is 0 or 1.

In another aspect, the compound of formula (II) is of formula (IIf):

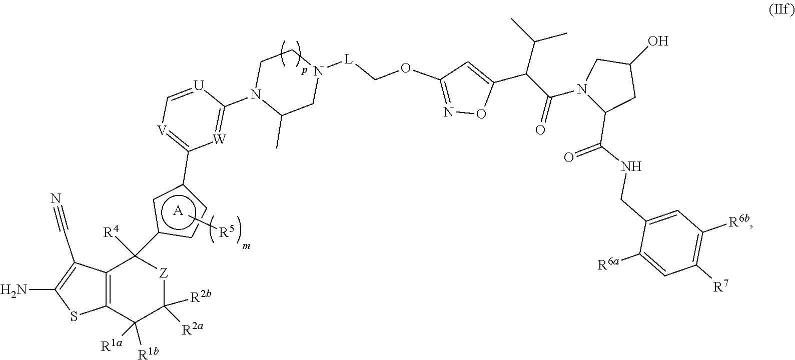

wherein R^{1a}, R^{1b}, R^{2a}, R^{2b}, Z, R, ring A, R⁵, m, U, V, W, p, L, and R⁷ are as herein defined; R^{6a} and R^{6b} are each independently selected from the group consisting of hydrogen, halogen and C₁₋₃alkyl.

In one aspect of the compound of formula (IIf), R^{6a} is hydrogen or halogen.

In another aspect of the compound of formula (IIf), R^{6a} is hydrogen or fluorine.

In another aspect of the compound of formula (IIf), R^{6a} is hydrogen.

In another aspect of the compound of formula (IIf), R^{6a} is fluorine.

In another aspect of the compound of formula (IIf), R^{6b} is selected from the group consisting of hydrogen, fluorine, chlorine and methyl.

In another aspect of the compound of formula (IIf), R^{6a} is hydrogen or halogen and R^{1b} is hydrogen, halogen or C₁₋₃alkyl.

In another aspect of the compound of formula (IIf), R^{6a} is hydrogen and R^{6b} is hydrogen, halogen and C₁₋₃alkyl.

In another aspect of the compound of formula (IIf), R^{6a} and R^{6b} are each independently selected from the group consisting of hydrogen, fluorine, chlorine and methyl.

In another aspect of the compound of formula (IIf), R^{6a} is hydrogen or fluorine and R^{6b} is selected from the group consisting of hydrogen, fluorine, chlorine and methyl.

In another aspect of the compound of formula (IIf), R^{6a} is hydrogen and R^{6b} is selected from the group consisting of fluorine, chlorine and methyl.

In another aspect of the compound of formula (IIf), R^{1a} and R^{1b} are hydrogen.

In another aspect of the compound of formula (II), (II*), (II**), (II***), (II****), (IIa), (II*a), (IIb), (II*b), (IIc), (II*c), (IId), (II*d), (IIe), (II*e), or (IIf), R⁷ is selected from the group consisting of: chlorine, bromine, iso-propyl, —CN, thiazole, triazole and imidazole, wherein said thiazole, triazole or imidazole is optionally substituted with R⁸.

In another aspect of the compound of formula (II), (II*), (II**), (II***), (II****), (IIa), (II*a), (IIb), (II*b), (IIc), (II*c), (IId), (II*d), (IIe), (II*e), or (IIf), R⁷ is selected from the group consisting of: halogen, C₁₋₃alkyl, —CN, thiazole, triazole and imidazole, wherein said thiazole is substituted with R⁸.

In another aspect of the compound of formula (II), (II*), (II**), (II***), (II****), (IIa), (II*a), (IIb), (II*b), (IIc), (II*c), (IId), (II*d), (IIe), (II*e), or (IIf), R⁷ is selected from the group consisting of: halogen, C₁₋₃alkyl, —CN, thiazole, triazole and imidazole, wherein said thiazole is substituted with methyl or —CH₂OH.

In another aspect of the compound of formula (II), (II*), (II**), (II***), (II****), (IIa), (II*a), (IIb), (II*b), (IIc), (II*c), (IId), (II*d), (IIe), (II*e), or (IIf), R⁷ is selected from the group consisting of: chlorine, bromine, iso-propyl, —CN, thiazole, triazole and imidazole, wherein said thiazole is substituted with methyl or —CH₂OH.

In another aspect of the compound of formula (II), (II*), (II**), (II***), (II****), (IIa), (II*a), (IIb), (II*b), (IIc), (II*c), (IId), (II*d), (IIe), (II*e), or (IIf), R⁷ is selected from the group consisting of:
chlorine, bromine, iso-propyl, —CN,

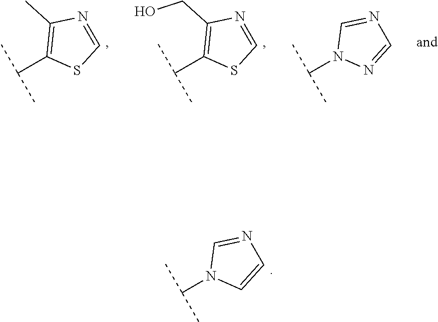

In another aspect of the compound of formula (II), (II*), (II**), (II***), (II****), (IIa), (II*a), (IIb), (II*b), (IIc), (II*c), (IId), (II*d), (IIe), (II*e), or (IIf), R⁸ is methyl or —CH₂OH.

In another aspect of the compound of formula (II), (II*), (II**), (II***), (II****), (IIa), (II*a), (IIb), (II*b), (IIc), (II*c), (IId), (II*d), (IIe), (II*e), or (IIf), R⁸ is methyl.

Preferred embodiments of compound of formula (I) or (II) are represented by compounds of formulas I-1 to I-27, as defined hereinbelow, including any stereoisomer thereof.

The present invention also refers to a process to make a compounds of formula (II) with the process of Scheme 3 or 4:

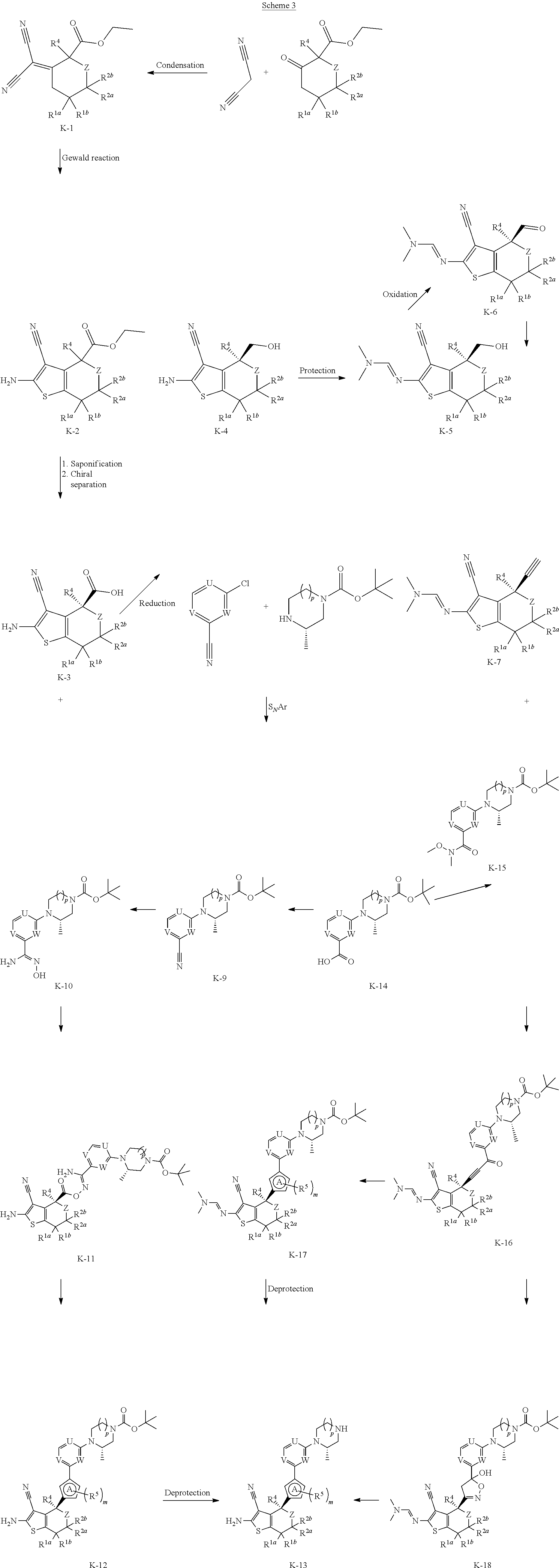

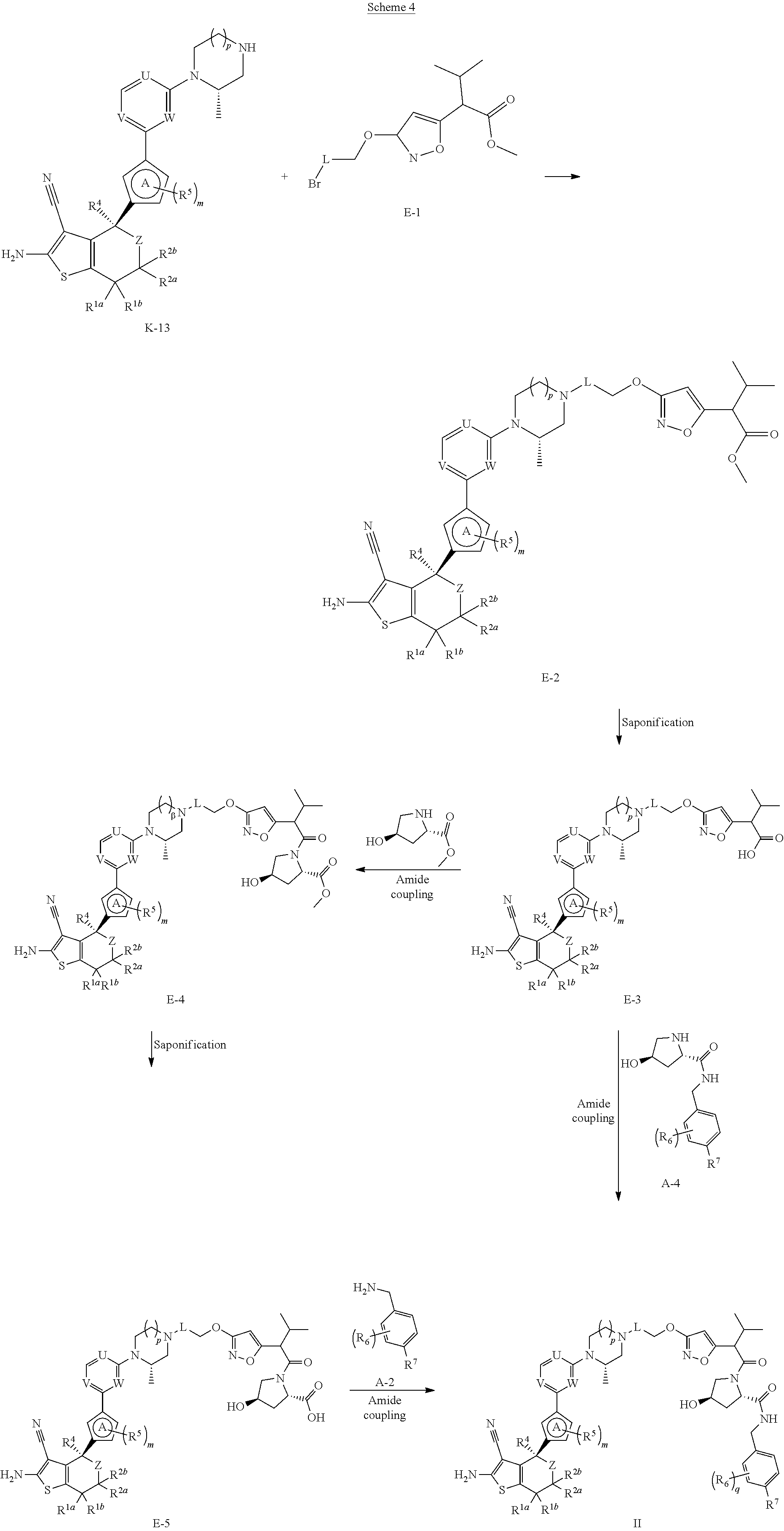

It is a further object of the present invention a compound selected from the group consisting of:

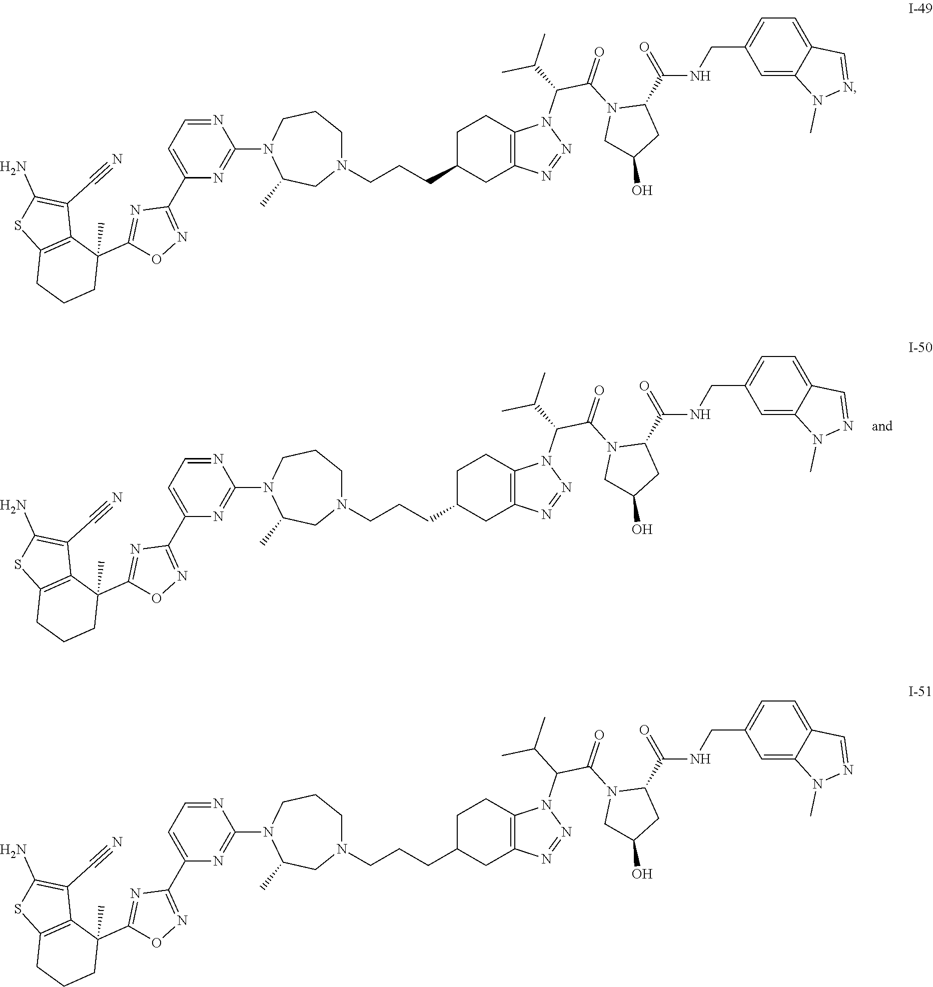 [SMILES: CC(C)[C@H](C(=O)N1C[C@H](O)C[C@H]1C(=O)NCc1ccc2cnn(C)c2c1)n1nnc2c1CC[C@H](CCCN1CCCN(c3nccc(-c4noc([C@@]5(C)CCCc6sc(N)c(C#N)c65)n4)n3)[C@@H](C)C1)C2 ; CC(C)[C@H](C(=O)N1C[C@H](O)C[C@H]1C(=O)NCc1ccc2cnn(C)c2c1)n1nnc2c1CC[C@@H](CCCN1CCCN(c3nccc(-c4noc([C@@]5(C)CCCc6sc(N)c(C#N)c65)n4)n3)[C@@H](C)C1)C2 ; "and" ; CC(C)C(C(=O)N1C[C@H](O)C[C@H]1C(=O)NCc1ccc2cnn(C)c2c1)n1nnc2c1CCC(CCCN1CCCN(c3nccc(-c4noc([C@@]5(C)CCCc6sc(N)c(C#N)c65)n4)n3)[C@@H](C)C1)C2]

or a pharmaceutically acceptable salt or a stereoisomer thereof.

The present invention further relates to hydrates, solvates, polymorphs, metabolites, derivatives, stereoisomers and prodrugs of a compound of formula (II), (II*), (II**), (II***), (II****), (IIa), (II*a), (IIb), (II*b), (IIc), (II*c), (IId), (II*d), (IIe), (II*e), or (IIf), (including all aspects and preferred embodiments thereof).

The present invention further relates to a hydrate of a compound of formula (II), (II*), (II**), (II***), (II****), (IIa), (II*a), (IIb), (II*b), (IIc), (II*c), (IId), (II*d), (IIe), (II*e), or (IIf), (including all aspects and preferred embodiments thereof).

The present invention further relates to a solvate of a compound of formula (II), (II*), (II**), (II***), (II****), (IIa), (II*a), (IIb), (II*b), (IIc), (II*c), (IId), (II*d), (IIe), (II*e), or (IIf), (including all aspects and preferred embodiments thereof).

Compounds of formula (11), (II*), (II**), (II***), (II****), (IIa), (II*a), (IIb), (II*b), (IIc), (II*c), (IId), (II*d), (IIe), (II*e), or (IIf), (including all aspects and preferred embodiments thereof) which e.g. bear ester groups are potential prodrugs the ester being cleaved under physiological conditions and are also part of the invention.

The present invention further relates to a pharmaceutically acceptable salt of a compound of formula (II), (II*), (II**), (II***), (II****), (IIa), (II*a), (IIb), (II*b), (IIc), (II*c), (IId), (II*d), (IIe), (II*e), or (IIf) (including all aspects and preferred embodiments thereof).

### Medical Uses—Methods of Treatment

### Indications—Patient Populations

The present invention is directed to compounds inhibiting or degrading KRAS, preferably KRAS mutated at residue 12, such as KRAS G12C, KRAS G12D, KRAS G12V, KRAS G12A and KRAS G12R inhibitors, preferably inhibitors of KRAS G12C and/or KRAS G12D, or inhibitors selective for KRAS G12D, as well as compounds inhibiting KRAS wildtype, preferably amplified, KRAS mutated at residue 13, such as KRAS G13D, or KRAS mutated at residue 61, such as KRAS Q61H. In particular, compounds of the invention can be useful in the treatment and/or prevention of diseases and/or conditions dependent on or mediated by KRAS, preferably by KRAS mutated at residue 12, e.g. KRAS G12C, KRAS G12D, KRAS G12V, more preferably G12D, or by an amplification of KRAS wildtype, or by KRAS mutated at residue 13, e.g. KRAS G13D, or by KRAS mutated at residue 61, such as KRAS Q61H. Thus, in a further aspect the invention relates to a compound of the invention—or a pharmaceutically acceptable salt thereof—for use as a medicament.

In a further aspect the invention relates to a compound of the invention—or a pharmaceutically acceptable salt thereof—for use in a method of treatment of the human or animal body.

In a further aspect the invention relates to a compound of the invention—or a pharmaceutically acceptable salt thereof—for use in the treatment and/or prevention of a disease and/or condition mediated by KRAS, preferably by KRAS mutated at residue 12, e.g. KRAS G12C, KRAS G12D, KRAS G12V, more preferably G12D, or by an amplification of KRAS wildtype, or by KRAS mutated at residue 13, e.g. KRAS G13D.

In a further aspect the invention relates to the use of a compound of the invention—or a pharmaceutically acceptable salt thereof—in the manufacture of a medicament for the treatment and/or prevention of a disease and/or condition mediated by KRAS, preferably by KRAS mutated at residue 12, e.g. KRAS G12C, KRAS G12D, KRAS G12V, more preferably G12D, or by an amplification of KRAS wildtype, or by KRAS mutated at residue 13, e.g. KRAS G13D.

In a further aspect the invention relates to a method for the treatment and/or prevention of a disease and/or condition mediated by KRAS, preferably by KRAS mutated at residue 12, e.g. KRAS G12C, KRAS G12D, KRAS G12V, more preferably G12D, or by an amplification of KRAS wildtype, or by KRAS mutated at residue 13, e.g. KRAS G13D, comprising administering a therapeutically effective amount of a compound of the invention—or a pharmaceutically acceptable salt thereof—to a human being.

In a further aspect the invention relates to a compound of the invention—or a pharmaceutically acceptable salt thereof—for use in the treatment and/or prevention of cancer.

In a further aspect the invention relates to a compound of the invention—or a pharmaceutically acceptable salt thereof—for use in a method of treatment and/or prevention of cancer in the human or animal body.

In a further aspect the invention relates to the use of a compound of the invention—or a pharmaceutically acceptable salt thereof—in the manufacture of a medicament for the treatment and/or prevention of cancer.

In a further aspect the invention relates to a method for the treatment and/or prevention of cancer comprising administering a therapeutically effective amount of a compound of the invention—or a pharmaceutically acceptable salt thereof—to a human being.

Preferably, the cancer as defined herein (above or below) comprises a KRAS mutation. In particular, KRAS mutations include e.g. mutations of the KRAS gene and of the KRAS protein, such as overexpressed KRAS, amplified KRAS or KRAS, KRAS mutated at residue 12, KRAS mutated at residue 13, KRAS mutated at residue 61, KRAS mutated at residue 146, in particular KRAS G12A, KRAS G12C, KRAS G12D, KRAS G12V, KRAS G12S, KRAS G13C, KRAS G13D, KRAS G13V, KRAS Q61H, KRAS Q61E, KRAS Q61P, KRAS A146P, KRAS A146T, KRAS A146V. KRAS may present one or more of these mutations/alterations.

Preferably, the cancer as defined herein (above or below) comprises a BRAF mutation in addition or in alternative to the KRAS mutation. Said BRAF mutation is in particular a class Ill BRAF mutation, e.g. as defined in Z. Yao, Nature, 2017, 548, 234-238.

Preferably, the cancer as defined herein (above or below) comprises a mutation in a receptor tyrosine kinase (RTK), including EGFR, MET and ERBB2 mutations, in addition or in alternative to the KRAS mutation.

In a further aspect the invention relates to a compound of the invention—or a pharmaceutically acceptable salt thereof—for use in the treatment and/or prevention of cancer, wherein the cancer comprises a KRAS mutation, said KRAS mutation being preferably selected from the group consisting of: KRAS G12C, KRAS G12D, KRAS G12V, KRAS G13D; or an amplification of KRAS wildtype, amplification of the KRAS gene or overexpression of KRAS.

In a further aspect the invention relates to the use of a compound of the invention—or a pharmaceutically acceptable salt thereof—in the manufacture of a medicament for the treatment and/or prevention of cancer, wherein the cancer comprises a KRAS mutation, said KRAS mutation being preferably selected from the group consisting of: KRAS G12C, KRAS G12D, KRAS G12V, KRAS G13D; or an amplification of KRAS wildtype, amplification of the KRAS gene or overexpression of KRAS.

In a further aspect the invention relates to a method for the treatment and/or prevention of cancer comprising administering a therapeutically effective amount of a compound of the invention—or a pharmaceutically acceptable salt thereof—to a human being, wherein the cancer comprises a KRAS mutation, said KRAS mutation being preferably selected from the group consisting of: KRAS G12C, KRAS G12D, KRAS G12V, KRAS G13D; or an amplification of KRAS wildtype, amplification of the KRAS gene or overexpression of KRAS.

In a further aspect the invention relates to a compound of the invention—or a pharmaceutically acceptable salt thereof—for use in the treatment and/or prevention of cancer, wherein the cancer comprises a KRAS G12D mutation.

In a further aspect the invention relates to a compound of the invention—or a pharmaceutically acceptable salt thereof—for use in the treatment and/or prevention of cancer, wherein the cancer comprises a KRAS G12V mutation.

In a further aspect the invention relates to a compound of the invention—or a pharmaceutically acceptable salt thereof—for use in the treatment and/or prevention of cancer, wherein the cancer comprises a KRAS G13D mutation.

In a further aspect the invention relates to a compound of the invention—or a pharmaceutically acceptable salt thereof—for use in the treatment and/or prevention of cancer, wherein the cancer comprises wildtype amplified KRAS.

In a further aspect the invention relates to a compound of the invention—or a pharmaceutically acceptable salt thereof—for use in a method of inhibiting or degrading KRAS, wherein KRAS can be wild-type, mutated or amplified as defined herein.

In a further aspect the invention relates to the use of a compound of the invention—or a pharmaceutically acceptable salt thereof—in the manufacture of a medicament for use in a method of inhibiting or degrading KRAS, wherein KRAS can be wild-type, amplified or mutated as herein defined.

In a further aspect the invention relates to a method for inhibiting or degrading KRAS, wherein KRAS can be wild-type, amplified or mutated as herein defined, comprising administering a therapeutically effective amount of a compound of the invention—or a pharmaceutically acceptable salt thereof—to a human being.

In a further aspect the invention relates to a compound the invention—or a pharmaceutically acceptable salt thereof—for use in a method of degrading or inducing degradation of KRAS, wherein KRAS can be wild-type, amplified or mutated as herein defined above.

In a further aspect the invention relates to the use of a compound of the invention—or a pharmaceutically acceptable salt thereof—in the manufacture of a medicament for use in a method of degrading or inducing degradation of KRAS, wherein KRAS can be wild-type, amplified or mutated as herein defined.

In a further aspect the invention relates to a method for degrading or inducing degradation of KRAS, wherein KRAS can be wild-type, amplified or mutated as herein defined, comprising administering a therapeutically effective amount of a compound of the invention—or a pharmaceutically acceptable salt thereof—to a human being.

Another aspect is based on identifying a link between the KRAS status of a patient and potential susceptibility to treatment with a compound of the invention—or a pharmaceutically acceptable salt thereof. A KRAS inhibitor or degrader, such as a compound of the invention—or a pharmaceutically acceptable salt thereof—may then advantageously be used to treat patients with a disease dependent on KRAS, who may be resistant to other therapies. This therefore provides opportunities, methods and tools for selecting patients for treatment with a compound of the invention, particularly cancer patients. The selection is based on whether the tumor cells to be treated possess wild-type, preferably amplified, or KRAS mutated at residue 12, preferably G12C, G12D or G12V gene, or KRAS mutated at residue 13, preferably G13D gene. The KRAS gene status could therefore be used as a biomarker to indicate that selecting treatment with a compound of the invention may be advantageous.

According to one aspect, there is provided a method for selecting a patient for treatment with a compound of the invention—or a pharmaceutically acceptable salt thereof, the method comprising
providing a tumor cell-containing sample from a patient;
determining whether the KRAS gene in the patient's tumor cell-containing sample encodes for wild-type (glycine at position 12) or mutant (cysteine, aspartic acid, valine, alanine or aginine at position 12, aspartic acid at position 13, amplification and/or overexpression) KRAS protein; and
selecting a patient for treatment with said compound based thereon.

The method may include or exclude the actual patient sample isolation step.

In one aspect, the patient is selected for treatment with a compound of the invention—or a pharmaceutically acceptable salt thereof—if the tumor cell DNA has a mutant KRAS gene.

According to another aspect, there is provided a compound of the invention—or a pharmaceutically acceptable salt thereof—for use in treating a cancer with tumor cells harbouring a KRAS mutation or an amplification of KRAS wildtype.

According to another aspect, there is provided a compound of the invention—or a pharmaceutically acceptable salt thereof—for use in treating a cancer with tumor cells harbouring a G12C mutant, G12D mutant, G12V mutant or G13D mutant KRAS gene or an amplification of KRAS wildtype.

According to another aspect, there is provided a compound of the invention—or a pharmaceutically acceptable salt thereof—for use in treating a cancer with tumor cells harbouring a G12D mutant KRAS gene.

According to another aspect, there is provided a compound of the invention—or a pharmaceutically acceptable salt thereof—for use in treating a cancer with tumor cells harbouring a G12V mutant KRAS gene.

According to another aspect, there is provided a compound of the invention—or a pharmaceutically acceptable salt thereof—for use in treating a cancer with tumor cells harbouring a G13D mutant KRAS gene.

According to another aspect, there is provided a compound of the invention—or a pharmaceutically acceptable salt thereof—for use in treating a cancer with tumor cells harbouring wildtype amplified KRAS or overexpressed KRAS.

According to another aspect, there is provided a method of treating a cancer with tumor cells harbouring a G12C mutant, G12D mutant, G12V mutant, G12A mutant, G13D mutant or G12R mutant KRAS gene or an amplification of KRAS wildtype gene comprising administering an effective amount of a compound of the invention—or a pharmaceutically acceptable salt thereof—to a human being.

According to another aspect, there is provided a method of treating a cancer with tumor cells harbouring a G12C mutant, G12D mutant, G12V mutant, G12A mutant, G13D mutant or G12R mutant KRAS gene or an amplification of KRAS wildtype gene comprising administering an effective amount of a compound of the invention—or a pharmaceutically acceptable salt thereof.

Determining whether a tumor or cancer comprises a KRAS mutation can be undertaken by assessing the nucleotide sequence encoding the KRAS protein, by assessing the amino acid sequence of the KRAS protein, or by assessing the characteristics of a putative KRAS mutant protein. The sequence of wild-type human KRAS is known in the art. Methods for detecting a mutation in a KRAS nucleotide sequence are known by those of skill in the art. These methods include, but are not limited to, polymerase chain reaction-restriction fragment length polymorphism (PCR-RFLP) assays, polymerase chain reaction-single strand conformation polymorphism (PCR-SSCP) assays, real-time PCR assays, PCR sequencing, mutant allele-specific PCR amplification (MASA) assays, direct sequencing, primer extension reactions, electrophoresis, oligonucleotide ligation assays, hybridization assays, TaqMan assays, SNP genotyping assays, high resolution melting assays and microarray analyses. In some embodiments, samples are evaluated for KRAS mutations by real-time PCR. In real-time PCR, fluorescent probes specific for the KRAS mutation can be used. When a mutation is present, the probe binds and fluorescence is detected. In some embodiments, the KRAS mutation is identified using a direct sequencing method of specific regions (e.g. exon 2 and/or exon 3) in the KRAS gene. This technique will identify all possible mutations in the region sequenced. Methods for detecting a mutation in a KRAS, protein are known by those of skill in the art and may not only be applied to identify presence of mutated/altered KRAS at baseline but also to monitor response to treatment in particular treatment related depletion of WT or mutated KRAS from tumor samples. These methods include, but are not limited to, detection of a KRAS mutant using a binding agent (e.g. an antibody) which may also be specific for the mutant protein, protein electrophoresis, Western blotting, direct peptide sequencing and detection of wild type or mutated KRAS by mass spectrometry-based approaches.

Methods for determining whether a tumor or cancer comprises a KRAS mutation can use a variety of samples. In some embodiments, the sample is taken from a subject having a tumor or cancer. In some embodiments, the sample is a fresh tumor/cancer sample. In some embodiments, the sample is a frozen tumor/cancer sample. In some embodiments, the sample is a formalin-fixed paraffin-embedded sample. In some embodiments, the sample is processed to a cell lysate. In some embodiments, the sample is processed to DNA or RNA. In some embodiments the sample is a liquid biopsy and the test is done on a sample of blood to look for cancer cells from a tumor that are circulating in the blood or for pieces of DNA from tumor cells that are in the blood.

Preferably, the disease/condition/cancer/tumors/cancer cells to be treated/prevented with a compound of the invention—or a pharmaceutically acceptable salt thereof—according to the methods and uses as herein (above and below) defined and disclosed is selected from the group consisting of pancreatic cancer, lung cancer, colorectal cancer, cholangiocarcinoma, appendiceal cancer, multiple myeloma, melanoma, uterine cancer, endometrial cancer, thyroid cancer, acute myeloid leukaemia, bladder cancer, urothelial cancer, gastric cancer, esophageal cancer, gastroesophageal cancer, cervical cancer, head and neck squamous cell carcinoma, diffuse large B cell lymphoma, chronic lymphocytic leukaemia, hepatocellular cancer, breast cancer, ovarian cancer, prostate cancer, glioblastoma, renal cancer and sarcomas.

Preferably, the disease/condition/cancer/tumors/cancer cells to be treated/prevented with a compound of the invention—or a pharmaceutically acceptable salt thereof—according to the methods and uses as herein (above and below) defined and disclosed is selected from the group consisting of: pancreatic cancer, lung cancer, ovarian cancer, colorectal cancer (CRC), gastric cancer, gastroesophageal junction cancer (GEJC) and esophageal cancer. In another aspect, the disease/condition/cancer/tumors/cancer cells to be treated/prevented with a compound of the invention—or a pharmaceutically acceptable salt thereof—according to the methods and uses as herein (above and below) defined and disclosed is selected from the group consisting of pancreatic cancer (preferably pancreatic ductal adenocarcinoma (PDAC)), lung cancer (preferably non-small cell lung cancer (NSCLC)), gastric cancer, cholangiocarcinoma and colorectal cancer (preferably colorectal adenocarcinoma). Preferably, said pancreatic cancer, lung cancer, cholangiocarcinoma, colorectal cancer (CRC), pancreatic ductal adenocarcinoma (PDAC), non-small cell lung cancer (NSCLC) or colorectal adenocarcinoma comprises a KRAS mutation, in particular a KRAS G12D or KRAS G12V mutation. Preferably (in alternative or in combination with the previous preferred embodiment), said non-small cell lung cancer (NSCLC) comprises a mutation (in particular a loss-of-function mutation) in the NF1 gene.

In another aspect, the disease/condition/cancer/tumors/cancer cells to be treated/prevented with a compound of the invention—or a pharmaceutically acceptable salt thereof—according to the methods and uses as herein (above and below) defined and disclosed is gastric cancer, ovarian cancer or esophageal cancer, said gastric cancer or esophageal cancer being preferably selected from the group consisting of: gastric adenocarcinoma (GAC), esophageal adenocarcinoma (EAC) and gastroesophageal junction cancer (GEJC). Preferably, said gastric cancer, ovarian cancer, esophageal cancer, gastric adenocarcinoma (GAC), esophageal adenocarcinoma (EAC) or gastroesophageal junction cancer (GEJC) comprises a KRAS mutation or wildtype amplified KRAS.

Particularly preferred, the cancer to be treated/prevented with a compound of the invention—or a pharmaceutically acceptable salt thereof—according to the methods and uses as herein (above and below) defined and disclosed is selected from the group consisting of:
lung adenocarcinoma (preferably non-small cell lung cancer (NSCLC)) harboring at least one KRAS mutation, in particular KRAS wild type amplification;
colorectal adenocarcinoma harboring at least one KRAS mutation, in particular KRAS wild type amplification;
pancreatic adenocarcinoma (preferably pancreatic ductal adenocarcinoma (PDAC)) harboring at least one KRAS mutation, in particular KRAS wild type amplification;
gastric cancer harboring at least one KRAS mutation, in particular KRAS wild type amplification;
esophageal cancer harboring at least one KRAS mutation, in particular KRAS wild type amplification;
gastroesophageal junction cancer harboring at least one KRAS mutation, in particular KRAS wild type amplification.

Preferably, “cancer” as used herein (above or below) includes drug-resistant cancer and cancer that has failed one, two or more lines of mono- or combination therapy with one or more anti-cancer agents. In particular, “cancer” (and any embodiment thereof) refers to any cancer (especially the cancer species defined hereinabove and hereinbelow) that is resistant to treatment with a KRAS G12C inhibitor.

Different resistance mechanisms have already been reported. For example, the following articles describe resistance in patients following treatment with a KRAS G12C inhibitor: (i) Awad M M, Liu S, Rybkin, II, Arbour K C, Dilly J, Zhu V W, et al. Acquired resistance to KRAS(G12C) inhibition in cancer. N Engl J Med 2021; 384:2382-93 and (ii) Tanaka N, Lin J J, Li C, Ryan M B, Zhang J, Kiedrowski L A, et al. Clinical acquired resistance to KRAS(G12C) inhibition through a novel KRAS switch-II pocket mutation and polyclonal alterations converging on RAS-MAPK reactivation. Cancer Discov 2021; 11:1913-22.

In another aspect the disease/condition/cancer/tumors/cancer cells to be treated/prevented with a compound of the invention—or a pharmaceutically acceptable salt thereof—according to the methods and uses as herein (above and below) defined and disclosed is a RASopathy, preferably selected from the group consisting of Neurofibromatosis type 1 (NF1), Noonan Syndrome (NS), Noonan Syndrome with Multiple Lentigines (NSML) (also referred to as LEOPARD syndrome), Capillary Malformation-Arteriovenous Malformation Syndrome (CM-AVM), Costello Syndrome (CS), Cardio-Facio-Cutaneous Syndrome (CFC), Legius Syndrome (also known as NF1-like Syndrome) and Hereditary gingival fibromatosis.

Additionally, the following cancers, tumors and other proliferative diseases may be treated with compounds of the invention—or a pharmaceutically acceptable salt thereof—without being restricted thereto. Preferably, the methods of treatment, methods, uses, compounds for use and pharmaceutical compositions for use as disclosed herein (above and below) are applied in treatments of diseases/conditions/cancers/tumors which (i.e. the respective cells) harbour a KRAS mutation (including at position 12 (preferably a G12C, G12D, G12V, G12A, G12R mutation) or an amplification of KRAS wild-type) alternatively they have been identified to harbour a KRAS mutation at position 12 (preferably a G12C, G12D, G12V, G12A, G12R mutation) as herein described and/or referred or an amplification of KRAS wildtype:
cancers/tumors/carcinomas of the head and neck: e.g. tumors/carcinomas/cancers of the nasal cavity, paranasal sinuses, nasopharynx, oral cavity (including lip, gum, alveolar ridge, retromolar trigone, floor of mouth, tongue, hard palate, buccal mucosa), oropharynx (including base of tongue, tonsil, tonsillar pilar, soft palate, tonsillar fossa, pharyngeal wall), middle ear, larynx (including supraglottis, glottis, subglottis, vocal cords), hypopharynx, salivary glands (including minor salivary glands);
cancers/tumors/carcinomas of the lung: e.g. non-small cell lung cancer (NSCLC) (squamous cell carcinoma, spindle cell carcinoma, adenocarcinoma, large cell carcinoma, clear cell carcinoma, bronchioalveolar), small cell lung cancer (SCLC) (oat cell cancer, intermediate cell cancer, combined oat cell cancer);
neoplasms of the mediastinum: e.g. neurogenic tumors (including neurofibroma, neurilemoma, malignant schwannoma, neurosarcoma, ganglioneuroblastoma, ganglioneuroma, neuroblastoma, pheochromocytoma, paraganglioma), germ cell tumors (including seminoma, teratoma, non-seminoma), thymic tumors (including thymoma, thymolipoma, thymic carcinoma, thymic carcinoid), mesenchymal tumors (including fibroma, fibrosarcoma, lipoma, liposarcoma, myxoma, mesothelioma, leiomyoma, leiomyosarcoma, rhabdomyosarcoma, xanthogranuloma, mesenchymoma, hemangioma, hemangioendothelioma, hemangiopericytoma, lymphangioma, lymphangiopericytoma, lymphangiomyoma);
cancers/tumors/carcinomas of the gastrointestinal (GI) tract: e.g. tumors/carcinomas/cancers of the esophagus (e.g. esophageal cancer, gastroesophageal junction cancer), stomach (gastric cancer), pancreas, liver and biliary tree (including hepatocellular carcinoma (HCC), e.g. childhood HCC, fibrolamellar HCC, combined HCC, spindle cell HCC, clear cell HCC, giant cell HCC, carcinosarcoma HCC, sclerosing HCC; hepatoblastoma; cholangiocarcinoma; cholangiocellular carcinoma; hepatic cystadenocarcinoma; angiosarcoma, hemangioendothelioma, leiomyosarcoma, malignant schwannoma, fibrosarcoma, Klatskin tumor), gall bladder, extrahepatic bile ducts, small intestine (including duodenum, jejunum, ileum), large intestine (including cecum, colon, rectum, anus; colorectal cancer, gastrointestinal stroma tumor (GIST)), genitourinary system (including kidney, e.g. renal pelvis, renal cell carcinoma (RCC), nephroblastoma (Wilms' tumor), hypernephroma, Grawitz tumor; ureter; urinary bladder, e.g. urachal cancer, urothelial cancer; urethra, e.g. distal, bulbomembranous, prostatic; prostate (androgen dependent, androgen independent, castration resistant, hormone independent, hormone refractory), penis);
cancers/tumors/carcinomas of the testis: e.g. seminomas, non-seminomas, gynecologic cancers/tumors/carcinomas: e.g. tumors/carcinomas/cancers of the ovary, fallopian tube, peritoneum, cervix, vulva, vagina, uterine body (including endometrium, fundus);
cancers/tumors/carcinomas of the breast: e.g. mammary carcinoma (infiltrating ductal, colloid, lobular invasive, tubular, adenocystic, papillary, medullary, mucinous), hormone receptor positive breast cancer (estrogen receptor positive breast cancer, progesterone receptor positive breast cancer), Her2 positive breast cancer, triple negative breast cancer, Paget's disease of the breast;
cancers/tumors/carcinomas of the endocrine system: e.g. tumors/carcinomas/cancers of the endocrine glands, thyroid gland (thyroid carcinomas/tumors; papillary, follicular, anaplastic, medullary), parathyroid gland (parathyroid carcinoma/tumor), adrenal cortex (adrenal cortical carcinoma/tumors), pituitary gland (including prolactinoma, craniopharyngioma), thymus, adrenal glands, pineal gland, carotid body, islet cell tumors, paraganglion, pancreatic endocrine tumors (PET; non-functional PET, PPoma, gastrinoma, insulinoma, VIPoma, glucagonoma, somatostatinoma, GRFoma, ACTHoma), carcinoid tumors;
sarcomas of the soft tissues: e.g. fibrosarcoma, fibrous histiocytoma, liposarcoma, leiomyosarcoma, rhabdomyosarcoma, angiosarcoma, lymphangiosarcoma, Kaposi's sarcoma, glomus tumor, hemangiopericytoma, synovial sarcoma, giant cell tumor of tendon sheath, solitary fibrous tumor of pleura and peritoneum, diffuse mesothelioma, malignant peripheral nerve sheath tumor (MPNST), granular cell tumor, clear cell sarcoma, melanocytic schwannoma, plexosarcoma, neuroblastoma, ganglioneuroblastoma, neuroepithelioma, extraskeletal Ewing's sarcoma, paraganglioma, extraskeletal chondrosarcoma, extraskeletal osteosarcoma, mesenchymoma, alveolar soft part sarcoma, epithelioid sarcoma, extrarenal rhabdoid tumor, desmoplastic small cell tumor; sarcomas of the bone: e.g. myeloma, reticulum cell sarcoma, chondrosarcoma (including central, peripheral, clear cell, mesenchymal chondrosarcoma), osteosarcoma (including parosteal, periosteal, high-grade surface, small cell, radiation-induced osteosarcoma, Paget's sarcoma), Ewing's tumor, malignant giant cell tumor, adamantinoma, (fibrous) histiocytoma, fibrosarcoma, chordoma, small round cell sarcoma, hemangioendothelioma, hemangiopericytoma, osteochondroma, osteoid osteoma, osteoblastoma, eosinophilic granuloma, chondroblastoma;
mesothelioma: e.g. pleural mesothelioma, peritoneal mesothelioma;
cancers of the skin: e.g. basal cell carcinoma, squamous cell carcinoma, Merkel's cell carcinoma, melanoma (including cutaneous, superficial spreading, lentigo maligna, acral lentiginous, nodular, intraocular melanoma), actinic keratosis, eyelid cancer; neoplasms of the central nervous system and brain: e.g. astrocytoma (cerebral, cerebellar, diffuse, fibrillary, anaplastic, pilocytic, protoplasmic, gemistocytary), glioblastoma, gliomas, oligodendrogliomas, oligoastrocytomas, ependymomas, ependymoblastomas, choroid plexus tumors, medulloblastomas, meningiomas, schwannomas, hemangioblastomas, hemangiomas, hemangiopericytomas, neuromas, ganglioneuromas, neuroblastomas, retinoblastomas, neurinomas (e.g. acoustic), spinal axis tumors;
lymphomas and leukemias: e.g. B-cell non-Hodgkin lymphomas (NHL) (including small lymphocytic lymphoma (SLL), lymphoplasmacytoid lymphoma (LPL), mantle cell lymphoma (MCL), follicular lymphoma (FL), diffuse large cell lymphoma (DLCL), Burkitt's lymphoma (BL)), T-cell non-Hodgkin lymphomas (including anaplastic large cell lymphoma (ALCL), adult T-cell leukemia/lymphoma (ATLL), cutaneous T-cell lymphoma (CTCL), peripheral T-cell lymphoma (PTCL)), lymphoblastic T-cell lymphoma (T-LBL), adult T-cell lymphoma, lymphoblastic B-cell lymphoma (B-LBL), immunocytoma, chronic B-cell lymphocytic leukemia (B-CLL), chronic T-cell lymphocytic leukemia (T-CLL) B-cell small lymphocytic lymphoma (B-SLL), cutaneous T-cell lymphoma (CTLC), primary central nervous system lymphoma (PCNSL), immunoblastoma, Hodgkin's disease (HD) (including nodular lymphocyte predominance HD (NLPHD), nodular sclerosis HD (NSHD), mixed-cellularity HD (MCHD), lymphocyte-rich classic HD, lymphocyte-depleted HD (LDHD)), large granular lymphocyte leukemia (LGL), chronic myelogenous leukemia (CML), acute myelogenous/myeloid leukemia (AML), acute lymphatic/lymphoblastic leukemia (ALL), acute promyelocytic leukemia (APL), chronic lymphocytic/lymphatic leukemia (CLL), prolymphocytic leukemia (PLL), hairy cell leukemia, chronic myelogenous/myeloid leukemia (CML), myeloma, plasmacytoma, multiple myeloma (MM), plasmacytoma, myelodysplastic syndromes (MDS), chronic myelomonocytic leukemia (CMML);
cancers of unknown primary site (CUP);

All cancers/tumors/carcinomas mentioned above which are characterized by their specific location/origin in the body are meant to include both the primary tumors and the metastatic tumors derived therefrom.

All cancers/tumors/carcinomas mentioned above may be further differentiated by their histopathological classification:
Epithelial cancers, e.g. squamous cell carcinoma (SCC) (carcinoma in situ, superficially invasive, verrucous carcinoma, pseudosarcoma, anaplastic, transitional cell, lymphoepithelial), adenocarcinoma (AC) (well-differentiated, mucinous, papillary, pleomorphic giant cell, ductal, small cell, signet-ring cell, spindle cell, clear cell, oat cell, colloid, adenosquamous, mucoepidermoid, adenoid cystic), mucinous cystadenocarcinoma, acinar cell carcinoma, large cell carcinoma, small cell carcinoma, neuroendocrine tumors (small cell carcinoma, paraganglioma, carcinoid); oncocytic carcinoma;
Nonepithilial cancers, e.g. sarcomas (fibrosarcoma, chondrosarcoma, rhabdomyosarcoma, leiomyosarcoma, hemangiosarcoma, giant cell sarcoma, lymphosarcoma, fibrous histiocytoma, liposarcoma, angiosarcoma, lymphangiosarcoma, neurofibrosarcoma), lymphoma, melanoma, germ cell tumors, hematological neoplasms, mixed and undifferentiated carcinomas;

The compounds of the invention may be used in therapeutic regimens in the context of first line, second line, or any further line treatments.

The compounds of the invention may be used for the prevention, short-term or long-term treatment of the above-mentioned diseases/conditions/cancers/tumors, optionally also in combination with radiotherapy and/or surgery.

The methods of treatment, methods, uses and compounds for use as disclosed herein (above and below) can be performed with any compound of the invention as disclosed or defined herein and with any pharmaceutical composition or kit comprising a compound of the invention.

### Combination Treatment

The compounds of the invention—or the pharmaceutically acceptable salts thereof—and the pharmaceutical compositions comprising such compounds or salts may also be co-administered with other pharmacologically active substances, e.g. with other anti-neoplastic compounds (e.g. chemotherapy), or used in combination with other treatments, such as radiation or surgical intervention, either as an adjuvant prior to surgery or post-operatively. Preferably, the pharmacologically active substance(s) for co-administration is/are (an) anti-neoplastic compound(s).

Thus, in a further aspect the invention relates to a compound of the invention—or a pharmaceutically acceptable salt thereof—for use as hereinbefore defined wherein said compound is administered before, after or together with one or more other pharmacologically active substance(s).

In a further aspect the invention relates to a compound of the invention—or a pharmaceutically acceptable salt thereof—for use as hereinbefore defined, wherein said compound is administered in combination with one or more other pharmacologically active substance(s).

In a further aspect the invention relates to the use of a compound of the invention—or a pharmaceutically acceptable salt thereof—as hereinbefore defined wherein said compound is to be administered before, after or together with one or more other pharmacologically active substance(s).

In a further aspect the invention relates to a method (e.g. a method for the treatment and/or prevention) as hereinbefore defined wherein the compound of the invention—or a pharmaceutically acceptable salt thereof— is administered before, after or together with a therapeutically effective amount of one or more other pharmacologically active substance(s).

In a further aspect the invention relates to a method (e.g. a method for the treatment and/or prevention) as hereinbefore defined wherein the compound of the invention—or a pharmaceutically acceptable salt thereof— is administered in combination with a therapeutically effective amount of one or more other pharmacologically active substance(s).

In a further aspect the invention relates to a method for the treatment and/or prevention of cancer comprising administering to a patient in need thereof a therapeutically effective amount of a compound of the invention—or a pharmaceutically acceptable salt thereof—and a therapeutically effective amount of one or more other pharmacologically active substance(s), wherein the compound of the invention—or a pharmaceutically acceptable salt thereof— is administered simultaneously, concurrently, sequentially, successively, alternately or separately with the one or more other pharmacologically active substance(s). In a further aspect the invention relates to a method for the treatment and/or prevention of cancer comprising administering to a patient in need thereof a therapeutically effective amount of an inhibitor of a KRAS mutated at residue 12 or 13, such as KRAS G12C, KRAS G12D, KRAS G12V, KRAS G12A, KRAS G13D and/or KRAS G12R inhibitors, preferably KRAS G12C, KRAS G12D or selective KRAS G12D inhibitors—or a pharmaceutically acceptable salt thereof— and a therapeutically effective amount of one or more other pharmacologically active substance(s), wherein the inhibitor—or a pharmaceutically acceptable salt thereof— is administered in combination with the one or more other pharmacologically active substance(s).

In a further aspect the invention relates to a method for the treatment and/or prevention of cancer comprising administering to a patient in need thereof a therapeutically effective amount of a KRAS inhibitor or degrader (preferably a pan-KRAS inhibitor or degrader) and a therapeutically effective amount of one or more other pharmacologically active substance(s), wherein the KRAS inhibitor or degrader (preferably the pan-KRAS inhibitor or degrader) is administered in combination with the one or more other pharmacologically active substance(s).

In a further aspect the invention relates to a compound of the invention—or a pharmaceutically acceptable salt thereof—for use in the treatment and/or prevention of cancer, wherein the compound of the invention—or a pharmaceutically acceptable salt thereof— is administered simultaneously, concurrently, sequentially, successively, alternately or separately with the one or more other pharmacologically active substance(s).

In a further aspect the invention relates to a KRAS inhibitor or degrader (preferably a pan-KRAS inhibitor or degrader) for use in the treatment and/or prevention of cancer, wherein KRAS inhibitor or degrader (preferably a pan-KRAS inhibitor or degrader) is administered in combination with the one or more other pharmacologically active substance(s).

In a further aspect the invention relates to an inhibitor or degrader of KRAS wildtype amplified or overexpressed—or a pharmaceutically acceptable salt thereof—for use in the treatment and/or prevention of cancer, wherein the inhibitor or degrader—or a pharmaceutically acceptable salt thereof— is administered in combination with the one or more other pharmacologically active substance(s).

In a further aspect the invention relates to a kit comprising
a first pharmaceutical composition or dosage form comprising a compound of the invention—or a pharmaceutically acceptable salt thereof— and, optionally, one or more pharmaceutically acceptable excipient(s), and
a second pharmaceutical composition or dosage form comprising another pharmacologically active substance, and, optionally, one or more pharmaceutically acceptable excipient(s),

for use in the treatment and/or prevention of cancer, wherein the first pharmaceutical composition is to be administered simultaneously, concurrently, sequentially, successively, alternately or separately with the second and/or additional pharmaceutical composition or dosage form.

In one aspect such kit for said use comprises a third pharmaceutical composition or dosage form comprising a third pharmaceutical composition or dosage form comprising still another pharmacologically active substance, and, optionally, one or more pharmaceutically acceptable excipient(s).

In a further embodiment of the invention the components (i.e. the combination partners) of the combinations, kits, uses, methods and compounds for use according to the invention (including all embodiments) are administered simultaneously.

In a further embodiment of the invention the components (i.e. the combination partners) of the combinations, kits, uses, methods and compounds for use according to the invention (including all embodiments) are administered concurrently.

In a further embodiment of the invention the components (i.e. the combination partners) of the combinations, kits, uses, methods and compounds for use according to the invention (including all embodiments) are administered sequentially.

In a further embodiment of the invention the components (i.e. the combination partners) of the combinations, kits, uses, methods and compounds for use according to the invention (including all embodiments) are administered successively.

In a further embodiment of the invention the components (i.e. the combination partners) of the combinations, kits, uses, methods and compounds for use according to the invention (including all embodiments) are administered alternately.

In a further embodiment of the invention the components (i.e. the combination partners) of the combinations, kits, uses, methods and compounds for use according to the invention (including all embodiments) are administered separately.

The pharmacologically active substance(s) to be used together/in combination with the compound of the invention—or a pharmaceutically acceptable salt thereof—or in the medical uses, uses, methods of treatment and/or prevention, pharmaceutical compositions as herein (above and below) defined can be selected from any one or more of the following (preferably there is one or two additional pharmacologically active substance used in all these embodiments):
1. an inhibitor of EGFR and/or ErbB2 (HER2) and/or ErbB3 (HER3) and/or ErbB4 (HER4) or of any mutants thereof
   a. irreversible inhibitors: e.g. afatinib, dacomitinib, canertinib, neratinib, avitinib, poziotinib, AV 412, PF-6274484, HKI 357, olmutinib, osimertinib, almonertinib, nazartinib, lazertinib, pelitinib;
   b. reversible inhibitors: e.g. erlotinib, gefitinib, icotinib, sapitinib, lapatinib, varlitinib, vandetanib, TAK-285, AEE788, BMS599626/AC-480, GW 583340;
   c. anti-EGFR antibodies: e.g. necitumumab, panitumumab, cetuximab, amivantamab;
   d. anti-HER2 antibodies: e.g. pertuzumab, trastuzumab, trastuzumab emtansine;
   e. inhibitors of mutant EGFR;
   f. an inhibitor of HER2 with exon 20 mutations;
   g. preferred irreversible inhibitor is afatinib;
   h. preferred anti-EGFR antibody is cetuximab.
2. an inhibitor of MEK and/or of mutants thereof
   a. e.g. trametinib, cobimetinib, binimetinib, selumetinib, refametinib;
   b. preferred is trametinib
   c. a MEK inhibitor as disclosed in WO 2013/136249;
   d. a MEK inhibitor as disclosed in WO 2013/136254
3. an inhibitor of SOS1 and/or of any mutants thereof (i.e. a compound that modulates/inhibits the GEF functionality of SOS1, e.g. by binding to SOS1 and preventing protein-protein interaction between SOS1 and a (mutant) Ras protein, e.g. KRAS)
   a. e.g. BAY-293;
   b. a SOS1 inhibitor as disclosed in WO 2018/115380;
   c. a SOS1 inhibitor as disclosed in WO 2019/122129;
   d. a SOS1 inhibitor as disclosed in WO 2020/180768, WO 2020/180770, WO 2018/172250 and WO 2019/201848.
4. an inhibitor of YAP1, WWTR1, TEAD1, TEAD2, TEAD3 and/or TEAD4
   a. reversible inhibitors of TEAD transcription factors (e.g. disclosed in WO 2018/204532);
   b. irreversible inhibitors of TEAD transcription factors (e.g. disclosed in WO 2020/243423);
   c. protein-protein interaction inhibitors of the YAP/TAZ::TEAD interaction (e.g. disclosed in WO 2021/186324);
   d. inhibitors of TEAD palmitoylation.
5. an oncolytic virus
6. a RAS vaccine
   a. e.g. TG02 (Targovax).
7. a cell cycle inhibitor
   a. e.g. inhibitors of CDK4/6 and/or of any mutants thereof
      i. e.g. palbociclib, ribociclib, abemaciclib, trilaciclib, PF-06873600;
      ii. preferred are palbociclib and abemaciclib;
      iii. most preferred is abemaciclib.
   b. e.g. vinca alkaloids
      i. e.g. vinorelbine.
   c. e.g. inhibitors of Aurora kinase and/or of any mutants thereof
      i. e.g. alisertib, barasertib.
8. an inhibitor of PTK2 (=FAK) and/or of any mutants thereof
   a. e.g. TAE226, BI 853520.
9. an inhibitor of SHP2 and/or of any mutants thereof
   a. e.g. SHP099, TNO155, RMC-4550, RMC-4630, IACS-13909.
10. an inhibitor of PI3 kinase (=PI3K) and/or of any mutants thereof
   a. e.g. inhibitors of PI3Kα and/or of any mutants thereof
      i. e.g. alpelisib, serabelisib, GDC-0077, HH-CYH33, AMG 511, buparlisib, dactolisib, pictilisib, taselisib.
11. an inhibitor of FGFR1 and/or FGFR2 and/or FGFR3 and/or of any mutants thereof
   a. e.g. ponatinib, infigratinib, nintedanib.
12. an inhibitor of AXL and/or of any mutants thereof
13. a taxane
   a. e.g. paclitaxel, nab-paclitaxel, docetaxel;
   b. preferred is paclitaxel.
14. a platinum-containing compound
   a. e.g. cisplatin, carboplatin, oxaliplatin
   b. preferred is oxaliplatin.
15. an anti-metabolite
   a. e.g. 5-fluorouracil, capecitabine, floxuridine, cytarabine, gemcitabine, pemetrexed, combination of trifluridine and tipiracil (=TAS102);
   b. preferred is 5-fluorouracil.
16. an immunotherapeutic agent
   a. e.g. an immune checkpoint inhibitor
      i. e.g. an anti-CTLA4 mAb, anti-PD1 mAb, anti-PD-L1 mAb, anti-PD-L2 mAb, anti-LAG3 mAb, anti-TIM3 mAb;
      ii. preferred is an anti-PD1 mAb;
      iii. e.g. ipilimumab, nivolumab, pembrolizumab, tislelizumab atezolizumab, avelumab, durvalumab, pidilizumab, PDR-001 (=spartalizumab), AMG-404, ezabenlimab;
      iv. preferred are nivolumab, pembrolizumab, ezabenlimab and PDR-001 (=spartalizumab);
      v. most preferred is ezabenlimab, pembrolizumab and nivolumab.
17. a topoisomerase inhibitor
   a. e.g. irinotecan, liposomal irinotecan (nal-IRI), topotecan, etoposide;
   b. most preferred is irinotecan and liposomal irinotecan (nal-IRI).
18. an inhibitor of A-Raf and/or B-Raf and/or C-Raf and/or of any mutants thereof
   a. e.g. encorafenib, dabrafenib, vemurafenib, PLX-8394, RAF-709 (=example 131 in WO 2014/151616), LXH254, sorafenib, LY-3009120 (=example 1 in WO 2013/134243), lifirafenib, TAK-632, agerafenib, CCT196969, RO5126766, RAF265.
19. an inhibitor of mTOR
   a. e.g. rapamycin, temsirolimus, everolimus, ridaforolimus, zotarolimus, sapanisertib, Torin 1, dactolisib, GDC-0349, VS-5584, vistusertib, AZD8055.
20. an epigenetic regulator
   a. e.g. a BET inhibitor
      i. e.g. JQ-1, GSK 525762, OTX-015, CPI-0610, TEN-010, OTX-015, PLX51107, ABBV-075, ABBV-744, BMS986158, TGI-1601, CC-90010, AZD5153, I-BET151, BI 894999.
21. an inhibitor of IGF1/2 and/or of IGF1-R and/or of any mutants thereof
   a. e.g. xentuzumab (antibody 60833 in WO 2010/066868), MEDI-573 (=dusigitumab), linsitinib.
22. an inhibitor of a Src family kinase and/or of any mutants thereof
   a. e.g. an inhibitor of a kinase of the SrcA subfamily and/or of any mutants thereof, i.e. an inhibitor of Src, Yes, Fyn, Fgr and/or of any mutants thereof;
   b. e.g. an inhibitor of a kinase of the SrcB subfamily and/or of any mutants thereof, i.e. an inhibitor of Lck, Hck, Blk, Lyn and/or of any mutants thereof;
   c. e.g. an inhibitor of a kinase of the Frk subfamily and/or of any mutants thereof, i.e. an inhibitor of Frk and/or of any mutants thereof;
   d. e.g. dasatinib, ponatinib, bosutinib, vandetanib, KX-01, saracatinib, KX2-391, SU 6656, WH-4-023.
23. an apoptosis regulator
   a. e.g. an MDM2 inhibitor, e.g. an inhibitor of the interaction between p53 (preferably functional p53, most preferably wt p53) and MDM2 and/or of any mutants thereof;
      i. e.g. HDM-201, NVP-CGM097, RG-7112, MK-8242, RG-7388, SAR405838, AMG-232, DS-3032, RG-7775, APG-115;
      ii. preferred are HDM-201, RG-7388 and AMG-232;
      iii. an MDM2 inhibitor as disclosed in WO 2015/155332;
      iv. an MDM2 inhibitor as disclosed in WO 2016/001376;
      v. an MDM2 inhibitor as disclosed in WO 2016/026937;
      vi. an MDM2 inhibitor as disclosed in WO 2017/060431;
   b. e.g. a PARP inhibitor;
   c. e.g. an MCL-1 inhibitor;
      i. e.g. AZD-5991, AMG-176, AMG-397, S64315, S63845, A-1210477;
24. an inhibitor of c-MET and/or of any mutants thereof
   a. e.g. savolitinib, cabozantinib, foretinib;
   b. MET antibodies, e.g. emibetuzumab, amivantamab;
25. an inhibitor of ERK and/or of any mutants thereof
   a. e.g. ulixertinib, LTT462;
26. an inhibitor of farnesyl transferase and/or of any mutants thereof
   a. e.g. tipifarnib;

In a further embodiment of the (combined) use and method (e.g. method for the treatment and/or prevention) as hereinbefore described one other pharmacologically active substance is to be administered before, after or together with the compound of the invention, wherein said one other pharmacologically active substance is
a SOS1 inhibitor; or
a MEK inhibitor; or
trametinib, or
an anti-PD-1 antibody; or
ezabenlimab; or
cetuximab; or
afatinib; or
standard of care (SoC) in a given indication; or
a PI3 kinase inhibitor; or
an inhibitor of TEAD palmitoylation; or
a YAP/TAZ::TEAD inhibitor.

In a further embodiment of the (combined) use and method (e.g. method for the treatment and/or prevention) as hereinbefore described one other pharmacologically active substance is to be administered in combination with the compound of the invention—or a pharmaceutically acceptable salt thereof—wherein said one other pharmacologically active substance is
a SOS1 inhibitor; or
a MEK inhibitor; or
trametinib; or
an anti-PD-1 antibody; or
ezabenlimab; or
cetuximab; or
afatinib; or
standard of care (SoC) in a given indication; or
a PI3 kinase inhibitor; or
an inhibitor of TEAD palmitoylation; or
a YAP/TAZ::TEAD inhibitor.

In a further aspect of the (combined) use and method (e.g. method for the treatment and/or prevention) as hereinbefore described two other pharmacologically active substances are to be administered before, after or together with the compound of the invention, wherein said two other pharmacologically active substances are
a MEK inhibitor and a SOS1 inhibitor; or
trametinib and a SOS1 inhibitor; or
an anti-PD-1 antibody (preferably ezabenlimab) and an anti-LAG-3 antibody; or
an anti-PD-1 antibody (preferably ezabenlimab) and a SOS1 inhibitor; or
a MEK inhibitor and an inhibitor selected from the group consisting of an EGFR inhibitor and/or ErbB2 (HER2) inhibitor and/or inhibitor of any mutants thereof; or
a SOS1 inhibitor and an inhibitor selected from the group consisting of an EGFR inhibitor and/or ErbB2 (HER2) inhibitor and/or inhibitor of any mutants thereof; or
a MEK inhibitor and afatinib; or
a MEK inhibitor and cetuximab; or
trametinib and afatinib; or
trametinib and cetuximab; or
a SOS1 inhibitor and afatinib; or
a SOS1 inhibitor and cetuximab; or
a SOS1 inhibitor and an inhibitor of TEAD palmitoylation; or
a SOS1 inhibitor and a YAP/TAZ::TEAD inhibitor.

In a further aspect of the (combined) use and method (e.g. method for the treatment and/or prevention) as hereinbefore described two other pharmacologically active substances are to be administered in combination with the compound of the invention wherein said two other pharmacologically active substances are
a MEK inhibitor and a SOS1 inhibitor; or
trametinib and a SOS1 inhibitor; or
an anti-PD-1 antibody (preferably ezabenlimab) and an anti-LAG-3 antibody; or
an anti-PD-1 antibody (preferably ezabenlimab) and a SOS1 inhibitor; or
a MEK inhibitor and an inhibitor selected from the group consisting of an EGFR inhibitor and/or ErbB2 (HER2) inhibitor and/or inhibitor of any mutants thereof; or
a SOS1 inhibitor and an inhibitor selected from the group consisting of an EGFR inhibitor and/or ErbB2 (HER2) inhibitor and/or inhibitor of any mutants thereof; or
a MEK inhibitor and afatinib; or
a MEK inhibitor and cetuximab; or
trametinib and afatinib; or
trametinib and cetuximab; or
a SOS1 inhibitor and afatinib; or
a SOS1 inhibitor and an inhibitor of TEAD palmitoylation; or
a SOS1 inhibitor and a YAP/TAZ::TEAD inhibitor.

Additional pharmacologically active substance(s) which can also be used together/in combination with the compound of the invention—or a pharmaceutically acceptable salt thereof—or in the medical uses, uses, methods of treatment and/or prevention, pharmaceutical compositions, kits as herein (above and below) defined include, without being restricted thereto, hormones, hormone analogues and antihormones (e.g. tamoxifen, toremifene, raloxifene, fulvestrant, megestrol acetate, flutamide, nilutamide, bicalutamide, aminoglutethimide, cyproterone acetate, finasteride, buserelin acetate, fludrocortisone, fluoxymesterone, medroxyprogesterone, octreotide), aromatase inhibitors (e.g. anastrozole, letrozole, liarozole, vorozole, exemestane, atamestane), LHRH agonists and antagonists (e.g. goserelin acetate, luprolide), inhibitors of growth factors and/or of their corresponding receptors (growth factors such as for example platelet derived growth factor (PDGF), fibroblast growth factor (FGF), vascular endothelial growth factor (VEGF), epidermal growth factor (EGF), insuline-like growth factors (IGF), human epidermal growth factor (HER, e.g. HER2, HER3, HER4) and hepatocyte growth factor (HGF) and/or their corresponding receptors), inhibitors are for example (anti-)growth factor antibodies, (anti-)growth factor receptor antibodies and tyrosine kinase inhibitors, such as for example cetuximab, gefitinib, afatinib, nintedanib, imatinib, lapatinib, bosutinib, bevacizumab and trastuzumab); antimetabolites (e.g. antifolates such as methotrexate, raltitrexed, pyrimidine analogues such as 5-fluorouracil (5-FU), ribonucleoside and deoxyribonucleoside analogues, capecitabine and gemcitabine, purine and adenosine analogues such as mercaptopurine, thioguanine, cladribine and pentostatin, cytarabine (ara C), fludarabine); antitumor antibiotics (e.g. anthracyclins such as doxorubicin, doxil (pegylated liposomal doxorubicin hydrochloride, myocet (non-pegylated liposomal doxorubicin), daunorubicin, epirubicin and idarubicin, mitomycin-C, bleomycin, dactinomycin, plicamycin, streptozocin); platinum derivatives (e.g. cisplatin, oxaliplatin, carboplatin); alkylation agents (e.g. estramustin, meclorethamine, melphalan, chlorambucil, busulphan, dacarbazin, cyclophosphamide, ifosfamide, temozolomide, nitrosoureas such as for example carmustin and lomustin, thiotepa); antimitotic agents (e.g. Vinca alkaloids such as for example vinblastine, vindesin, vinorelbin and vincristine; and taxanes such as paclitaxel, docetaxel); angiogenesis inhibitors (e.g. tasquinimod), tubuline inhibitors; DNA synthesis inhibitors, PARP inhibitors, topoisomerase inhibitors (e.g. epipodophyllotoxins such as for example etoposide and etopophos, teniposide, amsacrin, topotecan, irinotecan, mitoxantrone), serine/threonine kinase inhibitors (e.g. PDK 1 inhibitors, Raf inhibitors, A-Raf inhibitors, B-Raf inhibitors, C-Raf inhibitors, mTOR inhibitors, mTORC1/2 inhibitors, PI3K inhibitors, PI3Kα inhibitors, dual mTOR/PI3K inhibitors, STK 33 inhibitors, AKT inhibitors, PLK 1 inhibitors, inhibitors of CDKs, Aurora kinase inhibitors), tyrosine kinase inhibitors (e.g. PTK2/FAK inhibitors), protein protein interaction inhibitors (e.g. IAP inhibitors/SMAC mimetics, Mcl-1, MDM2/MDMX), MEK inhibitors, ERK inhibitors, FLT3 inhibitors, BRD4 inhibitors, IGF-1R inhibitors, TRAILR2 agonists, Bcl-xL inhibitors, Bcl-2 inhibitors (e.g. venetoclax), Bcl-2/Bcl-xL inhibitors, ErbB receptor inhibitors, BCR-ABL inhibitors, ABL inhibitors, Src inhibitors, rapamycin analogs (e.g. everolimus, temsirolimus, ridaforolimus, sirolimus), androgen synthesis inhibitors, androgen receptor inhibitors, DNMT inhibitors, HDAC inhibitors, ANG1/2 inhibitors, CYP17 inhibitors, radiopharmaceuticals, proteasome inhibitors (e.g. carfilzomib), immunotherapeutic agents such as immune checkpoint inhibitors (e.g. CTLA4, PD1, PD-L1, PD-L2, LAG3, and TIM3 binding molecules/immunoglobulins, such as e.g. ipilimumab, nivolumab, pembrolizumab), ADCC (antibody-dependent cell-mediated cytotoxicity) enhancers (e.g. anti-CD33 antibodies, anti-CD37 antibodies, anti-CD20 antibodies), t-cell engagers (e.g. bi-specific T-cell engagers (BiTEs®) like e.g. CD3×BCMA, CD3×CD33, CD3×CD19), PSMA×CD3), tumor vaccines, immunomodulator, e.g. STING agonist, and various chemotherapeutic agents such as amifostin, anagrelid, clodronat, filgrastin, interferon, interferon alpha, leucovorin, procarbazine, levamisole, mesna, mitotane, pamidronate and porfimer.

It is to be understood that the combinations, compositions, kits, methods, uses, pharmaceutical compositions or compounds for use according to this invention may envisage the simultaneous, concurrent, sequential, successive, alternate or separate administration of the active ingredients or components. It will be appreciated that the compound of the invention and the one or more other pharmacologically active substance(s) can be administered formulated either dependently or independently, such as e.g. the compound of the invention and the one or more other pharmacologically active substance(s) may be administered either as part of the same pharmaceutical composition/dosage form or, preferably, in separate pharmaceutical compositions/dosage forms.

In this context, “combination” or “combined” within the meaning of this invention includes, without being limited, a product that results from the mixing or combining of more than one active ingredient and includes both fixed and non-fixed (e.g. free) combinations (including kits) and uses, such as e.g. the simultaneous, concurrent, sequential, successive, alternate or separate use of the components or ingredients. The term “fixed combination” means that the active ingredients are administered to a patient simultaneously in the form of a single entity or dosage. The term “non-fixed combination” means that the active ingredients are administered to a patient as separate entities either simultaneously, concurrently or sequentially with no specific time limits, wherein such administration provides therapeutically effective levels of the compounds in the body of the patient.

The administration of the compound of the invention and the one or more other pharmacologically active substance(s) may take place by co-administering the active components or ingredients, such as e.g. by administering them simultaneously or concurrently in one single or in two or more separate formulations or dosage forms. Alternatively, the administration of the compound of the invention and the one or more other pharmacologically active substance(s) may take place by administering the active components or ingredients sequentially or in alternation, such as e.g. in two or more separate formulations or dosage forms.

For example, simultaneous administration includes administration at substantially the same time. This form of administration may also be referred to as “concomitant” administration. Concurrent administration includes administering the active agents within the same general time period, for example on the same day(s) but not necessarily at the same time. Alternate administration includes administration of one agent during a time period, for example over the course of a few days or a week, followed by administration of the other agent(s) during a subsequent period of time, for example over the course of a few days or a week, and then repeating the pattern for one or more cycles. Sequential or successive administration includes administration of one agent during a first time period (for example over the course of a few days or a week) using one or more doses, followed by administration of the other agent(s) during a second and/or additional time period (for example over the course of a few days or a week) using one or more doses. An overlapping schedule may also be employed, which includes administration of the active agents on different days over the treatment period, not necessarily according to a regular sequence. Variations on these general guidelines may also be employed, e.g. according to the agents used and the condition of the subject.

### Pharmaceutical Compositions—Kits

It is a further object of the invention a pharmaceutical composition comprising a compound of the invention—or a pharmaceutically acceptable salt thereof— and one or more pharmaceutically acceptable excipient(s).

In one aspect, said pharmaceutical composition optionally comprises one or more other pharmacologically active substance(s). Said one or more other pharmacologically active substance(s) may be the pharmacologically active substances or combination partners herein defined.

Suitable pharmaceutical compositions for administering the compounds according to the invention will be apparent to those with ordinary skill in the art and include for example tablets, pills, capsules, suppositories, lozenges, troches, solutions, suspensions—particularly solutions, suspensions or other mixtures for parenteral administration (s.c., i.v., i.m., etc. . . . ) and infusion (injectables)—elixirs, syrups, sachets, emulsions, inhalatives or dispersible powders. The content of the compounds of the invention should be in the range from 0.1 to 90 wt.-%, preferably 0.5 to 50 wt.-% of the composition as a whole, i.e. in amounts which are sufficient to achieve the dosage range specified below. The doses specified may, if necessary, be given several times a day.

Suitable tablets may be obtained, for example, by mixing the compounds of the invention with known pharmaceutically acceptable excipients, for example inert diluents, carriers, disintegrants, adjuvants, surfactants, binders and/or lubricants. The tablets may also comprise several layers.

Coated tablets may be prepared accordingly by coating cores produced analogously to the tablets with excipients normally used for tablet coatings, for example collidone or shellac, gum arabic, talc, titanium dioxide or sugar. To achieve delayed release or prevent incompatibilities the core may also consist of a number of layers. Similarly the tablet coating may consist of a number of layers to achieve delayed release, possibly using the excipients mentioned above for the tablets.

Syrups or elixirs containing one or more compounds of the invention or combinations with one or more other pharmaceutically active substance(s) may additionally contain excipients like a sweetener such as saccharine, cyclamate, glycerol or sugar and a flavour enhancer, e.g. a flavouring such as vanillin or orange extract. They may also contain excipients like suspension adjuvants or thickeners such as sodium carboxymethyl cellulose, wetting agents such as, for example, condensation products of fatty alcohols with ethylene oxide, or preservatives such as p-hydroxybenzoates.

Solutions for injection and infusion are prepared in the usual way, e.g. with the addition of excipients like isotonic agents, preservatives such as p-hydroxybenzoates, or stabilisers such as alkali metal salts of ethylenediamine tetraacetic acid, optionally using emulsifiers and/or dispersants, whilst if water is used as the diluent, for example, organic solvents may optionally be used as solvating agents or dissolving aids, and transferred into injection vials or ampoules or infusion bottles.

Capsules containing one or more compounds of the invention or combinations with one or more other pharmaceutically active substance(s) may for example be prepared by mixing the compounds/active substance(s) with inert excipients such as lactose or sorbitol and packing them into gelatine capsules.

Suitable suppositories may be made for example by mixing with excipients provided for this purpose such as neutral fats or polyethyleneglycol or the derivatives thereof.

Excipients which may be used include, for example, water, pharmaceutically acceptable organic solvents such as paraffins (e.g. petroleum fractions), vegetable oils (e.g. groundnut or sesame oil), mono- or polyfunctional alcohols (e.g. ethanol or glycerol), carriers such as e.g. natural mineral powders (e.g. kaolins, clays, talc, chalk), synthetic mineral powders (e.g. highly dispersed silicic acid and silicates), sugars (e.g. cane sugar, lactose and glucose), emulsifiers (e.g. lignin, spent sulfite liquors, methylcellulose, starch and polyvinylpyrrolidone) and lubricants (e.g. magnesium stearate, talc, stearic acid and sodium lauryl sulfate).

The pharmaceutical compositions are administered by the usual methods, preferably by oral or transdermal route, most preferably by oral route. For oral administration the tablets may of course contain, apart from the above-mentioned excipients, additional excipients such as sodium citrate, calcium carbonate and dicalcium phosphate together with various excipients such as starch, preferably potato starch, gelatine and the like. Moreover, lubricants such as magnesium stearate, sodium lauryl sulfate and talc may be used at the same time for the tabletting process. In the case of aqueous suspensions the active substances may be combined with various flavour enhancers or colourings in addition to the excipients mentioned above.

For parenteral use, solutions of the active substances with suitable liquid excipients may be used.

The dosage range of the compounds of the invention applicable per day is usually from 1 mg to 2000 mg, preferably from 250 to 1250 mg.

However, it may sometimes be necessary to depart from the amounts specified, depending on the body weight, age, the route of administration, severity of the disease, the individual response to the drug, the nature of its formulation and the time or interval over which the drug is administered (continuous or intermittent treatment with one or multiple doses per day). Thus, in some cases it may be sufficient to use less than the minimum dose given above, whereas in other cases the upper limit may have to be exceeded. When administering large amounts it may be advisable to divide them up into a number of smaller doses spread over the day.

Thus, in a further aspect the invention relates to a pharmaceutical composition comprising at least one (preferably one) compound of the invention—or a pharmaceutically acceptable salt thereof— and one or more pharmaceutically acceptable excipient(s).

The compounds of the invention and the pharmaceutical compositions comprising such compound and salts may also be co-administered with other pharmacologically active substances, e.g. with other anti-neoplastic compounds (e.g. chemotherapy), i.e. used in combination (see combination treatment further above).

The elements of such combinations may be administered (whether dependently or independently) by methods customary to the skilled person and as they are used in monotherapy, e.g. by oral, enteral, parenteral (e.g., intramuscular, intraperitoneal, intravenous, transdermal or subcutaneous injection, or implant), nasal, vaginal, rectal, or topical routes of administration and may be formulated, alone or together, in suitable dosage unit formulations containing conventional non-toxic pharmaceutically acceptable excipients appropriate for each route of administration.

The combinations may be administered at therapeutically effective single or divided daily doses. The active components of the combinations may be administered in such doses which are therapeutically effective in monotherapy, or in such doses which are lower than the doses used in monotherapy, but when combined result in a desired (joint) therapeutically effective amount.

However, when the combined use of the two or more active substances or principles leads to a synergistic effect, it may also be possible to reduce the amount of one, more or all of the substances or principles to be administered, while still achieving the desired therapeutic action. This may for example be useful for avoiding, limiting or reducing any unwanted side-effects that are associated with the use of one or more of the substances or principles when they are used in their usual amounts, while still obtaining the desired pharmacological or therapeutic effect.

In a further aspect the invention also relates to a pharmaceutical preparation comprising a compound of the invention—or a pharmaceutically acceptable salt thereof— and one or more (preferably one or two, most preferably one) other pharmacologically active substance(s).

Thus, in a further aspect the invention also relates to a pharmaceutical composition comprising a compound of the invention—or a pharmaceutically acceptable salt thereof—and one or more (preferably one or two, most preferably one) other pharmacologically active substance(s).

Pharmaceutical compositions to be co-administered or used in combination can also be provided in the form of a kit.

Thus, in a further aspect the invention also relates to a kit comprising
a first pharmaceutical composition or dosage form comprising a compound of the invention and, optionally, one or more pharmaceutically acceptable excipient(s), and
a second pharmaceutical composition or dosage form comprising another pharmacologically active substance and, optionally, one or more pharmaceutically acceptable excipient(s).

In one aspect such kit comprises a third pharmaceutical composition or dosage form comprising still another pharmacologically active substance and, optionally, one or more pharmaceutically acceptable excipient(s).

### Definitions

Terms not specifically defined herein should be given the meanings that would be given to them by one of skill in the art in light of the disclosure and the context. As used in the specification, however, unless specified to the contrary, the following terms have the meaning indicated and the following conventions are adhered to.

The use of the prefix C_{x-y}, wherein x and y each represent a positive integer (x<y), indicates that the chain or ring structure, specified and mentioned in direct association, may consist of a maximum of y and a minimum of x carbon atoms.

The indication of the number of members in groups that contain one or more heteroatom(s) (e.g. heterocycloalkyl, ring, etc.) relates to the total number of atoms of all the ring members or the total of all the ring and carbon chain members.

In groups like HO, H₂N, (O)S, (O)₂S, NC (cyano), HOOC, F₃C or the like, the skilled artisan can see the radical attachment point(s) to the molecule from the free valences of the group itself.

In case a compound herein described is depicted in the form of a chemical name and as a formula, in case of any discrepancy the formula shall prevail.

The expression “compound of the invention” and grammatical variants thereof comprises compounds of formula (I), (I*), (I**), (I***), (I****), (Ia), (Ib), (Ic), (Id), (Ie), (If), (Ig), (Ih), (II), (II*), (II**), (II***), (II****), (IIa), (II*a), (IIb), (II*b), (IIc), (II*c), (IId), (II*d), (IIe), (II*e) and (IIf) as well as compounds I-49, I-50 and I-51, including all salts, aspects and preferred embodiments thereof as herein defined. Any reference to a compound of the invention or to a compound of formula (I), (I*), (I**), (I***), (I****), (Ia), (Ib), (Ic), (Id), (Ie), (If), (Ig), (Ih), (II), (II*), (II**), (II***), (II****), (IIa), (II*a), (IIb), (II*b), (IIc), (II*c), (IId), (II*d), (IIe), (II*e) and (IIf) is intended to include a reference to the respective (sub)aspects and embodiments. A dotted line ( [symbol] ) or a squiggly line ( [symbol] ) may be used in sub-formulas to indicate the atom or bond which is connected to the core molecule as defined. In certain cases, the substituent(s) of the core molecule to which the sub-formula is connected to may be specified, in particular on the side of the dotted line or squiggly line opposite to the side of the sub-formula.

The term “C₁₋ₓ-alkyl”, wherein x is an integer selected from 2, 3, 4, 5 or 6, preferably 3, 4, or 6, either alone or in combination with another radical, denotes an acyclic, saturated, branched or linear hydrocarbon radical with 1 to x C atoms. For example the term C₁₋₆-alkyl embraces the radicals H₃C—, H₃C—CH₂—, H₃C—CH₂—CH₂—, H₃C—CH(CH₃)—, H₃C—CH₂—CH₂—CH₂—, H₃C—CH₂—CH(CH₃)—, H₃C—CH(CH₃)—CH₂—, H₃C—C(CH₃)₂—, H₃C—CH₂—CH₂—CH₂—CH₂—, H₃C—CH₂—CH₂—CH(CH₃)—, H₃C—CH₂—CH(CH₃)—CH₂—, H₃C—CH(CH₃)—CH₂—CH₂—, H₃C—CH₂—C(CH₃)₂—, H₃C—C(CH₃)₂—CH₂—, H₃C—CH(CH₃)—CH(CH₃)—, H₃C—CH₂—CH(CH₂CH₃)—, etc. Preferably, C₁₋ₓ-alkyl refers to methyl, iso-propyl or tert-butyl.

The term “C₁₋ₓ-alkylene” wherein x is an integer selected from 2, 3, 4, 5, 6, 7 or 8, preferably 3, 4 or 6, either alone or in combination with another radical, denotes an acyclic, saturated, branched or linear chain divalent alkyl radical containing from 1 to x carbon atoms. For example the term C₁₋₄-alkylene includes —CH₂—, —CH₂—CH₂—, —CH(CH₃)—, —CH₂—CH₂—CH₂—, —C(CH₃)₂—, —CH(CH₂CH₃)—, —CH(CH₃)—CH₂—, —CH₂—CH(CH₃)—, —CH₂—CH₂—CH₂—CH₂—, —CH₂—CH₂—CH(CH₃)—, —CH(CH₃)—CH₂—CH₂—, —CH₂—CH(CH₃)—CH₂—, —CH₂—C(CH₃)₂—, —C(CH₃)₂—CH₂—, —CH(CH₃)—CH(CH₃)—, —CH₂—CH(CH₂CH₃), —CH(CH₂CH₃)—CH₂—, —CH(CH₂CH₂CH₃)—, —CH(CH(CH₃))₂—, —C(CH₃)(CH₂CH₃)—, etc.

By the terms propyl, butyl, pentyl, hexyl, heptyl, octyl, nonyl, decyl etc. without any further definition are meant saturated hydrocarbon groups with the corresponding number of carbon atoms, wherein all isomeric forms are included.

The above definition for alkyl also applies if alkyl is a part of another (combined) group such as for example cyano-C₁₋₆alkylamino or C_{x-y}alkyloxy. C_{x-y}alkyloxy is sometimes abbreviated as C_{x-y}alkoxy.

The term “alkoxylene” denotes a bivalent alkoxy radical.

Unlike alkyl, alkenyl consists of at least two carbon atoms, wherein at least two adjacent carbon atoms are joined together by a C—C double bond and a carbon atom can only be part of one C—C double bond. If in an alkyl as hereinbefore defined having at least two carbon atoms, two hydrogen atoms on adjacent carbon atoms are formally removed and the free valencies are saturated to form a second bond, the corresponding alkenyl is formed.

Examples of alkenyl are vinyl (ethenyl), prop-1-enyl, allyl (prop-2-enyl), isopropenyl, but-1-enyl, but-2-enyl, but-3-enyl, 2-methyl-prop-2-enyl, 2-methyl-prop-1-enyl, 1-methyl-prop-2-enyl, 1-methyl-prop-1-enyl, 1-methylidenepropyl, pent-1-enyl, pent-2-enyl, pent-3-enyl, pent-4-enyl, 3-methyl-but-3-enyl, 3-methyl-but-2-enyl, 3-methyl-but-1-enyl, hex-1-enyl, hex-2-enyl, hex-3-enyl, hex-4-enyl, hex-5-enyl, 2,3-dimethyl-but-3-enyl, 2,3-dimethyl-but-2-enyl, 2-methylidene-3-methylbutyl, 2,3-dimethyl-but-1-enyl, hexa-1,3-dienyl, hexa-1,4-dienyl, penta-1,4-dienyl, penta-1,3-dienyl, buta-1,3-dienyl, 2,3-dimethylbuta-1,3-diene etc.

By the generic terms propenyl, butenyl, pentenyl, hexenyl, butadienyl, pentadienyl, hexadienyl, heptadienyl, octadienyl, nonadienyl, decadienyl etc. without any further definition are meant all the conceivable isomeric forms with the corresponding number of carbon atoms, i.e. propenyl includes prop-1-enyl and prop-2-enyl, butenyl includes but-1-enyl, but-2-enyl, but-3-enyl, 1-methyl-prop-1-enyl, 1-methyl-prop-2-enyl etc.

Alkenyl may optionally be present in the cis or trans or E or Z orientation with regard to the double bond(s).

The above definition for alkenyl also applies when alkenyl is part of another (combined) group such as for example in C_{x-y}alkenylamino or C_{x-y}alkenyloxy.

Unlike alkylene, alkenylene consists of at least two carbon atoms, wherein at least two adjacent carbon atoms are joined together by a C—C double bond and a carbon atom can only be part of one C—C double bond. If in an alkylene as hereinbefore defined having at least two carbon atoms, two hydrogen atoms at adjacent carbon atoms are formally removed and the free valencies are saturated to form a second bond, the corresponding alkenylene is formed.

Examples of alkenylene are ethenylene, propenylene, 1-methylethenylene, butenylene, 1-methylpropenylene, 1,1-dimethylethenylene, 1,2-dimethylethenylene, pentenylene, 1,1-dimethylpropenylene, 2,2-dimethylpropenylene, 1,2-dimethylpropenylene, 1,3-dimethylpropenylene, hexenylene etc.

By the generic terms propenylene, butenylene, pentenylene, hexenylene etc. without any further definition are meant all the conceivable isomeric forms with the corresponding number of carbon atoms, i.e. propenylene includes 1-methylethenylene and butenylene includes 1-methylpropenylene, 2-methylpropenylene, 1,1-dimethylethenylene and 1,2-dimethylethenylene.

Alkenylene may optionally be present in the cis or trans or E or Z orientation with regard to the double bond(s).

The above definition for alkenylene also applies when alkenylene is a part of another (combined) group as for example in HO-C_{x-y}alkenyleneamino or H₂N-C_{x-y}alkenyleneoxy.

Unlike alkyl, alkynyl consists of at least two carbon atoms, wherein at least two adjacent carbon atoms are joined together by a C—C triple bond. If in an alkyl as hereinbefore defined having at least two carbon atoms, two hydrogen atoms in each case at adjacent carbon atoms are formally removed and the free valencies are saturated to form two further bonds, the corresponding alkynyl is formed.

Examples of alkynyl are ethynyl, prop-1-ynyl, prop-2-ynyl, but-1-ynyl, but-2-ynyl, but-3-ynyl, 1-methyl-prop-2-ynyl, pent-1-ynyl, pent-2-ynyl, pent-3-ynyl, pent-4-ynyl, 3-methyl-but-1-ynyl, hex-1-ynyl, hex-2-ynyl, hex-3-ynyl, hex-4-ynyl, hex-5-ynyl etc.

By the generic terms propynyl, butynyl, pentynyl, hexynyl, heptynyl, octynyl, nonynyl, decynyl etc. without any further definition are meant all the conceivable isomeric forms with the corresponding number of carbon atoms, i.e. propynyl includes prop-1-ynyl and prop-2-ynyl, butynyl includes but-1-ynyl, but-2-ynyl, but-3-ynyl, 1-methyl-prop-1-ynyl, 1-methyl-prop-2-ynyl, etc.

If a hydrocarbon chain carries both at least one double bond and also at least one triple bond, by definition it belongs to the alkynyl subgroup.

The above definition for alkynyl also applies if alkynyl is part of another (combined) group, as for example in C_{x-y}alkynylamino or C_{x-y}alkynyloxy.

Unlike alkylene, alkynylene consists of at least two carbon atoms, wherein at least two adjacent carbon atoms are joined together by a C—C triple bond. If in an alkylene as hereinbefore defined having at least two carbon atoms, two hydrogen atoms in each case at adjacent carbon atoms are formally removed and the free valencies are saturated to form two further bonds, the corresponding alkynylene is formed.

Examples of alkynylene are ethynylene, propynylene, 1-methylethynylene, butynylene, 1-methylpropynylene, 1,1-dimethylethynylene, 1,2-dimethylethynylene, pentynylene, 1,1-dimethylpropynylene, 2,2-dimethylpropynylene, 1,2-dimethylpropynylene, 1,3-dimethylpropynylene, hexynylene etc.

By the generic terms propynylene, butynylene, pentynylene, hexynylene etc. without any further definition are meant all the conceivable isomeric forms with the corresponding number of carbon atoms, i.e. propynylene includes 1-methylethynylene and butynylene includes 1-methylpropynylene, 2-methylpropynylene, 1,1-dimethylethynylene and 1,2-dimethylethynylene.

The above definition for alkynylene also applies if alkynylene is part of another (combined) group, as for example in HO-C_{x-y}alkynyleneamino or H₂N-C_{x-y}alkynyleneoxy.

By heteroatoms are meant oxygen, nitrogen and sulphur atoms.

Haloalkyl is derived from the previously defined alkyl by replacing one or more hydrogen atoms of the hydrocarbon chain independently of one another by halogen atoms, which may be identical or different.

Examples of haloalkyl are —CF₃, —CHF₂, —CH₂F, —CF₂CF₃, —CHFCF₃, —CH₂CF₃, —CF₂CH₃, —CHFCH₃, —CF₂CF₂CF₃, —CF₂CH₂CH₃, —CF═CF₂, —CCl═CH₂, —CBr═CH₂, —C≡C—CF₃, —CHFCH₂CH₃, —CHFCH₂CF₃ etc.

Halogen denotes fluorine, chlorine, bromine and iodine.

C₃₋ₖ-cycloalkyl, wherein k is 4 or 5, either alone or in combination with another radical, denotes a cyclic, saturated, unbranched hydrocarbon radical with 3 to k C atoms. For example the term C₃₋₅-cycloalkyl includes cyclopropyl, cyclobutyl and cyclopentyl. The C₃₋ₖ-cycloalkyl may be linked as a substituent to the molecule via every suitable position of the ring system.

The above definition for cycloalkyl also applies if cycloalkyl is part of another (combined) group as for example in C_{x-y}cycloalkylamino, C_{x-y}cycloalkyloxy or C_{x-y}cycloalkylalkyl.

Heterocycloalkyl means a saturated or unsaturated mono- or polycyclic ring system optionally comprising aromatic rings, containing one or more heteroatoms selected from N, O, S, SO or SO₂ consisting of the specified number of atoms, wherein none of the heteroatoms is part of the aromatic ring (if present). The term “heterocycloalkyl” is intended to include all the possible isomeric forms.

By unsaturated is meant that there is at least one double bond in the ring system in question, but no (hetero)aromatic system is formed.

If a heterocycloalkyl is substituted, the substitutions may take place independently of one another, in the form of mono- or polysubstitutions in each case, on all the hydrogen-carrying carbon and/or nitrogen atoms. Heterocycloalkyl itself may be linked as a substituent to the molecule via every suitable position of the ring system. Substituents on heterocycloalkyl do not count for the number of members of a heterocycloalkyl.

Thus, the term “heterocycloalkyl” includes the following exemplary structures (not depicted as radicals as each form is optionally attached through a covalent bond to any atom so long as appropriate valences are maintained):

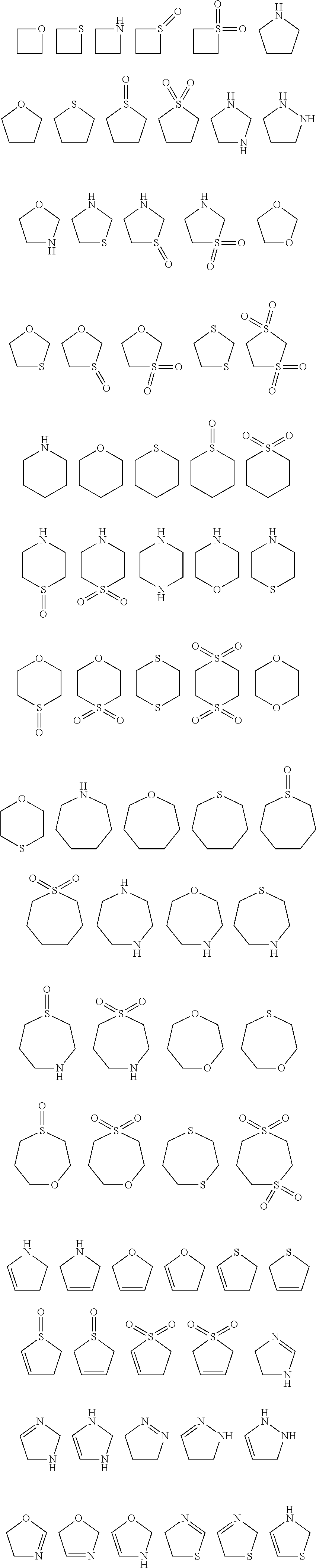

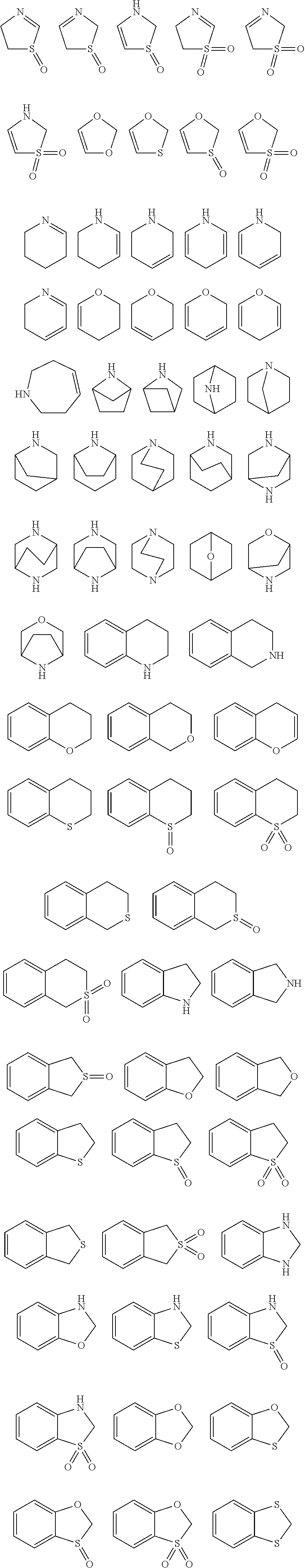

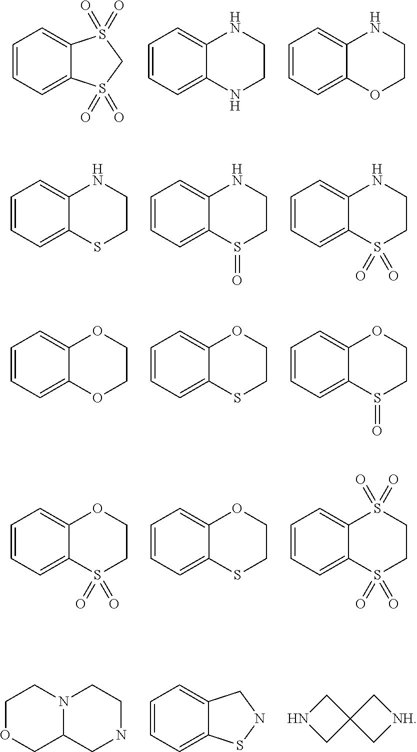

The term heterocycloalkylene is also derived from the previously defined heterocycloalkyl. Heterocycloalkylene, unlike heterocycloalkyl, is bivalent and requires two binding partners. Formally, the second valency is obtained by removing a hydrogen atom from a heterocycloalkylene. Corresponding groups are for example:
piperidinyl and

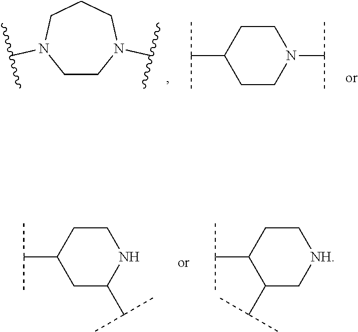

The term heteroaryl means a mono- or polycyclic ring system, comprising at least one aromatic ring, containing one or more heteroatoms selected from N, O, S, SO or SO₂, consisting of 5 to 14 ring atoms wherein at least one of the heteroatoms is part of an aromatic ring. The term “heteroaryl” is intended to include all the possible isomeric forms.

Thus, the term “heteroaryl” includes the following exemplary structures (not depicted as radicals as each form is optionally attached through a covalent bond to any atom so long as appropriate valences are maintained):

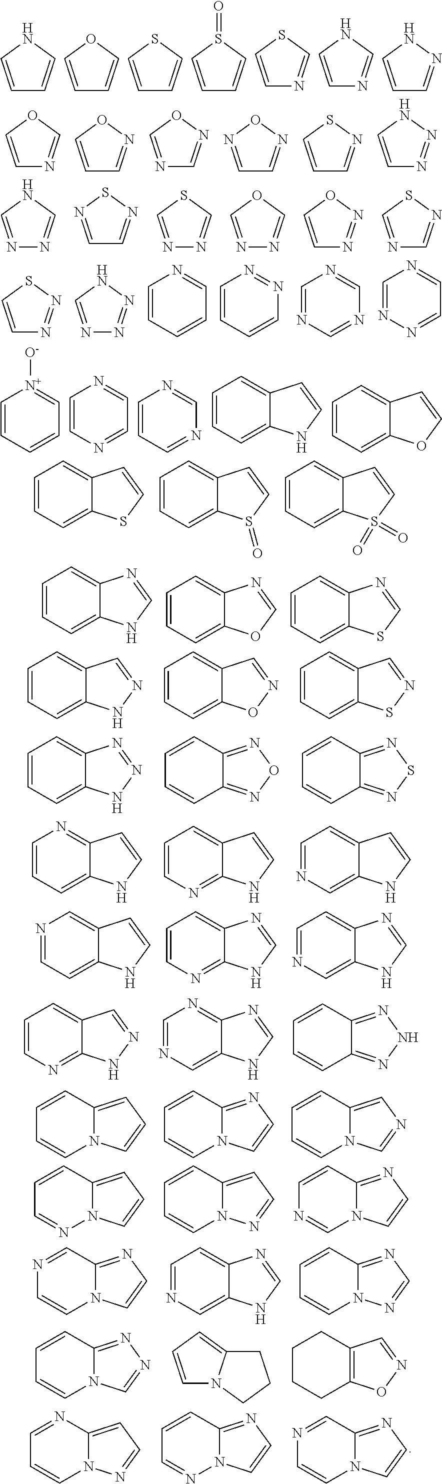

In particular, heteroarylene refers to pyrrole, furan, thiophene, imidazole, pyrazole, oxazole, isoxazole, thiazole, isothiazole, oxadiazole, thiadiazole and triazole, all of which include all the possible isomeric forms and may be linked as a substituent to the molecule via every suitable position of the ring system.

The term heteroarylene is also derived from the previously defined heteroaryl. Heteroarylene, unlike heteroaryl, is bivalent and requires two binding partners. Formally, the second valency is obtained by removing a hydrogen atom from a heteroaryl. Corresponding groups are for example:
pyrrolyl and

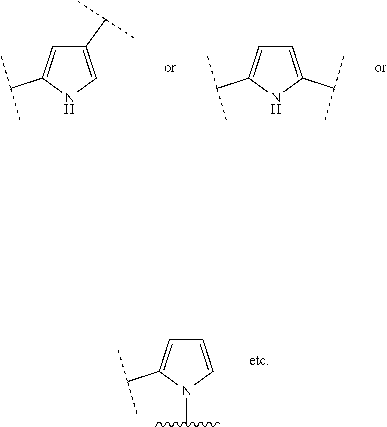

The above definition of heteroarylene also applies if heteroarylene is part of another (combined) group as for example in HO-heteroaryleneamino or H₂N-heteroaryleneoxy.

The term “substituted” as used herein, means that one or more hydrogens on the designated atom are replaced by a group selected from a defined group of substituents, provided that the designated atom's normal valence is not exceeded, and that the substitution results in a stable compound. Likewise, the term “substituted” may be used in connection with a chemical moiety instead of a single atom, e.g. “substituted alkyl”, or the like.

Unless specifically indicated, throughout the specification and the appended claims, a given chemical formula or name shall encompass tautomers and all stereo, optical and geometrical isomers (e.g. enantiomers, diastereomers, E/Z isomers etc. . . . ) and racemates thereof as well as mixtures in different proportions of the separate enantiomers, mixtures of diastereomers, or mixtures of any of the foregoing forms where such isomers and enantiomers exist, as well as salts, including pharmaceutically acceptable salts thereof and solvates thereof such as for instance hydrates including solvates and hydrates of the free compound or solvates and hydrates of a salt of the compound.

Unless specifically indicated, also “pharmaceutically acceptable salts” as defined in more detail below shall encompass solvates thereof such as for instance hydrates.

In general, substantially pure stereoisomers can be obtained according to synthetic principles known to a person skilled in the field, e.g. by separation of corresponding mixtures, by using stereochemically pure starting materials and/or by stereoselective synthesis. It is known in the art how to prepare optically active forms, such as by resolution of racemic forms or by synthesis, e.g. starting from optically active starting materials and/or by using chiral reagents.

Enantiomerically pure compounds of this invention or intermediates may be prepared via asymmetric synthesis, for example by preparation and subsequent separation of appropriate diastereomeric compounds or intermediates which can be separated by known methods (e.g. by chromatographic separation or crystallization) and/or by using chiral reagents, such as chiral starting materials, chiral catalysts or chiral auxiliaries.

Further, it is known to the person skilled in the art how to prepare enantiomerically pure compounds from the corresponding racemic mixtures, such as by chromatographic separation of the corresponding racemic mixtures on chiral stationary phases (this also applies to other mixtures of stereoisomers like mixtures of diasteremers, atropisomers, or others); or by resolution of a racemic mixture using an appropriate resolving agent, e.g. by means of diastereomeric salt formation of the racemic compound with optically active acids or bases, subsequent resolution of the salts and release of the desired compound from the salt; or by derivatization of the corresponding racemic compounds with optically active chiral auxiliary reagents, subsequent diastereomer separation and removal of the chiral auxiliary group; or by kinetic resolution of a racemate (e.g. by enzymatic resolution); by enantioselective crystallization from a conglomerate of enantiomorphous crystals under suitable conditions; or by (fractional) crystallization from a suitable solvent in the presence of an optically active chiral auxiliary.

The phrase “pharmaceutically acceptable” is employed herein to refer to those compounds, materials, compositions, and/or dosage forms which are, within the scope of sound medical judgement, suitable for use in contact with the tissues of human beings and animals without excessive toxicity, irritation, allergic response, or other problem or complication, and commensurate with a reasonable benefit/risk ratio.

As used herein “pharmaceutically acceptable salts” refers to derivatives of the disclosed compounds wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like.

For example, such salts include salts from benzenesulfonic acid, benzoic acid, citric acid, ethanesulfonic acid, fumaric acid, gentisic acid, hydrobromic acid, hydrochloric acid, maleic acid, malic acid, malonic acid, mandelic acid, methanesulfonic acid, 4-methyl-benzenesulfonic acid, phosphoric acid, salicylic acid, succinic acid, sulfuric acid and tartaric acid.

Further pharmaceutically acceptable salts can be formed with cations from ammonia, L-arginine, calcium, 2,2′-iminobisethanol, L-lysine, magnesium, N-methyl-D-glucamine, potassium, sodium and tris(hydroxymethyl)-aminomethane.

The pharmaceutically acceptable salts of the present invention can be synthesized from the parent compound which contains a basic or acidic moiety by conventional chemical methods. Generally, such salts can be prepared by reacting the free acid or base form of these compounds with a sufficient amount of the appropriate base or acid in water or in an organic diluent like ether, ethyl acetate, ethanol, isopropanol, or acetonitrile, or a mixture thereof.

Salts of other acids than those mentioned above which for example are useful for purifying or isolating the compounds of the present invention (e.g. trifluoro acetate salts), also comprise a part of the invention.

In a representation such as for example

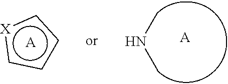

the letter A has the function of a ring designation in order to make it easier, for example, to indicate the attachment of the ring in question to other rings.

For bivalent groups in which it may be useful to determine which adjacent groups they bind and with which valency, the corresponding binding partners can be indicated at the end of a dotted line or in brackets where necessary for clarification purposes, as in the following representations:

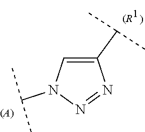

or (R²)—C(═O)NH—or (R²)—NHC(═O)—.

If such a clarification is missing then the bivalent group can bind in both directions, e.g., —C(═O)NH— also includes —NHC(═O)— (and vice versa).

Groups or substituents are frequently selected from among a number of alternative groups/substituents with a corresponding group designation (e.g. R^{a}, R^{b} etc). If such a group is used repeatedly to define a compound according to the invention in different parts of the molecule, it is pointed out that the various uses are to be regarded as totally independent of one another.

By a therapeutically effective amount for the purposes of this invention is meant a quantity of substance that is capable of obviating symptoms of illness or of preventing or alleviating these symptoms, or which prolong the survival of a treated patient.

### EXAMPLES

Other features and advantages of the present invention will become apparent from the following more detailed Examples which illustrate, by way of example, the principles of the invention.

### List of Abbreviations

| | |
|---|---|
| Ac | Acetyl |
| ACN | Acetonitrile |
| aq. | aquatic, aqueous |
| ATP | adenosine triphosphate |
| Bn | Benzyl |
| Boc | tert-butyloxycarbonyl |
| Bu | Butyl |
| C | Concentration |
| C | Concentration |
| Cbz | Carboxybenzyl |
| CDI | 1,1′-carbonyldiimidazole |
| cHexane | cyclohexane |
| D | day(s) |
| d | day(s) |
| TLC | thin layer chromatography |
| Davephos | 2-dimethylamino-2′- |
| | dicyclohexylaminophosphinobiphenyl |
| DBU | 1,8-Diazabicyclo(5.4.0)undec-7-ene |
| DCE | dichloro ethane |
| DCM | dichloro methane |
| de | diastereomeric excess |
| DEA | diethyl amine |
| DIPEA | N-ethyl-N,N-diisopropylamine (Hunig's base) |
| DMA | Dimethylacetamide |
| DMAP | 4-N,N-dimethylaminopyridine |
| DME | 1,2-dimethoxyethane |
| DMF | N,N-dimethylformamide |
| DMSO | Dimethylsulphoxide |
| DPPA | Diphenylphosphorylazide |
| dppf | 1.1′-bis(diphenylphosphino)ferrocene |
| EDCI | 1-ethyl-3-(3-dimethylaminopropyl)carbodiimide |
| EDTA | ethylenediaminetetraacetic acid |
| EGTA | ethyleneglycoltetraacetic acid |
| eq. | equivalent(s) |
| eq | equivalent(s) |
| ESI | electron spray ionization |
| Et | Ethyl |
| Et₂O | diethyl ether |
| EtOAc | ethyl acetate |
| EtOH | Ethanol |
| h | Hour |
| HATU | O-(7-azabenzotriazol-1-yl)- |
| | N,N,N′,N′-tetramethyl-uronium |
| | hexafluorophosphate |
| HOBT | 1-hydroxybenzotriazole hydrate |
| HPLC | high performance liquid chromatography |
| i | Iso |
| conc. | Concentrated |
| LC | liquid chromatography |
| LiHMDS | lithium bis(trimethylsilyl)amide |
| sln. | Solution |
| Me | Methyl |
| MeOH | Methanol |
| min | Minutes |
| MPLC | medium pressure liquid chromatography |
| MS | mass spectrometry |
| MTBE | methyl tert-butyl ether |
| NMM | N-methylmorpholine |
| NMP | N-methylpyrrolidone |
| NP | normal phase |
| n.a. | not available |
| PBS | phosphate-buffered saline |
| Ph | Phenyl |
| Pr | Propyl |
| PTSA | p-toluenesulfonic acid |
| Py | Pyridine |
| rac | Racemic |
| red. | Reduction |
| Rf (R_{f}) | retention factor |
| RP | reversed phase |
| RRLC | Rapid resolution liquid chromatography |
| RT | ambient temperature |
| rt | ambient temperature |
| SFC | supercritical fluid chromatography |
| S_{N} | nucleophilic substitution |
| TBAF | tetrabutylammonium fluoride |
| TBDMS | tert-butyldimethylsilyl |
| TBME | tert-butylmethylether |
| TBTU | O-(benzotriazol-1-yl)-N,N,N′,N′- |
| | tetramethyl-uronium tetrafluoroborate |
| tBu | tert-butyl |
| TEA | triethyl amine |
| temp. | Temperature |
| tert | Tertiary |
| Tf | Triflate |
| TFA | trifluoroacetic acid |
| THF | Tetrahydrofuran |
| TMS | Trimethylsilyl |
| t_{Ret.} | retention time (HPLC) |
| TRIS | tris(hydroxymethyl)-aminomethane |
| TsOH | p-toluenesulphonic acid |
| UPLC | ultra performance liquid chromatography |
| UV | Ultraviolet |
| wt | Weight |

### CHEMICAL EXAMPLES

Unless stated otherwise, all the reactions are carried out in commercially obtainable apparatus using methods that are commonly used in chemical laboratories. Starting materials that are sensitive to air and/or moisture are stored under protective gas and corresponding reactions and manipulations therewith are carried out under protective gas (nitrogen or argon).

If a compound is to be represented both by a structural formula and by its nomenclature, in the event of a conflict the structural formula is decisive.

### Chromatography

The thin layer chromatography is carried out on ready-made silica gel 60 TLC plates on glass (with fluorescence indicator F-254) made by Merck.

The preparative high pressure chromatography (RP HPLC) of the example compounds according to the invention is carried out on Agilent or Gilson systems with columns made by Waters (names: SunFire™ Prep C18, OBD™ 10 μm, 50×150 mm or SunFire™ Prep C18 OBD™ 5 μm, 30×50 mm or XBridge™ Prep C18, OBD™ 10 μm, 50×150 mm or XBridge™ Prep C18, OBD™ 5 μm, 30×150 mm or XBridge™ Prep C18, OBD™ 5 μm, 30×50 mm) and YMC (names: Actus-Triart Prep C18, 5 μm, 30×50 mm) and Chiralpak IE (5 μm, 250×20 mm)

Different gradients of H₂O/acetonitrile are used to elute the compounds, while for Agilent systems 5% acidic modifier (20 mL HCOOH to 1 L H₂O/acetonitrile (1/1)) is added to the water (acidic conditions). For Gilson systems the water is added 0.1% HCOOH.

For the chromatography under basic conditions for Agilent systems H₂O/acetonitrile gradients are used as well, while the water is made alkaline by addition of 5% basic modifier (50 g NH₄HCO₃+50 mL NH₃ (25% in H₂O) to 1 L with H₂O). For Gilson systems the water is made alkaline as follows: 5 mL NH₄HCO₃ solution (158 g in 1 L H₂O) and 2 mL NH₃ (28% in H₂O) are replenished to 1 L with H₂O. The Gilson system was also used under isocratic conditions (60% EtOH/40% EtOH+0.1% DEA)

The supercritical fluid chromatography (SFC) of the intermediates and example compounds according to the invention is carried out on a Agilent 1260 SFC-system, JASCO SFC-system or Sepiatec SFC-system or Waters Thar SFC-System or Waters UPC²-MS SFC-System

with the following colums: Chiralcel OJ (250×20 mm, 5 μm), Chiralpak AD-H (21×250 mm), 5 μm, Chiralpak AD (250×20 mm, 5 μm), Chiralpak AS (250×20 mm, 5 μm), Chiralpak IC (250×20 mm, 5 μm), Chiralpak IA (250×20 mm, 5 μm), Chiralcel OJ (250×20 mm, 5 μm), Chiralcel OD (250×20 mm, 5 μm), Chiralcel OX-3 (150×4.6 mm, 3 μm), Phenomenex Lux C2 (250×20 mm, 5 μm).

### Analytical SFC/UV-Spectrometry Methods

### SFC Method: SFC-1

SFC: Agilent 1260 (binary pump) SFC
Column: Chiralpak AD-H (250×4.6 mm), 5 μm
Flow: 2 ml/min
Mobile Phase: A: CO₂+B: MeOH
ABPR: 120 Bar
Temp: 37.5° C.
UV: 220 nm
Gradient 80% A+20% B (isocratic)
Stop time 10 min

The analytical HPLC (reaction control) of intermediate and final compounds is carried out using columns made by Waters (names: XBridge™ C18, 2.5 μm, 2.1×20 mm or XBridge™ C18, 2.5 μm, 2.1×30 mm or Aquity UPLC BEH C18, 1.7 μm, 2.1×50 mm) and YMC (names: Triart C18, 3.0 μm, 2.0×30 mm) and Phenomenex (names: Luna C18, 5.0 μm, 2.0×30 mm). The analytical equipment is also equipped with a mass detector in each case.

### HPLC-Mass Spectroscopy/UV-Spectrometry

The retention times/MS-ESI⁺ for characterizing the example compounds according to the invention are produced using an HPLC-MS apparatus (high performance liquid chromatography with mass detector). Compounds that elute at the injection peak are given the retention time t_{Ret.}=0.00.

### Method A

| HPLC | Agilent 1100 system | |
|---|---|---|
| MS | 1200Series LC/MSD(API-ES +/− | |
| | 3000 V, Quadrupol, G6140) | |
| MSD signal settings | Scan pos/neg 120-1500 m/z | |
| Detection signal | 315 nm (bandwidth 170 nm, reference off) | |
| Spectrum range | 230-400 nm | |
| Peak width | <0.01 min | |
| Column | Waters, Xbridge C18, 2.5 μm, 2.1 × 20 mm column | |
| Column temperature | 60° C. | |
| Solvent | A: 20 mM aq. NH₄HCO₃/NH3 pH 9 | |
| | B: ACN HPLC grade | |
| Flow | 1.00 mL/min | |
| Gradient | 0.00-1.50 min | 10% to 95% B |
| | 1.50-2.00 min | 95% B |
| | 2.00-2.10 min | 95% to 10% B |

### Method B

| HPLC | Agilent 1260 Series | |
|---|---|---|
| MS | Agilent LC/MSD Quadrupole | |
| Detection | MS: positive and negative mode | |
| Mass range | 100-1200 m/z | |
| Column | Waters X-Bridge BEH C18, | |
| | 2.5 μm, 2.1 × 30 mm XP | |
| Column temperature | 45° C. | |
| Solvent | A: 20 mM NH₄HCO₃/ | |
| | 30 mM NH₃ in H₂O; B: ACN (HPLC | |
| | grade) | |
| Flow | 1.40 mL/min | |
| Gradient | 0.00-1.00 min: | 15% B to 95% B |
| | 1.00-1.30 min: | 95% B |

### Method C

| HPLC | Agilent 1100/1200 system | |
|---|---|---|
| MS | 1200 Series LC/MSD | |
| | (MM-ES + APCI +/− 3000 V, | |
| | Quadrupol, G6130B) | |
| MSD signal settings | Scan pos/neg 150-750 | |
| Detection signal | UV 254 nm, 230 nm, | |
| | 214 nm (bandwidth 8, reference | |
| Spectrum | range: 190-400 nm; slit: 4 nm | |
| Peak width | >0.0031 min (0.063 s response time, 80 Hz) | |
| Column | Waters, Part. No. 186003389, XBridge BEH C18, | |
| | 2.5 μm, 2.1 × 30 mm) column | |
| Column temperature | 45° C. | |
| Solvent | A: 5 mM NH₄HCO₃/ | |
| | 18 mM NH3 in H2O (pH = 9.2) | |
| | B: ACN (HPLC grade) | |
| Flow | 1.4 mL/min | |
| Gradient | 0.0-1.0 min | 15% to 95% B |
| | 1.0-1.1 min | 95% B |

Stop time: 1.3 min

### Method D

| HPLC | Agilent 1100/1200 system | |
|---|---|---|
| MS | 1200 Series LC/MSD (API-ES +/− 3000/3500 V, | |
| | Quadrupol, G6140A) | |
| MSD signal settings | Scan pos/neg 150-750 | |
| Detection signal | UV 254 nm, 230 nm, 214 nm | |
| | (bandwidth 10, reference off) | |
| Spectrum | range: 190-400 nm; slit: 4 nm | |
| Peak width | >0.0031 min (0.063 s response time, 80 Hz) | |
| Column | YMC; Part. No. TA12S03-0302WT; Triart C18, | |
| | 30 × 2.0 mm column | |
| Column temperature | 45° C. | |
| Solvent | A: H2O + 0.11% formic acid | |
| | B: ACN + 0.1% formic acid (HPLC grade) | |
| Flow | 1.4 mL/min | |
| Gradient | 0.0-1.0 min | 15% to 95% B |
| | 1.0-1.1 min | 95% B |

| | |
|---|---|
| Stop time: | 1.23 min |

### Method E

| UPLC-MS | Waters Acquity-UPLC-SQ Detector-2 | |
|---|---|---|
| MSD signal settings | Scan pos & Neg 100-1500, | |
| | Source Voltage: Capillary Vol(kV)- 3.50, Cone(V): | |
| | Source Temp: Desolvation Temp(° C.): 350 | |
| | Source Gas Flow: Desolvation(L/Hr): | |
| | 750, Cone(L/Hr): 50 | |
| Detection signal | Diode Array | |
| Spectrum | Range: 200-400 nm; Resolution: 1.2 nm | |
| Sampling rate | 10 point/sec | |
| Column | AQUITY UPLC BEH C18 1.7 μm, 2.1 × 50 mm | |
| Column temperature | 35° C. | |
| Solvent | A: 0.07% formic acid in ACN | |
| | B: 0.07% formic acid in water | |
| Flow | 0.6 mL/min | |
| Gradient | 0.0-0.30 min | 97% B |
| | 0.30-2.20 min | 97% to 2% B |
| | 2.20-3.30 min | 2% B |
| | 3.30-4.50 min | 2% to 97% B |
| | 4.50-4.51 min | 97% B |

### Method F

| UPLC-MS | Waters Acquity-Binary Solvent Manager- | |
|---|---|---|
| | UPLC-SQ Detector-2 | |
| MSD signal settings | Scan pos & Neg 100-1500, | |
| | Source Voltage: Capillary | |
| | Vol(kV)- 3.50, Cone(V): 50 | |
| | Source Temp: Desolvation Temp(° C.): 350 | |
| | Source Gas Flow: Desolvation(L/Hr): | |
| | 750, Cone(L/Hr): 50 | |
| Detection signal | Diode Array | |
| Spectrum | Range: 200-400 nm; Resolution: 1.2 nm | |
| Sampling rate | 10 point/sec | |
| Column | AQUITY UPLC BEH C18 1.7 μm, 2.1 × 50 mm | |
| Column temperature | 35° C. | |
| Solvent | A: 0.07% formic acid in ACN | |
| | B: 0.07% formic acid in water | |
| Flow | 0.6 mL/min | |
| Gradient | 0.0-0.40 min | 97% B |
| | 0.40-2.50 min | 97% to 2% B |
| | 2.50-3.40 min | 2% B |
| | 3.40-3.50 min | 2% to 97% B |
| | 3.50-4.0 min | 97% B |

### Method G

| | |
|---|---|
| UPLC-MS | Shimadzu series LC-MS 2020 |
| | system with photodiode array |
| | detector |
| MSD signal settings | APCI and ESI positive/negative 100-1000 m/z |
| Detection signal | Photodiode Array |
| Spectrum | Range 200-400 nm; detection wavelength at 254 nm |
| Sampling rate | 40 Hz |
| Column | Hypersil Gold column 1.9 μm |
| | particle size, 2.1 × 50 mm; |
| Column temperature | 40° C. |
| Solvent | A: 0.1% FA in H₂O; B: 0.1% FA in MeCN |
| | B: 0.07% formic acid in water |
| Flow | 0.8 mL/min |
| Gradient | 0.0-3.0 min: 5%-95% B |

### Method H

| HPLC | Agilent 1100/1200 system | |
|---|---|---|
| MS | 1200 Series LC/MSD | |
| | (MM-ES + APCI +/− 3000 V, | |
| | Quadrupol, G6130B) | |
| MSD signal settings | Scan pos 700 - 1350 | |
| Column | Waters, Part. No. 186003389, XBridge | |
| | BEH C18, 2.5 μm, 2.1 × 30 mm) column | |
| eluant | A: 5 mM NH4HCO3/18 mM NH3 (pH = 9.2) | |
| | B: acetonitrile (HPLC grade) | |
| detection signal | UV 254 nm, 230 nm, 214 nm | |
| | (bandwidth 8, reference off) | |
| spectrum | range: 190-400 nm; slit: 4 nm | |
| peak width | >0.0031 min (0.063 s response time, 80 Hz) | |
| injection | 0.5 μL standard injection | |
| flow | 1.4 mL/min | |
| column temperature | 45° C. | |
| gradient | 0.0-1.0 min | 15% → 95% B |
| | 1.0-1.1 min | 95% B |
| Stop time: | 1.3 min | |

### HPLC/UV-Spectrometry

### Method I

| | |
|---|---|
| HPLC | Agilent 1100/1200 system |
| Column | Chiralpak; Part. No. 85394; IE, 5 μm; 150 × 2.1 mm |
| eluant | A: n-Heptan |
| | B: EtOH + 0.1% DEA |
| detection signal | UV 315 nm (bandwidth 170, reference off) |
| spectrum | range: 190-400 nm; slit: 4 nm |
| peak width | >0.0031 min (0.063 s response time, 80 Hz) |
| injection | 0.5 μL standard injection |
| flow | 1.2 mL/min |
| column temperature | 45° C. |
| isocrat | 70% B |
| Stop time: | 5 min |

### Method J

| HPLC | Agilent 1100/1200 system | |
|---|---|---|
| MS | 1200 Series LC/MSD | |
| | (API-ES +/− 3000/3500 V, | |
| | Quadrupol, G6140A) | |
| MSD signal settings | Scan pos/neg 700-1350 | |
| Detection signal | UV 254 nm, 230 nm, 214 nm | |
| | (bandwidth 10, reference off) | |
| Spectrum | range: 190-400 nm; slit: 4 nm | |
| Peak width | >0.0031 min (0.063 s response time, 80 Hz) | |
| Column | YMC; Part. No. TA12S03-0302WT; | |
| | Triart C18, 3 μm, 12 nm; | |
| | 30 × 2.0 mm column | |
| Column temperature | 45° C. | |
| Solvent | A: H2O + 0.11% formic acid | |
| | B: ACN + 0.1% formic acid (HPLC grade) | |
| Flow | 1.4 mL/min | |
| Gradient | 0.0-1.0 min | 15% to 95% B |
| | 1.0-1.1 min | 95% B |

| | |
|---|---|
| Stop time: | 1.23 min |

### Method K

| UPLC-MS | Waters Acquity-Binary Solvent Manager- | |
|---|---|---|
| | UPLC-SQ Detector-2 | |
| MSD signal settings | Scan pos & Neg 100-1500, | |
| | Source Voltage: Capillary | |
| | Vol(kV)- 3.50, Cone(V): 50 | |
| | Source Temp: Desolvation Temp(° C.): 350 | |
| | Source Gas Flow: Desolvation(L/Hr): | |
| | 700, Cone(L/Hr): 50 | |
| Detection signal | Diode Array | |
| Spectrum Range: | 200-400 nm; Resolution: 1.2 nm | |
| Sampling rate | 10 point/sec | |
| Column | AQUITY UPLC BEH C18 1.7 μm, 2.1 × 50 mm | |
| Column temperature | 35° C. | |
| Solvent | A: 0.07% formic acid in ACN | |
| | B: 0.07% formic acid in water | |
| Flow | 0.6 mL/min | |
| Gradient | 0.0-0.40 min | 97% B |
| | 0.40-2.50 min | 97% to 2% B |
| | 2.50-3.40 min | 2% B |
| | 3.40-3.50 min | 2% to 97% B |
| | 3.50-4.0 min | 97% B |

### Method L

| UPLC-MS | Waters Aquity H Class LC system coupled with | |
|---|---|---|
| | ZQ/3100/SQD2 mass spectrometer | |
| MSD signal settings | Scan pos & Neg 100-1500, | |
| Detection signal | Diode Array | |
| Spectrum | Range: 190-400 nm; Resolution: 1.2 nm | |
| Column | YMC triart C18 column (3 μm, 33 × 2.1 mm) | |
| Column temperature | 30° C. | |
| Solvent | A: 5 mM ammonium acetate in water/ | |
| | 0.05% formic acid in water | |
| | B: 5 mM ammonium acetate | |
| | in acetonitrile:water (90:10)/ | |
| | 0.05% formic acid in water | |
| Flow | 1 mL/min | |
| Gradient | 0.0-0.75 min | 2% B |
| | 0.75-1.00 min | 2% to 10% B |
| | 1.00-2.00 min | 10% to 98% B |
| | 2.00-2.50 min | 98% B |
| | 2.50-2.90 min | 98% to 2% B |
| | 2.90-3.00 min | 2% B |

### Method M

HPLC-MS Agilent 1200 HPLC with diode array detector; MS: Agilent 6130 ESI Mass Spectrometer
MSD Signal Settings: ESI positive, 100-1000 m/z
Column: Waters XBridge C18 column, 2.1×50 mm, 3.5 μm particle size
Solvent: A: 0.1% formic acid in water; B: 0.1% formic acid in ACN
Detection Signal: 254 nm, reference off
Spectrum Range: 190-400 nm
Peak Width: <0.01 min
Injection: 4.0 μL standard injection
Column Temperature: 35° C.
Flow: 0.7 ml/min
Gradient: 5-95% B, 3 minutes method

### Method N

HPLC-MS: Agilent 1100 Series; Bruker Microtof
MSD Signal Settings: ESI positive 100-1200 m/z
Column: Waters XBridge C18 column, 2.1×50 mm, 3.5 μm particle size
Solvent: A: 0.1% formic acid in water; B: 0.1% formic acid in ACN
Detection Signal: 254 nm, reference off
Spectrum Range: 190-400 nm
Peak Width: <0.01 min
Injection: 3.0 μL standard injection
Column Temperature: 35° C.
Flow: 0.6 ml/min
Gradient: 5-95% B, 8 minutes method

The compounds according to the present invention and their intermediates may be obtained using methods of synthesis which are known to the one skilled in the art and described in the literature of organic synthesis. Preferably, the compounds are obtained in analogous fashion to the methods of preparation explained more fully hereinafter, in which the substituents of the general formulae have the meanings given hereinbefore. These methods are intended as an illustration of the invention without restricting its subject matter and the scope of the compounds claimed to these examples. In some cases, the order in carrying out the reaction steps may be varied. Variants of the reaction methods that are known to the one skilled in the art but not described in detail here may also be used.

Where the preparation of starting compounds is not described, they are commercially obtainable or their synthesis is described in the prior art or they may be prepared analogously to known prior art compounds or methods described herein, i.e. it is within the skills of an organic chemist to synthesize these compounds. Substances described in the literature can be prepared according to the published methods of synthesis. Any functional groups in the starting materials or intermediates may be protected using conventional protecting groups. These protecting groups may be cleaved again at a suitable stage within the reaction sequence using methods familiar to the one skilled in the art. If a chemical structure in the following is depicted without exact configuration of a stereo center, e.g. of an asymmetrically substituted carbon atom, then both configurations shall be deemed to be included and disclosed in such a representation. The representation of a stereo center in racemic form shall always deem to include and disclose both enantiomers (if no other defined stereo center exists) or all other potential diastereomers and enantiomers (if additional, defined or undefined, stereo centers exist).

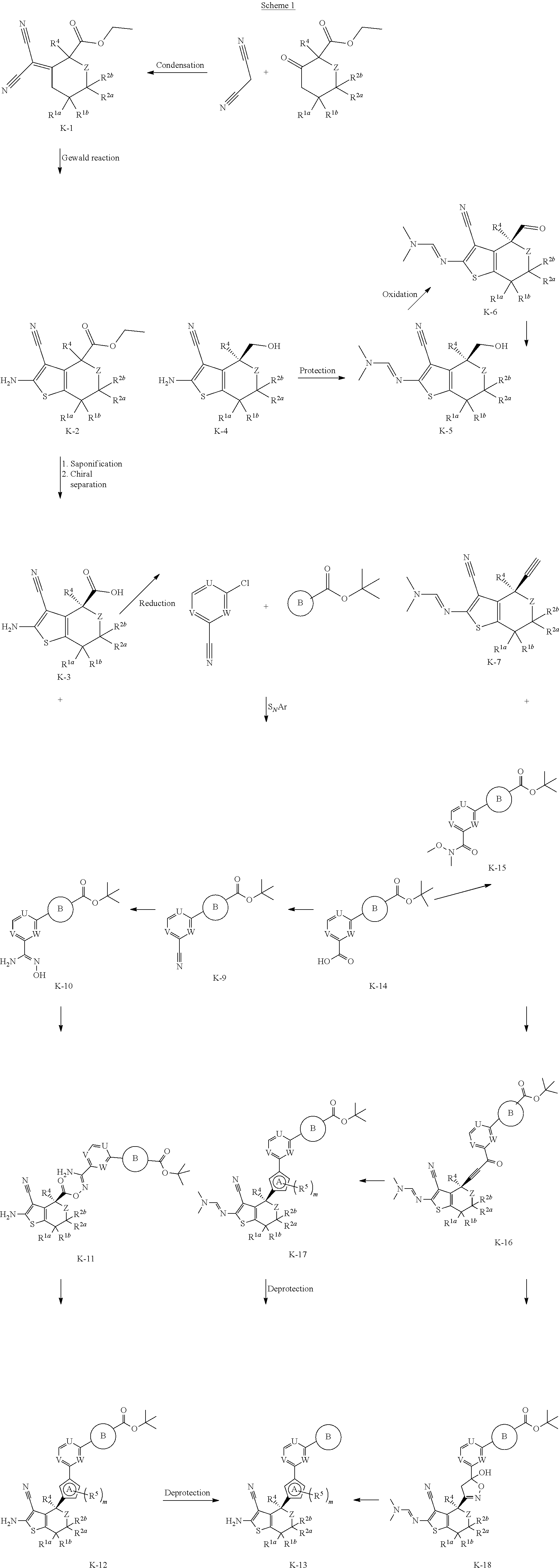

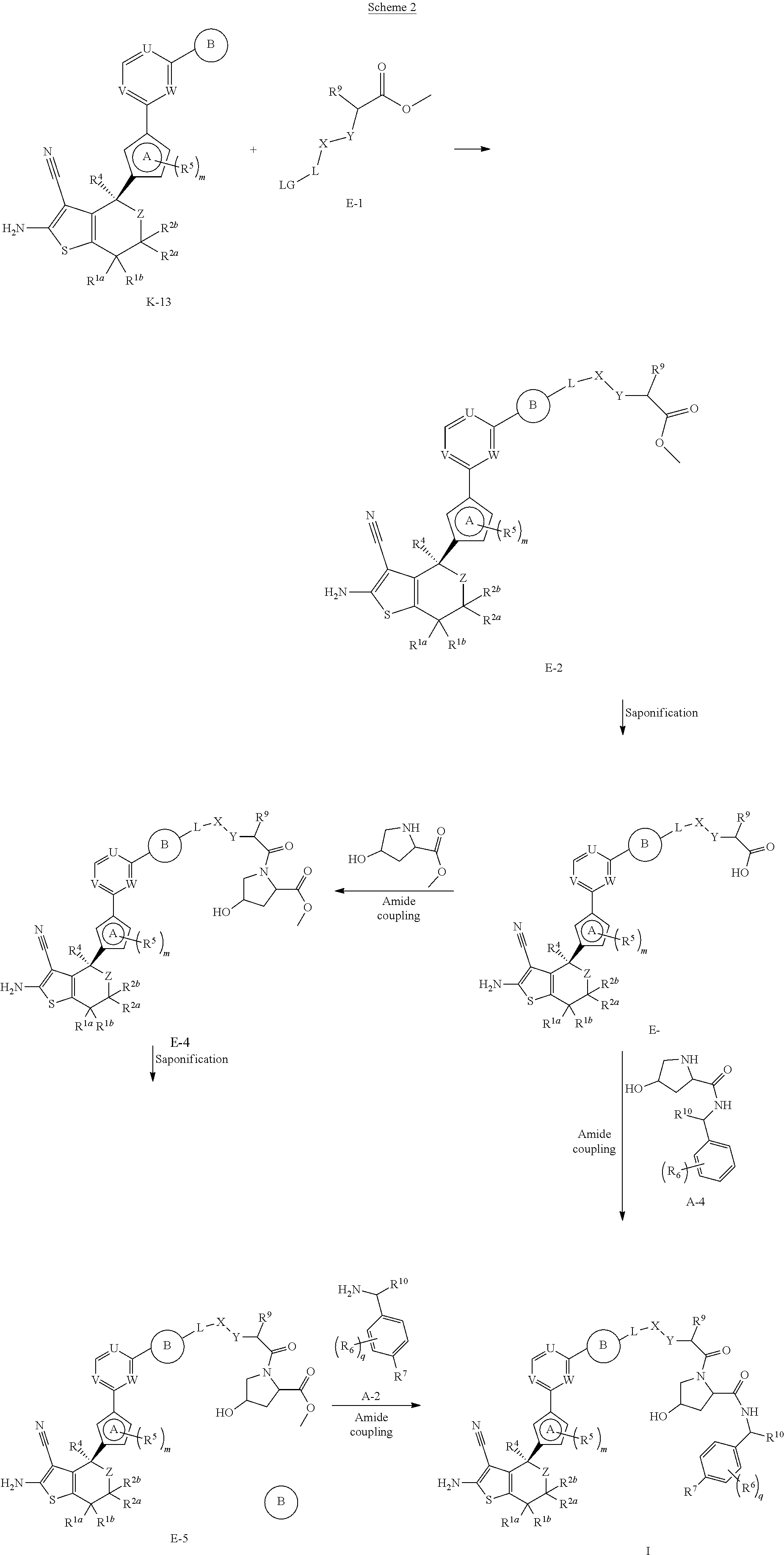

### Experimental Procedure for the Synthesis of K-1a

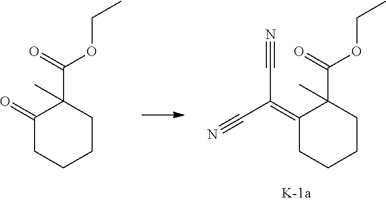 [SMILES: CCOC(=O)C1(C)CCCCC1=O ; CCOC(=O)C1(C)CCCCC1=C(C#N)C#N]

To a solution of ethyl 1-methyl-2-oxocyclohexane-1-carboxylate (108.00 g, 586.2 mmol) in toluene (1.03 L) is added malononitrile (58.04 g, 879.3 mmol, 1.5 eq.) followed by ammonium acetate (9.04 g, 117.2 mmol, 0.2 eq.) and acetic acid (13.41 mL, 234.5 mmol, 0.4 eq.) at rt. The mixture is stirred at 110° C. for 16 h. After complete conversion the mixture is diluted with EtOAc and washed with water and brine, dried over sodium sulfate and concentrated under reduced pressure to afford the crude product K-1a. This crude material is used for the next step without further purification (see also Naumann et al., Pharmazie 51 (1996), 4).

TABLE 1

| | | | | HPLC |
|---|---|---|---|---|
| # | structure | tᵣₑₜ [min] | [M + H]⁺ | method |
| K-1a | 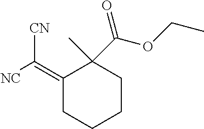 | n.a. | n.a. | — |

### Experimental Procedure for the Synthesis of K-2a

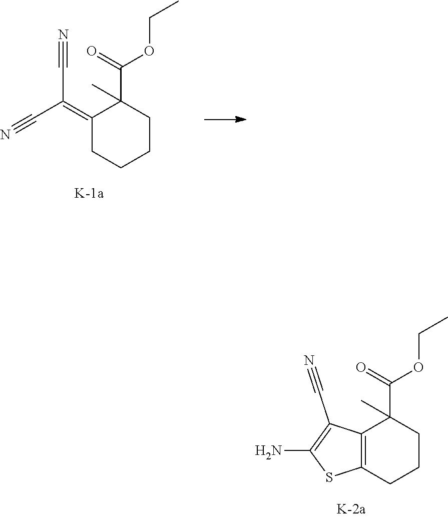 [SMILES: CCOC(=O)C1(C)CCCCC1=C(C#N)C#N ; CCOC(=O)C1(C)CCCc2sc(N)c(C#N)c21]

To a solution of K-1a (250.0 g, 1.1 mol) in DMF (3.0 L) is added sulphur (68.9 g, 2.2 mol, 2.0 eq.) and L-proline (24.8 g, 0.22 mol, 0.2 eq.) and the resulting mixture is stirred at 80° C. for 12 h. After complete conversion the mixture is partitioned between EtOAc and water and the organic layer is collected. The aqueous layer is further extracted with EtOAc and the combined organic layers are washed with water and brine, dried over sodium sulfate and concentrated under reduced pressure to afford the crude product. The crude product is purified through column chromatography yielding K-2a.

TABLE 2

| | | tᵣₑₜ | [M + | HPLC |
|---|---|---|---|---|
| # | structure | [min] | H]⁺ | method |
| K-2a | 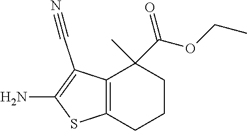 [SMILES: CCOC(=O)C1(C)CCCc2sc(N)c(C#N)c21] | 1.08 | 265 | A |

### Experimental Procedure for the Synthesis of K-3a

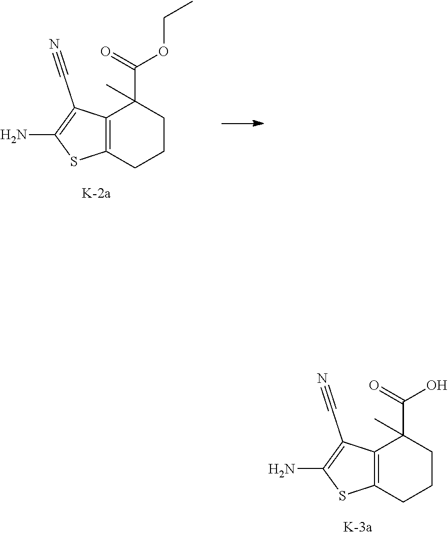 [SMILES: CCOC(=O)C1(C)CCCc2sc(N)c(C#N)c21 ; CC1(C(=O)O)CCCc2sc(N)c(C#N)c21]

K-2a (78.0 mg, 0.3 mmol, 1.0 eq.) is dissolved in EtOH (1.5 mL) and potassium hydroxide (4 M in water, 0.37 mL, 1.5 mmol, 5.0 eq.) is added. The mixture is stirred for 16 h at 78° C. After complete conversion, water and EtOAc is added to the reaction mixture, the pH of the aqueous phase is set to pH 4 using KHSO₄ solution (10% in water), and the product is extracted using EtOAc. The combined organic layers are dried, filtered and concentrated. The crude product is purified via acidic reversed phase chromatography (gradient elution: 20% to 90% acetonitrile in water) yielding K-3a.

The enantiomers can be separated with preparative SFC chromatography. For example K-3a into K-3b and its enantiomer. (Analytical SFC-Method SFC-1: tᵣₑₜ=4.9 min for K-3b, 7.9 min for other enantiomer).

TABLE 3

| | | | | HPLC |
|---|---|---|---|---|
| # | structure | tᵣₑₜ [min] | [M + H]⁺ | method |
| K-3a | 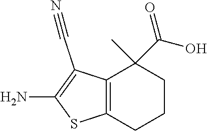 [SMILES: CC1(C(=O)O)CCCc2sc(N)c(C#N)c21] | 0.22 | 237 | A |
| K-3b | 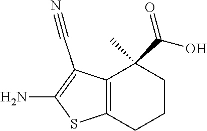 [SMILES: C[C@]1(C(=O)O)CCCc2sc(N)c(C#N)c21] | 0.25 | 237 | A |

### Experimental Procedure for the Synthesis of K-4a

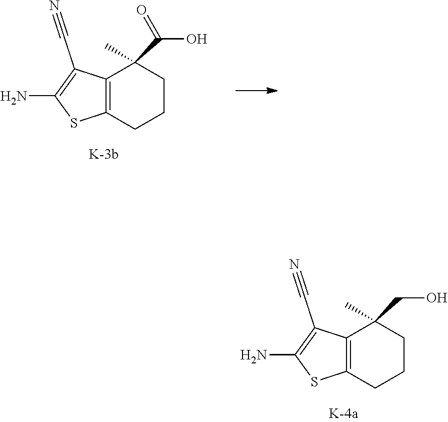 [SMILES: C[C@]1(C(=O)O)CCCc2sc(N)c(C#N)c21 ; C[C@]1(CO)CCCc2sc(N)c(C#N)c21]

To a solution K-3b (22.00 g, 93.11 mmol, 1.0 eq.) in THF (300 mL) is added CDI (17.12 g, 102.42 mmol, 1.1 eq.) and the mixture is stirred at 50° C. for 1 h. The mixture is cooled to rt and sodium borohydride (10.78 g, 279.32 mmol, 3.0 eq.) suspended in 5 mL water is slowly added to the reaction mixture (exothermic reaction). After the addition the mixture is stirred for 1 h and subsequently quenched by slow addition of water (250 mL). The THF is removed under vacuum and the resulting mixture is extracted with EtOAc (3×120 mL). The combined organic layer is washed with water (3×100 mL) and the organic layer is dried with MgSO₄. The solvents are removed under vacuum and the crude product K-4a is used for the next steps without further purification.

The following intermediates K-4 (table 4) are available in an analogous manner starting from different intermediates K-3. The crude product K-4 is purified by chromatography if necessary.

TABLE 4

| | | tᵣₑₜ | [M + | |
|---|---|---|---|---|
| # | structure | [min] | H]⁺ | HPLC method |
| K-4a | 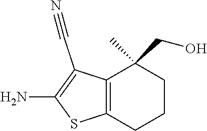 | 0.9 | 223 | A |

### Experimental Procedure for the Synthesis of K-5a

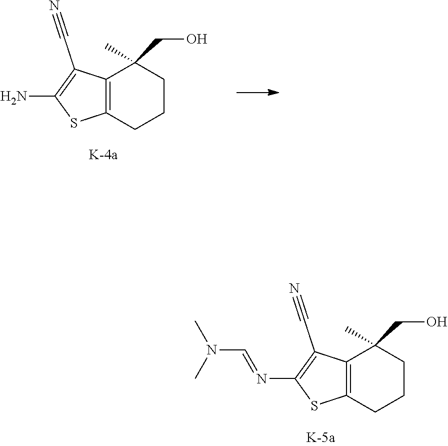 [SMILES: C[C@]1(CO)CCCc2sc(N)c(C#N)c21 ; CN(C)/C=N/c1sc2c(c1C#N)[C@@](C)(CO)CCC2]

K-4a (21.10 g, 75.93 mmol, 80% purity, 1.0 eq.) is mixed with N,N-dimethylformamide dimethyl acetal (57.6 g, 454.37 mmol, 94% purity, 6.0 eq.) and is irradiated in an ultrasound bath for 15 min until the mixture is a clear solution. Water (200 mL) is added and the reaction mixture is stirred for 30 min at rt until a precipitate forms. The precipitate is filtered and water (100 mL) is added. The mixture is irradiated in an ultrasound bath for 15 min and the precipitate is filtered. The precipitate is washed with isopropanol (25 mL) and dried under vacuum at 45° C. over night to give K-5a which is used for the next steps without further purification.

The following intermediates K-5 (table 4*) are available in an analogous manner starting from different intermediates K-4. The crude product K-5 is purified by chromatography if necessary.

TABLE 4*

| # | structure | tᵣₑₜ [min] | [M + H]⁺ | HPLC method |
|---|---|---|---|---|
| K-5a | 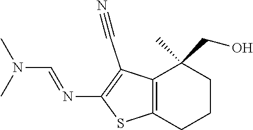 [SMILES: CN(C)/C=N/c1sc2c(c1C#N)[C@@](C)(CO)CCC2] | 1.11 | 278 | A |

### Experimental Procedure for the Synthesis of K-6a

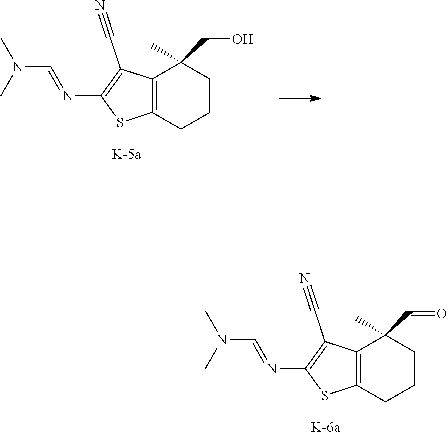 [SMILES: CN(C)/C=N/c1sc2c(c1C#N)[C@@](C)(CO)CCC2 ; CN(C)/C=N/c1sc2c(c1C#N)[C@@](C)(C=O)CCC2]

A solution of oxalyl chloride (12.2 mL, 144.20 mmol, 2.5 eq.) in DCM (120 mL) is cooled to −78° C. A solution of dry DMSO (18.44 mL, 259.57 mmol, 4.5 eq.) in DCM (60 mL) is added dropwise to the reaction mixture (exothermic reaction). The mixture is stirred for 30 min at −78° C. K-5a (16.00 g, 57.68 mmol, 1.0 eq.) is added slowly to the reaction mixture. The mixture is stirred for 30 min at −78° C. and trimethylamine (71.96 mL, 519.32 mmol, 9.0 eq.) is added dropwise. The reaction mixture is slowly warmed to rt and stirred for additional 2 h. Water and DCM is added to the mixture and the phases are separated. The aqueous layer is extracted two times with DCM and the combined organic layer is washed three times with water. The organic layer is dried with MgSO₄ and the solvents are removed under vacuum to give crude intermediate K-6a which is used without further purification in the next steps. The following intermediates K-6 (table 5) are available in an analogous manner starting from different intermediates K-5. The crude product K-6 is purified by chromatography if necessary.

TABLE 5

| # | structure | tᵣₑₜ [min] | [M + H]⁺ | HPLC method |
|---|---|---|---|---|
| K-6a | 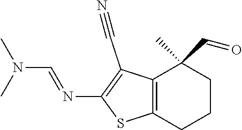 [SMILES: CN(C)/C=N/c1sc2c(c1C#N)[C@@](C)(C=O)CCC2] | 1.21 | 276 | A |

### Experimental Procedure for the Synthesis of K-7a

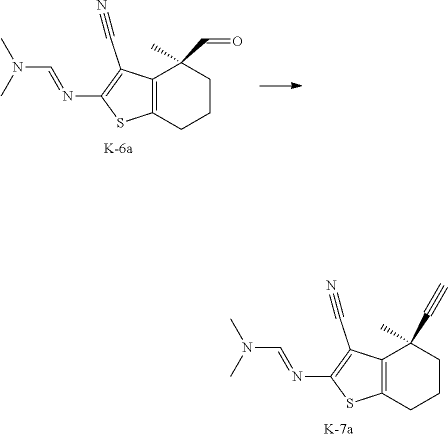 [SMILES: CN(C)/C=N/c1sc2c(c1C#N)[C@@](C)(C=O)CCC2 ; C#C[C@@]1(C)CCCc2sc(/N=C/N(C)C)c(C#N)c21]

A mixture of K-6a (15.90 g, 57.75 mmol, 1.0 eq.), Cs₂CO₃ (22.58 g, 69.26 mmol, 1.2 eq.) and MeOH (120 mL) is cooled to 0° C. and a solution of Bestmann-Ohira reagent (dimethyl (1-diazo-2-oxopropyl)phosphonate; 12.20 g, 63.52 mmol, 1.1 eq.) in MeOH (5 mL) is added dropwise to the reaction mixture. After 3 h at 0° C. the reaction mixture is slowly warmed to rt. After full conversion, the MeOH is removed under vacuum and water (500 mL) and EtOAc (500 mL) are added to the mixture. The phases are separated, and the aqueous layer is extracted two times with EtOAc. The combined organic layer is washed with water three times and dried over MgSO₄ and the solvents are removed under vacuum. The residue is mixed with diethyl ether and stirred for 30 min at rt. The mixture is cooled to 0° C. and stirred for additional 30 min before it is filtered and washed with small amounts of cold diethyl ether. The precipitate is dried under vacuum at 45° C. to give intermediate K-7a which is used for the next steps without further purification.

The following intermediates K-7 (table 6) are available in an analogous manner starting from different intermediates K-6. The crude product K-7 is purified by chromatography if

TABLE 6

| | | tᵣₑₜ | [M + | HPLC |
|---|---|---|---|---|
| # | structure | [min] | H]⁺ | method |
| K-7a | 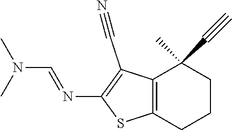 [SMILES: C#C[C@@]1(C)CCCc2sc(/N=C/N(C)C)c(C#N)c21] | 1.33 | 272 | A |

### Experimental Procedure for the Synthesis of K-20a

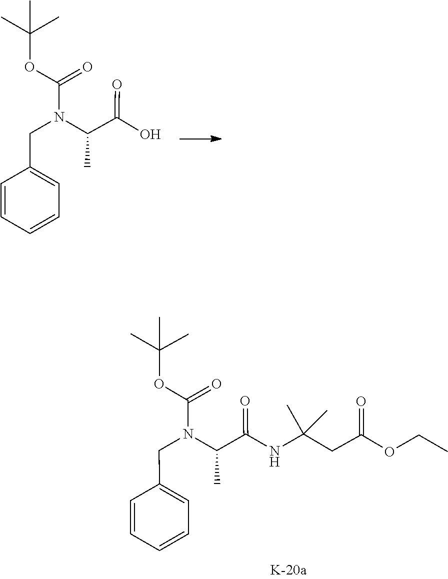 [SMILES: C[C@@H](C(=O)O)N(Cc1ccccc1)C(=O)OC(C)(C)C ; CCOC(=O)CC(C)(C)NC(=O)[C@H](C)N(Cc1ccccc1)C(=O)OC(C)(C)C]

To a stirred solution of (2S)-2-{benzyl[(tert-butoxy)carbonyl]amino}propanoic acid (24.00 g, 85.92 mmol, 1.0 eq.) in DCM (200 mL) is added 1-ethyl-3-(3-dimethylaminopropyl) carbodiimide hydrochloride (24.71 g, 128.88 mmol, 1.5 eq.), 1-hydroxy benzo triazole (19.74 g, 128.88 mmol, 1.5 eq.), and NEt₃ (47.81 mL, 343.67 mmol, 4.0 eq.). The reaction mixture is stirred at rt for 5 min. 3-amino-3-methyl-butyric acid ethyl ester hydrochloride (23.41 g, 128.88 mmol, 1.5 eq.) is added and the reaction mixture is stirred at rt for 16 h. After complete conversion the reaction mixture is diluted with DCM and washed with water. The organic layer is washed with brine, dried over Na₂SO₄ and concentrated under reduced pressure. The crude product is purified by column chromatography to obtain K-20a.

TABLE 7

| # | structure | tᵣₑₜ [min] | [M + H]⁺ | HPLC method |
|---|---|---|---|---|
| K-20a | 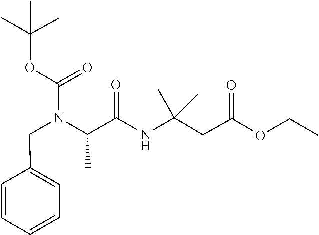 [SMILES: CCOC(=O)CC(C)(C)NC(=O)[C@H](C)N(Cc1ccccc1)C(=O)OC(C)(C)C] | 1.99 | 407 | L |

### Experimental Procedure for the Synthesis of K-21a

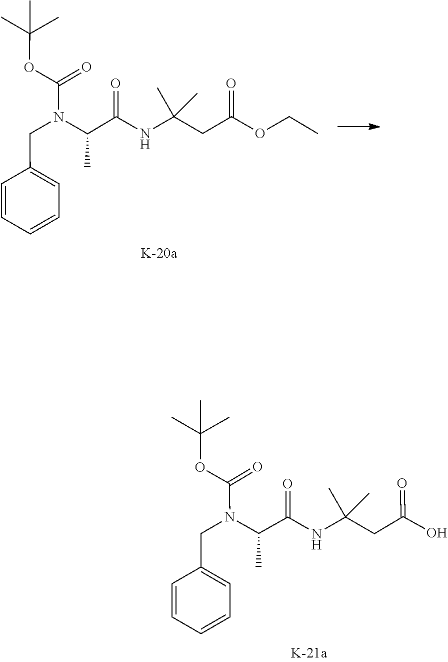

To a stirred solution of K-20a (16.00 g, 39.36 mmol, 1.0 eq.) in THF (40 mL) and water (20 mL) is added lithium hydroxide (2.48 g, 59.04 mmol, 1.5 eq.) and the reaction mixture is stirred at rt for 16 h. After complete conversion the reaction mixture is diluted with water and the organic layer is separated. The aqueous layer is washed with EtOAc. The aqueous layer is acidified with citric acid and extracted with EtOAc. The combined organic layer is dried over Na₂SO₄ and concentrated under reduced pressure to obtain the crude product. The crude product K-21a is used in the next step without further purification.

TABLE 8

| # | structure | tᵣₑₜ [min] | [M + H]⁺ | HPLC method |
|---|---|---|---|---|
| K-21a | 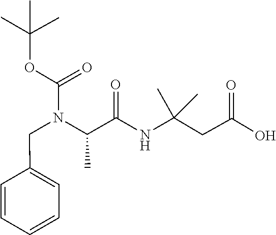 [SMILES: C[C@@H](C(=O)NC(C)(C)CC(=O)O)N(Cc1ccccc1)C(=O)OC(C)(C)C] | 1.60 | 279 | L |

### Experimental Procedure for the Synthesis of K-22a

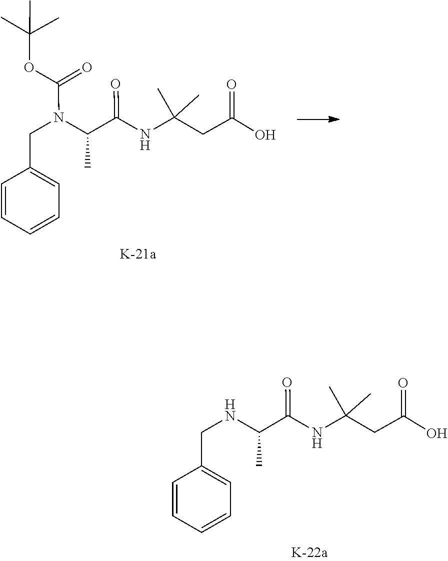 [SMILES: C[C@@H](C(=O)NC(C)(C)CC(=O)O)N(Cc1ccccc1)C(=O)OC(C)(C)C ; C[C@H](NCc1ccccc1)C(=O)NC(C)(C)CC(=O)O]

To a stirred solution of intermediate K-21a (20.00 g, 52.84 mmol, 1.0 eq.) in DCM (100 mL) is added HCl (200 mL, 4 N in 1,4 dioxane) and the reaction mixture is stirred at rt for 4 h. After complete conversion the volatiles are removed under reduced pressure to get the crude product. The crude product is triturated with pentane to afford intermediate K-22a which is used in next step without any other purification.

TABLE 9

| # | structure | tᵣₑₜ [min] | [M + H]⁺ | HPLC method |
|---|---|---|---|---|
| K-22a | 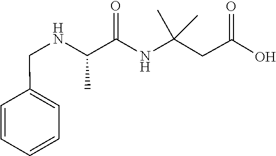 [SMILES: C[C@H](NCc1ccccc1)C(=O)NC(C)(C)CC(=O)O] | 1.30 | 279 | L |

### Experimental Procedure for the Synthesis of K-23a

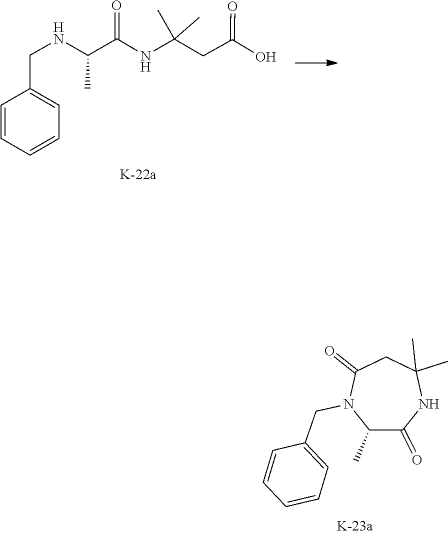 [SMILES: C[C@H](NCc1ccccc1)C(=O)NC(C)(C)CC(=O)O ; C[C@H]1C(=O)NC(C)(C)CC(=O)N1Cc1ccccc1]

To a stirred suspension of TBTU (25.95 g, 80.83 mmol, 1.5 eq.) in DCM (800 mL) is added a solution of intermediate K-22a (15.00 g, 53.89 mmol, 1.0 eq.) and DIPEA (28.73 mL, 161.67 mmol, 3.0 eq.) in DCM (800 mL) slowly at 0° C. and the reaction mixture is allowed to warm to rt and stirred for 16 h. The reaction mixture is quenched with water. The organic layer is separated, dried over Na₂SO₄, filtered, and concentrated under reduce pressure to get the crude product. The crude product is purified by column chromatography to give intermediate K-23a.

TABLE 10

| | | | | HPLC |
|---|---|---|---|---|
| # | structure | tᵣₑₜ [min] | [M + H]⁺ | method |
| K-23a | 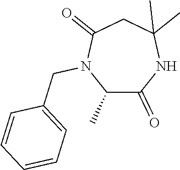 [SMILES: C[C@H]1C(=O)NC(C)(C)CC(=O)N1Cc1ccccc1] | 1.48 | 261 | L |

### Experimental Procedure for the Synthesis of K-24a

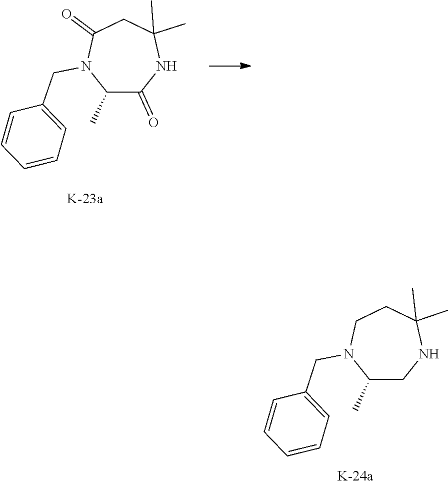 [SMILES: C[C@H]1C(=O)NC(C)(C)CC(=O)N1Cc1ccccc1 ; C[C@H]1CNC(C)(C)CCN1Cc1ccccc1]

To a stirred solution of intermediate K-23a (9.00 g, 34.57 mmol, 1.0 eq.) in THF (50.00 mL) is added lithium aluminum hydride solution (5.26 mL, 138.28 mmol, 4.0 eq, 1 M in THF) at 0° C. and the reaction mixture is allowed to warm to rt and stirred at rt for 10 min. The reaction mixture is heated to 80° C. and stirred for 16 h. The reaction mixture is cooled to 0° C. and carefully quenched by slow addition of saturated Na₂SO₄ solution. The phases are separated, and the aqueous layer is extracted with EtOAc. The combined organic layer is dried over Na₂SO₄, filtered, and concentrated under reduce pressure. The crude is purified by column chromatography to yield intermediate K-24a.

TABLE 11

| | | | | HPLC |
|---|---|---|---|---|
| # | structure | tᵣₑₜ [min] | [M + H]⁺ | method |
| K-24a | 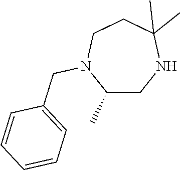 [SMILES: C[C@H]1CNC(C)(C)CCN1Cc1ccccc1] | 1.48 | 233 | L |

### Experimental Procedure for the Synthesis of K-25a

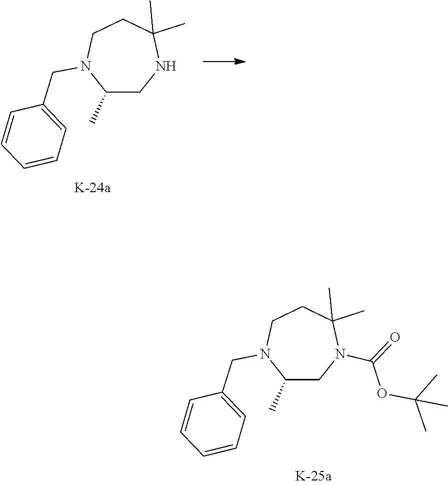 [SMILES: C[C@H]1CNC(C)(C)CCN1Cc1ccccc1 ; C[C@H]1CN(C(=O)OC(C)(C)C)C(C)(C)CCN1Cc1ccccc1]

To a stirred solution of intermediate K-24a (1.00 g, 4.13 mmol, 1.0 eq.) in DCM (10 mL) is added NEt₃ (3.43 mL, 24.78 mmol, 6.0 eq.) and Boc anhydride (2.00 g, 9.08 mmol, 2.2 eq.). The reaction mixture is stirred at rt for 16 h. The reaction mixture is concentrated under reduced pressure, dissolved in acetonitrile and purified by chromatography to yield intermediate K-25a.

TABLE 12

| # | structure | tᵣₑₜ [min] | [M + H]⁺ | HPLC method |
|---|---|---|---|---|
| K-25a | 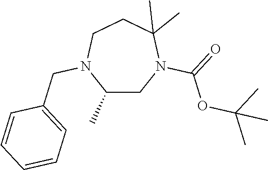 [SMILES: C[C@H]1CN(C(=O)OC(C)(C)C)C(C)(C)CCN1Cc1ccccc1] | 1.74 | 333 | A |

### Experimental Procedure for the Synthesis of K-26a

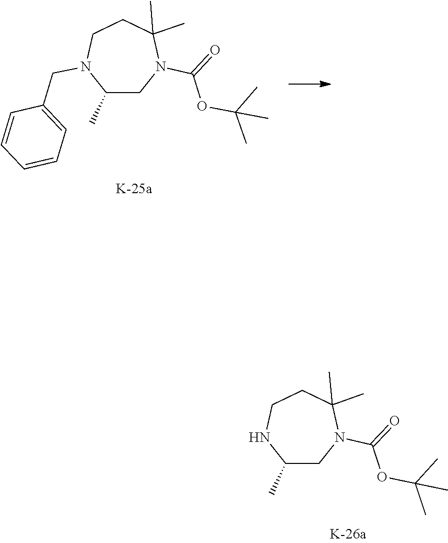 [SMILES: C[C@H]1CN(C(=O)OC(C)(C)C)C(C)(C)CCN1Cc1ccccc1 ; C[C@H]1CN(C(=O)OC(C)(C)C)C(C)(C)CCN1]

To a solution of intermediate K-25a (846 mg, 2.545 mmol, 1.0 eq) in MeOH (40 mL) in a hydrogenation reactor is added Pd/C (10%, 150.00 mg). The reaction mixture stirred under a pressure of 5 bar H₂ for 3 h. After complete conversion the reaction mixture is filtered, and the solvent is removed under reduced pressure to give intermediate K-26a.

TABLE 13

| | | | | HPLC |
|---|---|---|---|---|
| # | structure | tᵣₑₜ [min] | [M + H]⁺ | method |
| K-26a | 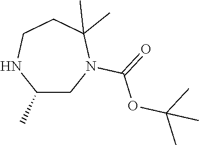 [SMILES: C[C@H]1CN(C(=O)OC(C)(C)C)C(C)(C)CCN1] | 1.09 | 243 | A |

### Experimental Procedure for the Synthesis of K-9a

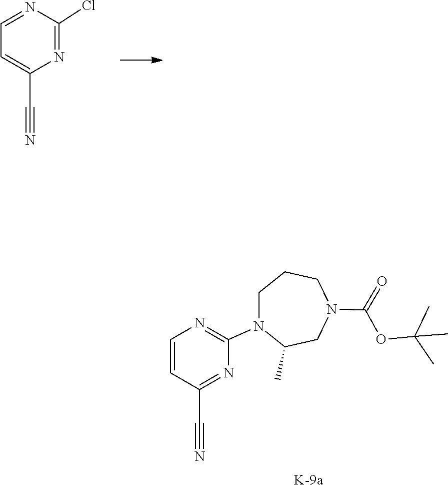 [SMILES: N#Cc1ccnc(Cl)n1 ; C[C@H]1CN(C(=O)OC(C)(C)C)CCCN1c1nccc(C#N)n1]

To a solution of (S)-tert-butyl-3-methyl-1,4-diazepane-1-carboxylate (846.0 mg, 214.30 mmol, 1.0 eq.) and 2-chloropyrimidine-4-carbonitrile (528.9 mg, 139.54 mmol, 1.0 eq) in DMSO (4 ml, 4.5 V) is added TEA (1.1 ml, 101.19 mmol, 2.0 eq.) at rt. The reaction mixture is stirred at 80° C. for 1 h. After complete conversion the reaction mixture is cooled to rt and water and EtOAc is added. The phases are separated. The organic layer is washed with water, dried over sodium sulfate, then filtered and concentrated under reduced pressure to the get crude product which is purified by chromatography to obtain K-9a.

The following intermediates K-9 (table 14) are available in an analogous manner using different nitriles or amines, e.g. K-26a to make K-9d. The crude product K-9 is purified by chromatography if necessary.

TABLE 14

| # | structure | tᵣₑₜ [min] | [M + H]⁺ | HPLC method |
|---|---|---|---|---|
| K-9a | 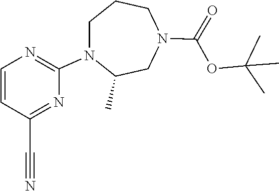 [SMILES: C[C@H]1CN(C(=O)OC(C)(C)C)CCCN1c1nccc(C#N)n1] | 1.38 | 262 [M + H = isobutene]⁺ | A |
| K-9b | 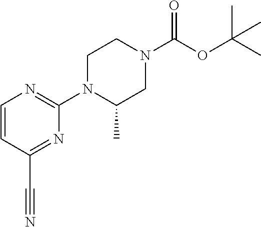 [SMILES: C[C@H]1CN(C(=O)OC(C)(C)C)CCN1c1nccc(C#N)n1] | 1.41 | 248 | A |
| K-9c | 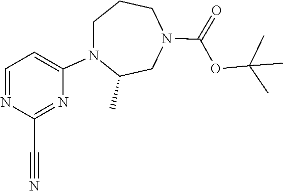 [SMILES: C[C@H]1CN(C(=O)OC(C)(C)C)CCCN1c1ccnc(C#N)n1] | 1.23 | 318 | A |
| K-9d | 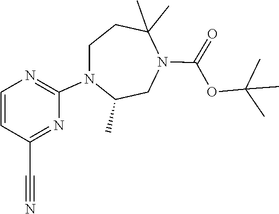 [SMILES: C[C@H]1CN(C(=O)OC(C)(C)C)C(C)(C)CCN1c1nccc(C#N)n1] | 2.39 | 346 | K |

### Experimental Procedure for the Synthesis of K-10a

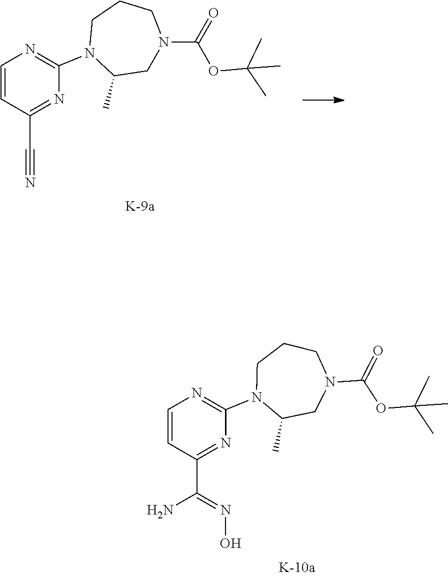 [SMILES: C[C@H]1CN(C(=O)OC(C)(C)C)CCCN1c1nccc(C#N)n1 ; C[C@H]1CN(C(=O)OC(C)(C)C)CCCN1c1nccc(/C(N)=N/O)n1]

To a solution of K-9a (33.85 g, 106.65 mmol, 1.0 eq) in EtOH (270 ml) is added hydroxylamine solution 50% in water (13.05 ml, 213.30 mmol, 2.0 eq) at rt. The reaction mixture is stirred at 60° C. for 1 h. After complete conversion the reaction mixture is concentrated under reduced pressure to afford K-10a which is used for the next step without further purification.

The following intermediates K-10 (table 15) are available in an analogous manner using different nitriles K-9. The crude product K-10 is purified by chromatography if necessary.

TABLE 15

| # | Structure | tᵣₑₜ [min] | [M + H]⁺ | HPLC method |
|---|---|---|---|---|
| K-10a | 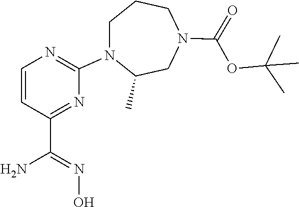 [SMILES: C[C@H]1CN(C(=O)OC(C)(C)C)CCCN1c1nccc(/C(N)=N/O)n1] | 1.12 | 351 | A |
| K-10b | 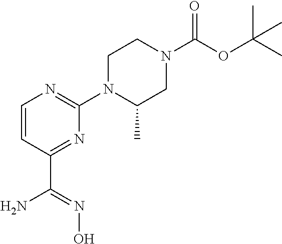 | 1.14 | 337 | A |
| K-10c | 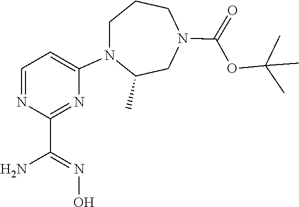 [SMILES: C[C@H]1CN(C(=O)OC(C)(C)C)CCCN1c1ccnc(/C(N)=N/O)n1] | 1.00 | 351 | A |
| K-10d | 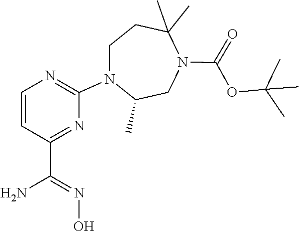 [SMILES: C[C@H]1CN(C(=O)OC(C)(C)C)C(C)(C)CCN1c1nccc(/C(N)=N/O)n1] | 2.22 | 379 | E |

### Experimental Procedure for the Synthesis of K-11a

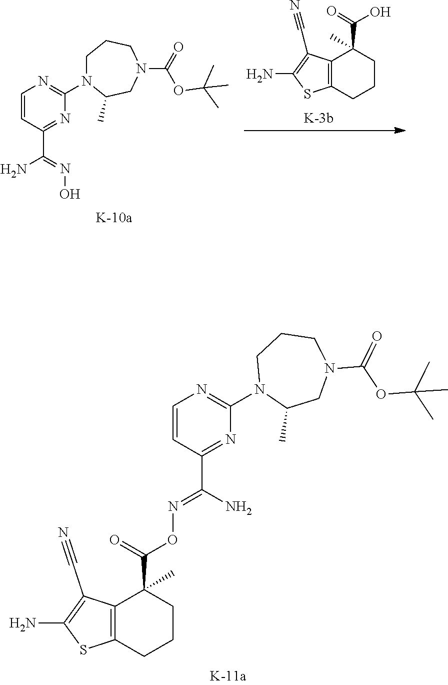 [SMILES: C[C@H]1CN(C(=O)OC(C)(C)C)CCCN1c1nccc(/C(N)=N/O)n1 ; C[C@]1(C(=O)O)CCCc2sc(N)c(C#N)c21 ; C[C@H]1CN(C(=O)OC(C)(C)C)CCCN1c1nccc(/C(N)=N/OC(=O)[C@@]2(C)CCCc3sc(N)c(C#N)c32)n1]

To a stirred solution of K-3b (2.53 g, 10.70 mmol, 1.0 eq.) in DMSO (10 ml) are added TEA (2.17 g, 21.40 mmol, 2.0 eq.) and O-(7-Azabenzotriazol-1-yl)-N,N,N′,N′-tetramethyluronium-hexafluorphosphat (HATU, 4.27 g, 11.24 mmol, 1.10 eq.) at rt. The mixture is stirred for 15 min at rt. K-10a (3.75 g, 10.70 mmol, 1.0 eq) is added at rt and stirred overnight. After complete conversion the reaction mixture is diluted with water and EtOAc. The phases are separated. The organic layer is washed with water, dried over sodium sulfate, filtered, and concentrated under reduced pressure to get the crude product. This crude material is purified by column chromatography to afford K-11a.

The following intermediates K-11 (table 16) are available in an analogous manner using different intermediates K-10. The crude product K-11 is purified by chromatography if necessary.

TABLE 16

| # | Structure | tᵣₑₜ [min] | [M + H]⁺ | HPLC method |
|---|---|---|---|---|
| K-11a | 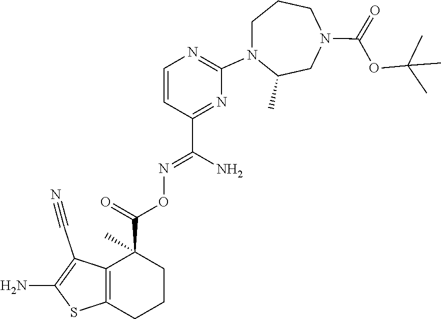 [SMILES: C[C@H]1CN(C(=O)OC(C)(C)C)CCCN1c1nccc(/C(N)=N/OC(=O)[C@@]2(C)CCCc3sc(N)c(C#N)c32)n1] | 1.43 | 513 [M + H − isobutene]⁺ | A |
| K-11b | 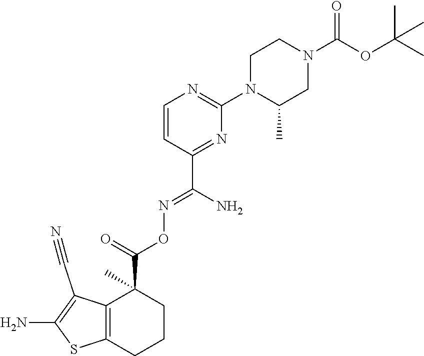 [SMILES: C[C@H]1CN(C(=O)OC(C)(C)C)CCN1c1nccc(/C(N)=N/OC(=O)[C@@]2(C)CCCc3sc(N)c(C#N)c32)n1] | 1.42 | 555 | A |
| K-11c | 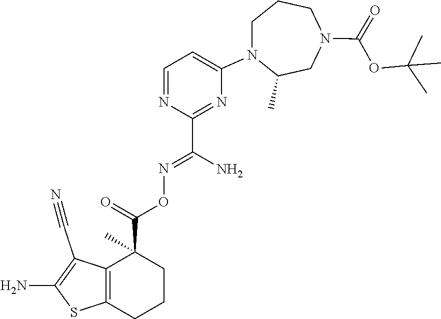 [SMILES: C[C@H]1CN(C(=O)OC(C)(C)C)CCCN1c1ccnc(/C(N)=N/OC(=O)[C@@]2(C)CCCc3sc(N)c(C#N)c32)n1] | 1.28 | 569 | A |
| K-11d | 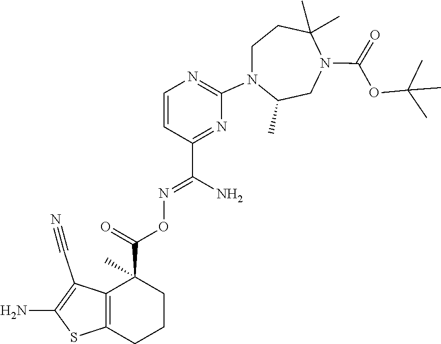 [SMILES: C[C@H]1CN(C(=O)OC(C)(C)C)C(C)(C)CCN1c1nccc(/C(N)=N/OC(=O)[C@@]2(C)CCCc3sc(N)c(C#N)c32)n1] | 2.18 | 597 | E |

### Experimental Procedure for the Synthesis of K-12a

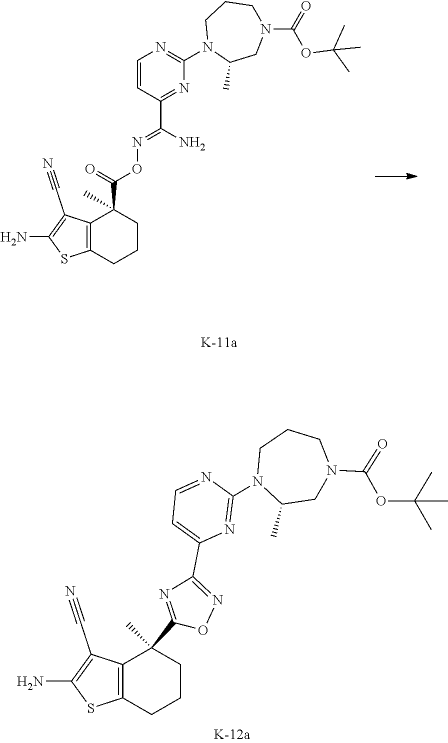 [SMILES: C[C@H]1CN(C(=O)OC(C)(C)C)CCCN1c1nccc(/C(N)=N/OC(=O)[C@@]2(C)CCCc3sc(N)c(C#N)c32)n1 ; C[C@H]1CN(C(=O)OC(C)(C)C)CCCN1c1nccc(-c2noc([C@@]3(C)CCCc4sc(N)c(C#N)c43)n2)n1]

To a stirred solution of K-11a (2.00 g, 3.51 mmol, 1.0 eq) in THF (40 mL) is added DBU (1.98 mL, 14.04 mmol, 4.0 eq) at rt. The reaction mixture is stirred at 70° C. overnight. After complete conversion the reaction mixture is concentrated under reduced pressure to get the crude product. The crude product is purified by column chromatography to afford K-12a.

The following intermediates K-12 (table 17) are available in an analogous manner using different intermediates K-11. The crude product K-12 is purified by chromatography if necessary.

TABLE 17

| # | structure | tᵣₑₜ [min] | [M + H]⁺ | HPLC method |
|---|---|---|---|---|
| K-12a | 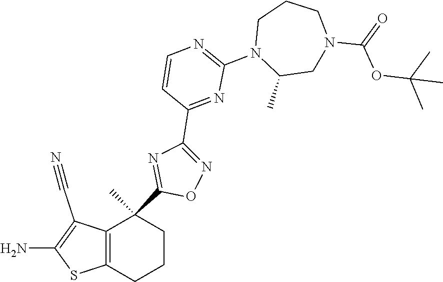 [SMILES: C[C@H]1CN(C(=O)OC(C)(C)C)CCCN1c1nccc(-c2noc([C@@]3(C)CCCc4sc(N)c(C#N)c43)n2)n1] | 1.51 | 551 | A |
| K-12b | 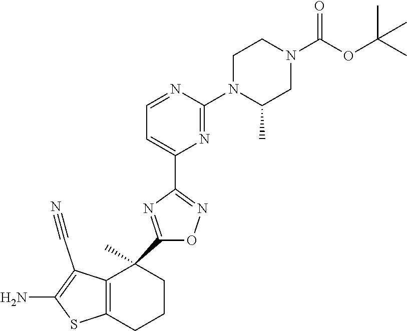 [SMILES: C[C@H]1CN(C(=O)OC(C)(C)C)CCN1c1nccc(-c2noc([C@@]3(C)CCCc4sc(N)c(C#N)c43)n2)n1] | 1.54 | 537 | A |
| K-12c | 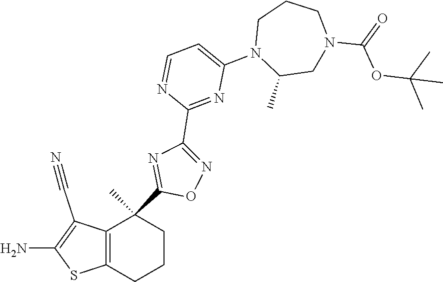 [SMILES: C[C@H]1CN(C(=O)OC(C)(C)C)CCCN1c1ccnc(-c2noc([C@@]3(C)CCCc4sc(N)c(C#N)c43)n2)n1] | 1.36 | 551 | A |
| K-12d | 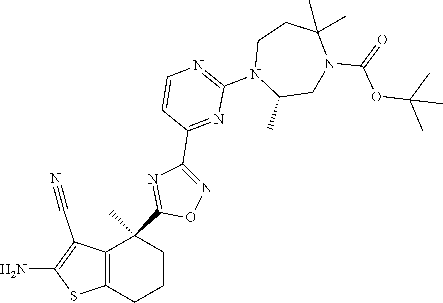 [SMILES: C[C@H]1CN(C(=O)OC(C)(C)C)C(C)(C)CCN1c1nccc(-c2noc([C@@]3(C)CCCc4sc(N)c(C#N)c43)n2)n1] | 2.53 | 579 | K |

### Experimental Procedure for the Synthesis of K-13a

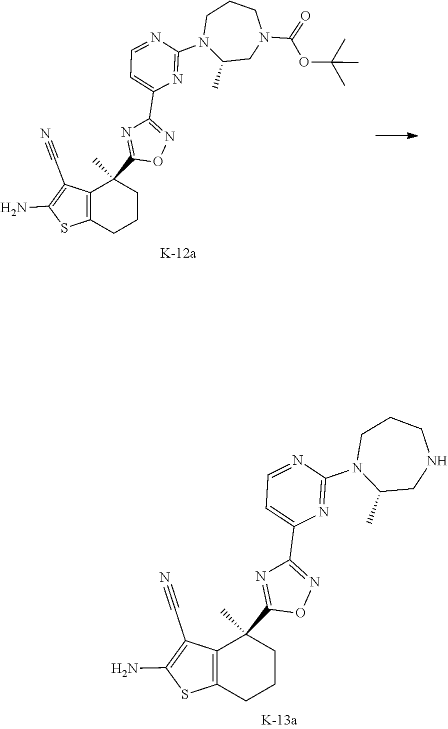 [SMILES: C[C@H]1CN(C(=O)OC(C)(C)C)CCCN1c1nccc(-c2noc([C@@]3(C)CCCc4sc(N)c(C#N)c43)n2)n1 ; C[C@H]1CNCCCN1c1nccc(-c2noc([C@@]3(C)CCCc4sc(N)c(C#N)c43)n2)n1]

To a stirred solution of K-12a (20.00 g, 34.52 mmol, 1.0 eq.) in MeOH (350 mL) is added conc. HCl (32.88 mL, 345.21 mmol, 10.0 eq.) at rt. The reaction mixture is stirred at 50° C. for 2 h. After complete conversion the reaction mixture is concentrated under reduced pressure and diluted with water. The aqueous phase is extracted with DCM. The combined organic layers are dried over sodium sulfate, filtered and concentrated under reduced pressure to afford K-13a which is used for the next step without further purification.

The following intermediates K-13 (table 18) are available in an analogous manner using different intermediates K-12. The crude product K-13 is purified by chromatography if necessary.

TABLE 18

| # | structure | tᵣₑₜ [min] | [M + H]⁺ | HPLC method |
|---|---|---|---|---|
| K-13a | 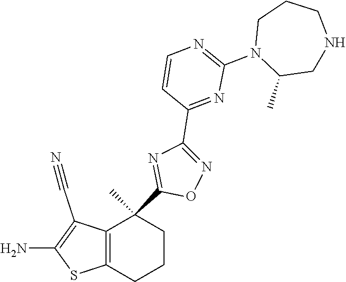 [SMILES: C[C@H]1CNCCCN1c1nccc(-c2noc([C@@]3(C)CCCc4sc(N)c(C#N)c43)n2)n1] | 1.21 | 451 | A |
| K-13b | 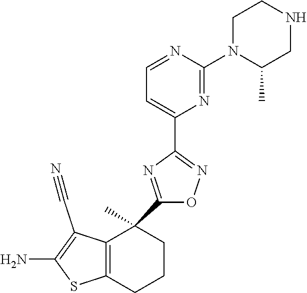 [SMILES: C[C@H]1CNCCN1c1nccc(-c2noc([C@@]3(C)CCCc4sc(N)c(C#N)c43)n2)n1] | 1.18 | 437 | A |
| K-13c | 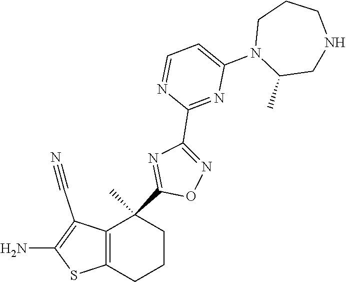 [SMILES: C[C@H]1CNCCCN1c1ccnc(-c2noc([C@@]3(C)CCCc4sc(N)c(C#N)c43)n2)n1] | 0.53 | 451 | C |
| K-13g | 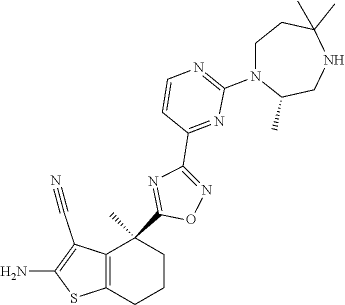 [SMILES: C[C@H]1CNC(C)(C)CCN1c1nccc(-c2noc([C@@]3(C)CCCc4sc(N)c(C#N)c43)n2)n1] | 0.70 | 479 | C |

### Experimental Procedure for the Synthesis of K-14a

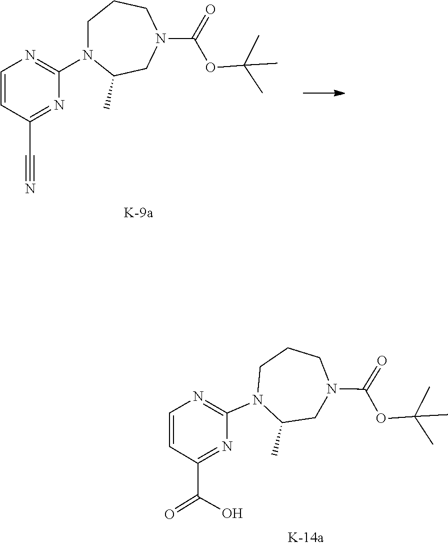 [SMILES: C[C@H]1CN(C(=O)OC(C)(C)C)CCCN1c1nccc(C#N)n1 ; C[C@H]1CN(C(=O)OC(C)(C)C)CCCN1c1nccc(C(=O)O)n1]

To a solution of K-9a (534 mg, 1.68 mmol, 1.0 eq.) in EtOH (4.0 ml) is added sodium hydroxide (4 M in water, 1.00 ml 40.0 mmol, 2.4 eq.) and stirred under reflux for 1 h. The reaction mixture is allowed to cool to rt and acidified with citric acid (5% in water). Water and EtOAc are added and the phases are separated. The organic layer is washed with water, dried over sodium sulfate, filtered and concentrated under reduced pressure to get the crude product K-14a (Table 19) which is used for the next steps without further purification.

TABLE 19

| # | Structure | tᵣₑₜ [min] | [M + H]⁺ | HPLC method |
|---|---|---|---|---|
| K-14a | 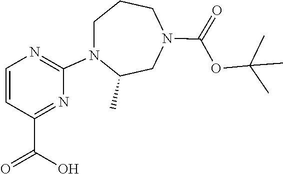 [SMILES: C[C@H]1CN(C(=O)OC(C)(C)C)CCCN1c1nccc(C(=O)O)n1] | 0.77 | 337 | A |

### Experimental Procedure for the Synthesis of K-15a

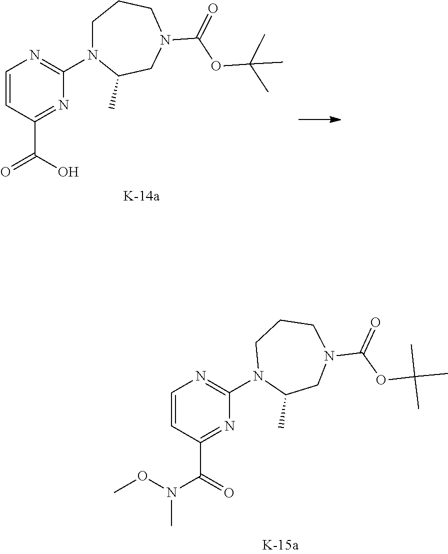

To a stirred solution of K-14a (481.0 mg, 1.43 mmol, 1.0 eq) in dry THF (3.0 mL, 7.5V) are added DIPEA (1.27 mL, 7.15 mmol, 5.0 eq) and HATU (598 mg, 1.57 mmol, 1.1 eq) and stirred at rt for 15 min. N,O-dimethyl hydroxyl amine hydrochloride (278.9 mg, 2.86 mmol, 2.0 eq) is added and stirred at rt for 1 h. After full conversion the reaction mixture is diluted with water and EtOAc and the phases are separated. The organic layer is washed with water, dried over sodium sulfate and concentrated under reduced pressure to get the crude K-15a (Table 20). The crude is purified by chromatography.

TABLE 20

| # | Structure | tᵣₑₜ [min] | [M + H]⁺ | HPLC method |
|---|---|---|---|---|
| K-15a | 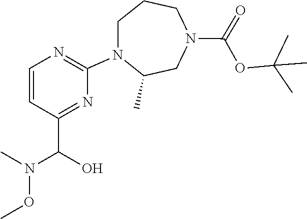 [SMILES: CON(C)C(O)c1ccnc(N2CCCN(C(=O)OC(C)(C)C)C[C@@H]2C)n1] | 1.22 | 380 | A |

### Experimental Procedure for the Synthesis of K-16a

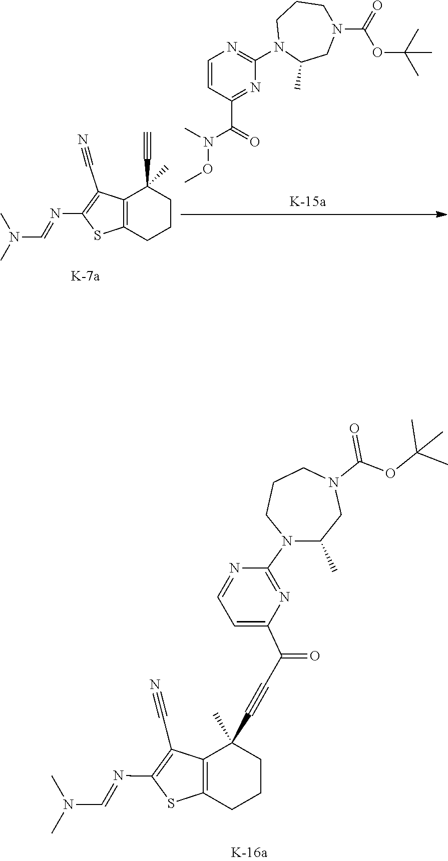

To a solution of K-7a (230 mg, 0.85 mmol, 1.0 eq.) and K-15a (389.0 mg, 1.025 mmol, 1.2 eq.) in THF (7 mL) is added LiHMDS (3.39 mL, 3.39 mmol, 4.00 eq., 1 M in THF) at −78° C. dropwise and stirred for 10 min at −78° C. After complete conversion the reaction is allowed to warm to rt, quenched with water and diluted with EtOAc. The phases are separated, and the aqueous layer is extracted three times with EtOAc. The combined organic layer is concentrated under reduced pressure. The residue is taken up in acetonitrile and water and purified by chromatography to give the desired product K-16a (table 21).

TABLE 21

| # | Structure | tᵣₑₜ [min] | [M + H]⁺ | HPLC method |
|---|---|---|---|---|
| K-16a | 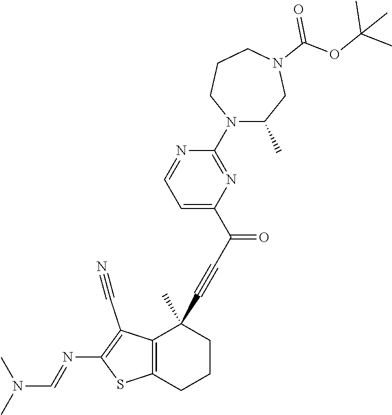 [SMILES: C[C@H]1CN(C(=O)OC(C)(C)C)CCCN1c1nccc(C(=O)C#C[C@@]2(C)CCCc3sc(/N=C/N(C)C)c(C#N)c32)n1] | 1.64 | 590 | A |

### Experimental Procedure for the Synthesis of K-17a

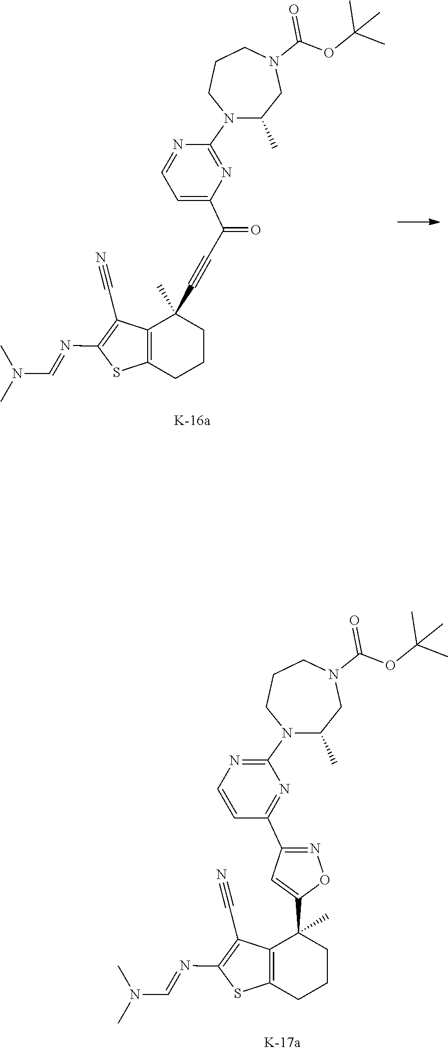

To a stirred solution of K-16a (100 mg, 0.17 mmol, 1.0 eq.) in EtOH (1 mL) is added hydroxylamine hydrochloride (60.1 mg, 0.85 mmol, 5.0 eq.) and stirred at rt for 2 h. After complete conversion the reaction is quenched with saturated sodium hydrogen carbonate solution and stirred at rt for 2 h. The mixture is diluted with acetonitrile and water, filtered and purified by chromatography to give the desired intermediate K-17a (table 22).

TABLE 22

| # | Structure | tᵣₑₜ [min] | [M + H]⁺ | HPLC method |
|---|---|---|---|---|
| K-17a | 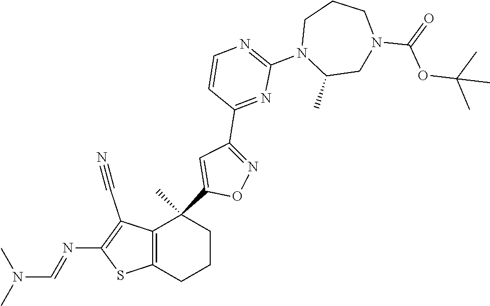 [SMILES: C[C@H]1CN(C(=O)OC(C)(C)C)CCCN1c1nccc(-c2cc([C@@]3(C)CCCc4sc(/N=C/N(C)C)c(C#N)c43)on2)n1] | 1.64 | 605 | A |

### Experimental Procedure for the Synthesis of K-18a

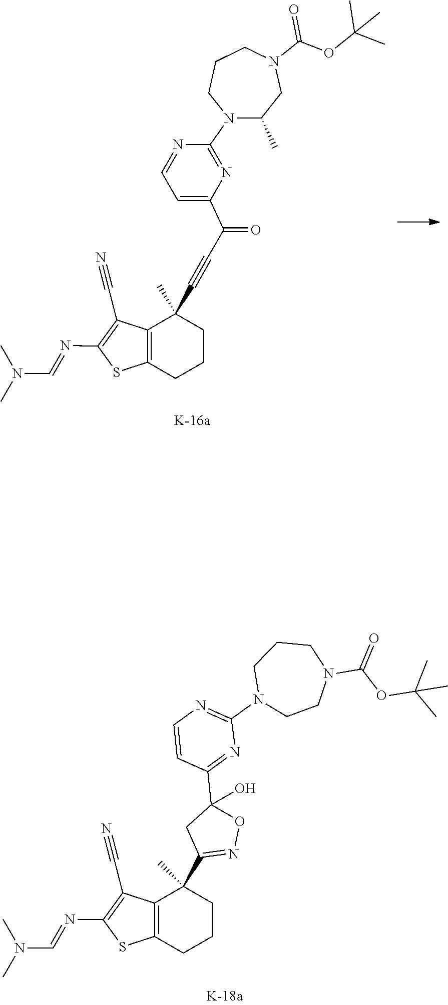

To a stirred solution of K-16a (45 mg, 0.076 mmol, 1.0 eq.) in ACN (0.5 mL) is added hydroxylamine solution (50% in water, 70.14 μL, 1.145 mmol, 15.0 eq.) and the reaction mixture is stirred at rt for 30 min. After completion of conversion the reaction mixture is diluted with acetonitrile and water, filtered and purified by chromatography to give the desired intermediate K-18a (table 23).

TABLE 23

| # | Structure | tᵣₑₜ [min] | [M + H]⁺ | HPLC method |
|---|---|---|---|---|
| K-18a | 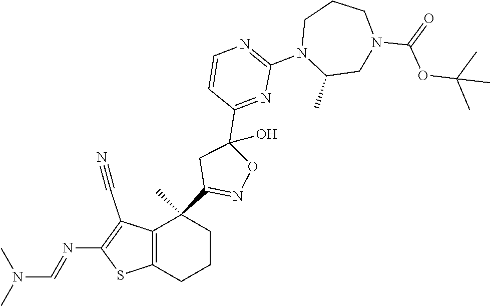 [SMILES: C[C@H]1CN(C(=O)OC(C)(C)C)CCCN1c1nccc(C2(O)CC([C@@]3(C)CCCc4sc(/N=C/N(C)C)c(C#N)c43)=NO2)n1] | 1.48 | 621 | A |

### Experimental Procedure for the Synthesis of K-17b

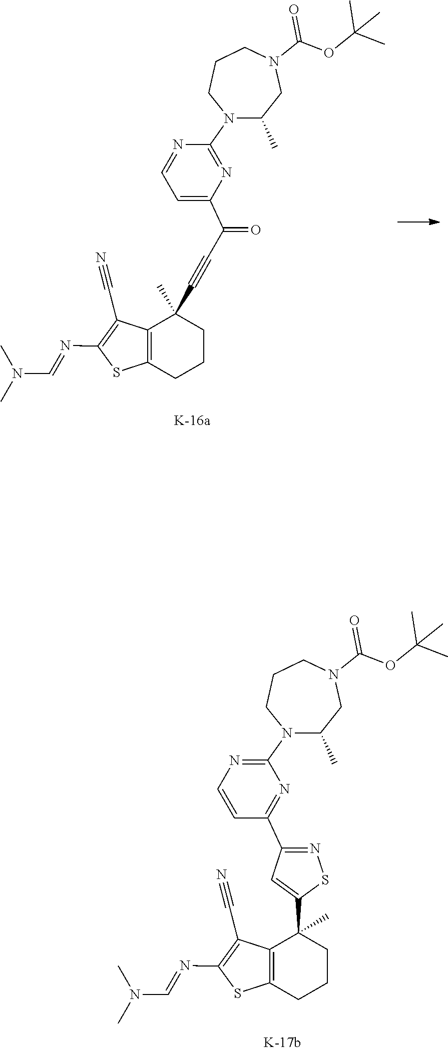

To a solution of K-16a (85.0 mg, 0.144 mmol, 1.0 eq.) in methanol (2 mL) is added hydroxylamine-O-sulfonic acid (32.6 mg, 0.288 mmol, 2.0 eq.). The reaction mixture is stirred for 40 min. Sodium hydrogencarbonate (30.3 mg, 0.360 mmol, 2.5 eq.) and sodium hydrogen sulfide (20.2 mg, 0.360 mmol, 2.5 eq.) are added to the reaction mixture and it is stirred for 18 h. Additional sodium hydrogencarbonate (36.3 mg, 0.432 mmol, 3.0 eq.) and sodium hydrogen sulfide (24.2 mg, 0.432 mmol, 3.0 eq.) are added and the reaction mixture is stirred at 60° C. for 2 hours. The reaction mixture is diluted with DCM and saturated aqueous sodium hydrogencarbonate solution. The phases are separated and the aqueous layer is extracted with DCM (5 times). The organic layers are combined and the solvent is removed under reduced pressure. The crude product is purified by chromatography to give intermediate K-17b (table 24).

TABLE 24

| # | Structure | tᵣₑₜ [min] | [M + H]⁺ | HPLC method |
|---|---|---|---|---|
| K-17b | 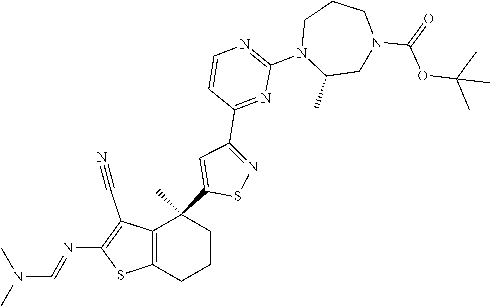 [SMILES: C[C@H]1CN(C(=O)OC(C)(C)C)CCCN1c1nccc(-c2cc([C@@]3(C)CCCc4sc(/N=C/N(C)C)c(C#N)c43)sn2)n1] | 1.69 | 581 | A |

### Experimental Procedure for the Synthesis of K-13d

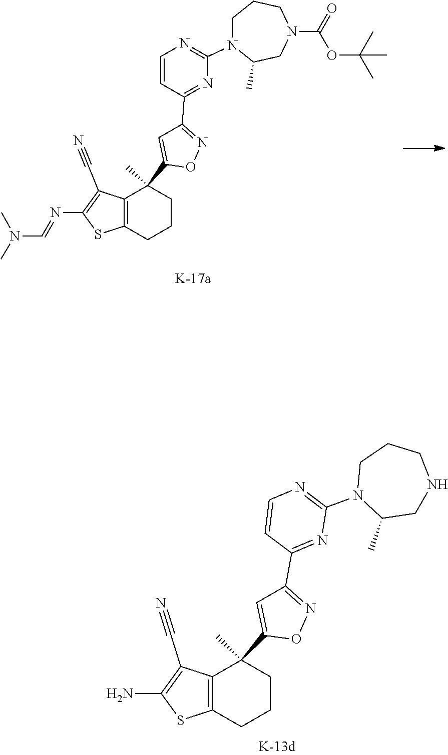 [SMILES: C[C@H]1CN(C(=O)OC(C)(C)C)CCCN1c1nccc(-c2cc([C@@]3(C)CCCc4sc(/N=C/N(C)C)c(C#N)c43)on2)n1 ; C[C@H]1CNCCCN1c1nccc(-c2cc([C@@]3(C)CCCc4sc(N)c(C#N)c43)on2)n1]

To a stirred solution of K-17a (114.0 mg, 0.19 mmol, 1.0 eq.) in THF (1 mL) is added HCl (2 M in water, 0.94 mL, 1.89 mmol, 10.0 eq.) at rt. The reaction mixture is stirred at 65° C. for 2 h. After complete conversion the reaction mixture is concentrated under reduced pressure, diluted with water and acetonitrile and brought to an alkaline pH by addition of 2 M NaOH. It is purified by reversed phase chromatography using basic conditions to yield K-13d.

The following intermediates K-13 (table 25) are available in an analogous manner using different intermediates K-17. The crude product K-17 is purified by chromatography if necessary.

TABLE 25

| # | structure | tᵣₑₜ [min] | [M + H]⁺ | HPLC method |
|---|---|---|---|---|
| K-13d | 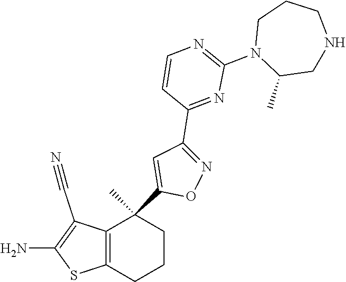 [SMILES: C[C@H]1CNCCCN1c1nccc(-c2cc([C@@]3(C)CCCc4sc(N)c(C#N)c43)on2)n1] | 1.26 | 450 | A |
| K-13e | 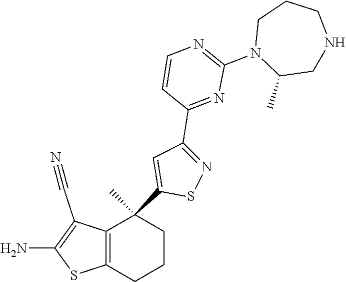 [SMILES: C[C@H]1CNCCCN1c1nccc(-c2cc([C@@]3(C)CCCc4sc(N)c(C#N)c43)sn2)n1] | 1.31 | 466 | A |

### Experimental Procedure for the Synthesis of K-13f

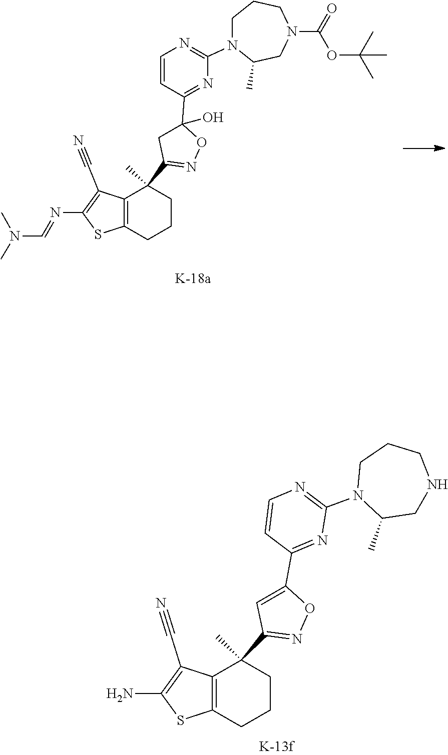 [SMILES: C[C@H]1CN(C(=O)OC(C)(C)C)CCCN1c1nccc(C2(O)CC([C@@]3(C)CCCc4sc(/N=C/N(C)C)c(C#N)c43)=NO2)n1 ; C[C@H]1CNCCCN1c1nccc(-c2cc([C@@]3(C)CCCc4sc(N)c(C#N)c43)no2)n1]

To a stirred solution of K-18a (40.0 mg, 0.06 mmol, 1.0 eq.) in THF (0.5 mL) is added HCl (2 M in water, 0.32 mL, 0.64 mmol, 10.0 eq.) at rt. The reaction mixture is stirred at 65° C. for 3 h. After complete conversion the reaction mixture is concentrated under reduced pressure, diluted with water and acetonitrile and basified by addition of NaOH (2 M in water). The mixture is purified by chromatography to yield K-13f (table 26).

TABLE 26

| # | structure | tᵣₑₜ [min] | [M + H]⁺ | HPLC method |
|---|---|---|---|---|
| K-13f | 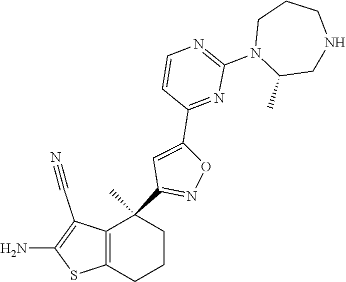 [SMILES: C[C@H]1CNCCCN1c1nccc(-c2cc([C@@]3(C)CCCc4sc(N)c(C#N)c43)no2)n1] | 1.26 | 450 | A |

### Experimental Procedure for the Synthesis of A-1a

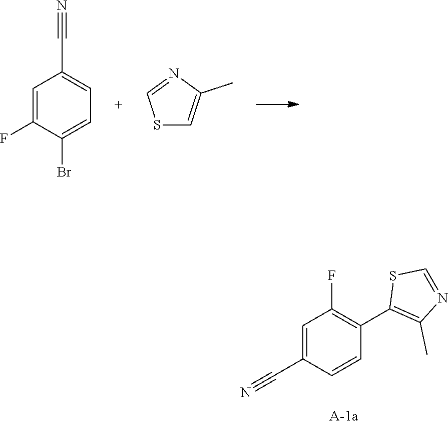

To a stirred solution of 4-bromo-3-fluorobenzonitrile (100 mg, 0.50 mmol, 1.0 eq.) and 4-methyl-thiazole (100 mg, 1.00 mmol, 2.0 eq.) in N,N-Dimethylacetamide (1.00 mL) is added potassium acetate (99 mg, 1.00 mmol, 2.0 eq.) and palladium(II)acetate (11 mg, 0.05 mmol, 0.1 eq.) at rt and the mixture is purged with argon for 15 min. The mixture is stirred and heated by microwave irradiation at 150° C. for 30 min. After complete conversion the reaction mixture is allowed to cool to room temperature and is diluted with water and EtOAc and stirred for 15 min. The phases are separated and the aqueous layer is extracted with EtOAc (3×). The combined organic layers are washed with saturated aqueous NaCl solution and the solvent is removed under reduced pressure. The crude product is purified by chromatography to obtain A-1a.

The following intermediates A-1 (table 27) are available in an analogous manner by using other corresponding bromides as starting material. The crude products A-1 are purified by chromatography if necessary.

TABLE 27

| | | | | HPLC |
|---|---|---|---|---|
| # | structure | tᵣₑₜ [min] | [M + H]⁺ | method |
| A-1a | 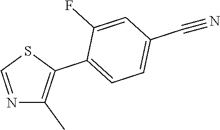 | 1.07 | 219 | A |
| A-1b | 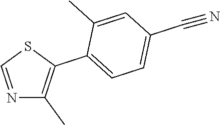 | 1.13 | 215 | A |
| A-1c | 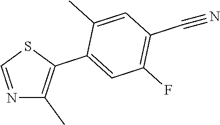 [SMILES: Cc1cc(C#N)c(F)cc1-c1scnc1C] | 1.18 | 233 | A |

### Experimental Procedure for the Synthesis of A-2a

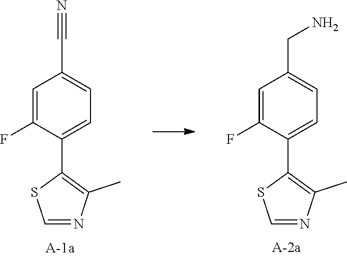

To a stirred solution of A-1a (530 mg, 2.43 mmol, 1.0 eq) in dry THF (20.0 mL) is added lithium aluminium hydride (2 M in THF, 3.64 mL, 7.29 mmol, 3.0 eq) dropwise at 0° C. The reaction mixture is slowly allowed to warm up to RT and stirred over night. After complete conversion the reaction mixture is cooled to 0° C., quenched with aqueous saturated ammonium chloride solution and then treated with aqueous saturated potassium sodium tartrate solution and stirred for 2 h. Aqueous NaOH solution (2 M) and EtOAc are added, the mixture is filtered through celite and the phases are separated. The aqueous layer is extracted with EtOAc. The combined organic layers are washed with brine, dried over sodium sulfate and concentrated under reduced pressure. The crude product is purified by column chromatography to yield A-2a.

The following intermediates A2 (table 28) are available in an analogous manner starting from different intermediates A1. The crude products A2 are purified by chromatography if necessary.

TABLE 28

| | | tᵣₑₜ | | HPLC |
|---|---|---|---|---|
| # | structure | [min] | [M + H]⁺ | method |
| A-2a | 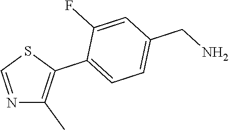 | 0.81 | 223 | A |
| A-2b | 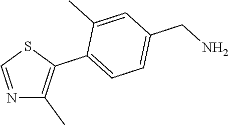 | 0.84 | 219 | A |
| A-2c | 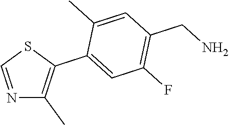 [SMILES: Cc1cc(CN)c(F)cc1-c1scnc1C] | 0.91 | 237 | A |

### Experimental Procedure for the Synthesis of A-2d

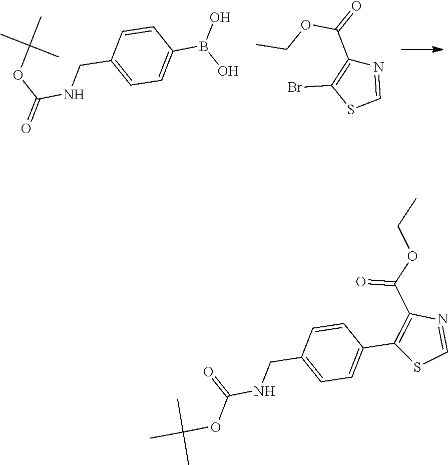

[4-({[(tert-butoxy)carbonyl]amino}methyl)phenyl]boronic acid (500 mg, 1.991 mmol), ethyl 5-bromo-1,3-thiazole-4-carboxylate (630 mg, 2.589 mmol, 1.3 eq.), sodium carbonate (848 mg, 7.965 mmol, 4.0 eq.) and tetrakis-(triphenylphosphin)-palladium (232 mg, 0.199 mmol, 0.1 eq.) are dissolved in dimethoxylethane (5 mL) and water (1.5 mL). The mixture is flushed with argon for 5 min at rt and then stirred at 90° C. for two hours. Water (0.5 mL) is added to the reaction mixture and it is stirred for two additional hours at 90° C. The reaction mixture is diluted with DCM and water and the phases are separated. The aqueous layer is extracted with two times with DCM and the combined organic layers are concentrated to dryness under reduced pressure and purified by chromatography to give ethyl 5-[4-({[(tert-butoxy)carbonyl]amino}methyl)phenyl]-1,3-thiazole-4-carboxylate (HPLC method A: tᵣₑₜ=1.25 min, [M+H]⁺=363)

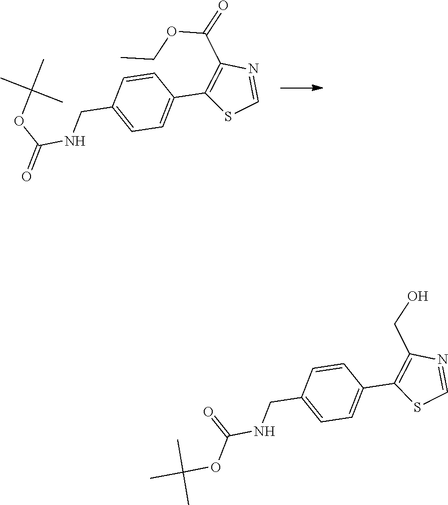 [SMILES: CCOC(=O)c1ncsc1-c1ccc(CNC(=O)OC(C)(C)C)cc1 ; CC(C)(C)OC(=O)NCc1ccc(-c2scnc2CO)cc1]

To a stirred solution of 5-[4-({[(tert-butoxy)carbonyl]amino}methyl)phenyl]-1,3-thiazole-4-carboxylate (580 mg, 1.60 mmol, 1.0 eq) in dry THF (5.0 mL) is added at 0° C. lithium borohydride (2 M in THF, 1.6 mL, 3.20 mmol, 2.0 eq) portionwise at 0° C. The reaction mixture was stirred at rt for 6 h. After complete conversion the reaction mixture is diluted with ice cold water and extracted with EtOAc. The combined organic layers are dried over sodium sulfate and concentrated under reduced pressure. The obtained crude product was purified by column chromatography to give tert-butyl N-({4-[4-(hydroxymethyl)-1,3-thiazol-5-yl]phenyl}methyl)carbamate (HPLC method A: tᵣₑₜ=1.09 min, [M+H]⁺=321)

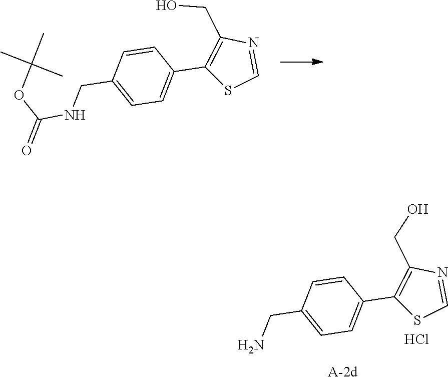 [SMILES: CC(C)(C)OC(=O)NCc1ccc(-c2scnc2CO)cc1 ; NCc1ccc(-c2scnc2CO)cc1 ; Cl]

tert-butyl N-({4-[4-(hydroxymethyl)-1,3-thiazol-5-yl]phenyl}methyl)carbamate (32 mg, 0.1 mmol, 1.0 eq.) is dissolved in methanol (0.32 mL) and HCl (4 M in dioxane, 0.12 mL) is added at 0° C. The reaction mixture is stirred for 2 h at rt. After complete conversion the reaction mixture is concentrated to dryness under reduced pressure to obtain crure A-2d (table 29) as HCl salt which is used without further purification.

TABLE 29

| | | tᵣₑₜ | | HPLC |
|---|---|---|---|---|
| # | structure | [min] | [M + H]⁺ | method |
| A-2d | 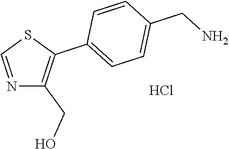 [SMILES: NCc1ccc(-c2scnc2CO)cc1 ; Cl] | 0.11 | 221 | C |

### Experimental Procedure for the Synthesis of A-5a

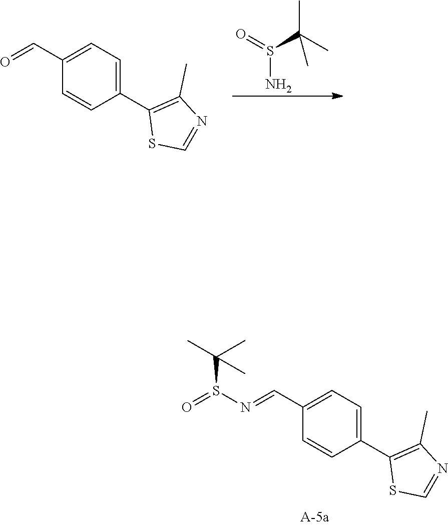

To a stirred solution of 4-(4-methylthiazol-5-yl)benzaldehyde (1.10 g, 5.41 mmol, 1.0 eq.) and (S)-2-methylpropane-2-sulfinamide (0.98 g, 8.12 mmol, 1.50 eq.) in dry THF (10 mL) is added Cs₂CO₃ (4.41 g, 13.5 mmol, 2.50 eq.) and the resulting mixture is stirred at 50° C. for 3 h. The reaction mixture is quenched with water (100 mL) and extracted with EtOAc (2×100 mL). The combined organic layer is dried over Na₂SO₄ filtered, concentrated under reduced pressure and purified by chromatography to give intermediate A-5a (table 30).

TABLE 30

| # | structure | tᵣₑₜ [min] | [M + H]⁺ | HPLC method |
|---|---|---|---|---|
| A-5a | 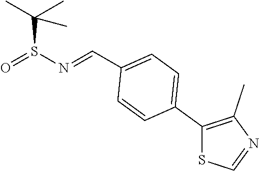 [SMILES: Cc1ncsc1-c1ccc(/C=N/[S@@](=O)C(C)(C)C)cc1] | 1.28 | 307 | A |

### Experimental Procedure for the Synthesis of A-6a

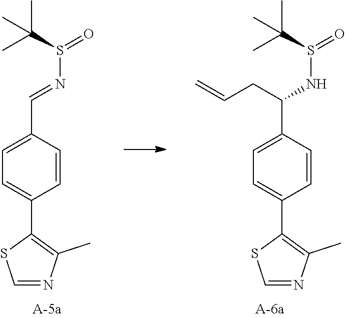 [SMILES: Cc1ncsc1-c1ccc(/C=N/[S@](=O)C(C)(C)C)cc1 ; C=CC[C@H](N[S@](=O)C(C)(C)C)c1ccc(-c2scnc2C)cc1]

To a stirred solution of intermediate A-5a (1.00 g, 3.263 mmol, 1.0 eq.) in THF (5 mL) at 0° C. is added allylmagnesium bromide (4.89 mL, 4.8945 mmol, 1.5 eq. 1.0 M in Et₂O). The reaction was warmed to rt and stirred for 16 h. The reaction mixture was diluted with ice water (10 mL) and the resulting precipitate was filtered and washed with water. The filtrate was extracted with DCM (2×10 mL) and the combined organic layer was dried over sodium sulfate, filtered and concentrated under reduced pressure. The crude product was purified by chromatography to give intermediate A-6a (table 31).

TABLE 31

| # | structure | tᵣₑₜ [min] | [M + H]⁺ | HPLC method |
|---|---|---|---|---|
| A-6a | 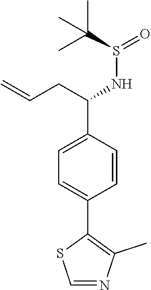 [SMILES: C=CC[C@H](N[S@](=O)C(C)(C)C)c1ccc(-c2scnc2C)cc1] | 1.25 | 349 | A |

### Experimental Procedure for the Synthesis of A-7a

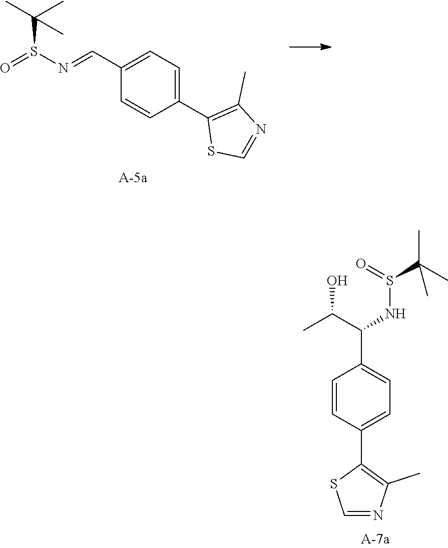 [SMILES: Cc1ncsc1-c1ccc(/C=N/[S@@](=O)C(C)(C)C)cc1 ; Cc1ncsc1-c1ccc([C@@H](N[S@@](=O)C(C)(C)C)[C@H](C)O)cc1]

A stirred solution of SmI₂ (0.1 M in THF, 27.4 mL, 2.74 mmol, 3.0 eq) is cooled to −78° C. and a solution of A-5a (280 mg, 0.91 mmol, 1.0 eq.) and ten drops of acetaldehyde in dry tBuOH (171.5 μL, 1.83 mmol, 2.0 eq) and dry THF (11.0 mL) is added in portions over 0.5 h at −78° C. and stirring is contiuend for 2 h at −78° C. The reaction mixture is quenched with 10% aqueous sodium thiosulfate solution and DCM is added. A white percipitate is formed wich is filtered of over Celite. The phases are separarted and the aqueous phase is extracted three times with DCM. The combined organic layer is dried over magnesium sulfate and concentrated under reduce pressure. The crude product is purified by chromatography to abtain A-7a (table 32) as the main product with a de of 78% which is improved to 98% by chiral chromatography.

TABLE 32

| # | structure | tᵣₑₜ [min] | [M + H]⁺ | HPLC method |
|---|---|---|---|---|
| A-7a | 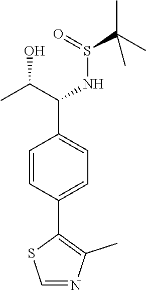 [SMILES: Cc1ncsc1-c1ccc([C@@H](N[S@@](=O)C(C)(C)C)[C@H](C)O)cc1] | 1.01 | 353 | A |

### Experimental Procedure for the Synthesis of A-8a

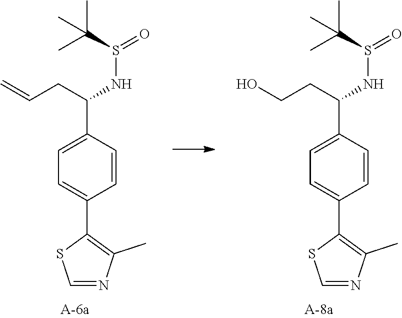 [SMILES: C=CC[C@H](N[S@](=O)C(C)(C)C)c1ccc(-c2scnc2C)cc1 ; Cc1ncsc1-c1ccc([C@H](CCO)N[S@](=O)C(C)(C)C)cc1]

A stirred solution of intermediate A-6a (1.00 g, 2.869 mmol, 1.0 eq.) in MeOH (10 mL) at −78° C. is purged with ozone gas for 30 min. Sodium borohydrate (0.370, 10.042 mmol, 3.5 eq.) is added at −78° C. and the mixture is allowed to warm to rt over night. The reaction mixture is quenched with ice cold water and extrachted with DCM. The organic layer is dried over sodium sulfate, filitered and concentrated under reduced preasue. The crude product is purified by chromatography to give intermediate A-8a (table 33).

TABLE 33

| # | structure | tᵣₑₜ [min] | [M + H]⁺ | HPLC method |
|---|---|---|---|---|
| A-8a | 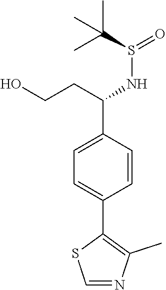 [SMILES: Cc1ncsc1-c1ccc([C@H](CCO)N[S@](=O)C(C)(C)C)cc1] | 0.97 | 353 | A |

### Experimental Procedure for the Synthesis of A-9a

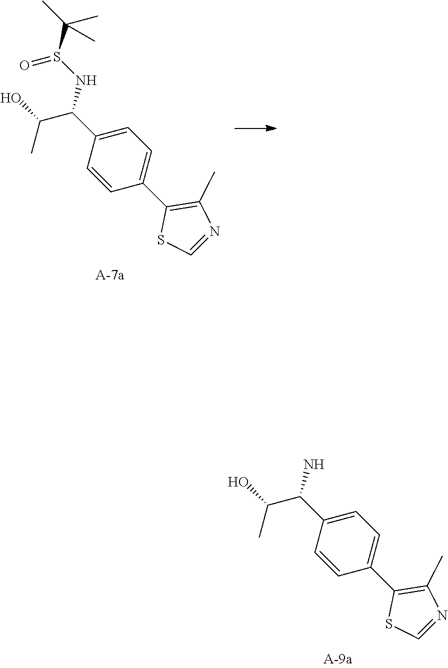 [SMILES: Cc1ncsc1-c1ccc([C@@H](N[S@@](=O)C(C)(C)C)[C@H](C)O)cc1 ; Cc1ncsc1-c1ccc([C@@H](N)[C@H](C)O)cc1]

To a stirred mixture of intermediate A-7a (105 mg, 0.298 mmol, 1.0 eq.) in THF (1.2 mL) is added HCl (0.6 mL, 2 M in H₂O). The mixture is stirred at rt for 3 h. The reaction is diluted with MeOH and purified with an ionexchange column chromatography (Isolute SPE 1 g column, SCX-2) to give intermediate A-9a.

The following intermediates A-9 (table 34) are available in an analogous manner starting from different intermediates A-7 or A-8.

TABLE 34

| | | tᵣₑₜ | | HPLC |
|---|---|---|---|---|
| # | structure | [min] | [M + H]⁺ | method |
| A-9a | 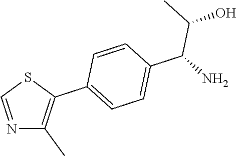 [SMILES: Cc1ncsc1-c1ccc([C@@H](N)[C@H](C)O)cc1] | 0.73 | 249 | A |
| A-9b | 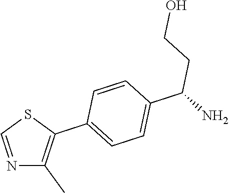 [SMILES: Cc1ncsc1-c1ccc([C@@H](N)CCO)cc1] | 0.26 | 249 | D |

### Experimental Procedure for the Synthesis of A-10a

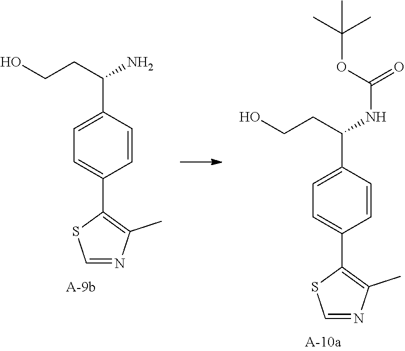 [SMILES: Cc1ncsc1-c1ccc([C@@H](N)CCO)cc1 ; Cc1ncsc1-c1ccc([C@H](CCO)NC(=O)OC(C)(C)C)cc1]

To a stirred solution of intermediate A-9b (600 mg, 2.11 mmol, 1.0 eq.) in 1,4 dioxane (6 mL) and H₂O (6 mL) is added NEt₃ (1.5 mL, 10.5 mmol, 5.0 eq.) and Boc anhydride (0.73 mL, 3.16 mmol, 1.50 eq.) and the mixture is stirred for 3 h at rt. The reaction mixture is quenched with ice cold water and extracted with DCM. The organic layer is separated and dried over sodium sulfate, filtered and cocentrated under reduced preasure. The crude eproduct is purified by chromatography to obtain intermediate A-10a (table 35).

TABLE 35

| # | structure | tᵣₑₜ [min] | [M + H]⁺ | HPLC method |
|---|---|---|---|---|
| A-10a | 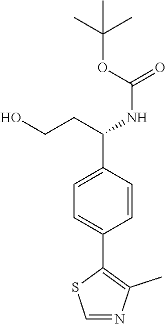 [SMILES: Cc1ncsc1-c1ccc([C@H](CCO)NC(=O)OC(C)(C)C)cc1] | 1.14 | 349 | A |

### Experimental Procedure for the Synthesis of A-11a

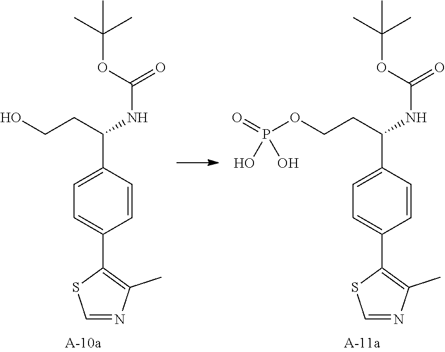 [SMILES: Cc1ncsc1-c1ccc([C@H](CCO)NC(=O)OC(C)(C)C)cc1 ; Cc1ncsc1-c1ccc([C@H](CCOP(=O)(O)O)NC(=O)OC(C)(C)C)cc1]

To a stirred solution of intermediate A-10a (100 mg, 0.274 mmol, 1.0 eq.) in dry THF (2 mL) and NEt₃ (475 μL, 3.289 mmol, 12 eq.) at 0° C. is slowly added a solution on phosphoroxychloride (250 μL, 2.741 mmol, 10 eq.) in dry THF (1 mL). The reaction mixture is stirred at 0° C. for 30 min. The mixture is quenced with water at 0° C. and warmed to rt. The mixture is diluted with acetonitrile and water, flitered and purified by reverse phase chromatography to obtain intermediate A-11a (table 36).

TABLE 36

| | | tᵣₑₜ | | HPLC |
|---|---|---|---|---|
| # | structure | [min] | [M + H]⁺ | method |
| A-11a | 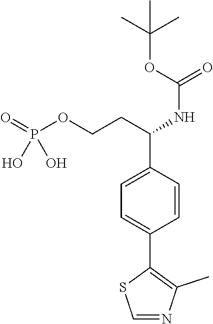 [SMILES: Cc1ncsc1-c1ccc([C@H](CCOP(=O)(O)O)NC(=O)OC(C)(C)C)cc1] | 0.39 | 429 | D |

### Experimental Procedure for the Synthesis of A-2e

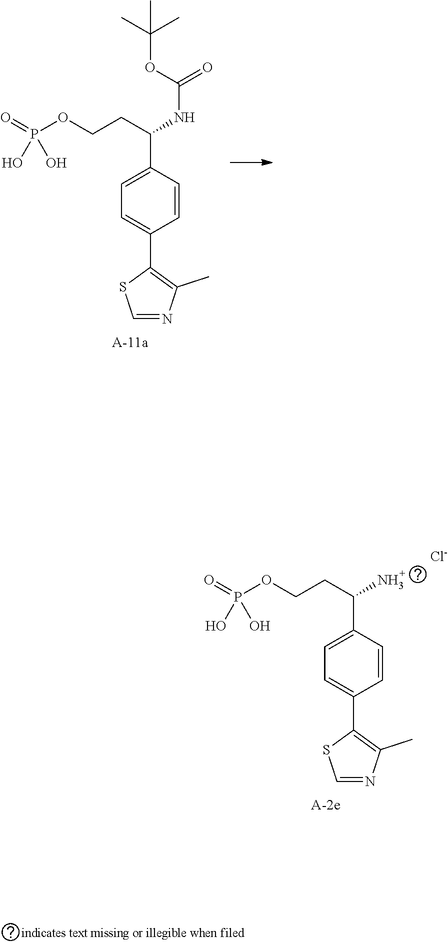 [SMILES: Cc1ncsc1-c1ccc([C@H](CCOP(=O)(O)O)NC(=O)OC(C)(C)C)cc1 ; [Cl-] ; Cc1ncsc1-c1ccc([C@@H](N)CCOP(=O)(O)O)cc1]

To a stirred solution of intermediate A-11a (15 mg, 0.035 mmol, 1.0 eq.) in MeOH (1 mL) at rt is added HCl (1 mL, 4 N in 1,4 dioxane) and the mixture is stirred for 1 h. The reaction mixture is concentrated under reduced preasure and the crude intermediate A-2e (table 37) is used without further prification for the next step.

TABLE 37

| | | tᵣₑₜ | | HPLC |
|---|---|---|---|---|
| # | structure | [min] | [M + H]⁺ | method |
| A-2e | 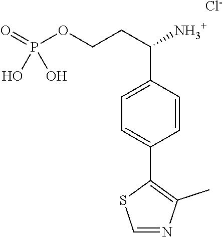 [SMILES: [Cl-] ; Cc1ncsc1-c1ccc([C@@H]([NH3+])CCOP(=O)(O)O)cc1] | 0.08 | 329 | C |

### Experimental Procedure for the Synthesis of A-12a

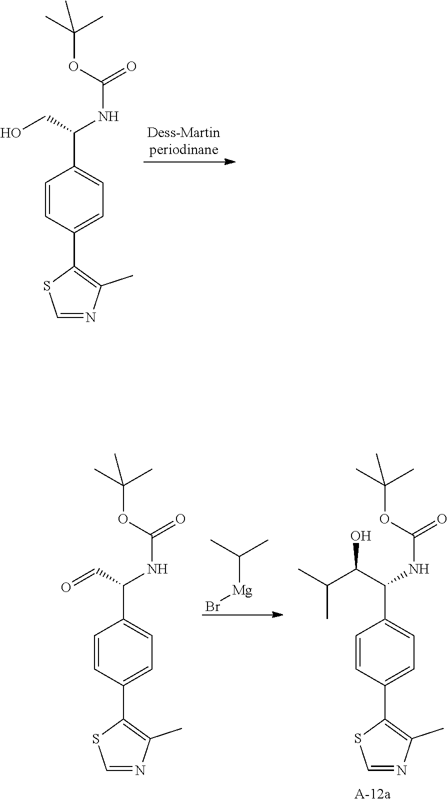

To a stirred solution of tert-butyl N-[(1R)-2-hydroxy-1-[4-(4-methyl-1,3-thiazol-5-yl)phenyl]ethyl]carbamate (500 mg, 1.42 mmol, 1.0 eq.) in THF (3.3 mL) is added Dess-Martin periodinane (723 mg, 1.70 mmol, 1.2 eq.) under argon atmosphere and stirred at rt for 1 h to form the intermediate tert-butyl N-[(1R)-1-[4-(4-methyl-1,3-thiazol-5-yl)phenyl]-2-oxoethyl]carbamate which was not isolated. The reaction mixture is filtered and cooled to −78° C. under argon. Isopropyl-magnesiumbromide (0.8 M in THF, 10.65 mL, 8.52 mmol, 6.0 eq.) is added dropwise over a perid of 1 h. The reaction mixture is stirred at −78° C. for 1 h. After conversion the reaction mixture is quenched with water and DCM is added and the mixture is filtered. The phases are seperated and the organic layer is dried over magnesium sulfate and concentrated under reduce pressure. The crude product is purified by chromatography to give intermediate A-12a (table 38).

TABLE 38

| # | structure | tᵣₑₜ [min] | [M + H]⁺ | HPLC method |
|---|---|---|---|---|
| A-12a | 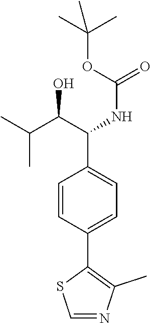 [SMILES: Cc1ncsc1-c1ccc([C@@H](NC(=O)OC(C)(C)C)[C@H](O)C(C)C)cc1] | 1.35 | 377 | A |

### Experimental Procedure for the Synthesis of A-2f

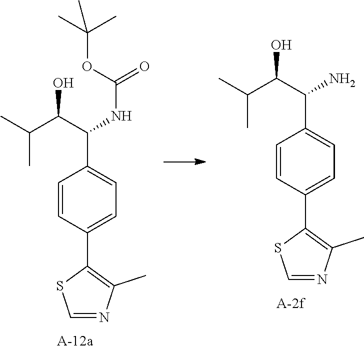 [SMILES: Cc1ncsc1-c1ccc([C@@H](NC(=O)OC(C)(C)C)[C@H](O)C(C)C)cc1 ; Cc1ncsc1-c1ccc([C@@H](N)[C@H](O)C(C)C)cc1]

To a stirred mixture of A-12a (78 mg, 0.207 mmol, 1.0 eq.) in THF (0.8 mL) is added HCl (259 μL, 1.04 mmol, 4.0 M in dioxane, 5.0 eq.) and the mixture is stirred at 60° C. for 2 h. Additional HCl (259 μL, 1.04 mmol, 4.0 M in dioxane, 5.0 eq.) is added and the mixture is stirred at 60° C. for additional 3 h at 60° C. The mixture is cooled to rt and stirred for 18 h at rt. The volatiles are removed under reduced pressure and the residue is dissolved in acetonitrile and H₂O and lyophilized. The crude product is purified by ion exchange chromatography (Isolute SPE 1 g column, SCX-2) to give intermediate A-2f (table 39).

TABLE 39

| # | structure | tᵣₑₜ [min] | [M + H]⁺ | HPLC method |
|---|---|---|---|---|
| A-2f | 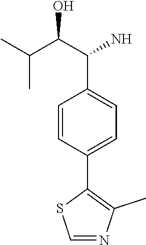 [SMILES: Cc1ncsc1-c1ccc([C@@H](N)[C@H](O)C(C)C)cc1] | 1.01 | 277 | A |

### Experimental Procedure for the Synthesis of A-2g

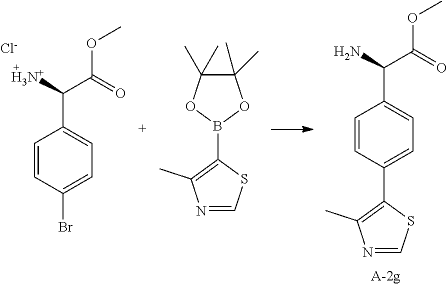

A suspension of methyl (2R)-2-amino-2-(4-bromophenyl)acetate hydro chloride (250 mg, 0.847 mmol, 1.0 eq.), 4-methyl-5-(4,4,5,5-tetramethyl-1,3,2-dioxaborolan-2-yl)-1,3-thiazole (397 mg, 1.693 mmol, 2.0 eq.), XPhos Pd G3 (73 mg, 0.085 mmol, 0.1 eq.) and caesium carbonate (1.3 g, 4.233 mmol, 5.0 eq.) in DME (2 mL) and H₂O (0.4 mL) is heated to 80° C. for 90 min under microwave irradiation. The reaction mixture is purified by chromatography to obtain intermediate A-2g (table 40).

TABLE 40

| # | structure | tᵣₑₜ [min] | [M + H]⁺ | HPLC method |
|---|---|---|---|---|
| A-2g | 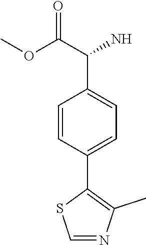 | 0.88 | 263 | A |

### Experimental Procedure for the Synthesis of A-3a

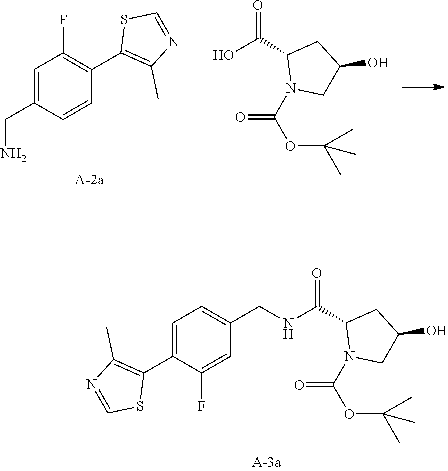 [SMILES: Cc1ncsc1-c1ccc(CN)cc1F ; CC(C)(C)OC(=O)N1C[C@H](O)C[C@H]1C(=O)O ; Cc1ncsc1-c1ccc(CNC(=O)[C@@H]2C[C@@H](O)CN2C(=O)OC(C)(C)C)cc1F]

To a stirred solution of A-2a (85 mg, 0.382 mol, 1.0 eq) in DMF (1 mL) is added (2S,4R)-methyl 4-hydroxypyrrolidine-2-carboxylate hydrochloride (100 mg, 0.421 mmol, 1.1 eq), HATU (163 mg, 0.421 0 mol, 1.1 eq) and DIPEA (0.27 mL, 1.53 mmol, 4.0 eq) at rt and the mixture is stirred at rt for 1 h. After complete conversion the reaction mixture is diluted with acetonitrile, filtered and purified by column chromatography yielding A-3a.

The following intermediates A3 (table 41) are available in an analogous manner starting from different intermediates A2.

TABLE 41

| # | structure | tᵣₑₜ [min] | [M + H]⁺ | HPLC method |
|---|---|---|---|---|
| A-3a | 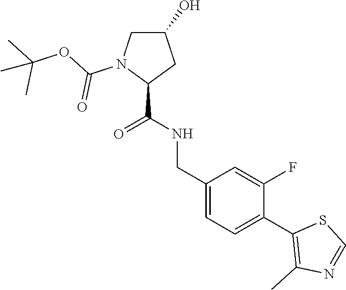 [SMILES: Cc1ncsc1-c1ccc(CNC(=O)[C@@H]2C[C@@H](O)CN2C(=O)OC(C)(C)C)cc1F] | 1.01 | 436 | A |
| A-3b | 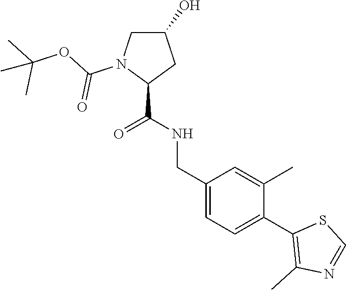 [SMILES: Cc1cc(CNC(=O)[C@@H]2C[C@@H](O)CN2C(=O)OC(C)(C)C)ccc1-c1scnc1C] | 1.04 | 432 | A |
| A-3c | 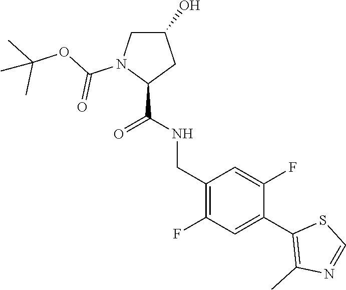 [SMILES: Cc1ncsc1-c1cc(F)c(CNC(=O)[C@@H]2C[C@@H](O)CN2C(=O)OC(C)(C)C)cc1F] | 1.07 | 450 | A |
| A-3d | 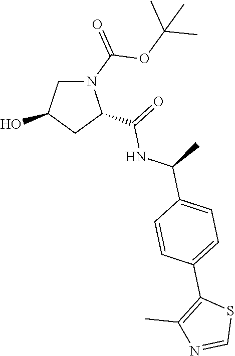 [SMILES: Cc1ncsc1-c1ccc([C@H](C)NC(=O)[C@@H]2C[C@@H](O)CN2C(=O)OC(C)(C)C)cc1] | 1.06 | 432 | A |
| A-3e | 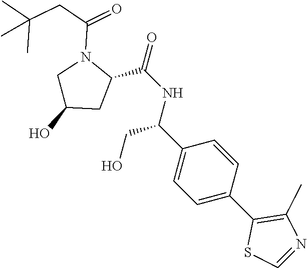 [SMILES: Cc1ncsc1-c1ccc([C@H](CO)NC(=O)[C@@H]2C[C@@H](O)CN2C(=O)CC(C)(C)C)cc1] | 0.92 | 448 | A |
| A-3f | 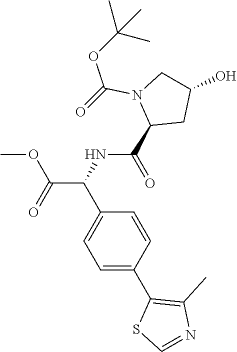 [SMILES: COC(=O)[C@H](NC(=O)[C@@H]1C[C@@H](O)CN1C(=O)OC(C)(C)C)c1ccc(-c2scnc2C)cc1] | 1.07 | 476 | A |

### Experimental Procedure for the Synthesis of A-4a

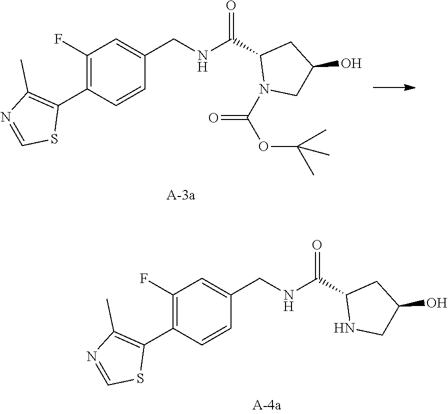 [SMILES: Cc1ncsc1-c1ccc(CNC(=O)[C@@H]2C[C@@H](O)CN2C(=O)OC(C)(C)C)cc1F ; Cc1ncsc1-c1ccc(CNC(=O)[C@@H]2C[C@@H](O)CN2)cc1F]

To a stirred solution of A-3a (105 mg, 0.24 mmol, 1.0 eq.) in THF (1.5 mL) is added HCl (2 M in water, 0.36 mL, 3.0 mmol, 3.0 eq) and stirred at 65° C. for 1 h. After complete conversion the reaction mixture is concentrated under reduced pressure and diluteted in ACN and carefully neutralized with aqueous sodium hydroxide solution (2 M). The mixture is purified by RP-chromatography to obtain A-4a.

The following intermediates A-4 (table 42) are available in an analogous manner starting from different intermediates A-3.

TABLE 42

| # | structure | tᵣₑₜ [min] | [M + H]⁺ | HPLC method |
|---|---|---|---|---|
| A-4a | 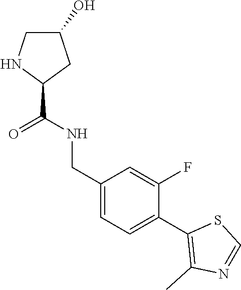 [SMILES: Cc1ncsc1-c1ccc(CNC(=O)[C@@H]2C[C@@H](O)CN2)cc1F] | 0.81 | 336 | A |
| A-4b | 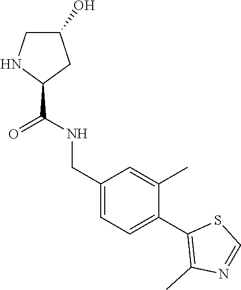 [SMILES: Cc1cc(CNC(=O)[C@@H]2C[C@@H](O)CN2)ccc1-c1scnc1C] | 0.85 | 332 | A |
| A-4c | 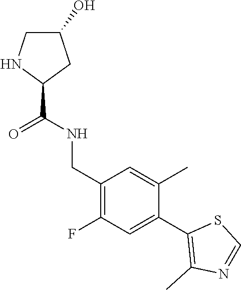 [SMILES: Cc1cc(CNC(=O)[C@@H]2C[C@@H](O)CN2)c(F)cc1-c1scnc1C] | 0.89 | 350 | A |
| A-4d | 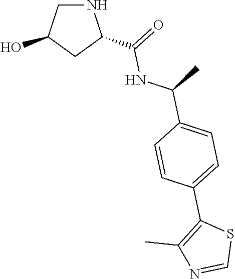 [SMILES: Cc1ncsc1-c1ccc([C@H](C)NC(=O)[C@@H]2C[C@@H](O)CN2)cc1] | 0.88 | 332 | A |
| A-4e | 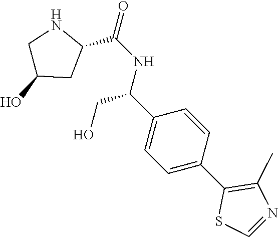 [SMILES: Cc1ncsc1-c1ccc([C@H](CO)NC(=O)[C@@H]2C[C@@H](O)CN2)cc1] | 0.66 | 348 | A |
| A-4f | 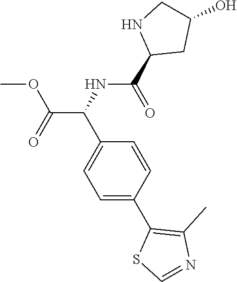 [SMILES: COC(=O)[C@H](NC(=O)[C@@H]1C[C@@H](O)CN1)c1ccc(-c2scnc2C)cc1] | 0.89 | 344 | A |

### Experimental Procedure for the Synthesis of E-1a

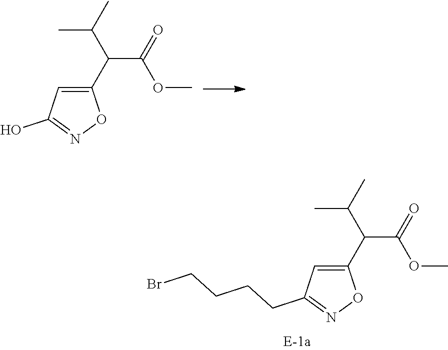 [SMILES: COC(=O)C(c1cc(O)no1)C(C)C ; COC(=O)C(c1cc(CCCCBr)no1)C(C)C]

To a stirred solution of methyl 2-(3-hydroxy-1,2-oxazol-5-yl)-3-methylbutanoate (15.00 g, 0.08 mol, 1.0 eq.) in DMF (75.0 mL) is added potassium carbonate (31.17 g, 0.23 mol, 3.0 eq.) at 0° C. 1,3-Dibromopropane (15.20 g, 0.08 mol, 1.0 eq) is added dropwise and the reaction mixture is stirred at 0° C. for 9 hours. After complete conversion the reaction mixture is quenched with water and extracted with EtOAc. The organic layer is washed with ice water, dried over sodium sulfate and concentrated under reduced pressure to give the crude product. The obtained crude compound is purified by chromatography to yield E-1a.

The following intermediates E-1 (table 43) are available in an analogous manner. The crude products E-1 are purified by chromatography if necessary.

TABLE 43

| # | structure | tᵣₑₜ [min] | [M + H]⁺ | HPLC method |
|---|---|---|---|---|
| E-1a | 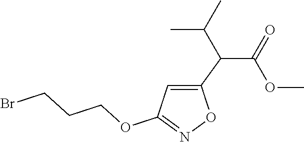 [SMILES: COC(=O)C(c1cc(OCCCBr)no1)C(C)C] | 1.96 | 320 | A |
| E-1b | 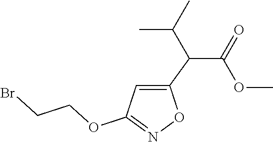 [SMILES: COC(=O)C(c1cc(OCCBr)no1)C(C)C] | 1.33 | 306 | A |
| E-1c | 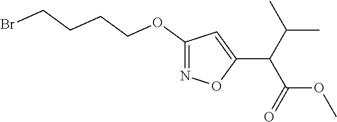 [SMILES: COC(=O)C(c1cc(OCCCCBr)no1)C(C)C] | 2.48 | 334/336 | E |
| E-1e | 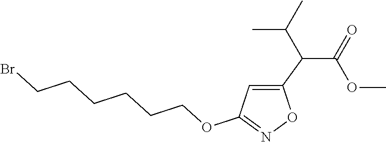 [SMILES: COC(=O)C(c1cc(OCCCCCCBr)no1)C(C)C] | 0.89 | 362/364 | C |
| E-1f | 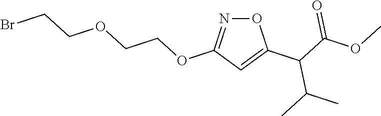 [SMILES: COC(=O)C(c1cc(OCCOCCBr)no1)C(C)C] | 0.81 | 350/352 | C |
| E-1g | 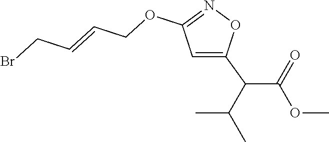 [SMILES: COC(=O)C(c1cc(OC/C=C/CBr)no1)C(C)C] | 0.80 | 332/334 | C |

### Experimental Procedure for the Synthesis of E-6a

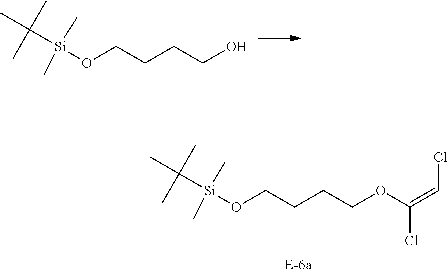 [SMILES: CC(C)(C)[Si](C)(C)OCCCCO ; CC(C)(C)[Si](C)(C)OCCCCO/C(Cl)=C\Cl]

To a stirred solution of 4-[(tert-butyldimethylsilyl)oxy]butan-1-ol (20.00 g, 0.10 mol, 1.0 eq.) in THF (200 ml) is added sodium hydride (2.81 g, 0.12 mol, 1.2 eq.) at 0° C. and stirred at rt for 1 h. To the reaction mixture is cooled to 0° C. and trichloroethylene (15.38 g, 0.12 mol, 1.2 eq.) is added at 0° C. The reaction mixture is warmed to rt and stirred for 48 h. The reaction mixture is quenched with ice cold water and the aqueous solution extracted with EtOAc (3 times). The combined organic layer is washed with water and brine, dried over sodium sulfate, filtered and concentrated under reduced pressure to afford the crude product. The crude product is purified by chromatography yielding E-6a.

The following intermediates E-6 (table 44) are available in an analogous manner using different alcohols as starting material. The crude product E-6 (table 44) is purified by chromatography if necessary.

TABLE 44

| # | structure | tᵣₑₜ [min] | [M + H]⁺ | HPLC method |
|---|---|---|---|---|
| E-6a | 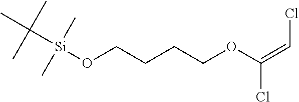 [SMILES: CC(C)(C)[Si](C)(C)OCCCCO/C(Cl)=C\Cl] | 3.08 | n.a. | E |
| E-6b | 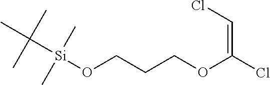 [SMILES: CC(C)(C)[Si](C)(C)OCCCO/C(Cl)=C\Cl] | n.a. | n.a. | n.a. |
| E-6c* | 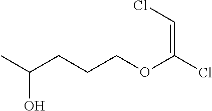 | 1.64 | n.a. | K |
| E-6d* | 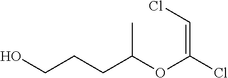 | 1.64 | n.a. | K |

*Intermediates E-6c and E-6d are obtained as a 3.5:1 mixture when using butane-1,3-diol as starting material and are used as mixture in the following step.

### Experimental Procedure for the Synthesis of E-7a

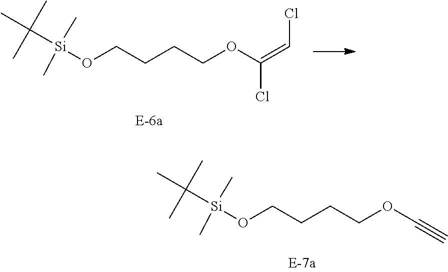 [SMILES: CC(C)(C)[Si](C)(C)OCCCCO/C(Cl)=C\Cl ; C#COCCCCO[Si](C)(C)C(C)(C)C]

To a stirred solution of intermediate E-6a (40.00 g, 0.13 mol, 1.0 eq.) in THF (800 ml) is added n-butyllithium solution (13 mL, 0.33 mol, 2.5 eq, 2.5 M in hexanes) at −78° C. and stirred at −40° C. for 2 h. After complete conversion the mixture is quenched with saturated ammonium chloride solution and diluted with ice cold water. The aqueous solution extracted with EtOAc (3×). The combined organic layer is washed with water and brine, dried over sodium sulfate, filtered, and concentrated under reduced pressure to afford the crude product. The crude product is purified by column chromatography yielding intermediate E-7a.

The following intermediates E-7 (table 45) are available in an analogous manner using different intermediates E-6. The crude product E-7 is purified by chromatography if necessary.

TABLE 45

| | | | | HPLC |
|---|---|---|---|---|
| # | structure | tᵣₑₜ [min] | [M + H]⁺ | method |
| E-7a | 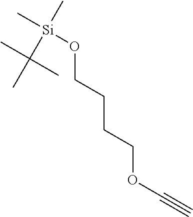 | 2.66 | n.a. | E |
| E-7b | 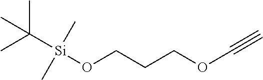 | n.a. | n.a. | n.a. |
| E-7c* | 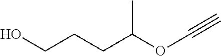 | n.a. | n.a. | n.a. |
| E-7d | 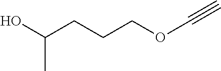 | n.a. | n.a. | n.a. |

*Intermediates E-7c and E-7d are obtained as a 3.5:1 mixture when using a mixture of E-6c and E-6d (3.5:1) as starting material and are used as mixture in the following step.

### Experimental Procedure for the Synthesis of E-8a

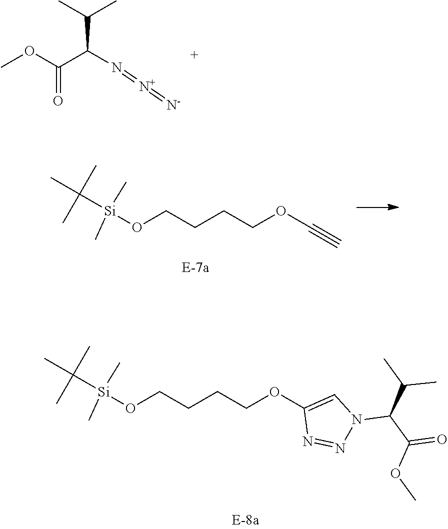

To a stirred solution of intermediate E-7a (5.81 g, 25.44 mmol, 1.0 eq.) in DMF (20 ml) and water (20 ml) is added methyl (2R)-2-azido-3-methylbutanoate (4.00 g, 25.44 mmol, 1.0 eq.), sodium L-ascorbate (2.52 g, 12.73 mmol, 0.5 eq) and copper(II) sulfate pentahydrate (0.88 g, 2.54 mmol, 0.1 eq) at rt. The reaction mixture is heated to 80° C. and stirred for 3 h. The reaction mixture was poured into water and the aqueous layer was extracted with EtOAc (3×). The combined organic layer is dried over sodium sulfate, filtered, and concentrated under reduced pressure to afford the crude product. The crude product is purified by chromatography to yield intermediate E-8a.

The following intermediates E-8 (table 46) are available in an analogous manner using different intermediates E-7 and/or azides. The crude product E-8 is purified by chromatography if necessary.

TABLE 46

| # | structure | tᵣₑₜ [min] | [M + H]⁺ | HPLC method |
|---|---|---|---|---|
| E-8a | 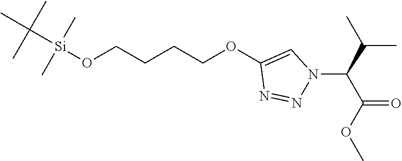 [SMILES: COC(=O)[C@H](C(C)C)n1cc(OCCCCO[Si](C)(C)C(C)(C)C)nn1] | 2.73 | 386 | K |
| E-8b | 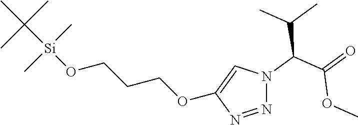 [SMILES: COC(=O)[C@H](C(C)C)n1cc(OCCCO[Si](C)(C)C(C)(C)C)nn1] | 2.45 | 372 | K |
| E-8c* | 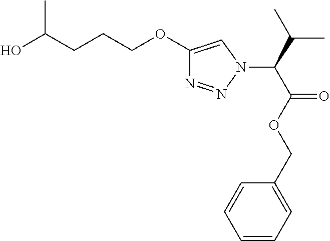 [SMILES: CC(O)CCCOc1cn([C@H](C(=O)OCc2ccccc2)C(C)C)nn1] | 2.23 | 362 | K |
| E-8d* | 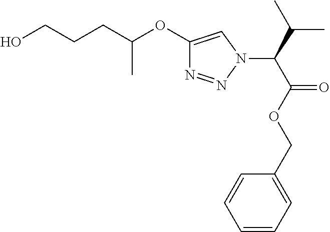 [SMILES: CC(CCCO)Oc1cn([C@H](C(=O)OCc2ccccc2)C(C)C)nn1] | 2.23 | 362 | K |
| E-8e | 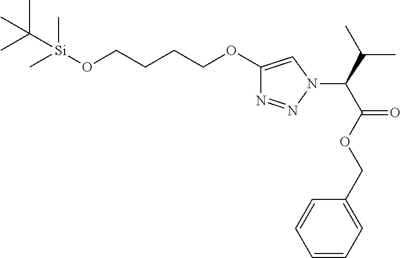 [SMILES: CC(C)[C@@H](C(=O)OCc1ccccc1)n1cc(OCCCCO[Si](C)(C)C(C)(C)C)nn1] | 1.2 | 348 | A |

*Intermediates E-8c and E-8d are obtained as a 3.5:1 mixture when using a mixture of E-7c and E-7d (3.5:1) as starting material and are used as mixture in the following step.

### Experimental Procedure for the Synthesis of E-9a

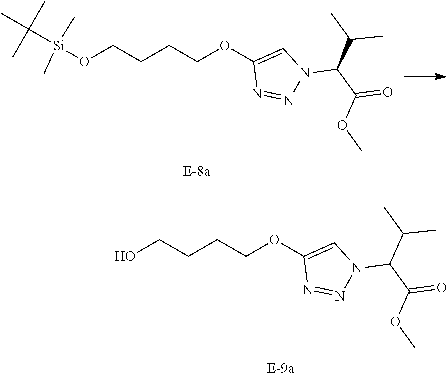 [SMILES: COC(=O)[C@H](C(C)C)n1cc(OCCCCO[Si](C)(C)C(C)(C)C)nn1 ; COC(=O)C(C(C)C)n1cc(OCCCCO)nn1]

Intermediate E-8a (1.85 g, 4.70 mmol, 1.0 eq.) is dissolved in MeOH (18.5 mL) and HCl (1.76 ml, 7.06 mmol, 1.5 eq, 4 N solution in 1,4 dioxane) is added. The reaction mixture is stirred at 45° C. for 30 min and the reaction mixture is concentrated to dryness. The crude product is purified by chromatography to yield Intermediate E-9a.

The following intermediates E-9 (table 47) are available in an analogous manner using different intermediates E-8. The crude product E-9 is purified by chromatography if necessary.

TABLE 47

| # | structure | tᵣₑₜ [min] | [M + H]⁺ | HPLC method |
|---|---|---|---|---|
| E-9a | 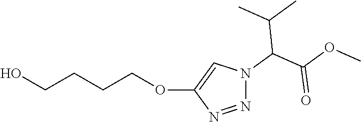 [SMILES: COC(=O)C(C(C)C)n1cc(OCCCCO)nn1] | 0.91 | 272 | A |
| E-9b | 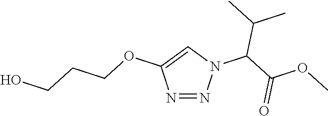 [SMILES: COC(=O)C(C(C)C)n1cc(OCCCO)nn1] | 0.82 | 258 | A |

### Experimental Procedure for the Synthesis of E-9d

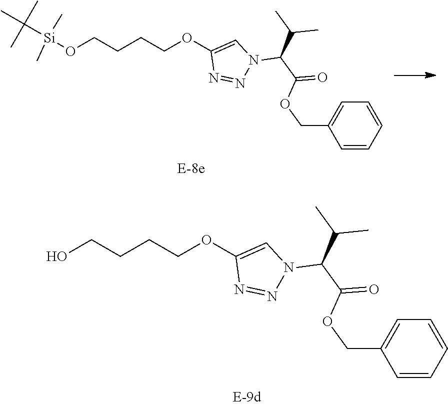 [SMILES: CC(C)[C@@H](C(=O)OCc1ccccc1)n1cc(OCCCCO[Si](C)(C)C(C)(C)C)nn1 ; CC(C)[C@@H](C(=O)OCc1ccccc1)n1cc(OCCCCO)nn1]

To a stirred solution of E-8e (60.0 g, 129.96 mmol, 1.0 eq.) in THF at 10° C. is added HF-pyridine (38.6 g, 389.88 mmol, 3.0 eq.) and the mixture is stirred at 10-20° C. for 2 h. To the mixture is added aqueous NaHCO₃ solution (1 M, 1.5 L) and the pH is adjusted to pH 7-8. The phases are separated, and the aqueous phase is extracted with ethyl acetate and the combined organic layers are dried aver Na₂SO₄. The crude product is purified by column chromatography to obtain intermediate E-9d.

TABLE 47b

| # | structure | tᵣₑₜ [min] | [M + H]⁺ | HPLC method |
|---|---|---|---|---|
| E-9d | 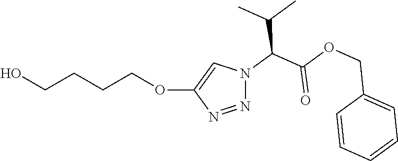 [SMILES: CC(C)[C@@H](C(=O)OCc1ccccc1)n1cc(OCCCCO)nn1] | n.a. | n.a. | n.a. |

### Experimental Procedure for the Synthesis of E-10a and E-10b

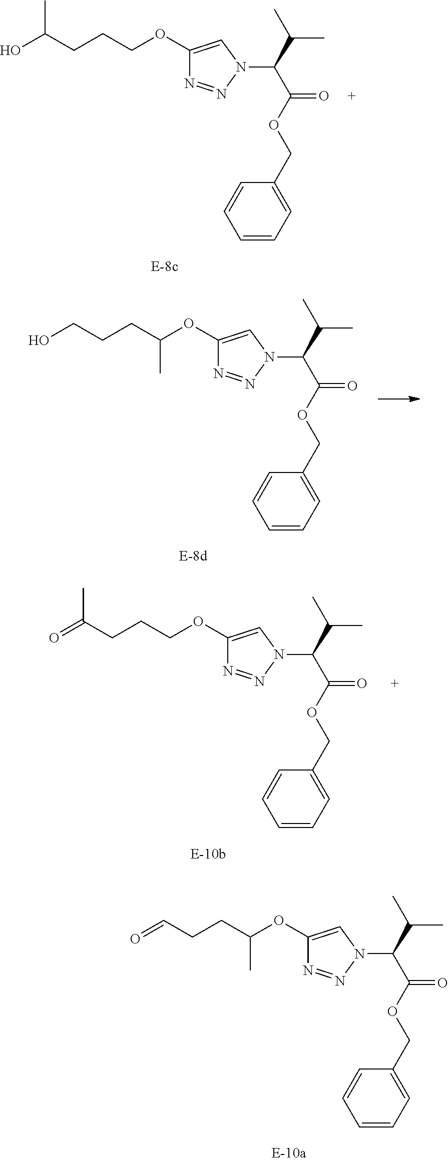 [SMILES: CC(O)CCCOc1cn([C@H](C(=O)OCc2ccccc2)C(C)C)nn1 ; CC(CCCO)Oc1cn([C@H](C(=O)OCc2ccccc2)C(C)C)nn1 ; CC(=O)CCCOc1cn([C@H](C(=O)OCc2ccccc2)C(C)C)nn1 ; CC(CCC=O)Oc1cn([C@H](C(=O)OCc2ccccc2)C(C)C)nn1]

To a stirred solution of a mixture of E-8c and E-8d (258 mg, 0.71 mmol, 1.0 eq, 3.5:1) in DCM (0.5 mL) is added Dess-Martin periodinane (302.7 mg, 0.71 mmol, 1.0 eq) and stirred at rt for 1 h. The reaction mixture is filtered and concentrated under reduce pressure. The crude product is purified by column chromatography to obtain intermediates E-10a and E-10b.

The following intermediates E-10 (table 48) are available in an analogous manner using different intermediates E-8 or E-9. The crude product E-10 is purified by chromatography if necessary.

TABLE 48

| # | structure | tᵣₑₜ [min] | [M + H]⁺ | HPLC method |
|---|---|---|---|---|
| E-10a | 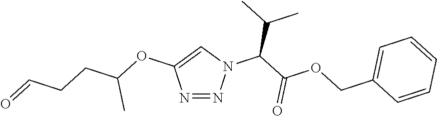 [SMILES: CC(CCC=O)Oc1cn([C@H](C(=O)OCc2ccccc2)C(C)C)nn1] | 1.34 | 360 | A |
| E-10b | 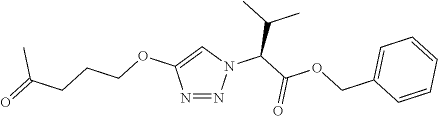 [SMILES: CC(=O)CCCOc1cn([C@H](C(=O)OCc2ccccc2)C(C)C)nn1] | 1.30 | 360 | A |
| E-10c | 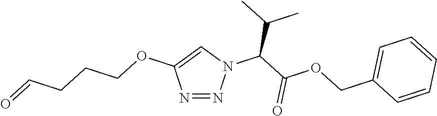 [SMILES: CC(C)[C@@H](C(=O)OCc1ccccc1)n1cc(OCCCC=O)nn1] | 1.28 | 346 | A |

### Experimental Procedure for the Synthesis of E-9c

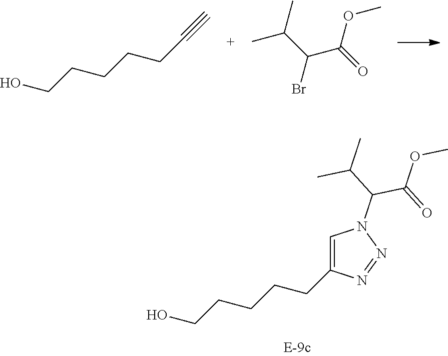

To a stirred solution of methyl 2-bromo-3-methylbutanoate (5.00 g, 0.026 mol, 1.0 eq.) at 0° C. in DMSO (50 mL) is added sodium azide (1.666 g, 0.026 mol, 1.0 eq.). The mixture is allowed to warm to rt and is stirred for 16 hours. The mixture is cooled to 0° C. and water (100 mL), sodium ascorbate (0.51 g, 003 mol, 0.1 eq.), copper(II) sulfate pentahydrate (1.28 g, 0.225 mol, 0.2 eq.), and hept-6-yn-1-ol (2.871 g, 0.026 mmol, 1.0 eq.) are added. The mixture is allowed to warm to rt and is stirred for 16 hours. The reaction mixture was poured into water (500 mL) and the mixture was extracted with ethyl acetate (3×400 mL). The combined organic layer is washed with saturated aqueous NaHCO₃ solution (2×200 mL), dried over Na₂SO₄, and concentrated under reduced pressure. The crude product is purified by chromatography to obtain intermediate E-9c (table 49).

TABLE 49

| # | structure | tᵣₑₜ [min] | [M + H]⁺ | HPLC method |
|---|---|---|---|---|
| E-9c | 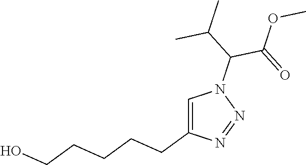 [SMILES: COC(=O)C(C(C)C)n1cc(CCCCCO)nn1] | 0.95 | 270 | A |

### Experimental Procedure for the Synthesis of E-11a

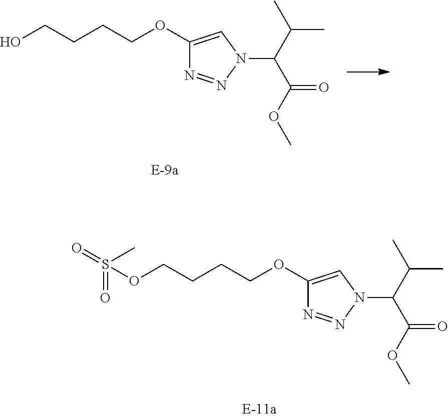 [SMILES: COC(=O)C(C(C)C)n1cc(OCCCCO)nn1 ; COC(=O)C(C(C)C)n1cc(OCCCCOS(C)(=O)=O)nn1]

To a stirred solution of E-9a (1.95 g, 7.19 mmol, 1.0 eq.) and Et₃N (2.00 mL, 14.37 mmol, 2.0 eq). in dichloroethane (20.0 mL) is added methansulfonyl chloride (0.97 mL, 12.46 mmol, 1.70 eq.) slowly at 0° C. and the reaction mixture is stirred at rt for 20 min. The reaction mixture is quenched with water and diluted with DCM. The layers are separated, and the aqueous layer is extracted with DCM, dried over sodium sulfate, filtered, and concentrated under reduce pressure to give the crude product. The crude compound is purified by chromatography to give in. using DCM and MeOH as solvent to yield E-11a.

The following intermediates E-11 (table 50) are available in an analogous manner starting from different intermediates E-9. The crude products E-11 are purified by chromatography if necessary.

TABLE 50

| # | structure | tᵣₑₜ [min] | [M + H]⁺ | HPLC method |
|---|---|---|---|---|
| E-11a | 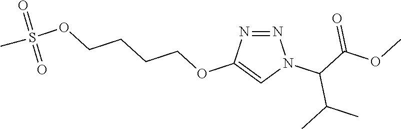 | 1.08 | 350 | A |
| E-11b | 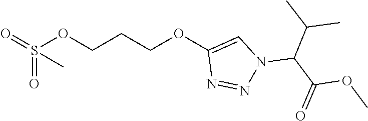 [SMILES: COC(=O)C(C(C)C)n1cc(OCCCOS(C)(=O)=O)nn1] | 1.00 | 336 | A |
| E-11c | 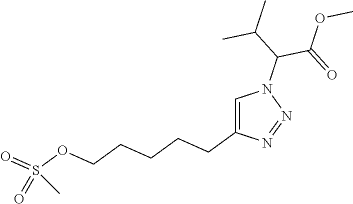 [SMILES: COC(=O)C(C(C)C)n1cc(CCCCCOS(C)(=O)=O)nn1] | 1.11 | 348 | A |

### Experimental Procedure for the Synthesis of E-2a

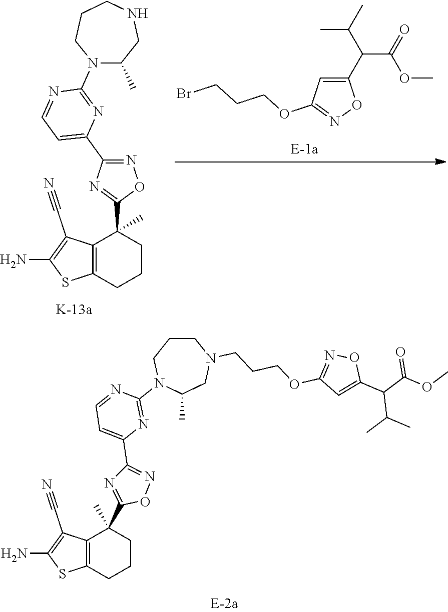 [SMILES: C[C@H]1CNCCCN1c1nccc(-c2noc([C@@]3(C)CCCc4sc(N)c(C#N)c43)n2)n1 ; COC(=O)C(c1cc(OCCCBr)no1)C(C)C ; COC(=O)C(c1cc(OCCCN2CCCN(c3nccc(-c4noc([C@@]5(C)CCCc6sc(N)c(C#N)c65)n4)n3)[C@@H](C)C2)no1)C(C)C]

To a stirred solution of K-13a (4.50 g, 9.99 mmol, 1.0 eq.) and E-1a (3.72 g, 11.03 mmol, 1.1 eq) in acetonitrile (45.0 mL) is added potassium carbonate (2.76 g, 19.98 mmol, 2.0 eq) and the mixture is stirred at 60° C. under argon for 22 h. After complete conversion the reaction mixture is allowed to cool to rt, filtered and the solid is washed with acetonitrile. The combined solution is concentrated under reduced pressure and purified by chromatography to give E-2a.

The following intermediates E-2 (table 51) are available in an analogous manner starting from different intermediates K-13 and E-1 or alternative bromides. The crude products E-2 are purified by chromatography if necessary.

TABLE 51

| | | tᵣₑₜ | | HPLC |
|---|---|---|---|---|
| # | structure | [min] | [M + H]⁺ | method |
| E-2a | 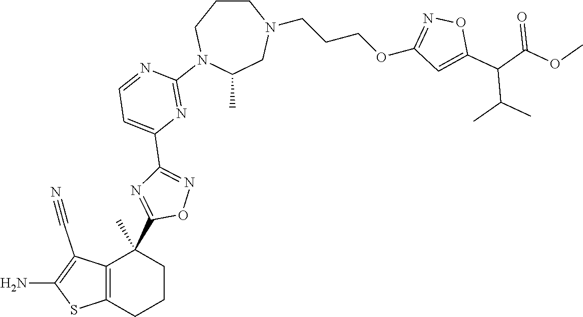 [SMILES: COC(=O)C(c1cc(OCCCN2CCCN(c3nccc(-c4noc([C@@]5(C)CCCc6sc(N)c(C#N)c65)n4)n3)[C@@H](C)C2)no1)C(C)C] | 1.63 | 690 | A |
| E-2b | 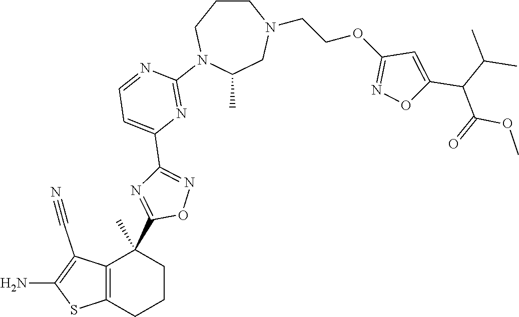 [SMILES: COC(=O)C(c1cc(OCCN2CCCN(c3nccc(-c4noc([C@@]5(C)CCCc6sc(N)c(C#N)c65)n4)n3)[C@@H](C)C2)no1)C(C)C] | 1.70 | 676 | G |
| E-2c | 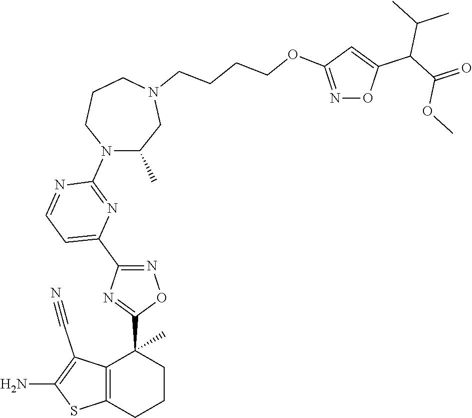 [SMILES: COC(=O)C(c1cc(OCCCCN2CCCN(c3nccc(-c4noc([C@@]5(C)CCCc6sc(N)c(C#N)c65)n4)n3)[C@@H](C)C2)no1)C(C)C] | 1.72 | 704 | A |
| E-2d | 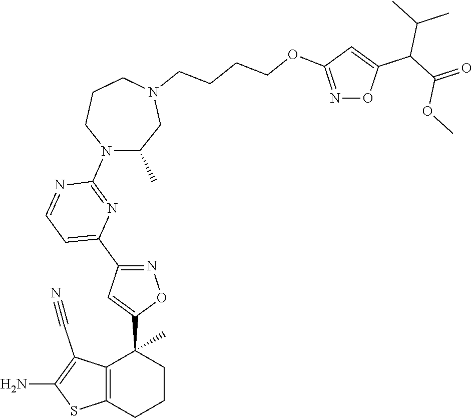 [SMILES: COC(=O)C(c1cc(OCCCCN2CCCN(c3nccc(-c4cc([C@@]5(C)CCCc6sc(N)c(C#N)c65)on4)n3)[C@@H](C)C2)no1)C(C)C] | 1.69 | 703 | A |
| E-2e | 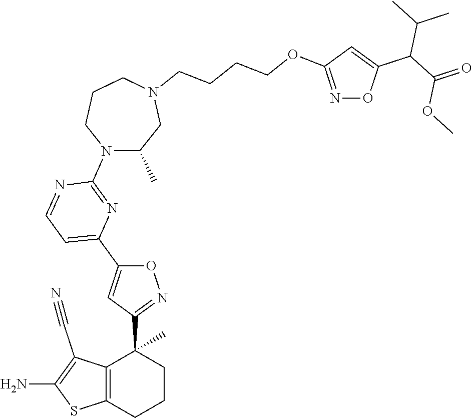 [SMILES: COC(=O)C(c1cc(OCCCCN2CCCN(c3nccc(-c4cc([C@@]5(C)CCCc6sc(N)c(C#N)c65)no4)n3)[C@@H](C)C2)no1)C(C)C] | 1.69 | 703 | A |
| E-2f | 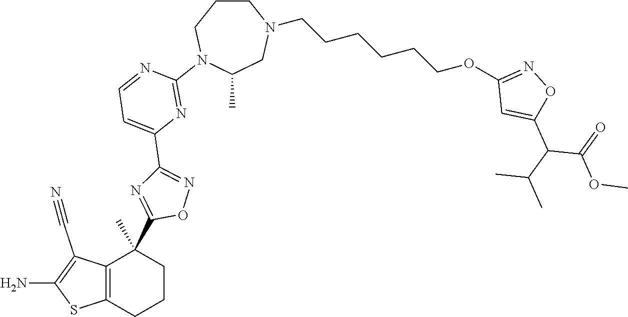 [SMILES: COC(=O)C(c1cc(OCCCCCCN2CCCN(c3nccc(-c4noc([C@@]5(C)CCCc6sc(N)c(C#N)c65)n4)n3)[C@@H](C)C2)no1)C(C)C] | 1.74 | 732 | A |
| E-2g | 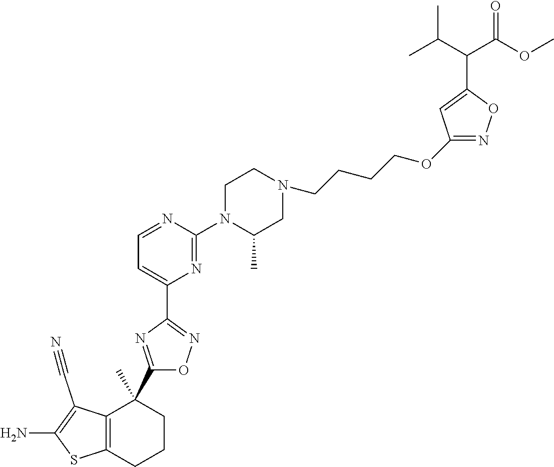 [SMILES: COC(=O)C(c1cc(OCCCCN2CCN(c3nccc(-c4noc([C@@]5(C)CCCc6sc(N)c(C#N)c65)n4)n3)[C@@H](C)C2)no1)C(C)C] | 1.69 | 690 | A |
| E-2h | 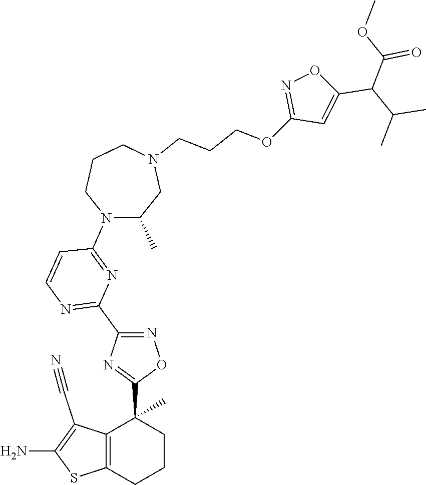 [SMILES: COC(=O)C(c1cc(OCCCN2CCCN(c3ccnc(-c4noc([C@@]5(C)CCCc6sc(N)c(C#N)c65)n4)n3)[C@@H](C)C2)no1)C(C)C] | 1.49 | 690 | A |
| E-2i | 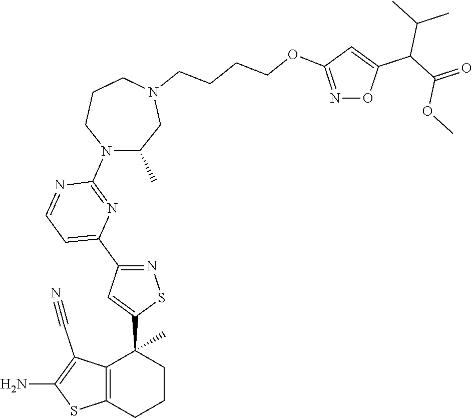 [SMILES: COC(=O)C(c1cc(OCCCCN2CCCN(c3nccc(-c4cc([C@@]5(C)CCCc6sc(N)c(C#N)c65)sn4)n3)[C@@H](C)C2)no1)C(C)C] | 1.74 | 719 | A |
| E-2j | 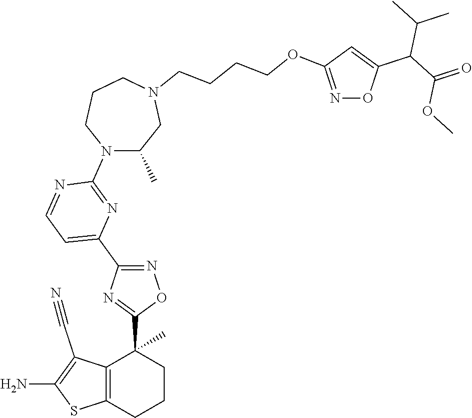 [SMILES: COC(=O)C(c1cc(OCCCCN2CCCN(c3nccc(-c4noc([C@@]5(C)CCCc6sc(N)c(C#N)c65)n4)n3)[C@@H](C)C2)no1)C(C)C] | 1.52 | 704 | A |
| E-2k | 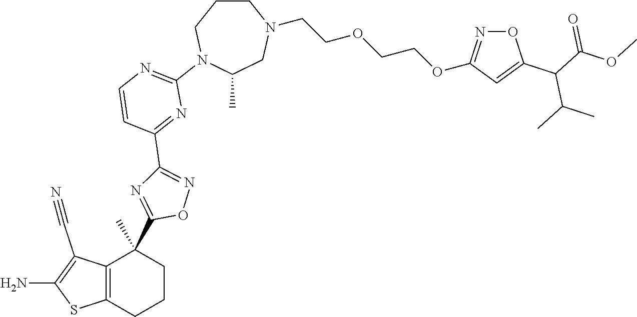 [SMILES: COC(=O)C(c1cc(OCCOCCN2CCCN(c3nccc(-c4noc([C@@]5(C)CCCc6sc(N)c(C#N)c65)n4)n3)[C@@H](C)C2)no1)C(C)C] | 1.58 | 720 | A |
| E-2l | 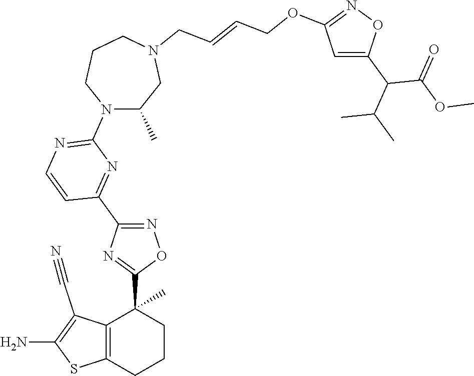 [SMILES: COC(=O)C(c1cc(OC/C=C/CN2CCCN(c3nccc(-c4noc([C@@]5(C)CCCc6sc(N)c(C#N)c65)n4)n3)[C@@H](C)C2)no1)C(C)C] | 1.64 | 702 | A |
| E-2s | 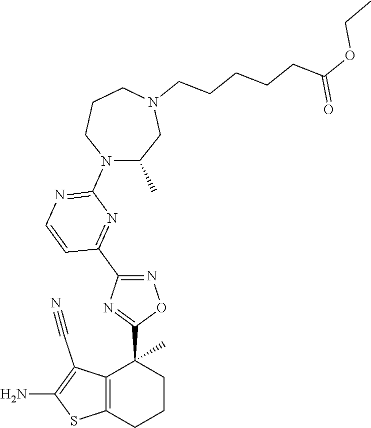 [SMILES: CCOC(=O)CCCCCN1CCCN(c2nccc(-c3noc([C@@]4(C)CCCc5sc(N)c(C#N)c54)n3)n2)[C@@H](C)C1] | 1.36 | 593 | M |
| E-2t | 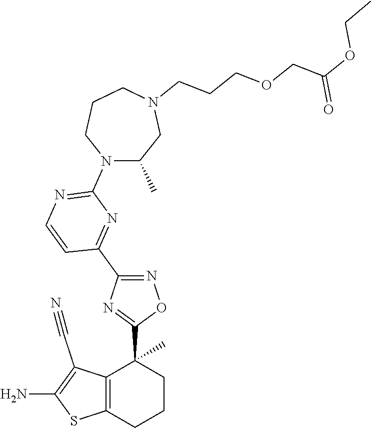 [SMILES: CCOC(=O)COCCCN1CCCN(c2nccc(-c3noc([C@@]4(C)CCCc5sc(N)c(C#N)c54)n3)n2)[C@@H](C)C1] | 1.32 | 595 | M |

### Experimental Procedure for the Synthesis of E-2m

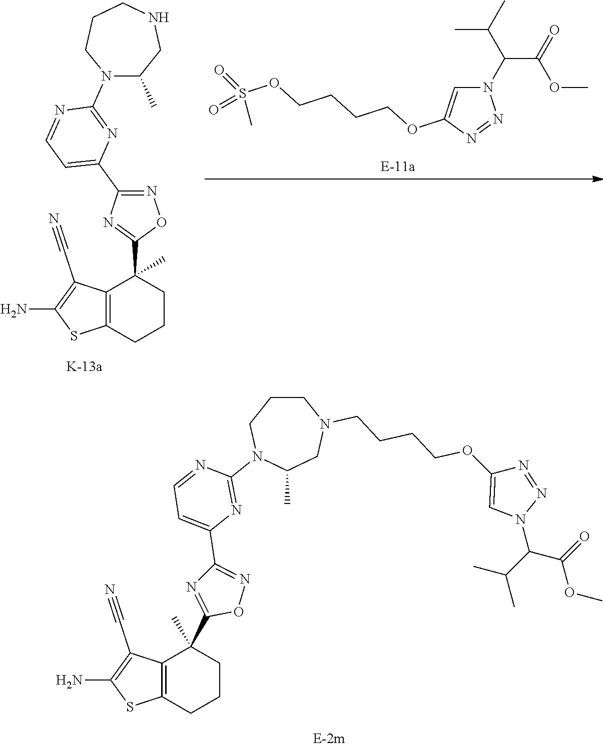 [SMILES: C[C@H]1CNCCCN1c1nccc(-c2noc([C@@]3(C)CCCc4sc(N)c(C#N)c43)n2)n1 ; COC(=O)C(C(C)C)n1cc(OCCCCOS(C)(=O)=O)nn1 ; COC(=O)C(C(C)C)n1cc(OCCCCN2CCCN(c3nccc(-c4noc([C@@]5(C)CCCc6sc(N)c(C#N)c65)n4)n3)[C@@H](C)C2)nn1]

To a stirred solution of K-13a (500 mg, 1.11 mmol, 1.0 eq.) and E-11a (659 mg, 1.89 mmol, 1.7 eq) in acetonitrile (45.0 mL) is added DIPEA (0-97 mL, 5.55 mmol, 5.0 eq) and the mixture is stirred at 65° C. under argon for 18 h. After complete conversion the reaction mixture is allowed to cool to rt, diluted with water and purified by RP chromatography to give E-2m.

The following intermediates E-2 (table 52) are available in an analogous manner starting from different intermediates K-13 and E-11. The crude products E-2 are purified by chromatography if necessary.

TABLE 52

| | | tᵣₑₜ | | HPLC |
|---|---|---|---|---|
| # | structure | [min] | [M + H]⁺ | method |
| E-2m | 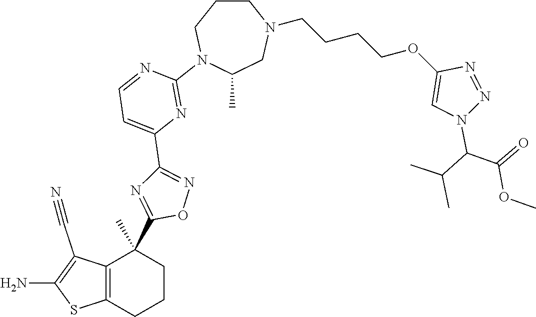 [SMILES: COC(=O)C(C(C)C)n1cc(OCCCCN2CCCN(c3nccc(-c4noc([C@@]5(C)CCCc6sc(N)c(C#N)c65)n4)n3)[C@@H](C)C2)nn1] | 1.54 | 704 | A |
| E-2n | 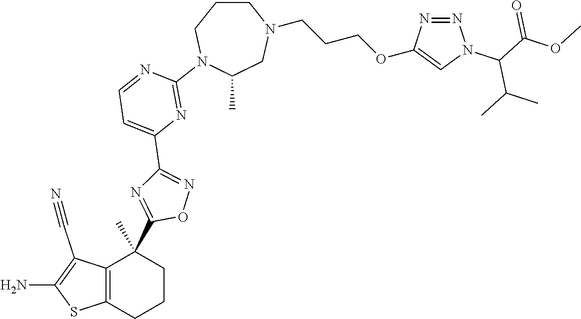 [SMILES: COC(=O)C(C(C)C)n1cc(OCCCN2CCCN(c3nccc(-c4noc([C@@]5(C)CCCc6sc(N)c(C#N)c65)n4)n3)[C@@H](C)C2)nn1] | 1.50 | 690 | A |
| E-2o | 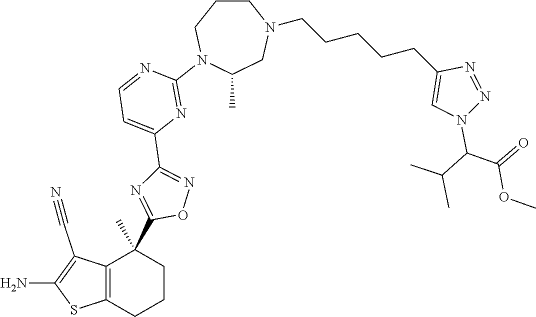 [SMILES: COC(=O)C(C(C)C)n1cc(CCCCCN2CCCN(c3nccc(-c4noc([C@@]5(C)CCCc6sc(N)c(C#N)c65)n4)n3)[C@@H](C)C2)nn1] | 1.56 | 702 | A |
| E-2s | 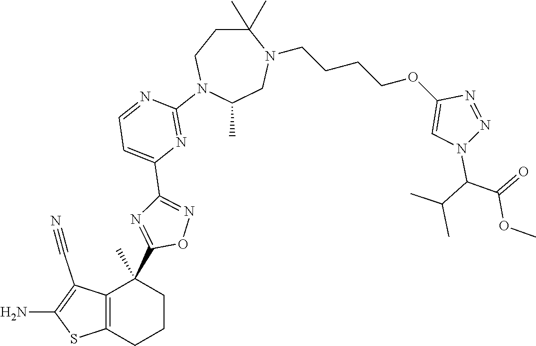 [SMILES: COC(=O)C(C(C)C)n1cc(OCCCCN2C[C@H](C)N(c3nccc(-c4noc([C@@]5(C)CCCc6sc(N)c(C#N)c65)n4)n3)CCC2(C)C)nn1] | 0.94 | 732 | H |

### Experimental Procedure for the Synthesis of E-2p

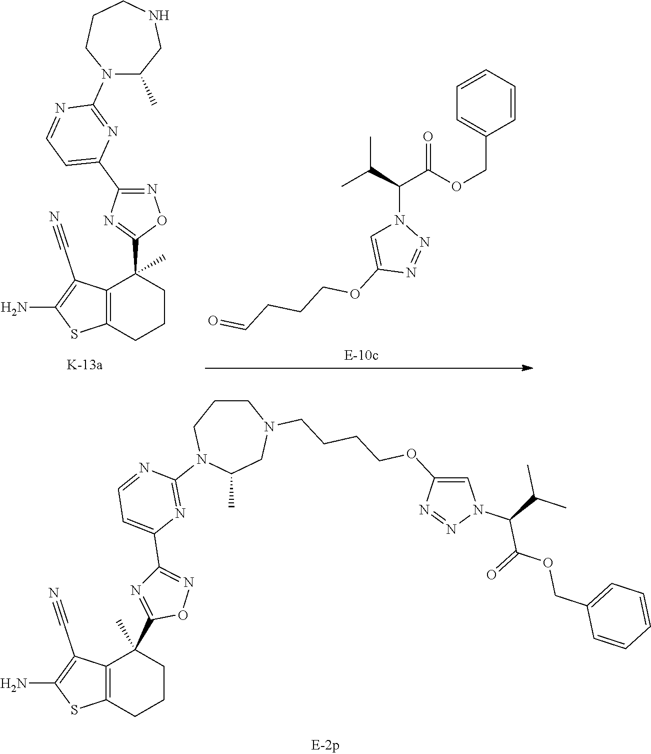 [SMILES: C[C@H]1CNCCCN1c1nccc(-c2noc([C@@]3(C)CCCc4sc(N)c(C#N)c43)n2)n1 ; CC(C)[C@@H](C(=O)OCc1ccccc1)n1cc(OCCCC=O)nn1 ; CC(C)[C@@H](C(=O)OCc1ccccc1)n1cc(OCCCCN2CCCN(c3nccc(-c4noc([C@@]5(C)CCCc6sc(N)c(C#N)c65)n4)n3)[C@@H](C)C2)nn1]

To a stirred solution of K-13a (200 mg, 0.444 mmol, 1.0 eq.) and sodium triacetoxyborohydride (235 mg, 1,110 mmol, 2.5 eq.) in DMF (3 mL) at RT is slowly added a solution of E-10c (153 mg, 0.444 mmol, 1.0 eq.) in DMF (1 mL). The reaction is stirred for 30 min before it is quenched by addition of water. The solvents are removed under reduced pressure and the crude product is purified by chromatography to obtain E-2p.

The following intermediates E-2 (table 53) are available in an analogous manner starting from different intermediates K-13 and E-10. The crude products E-2 are purified by chromatography if necessary.

TABLE 53

| | | tᵣₑₜ | | HPLC |
|---|---|---|---|---|
| # | structure | [min] | [M + H]⁺ | method |
| E-2p | 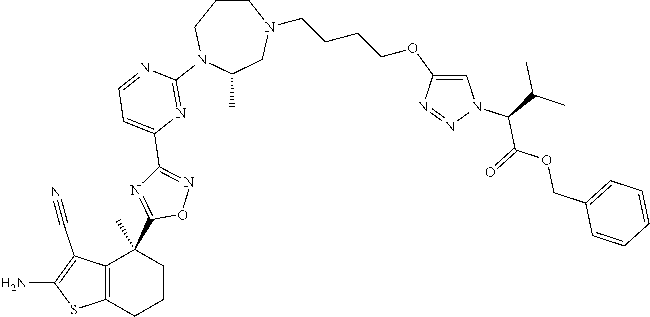 [SMILES: CC(C)[C@@H](C(=O)OCc1ccccc1)n1cc(OCCCCN2CCCN(c3nccc(-c4noc([C@@]5(C)CCCc6sc(N)c(C#N)c65)n4)n3)[C@@H](C)C2)nn1] | 1.66 | 780 | A |
| E-2q | 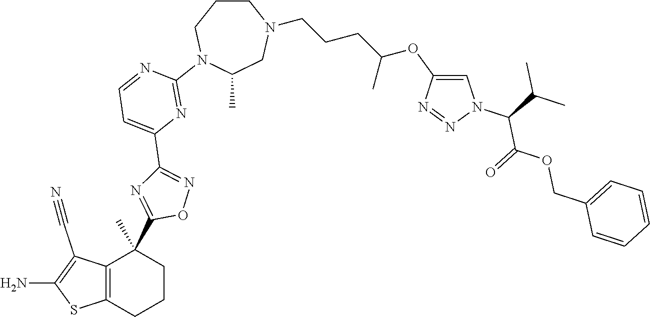 [SMILES: CC(CCCN1CCCN(c2nccc(-c3noc([C@@]4(C)CCCc5sc(N)c(C#N)c54)n3)n2)[C@@H](C)C1)Oc1cn([C@H](C(=O)OCc2ccccc2)C(C)C)nn1] | 0.96 | 794 | H |
| E-2r | 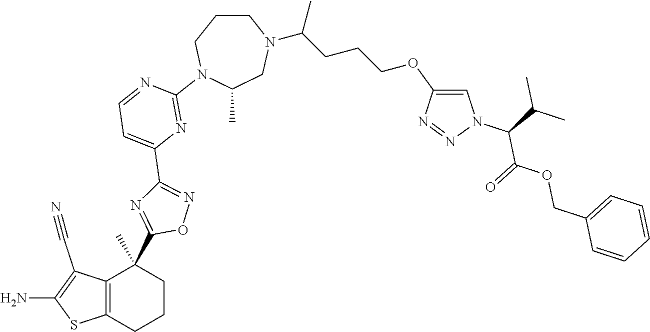 [SMILES: CC(C)[C@@H](C(=O)OCc1ccccc1)n1cc(OCCCC(C)N2CCCN(c3nccc(-c4noc([C@@]5(C)CCCc6sc(N)c(C#N)c65)n4)n3)[C@@H](C)C2)nn1] | 1.70 | 794 | A |

### Experimental Procedure for the Synthesis of E-3a

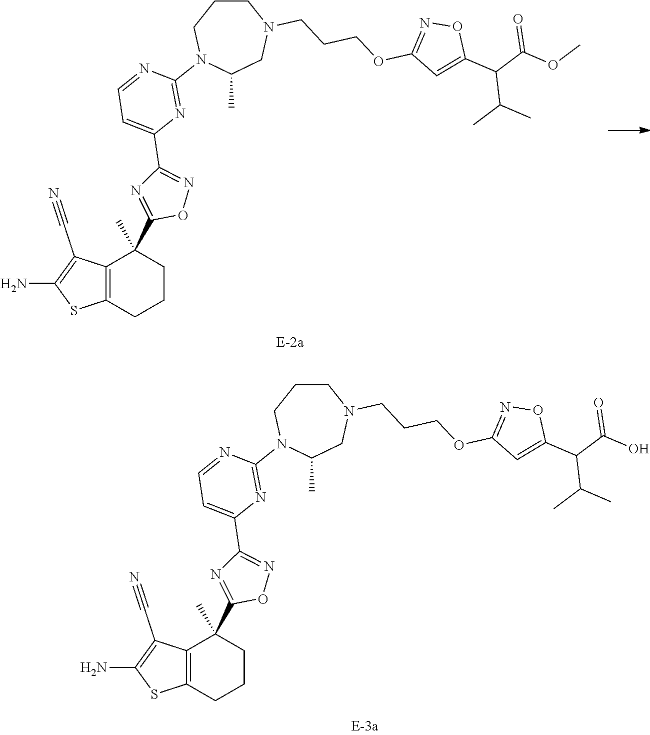 [SMILES: COC(=O)C(c1cc(OCCCN2CCCN(c3nccc(-c4noc([C@@]5(C)CCCc6sc(N)c(C#N)c65)n4)n3)[C@@H](C)C2)no1)C(C)C ; CC(C)C(C(=O)O)c1cc(OCCCN2CCCN(c3nccc(-c4noc([C@@]5(C)CCCc6sc(N)c(C#N)c65)n4)n3)[C@@H](C)C2)no1]

To a stirred solution of E-2a (4.26 g, 6.18 mmol, 1.0 eq.) in methanol (21.0 mL) is added sodium hydroxide solution (2 M in water, 6.18 mL, 12.35 mmol, 2.0 eq) and the reaction mixture is stirred at 45° C. for 1 h. After complete conversion the reaction mixture is concentrated under reduced pressure. The crude product is purified by chromatography yielding E-3a.

The following intermediates E-3 (table 54) are available in an analogous manner starting from different intermediates E-2. The crude products E-3 are purified by chromatography if necessary.

TABLE 54

| | | tᵣₑₜ | | HPLC |
|---|---|---|---|---|
| # | structure | [min] | [M + H]⁺ | method |
| E-3a | 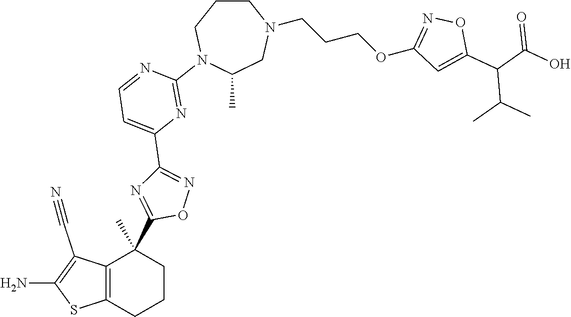 [SMILES: CC(C)C(C(=O)O)c1cc(OCCCN2CCCN(c3nccc(-c4noc([C@@]5(C)CCCc6sc(N)c(C#N)c65)n4)n3)[C@@H](C)C2)no1] | 1.14 | 676 | A |
| E-3b | 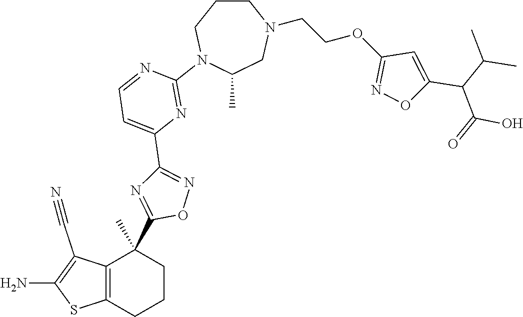 [SMILES: CC(C)C(C(=O)O)c1cc(OCCN2CCCN(c3nccc(-c4noc([C@@]5(C)CCCc6sc(N)c(C#N)c65)n4)n3)[C@@H](C)C2)no1] | n.a. | n.a. | n.a. |
| E-3c | 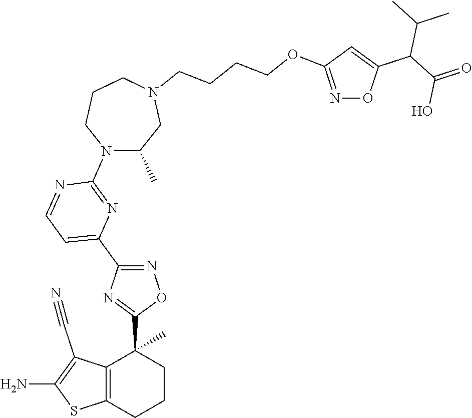 [SMILES: CC(C)C(C(=O)O)c1cc(OCCCCN2CCCN(c3nccc(-c4noc([C@@]5(C)CCCc6sc(N)c(C#N)c65)n4)n3)[C@@H](C)C2)no1] | 1.16 | 690 | A |
| E-3d | 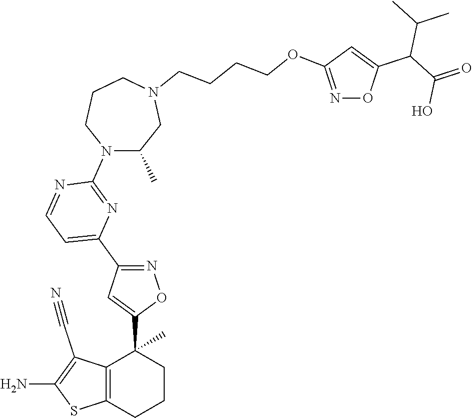 [SMILES: CC(C)C(C(=O)O)c1cc(OCCCCN2CCCN(c3nccc(-c4cc([C@@]5(C)CCCc6sc(N)c(C#N)c65)on4)n3)[C@@H](C)C2)no1] | 1.20 | 689 | A |
| E-3e | 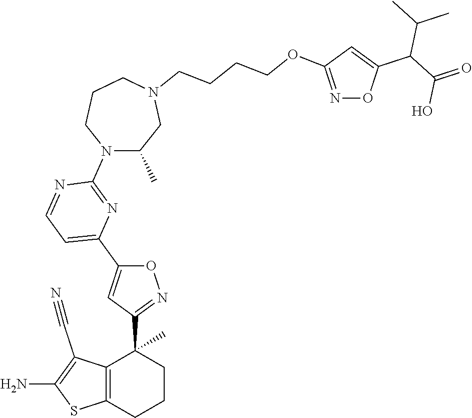 [SMILES: CC(C)C(C(=O)O)c1cc(OCCCCN2CCCN(c3nccc(-c4cc([C@@]5(C)CCCc6sc(N)c(C#N)c65)no4)n3)[C@@H](C)C2)no1] | 1.20 | 689 | A |
| E-3f | 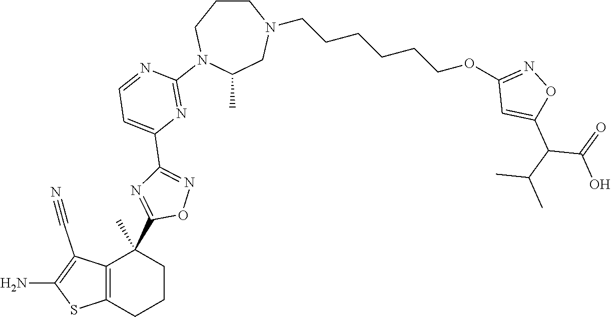 [SMILES: CC(C)C(C(=O)O)c1cc(OCCCCCCN2CCCN(c3nccc(-c4noc([C@@]5(C)CCCc6sc(N)c(C#N)c65)n4)n3)[C@@H](C)C2)no1] | 1.21 | 718 | A |
| E-3g | 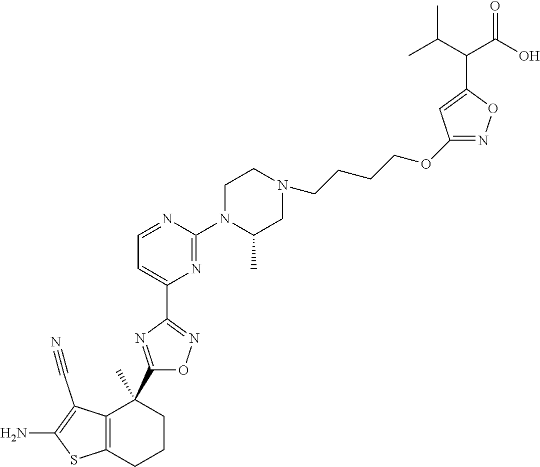 [SMILES: CC(C)C(C(=O)O)c1cc(OCCCCN2CCN(c3nccc(-c4noc([C@@]5(C)CCCc6sc(N)c(C#N)c65)n4)n3)[C@@H](C)C2)no1] | 0.59 | 676 | C |
| E-3h | 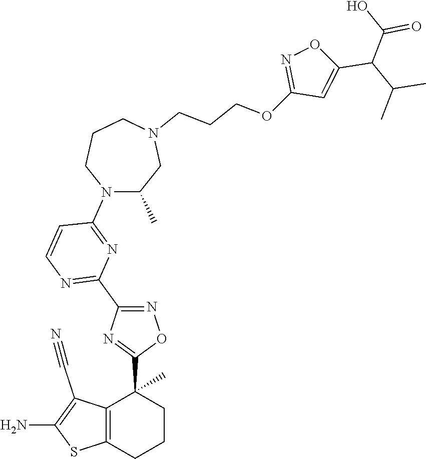 [SMILES: CC(C)C(C(=O)O)c1cc(OCCCN2CCCN(c3ccnc(-c4noc([C@@]5(C)CCCc6sc(N)c(C#N)c65)n4)n3)[C@@H](C)C2)no1] | 1.05 | 676 | A |
| E-3i | 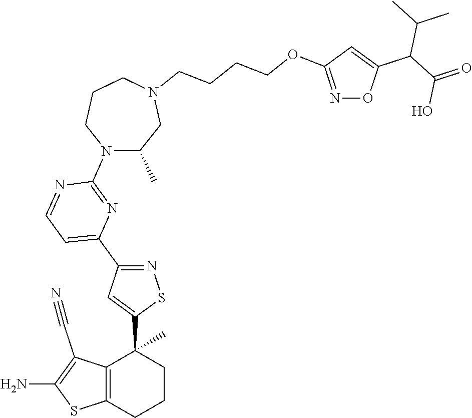 [SMILES: CC(C)C(C(=O)O)c1cc(OCCCCN2CCCN(c3nccc(-c4cc([C@@]5(C)CCCc6sc(N)c(C#N)c65)sn4)n3)[C@@H](C)C2)no1] | 1.22 | 705 | A |
| E-3j | 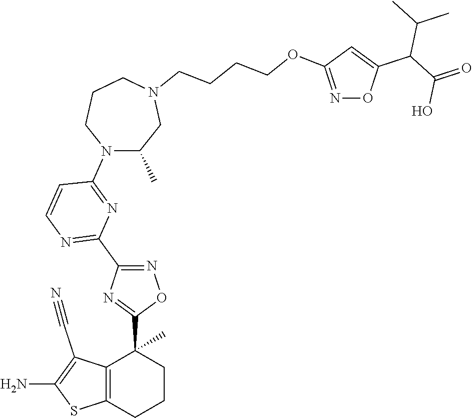 [SMILES: CC(C)C(C(=O)O)c1cc(OCCCCN2CCCN(c3ccnc(-c4noc([C@@]5(C)CCCc6sc(N)c(C#N)c65)n4)n3)[C@@H](C)C2)no1] | 1.06 | 690 | A |
| E-3k | 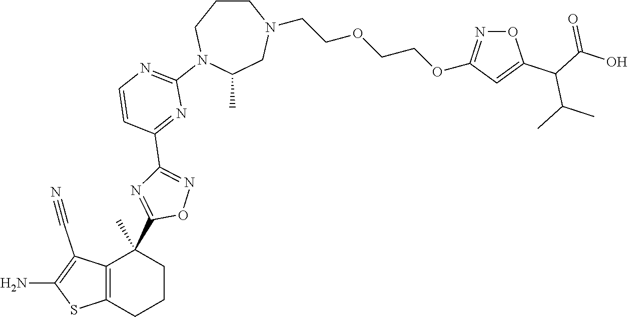 [SMILES: CC(C)C(C(=O)O)c1cc(OCCOCCN2CCCN(c3nccc(-c4noc([C@@]5(C)CCCc6sc(N)c(C#N)c65)n4)n3)[C@@H](C)C2)no1] | 1.12 | 706 | A |
| E-3l | 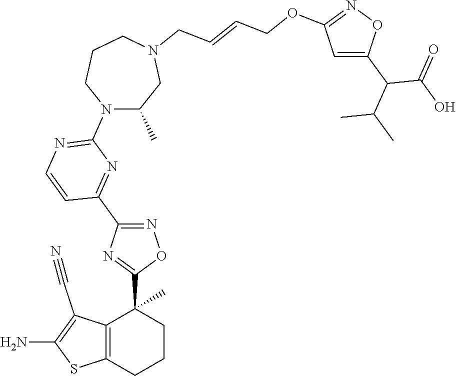 [SMILES: CC(C)C(C(=O)O)c1cc(OC/C=C/CN2CCCN(c3nccc(-c4noc([C@@]5(C)CCCc6sc(N)c(C#N)c65)n4)n3)[C@@H](C)C2)no1] | 0.57 | 688 | C |
| E-3m | 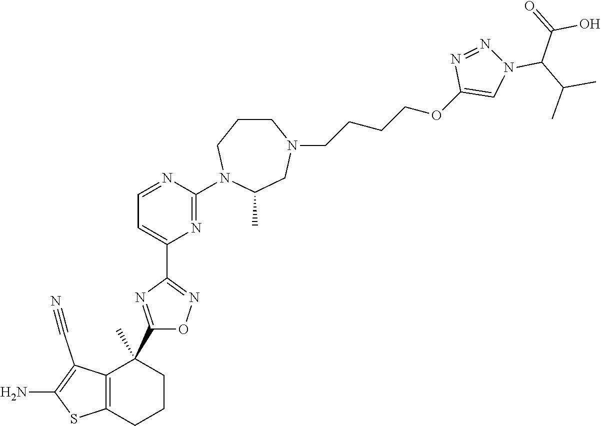 [SMILES: CC(C)C(C(=O)O)n1cc(OCCCCN2CCCN(c3nccc(-c4noc([C@@]5(C)CCCc6sc(N)c(C#N)c65)n4)n3)[C@@H](C)C2)nn1] | 1.12 | 690 | A |
| E-3n | 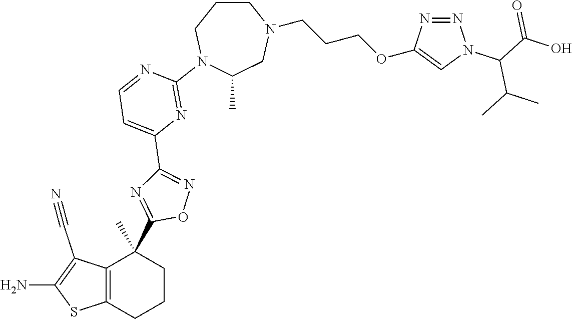 [SMILES: CC(C)C(C(=O)O)n1cc(OCCCN2CCCN(c3nccc(-c4noc([C@@]5(C)CCCc6sc(N)c(C#N)c65)n4)n3)[C@@H](C)C2)nn1] | 1.11 | 676 | A |
| E-3o | 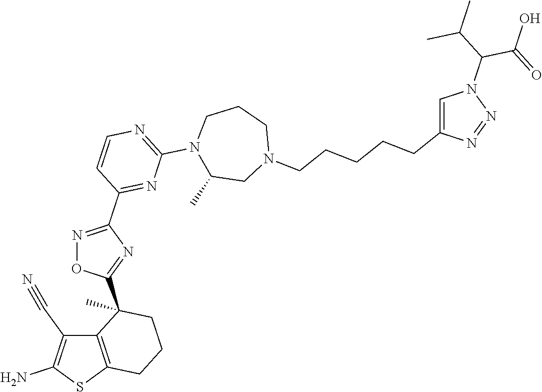 [SMILES: CC(C)C(C(=O)O)n1cc(CCCCCN2CCCN(c3nccc(-c4noc([C@@]5(C)CCCc6sc(N)c(C#N)c65)n4)n3)[C@@H](C)C2)nn1] | 1.16 | 688 | A |
| E-3s | 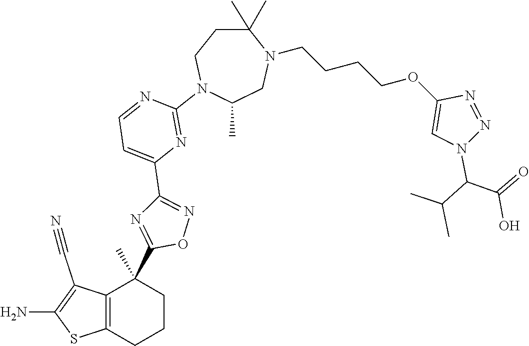 [SMILES: CC(C)C(C(=O)O)n1cc(OCCCCN2C[C@H](C)N(c3nccc(-c4noc([C@@]5(C)CCCc6sc(N)c(C#N)c65)n4)n3)CCC2(C)C)nn1] | 1.25 | 718 | A |

### Experimental Procedure for the Synthesis of E-3p

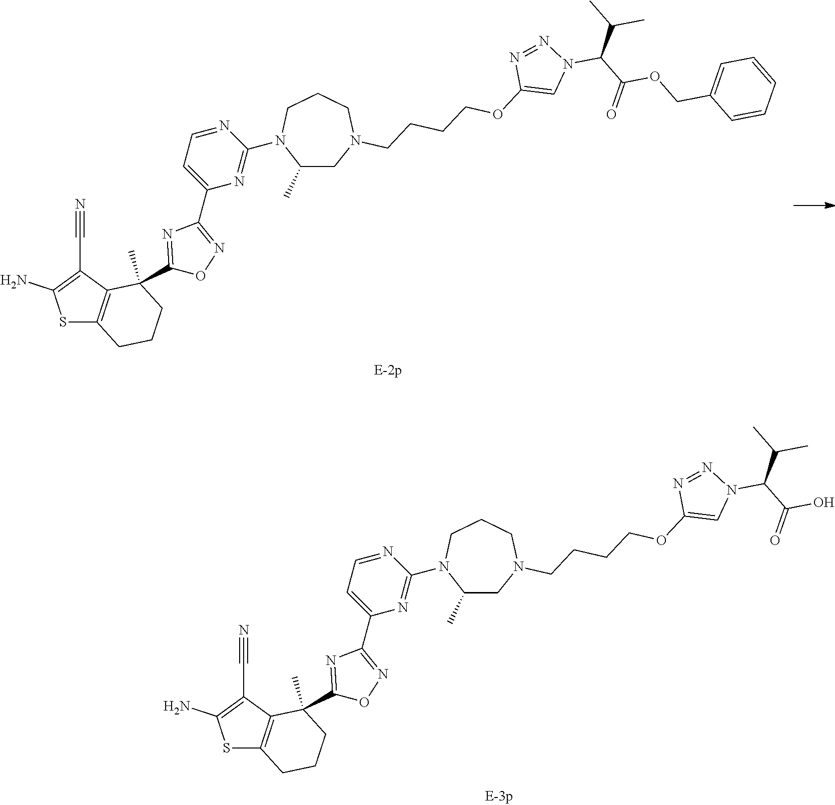 [SMILES: CC(C)[C@@H](C(=O)OCc1ccccc1)n1cc(OCCCCN2CCCN(c3nccc(-c4noc([C@@]5(C)CCCc6sc(N)c(C#N)c65)n4)n3)[C@@H](C)C2)nn1 ; CC(C)[C@@H](C(=O)O)n1cc(OCCCCN2CCCN(c3nccc(-c4noc([C@@]5(C)CCCc6sc(N)c(C#N)c65)n4)n3)[C@@H](C)C2)nn1]

To a solution of intermediate E-2p (1.33 g, 1.705 mmol, 1.0 eq.) in MeOH (25 mL) in a hydrogenation reactor is added Pd/C (10%, 350.00 mg). The reaction mixture stirred under a pressure of 8 bar H₂ for 8 h. After complete conversion the reaction mixture is filtered, and the solvent is removed under reduced pressure to give intermediate E-3p.

The following intermediates E-3 (table 55) are available in an analogous manner starting from different intermediates E-2. The crude products E-3 are purified by chromatography if necessary.

TABLE 55

| | | tᵣₑₜ | | HPLC |
|---|---|---|---|---|
| # | structure | [min] | [M + H]⁺ | method |
| E-3p | 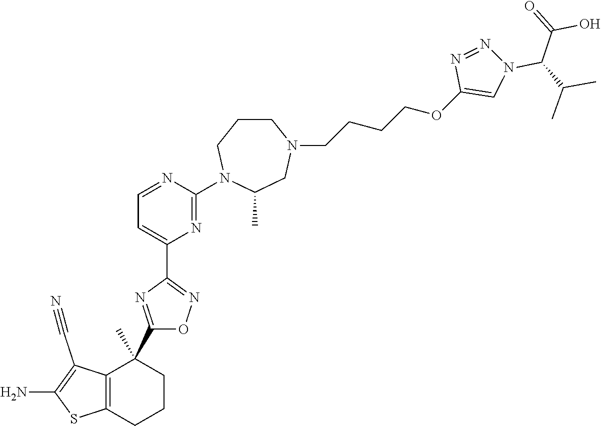 [SMILES: CC(C)[C@@H](C(=O)O)n1cc(OCCCCN2CCCN(c3nccc(-c4noc([C@@]5(C)CCCc6sc(N)c(C#N)c65)n4)n3)[C@@H](C)C2)nn1] | 1.12 | 690 | A |
| E-3q | 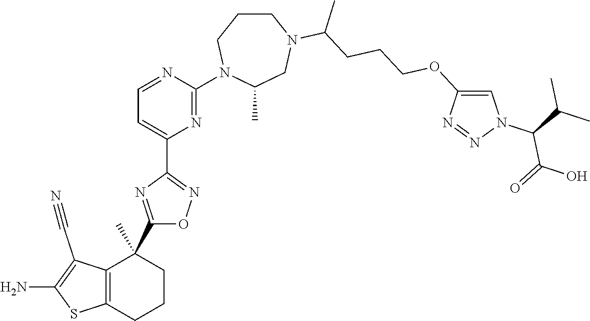 [SMILES: CC(C)[C@@H](C(=O)O)n1cc(OCCCC(C)N2CCCN(c3nccc(-c4noc([C@@]5(C)CCCc6sc(N)c(C#N)c65)n4)n3)[C@@H](C)C2)nn1] | 0.57 | 704 | J |
| E-3r | 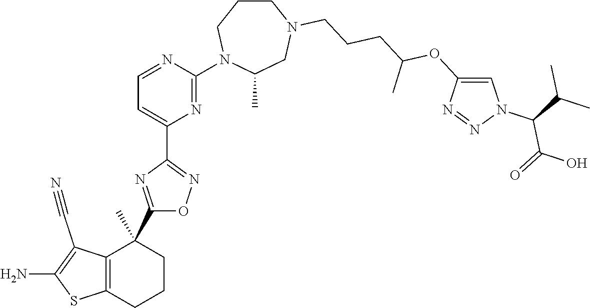 [SMILES: CC(CCCN1CCCN(c2nccc(-c3noc([C@@]4(C)CCCc5sc(N)c(C#N)c54)n3)n2)[C@@H](C)C1)Oc1cn([C@H](C(=O)O)C(C)C)nn1] | 0.58 | 704 | J |

### Experimental Procedure for the Synthesis of E-4a

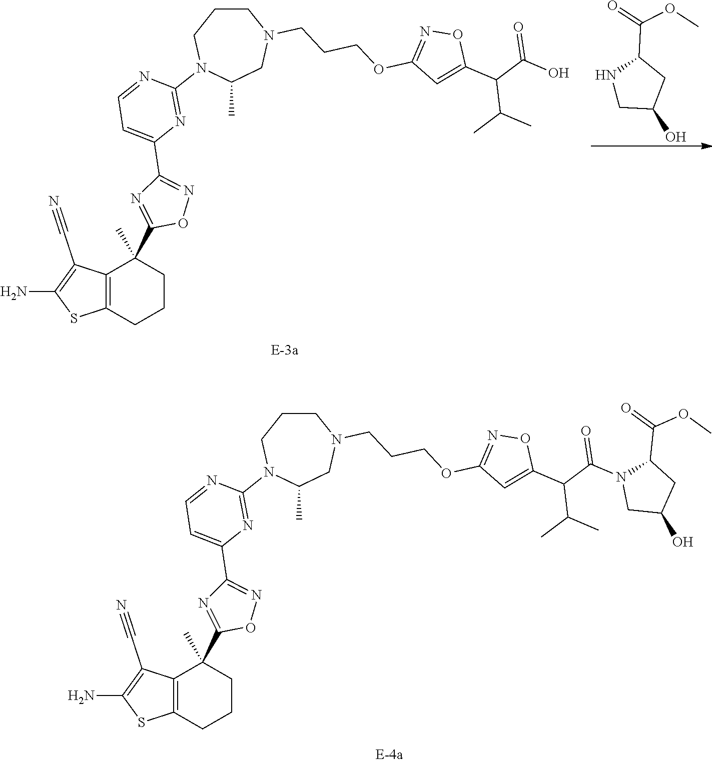

To a stirred solution of E-3a (4.38 g, 6.48 mmol, 1.0 eq.) and methyl (2S,4R)-4-hydroxypyrrolidine-2-carboxylate hydrochloride (1.61 g, 8.43 mmol, 1.3 eq.) and HATU (3.27 mg, 8.43 mmol, 1.3 eq.) in acetonitrile (35 mL) and DMSO (9.00 mL) is added DIPEA (5.66 mL, 32.41 mmol, 5.0 eq) and stirred at rt for 30 min. After complete conversion the reaction mixture is diluted with water and EtOAc. The layers are separated, and the organic layer is washed with water and brine, dried over magnesium sulfate and concentrated under reduced pressure to give the crude product. The crude product is purified by chromatography yielding E-4a.

The following intermediates E-4 (table 56) are available in an analogous manner starting from different intermediates E-3 and/or different protected pyrrolidines. The crude products E-4 are purified by chromatography if necessary.

TABLE 56

| | | tᵣₑₜ | | HPLC |
|---|---|---|---|---|
| # | structure | [min] | [M + H]⁺ | method |
| E-4a | 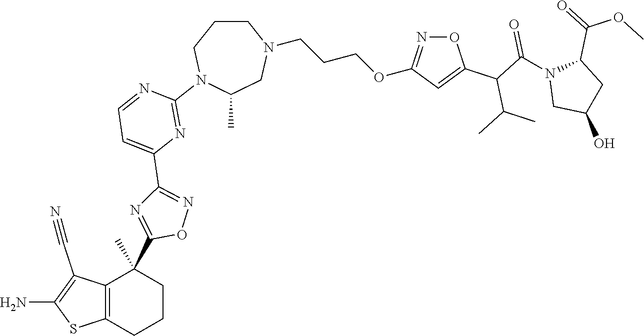 [SMILES: COC(=O)[C@@H]1C[C@@H](O)CN1C(=O)C(c1cc(OCCCN2CCCN(c3nccc(-c4noc([C@@]5(C)CCCc6sc(N)c(C#N)c65)n4)n3)[C@@H](C)C2)no1)C(C)C] | 1.43 | 803 | A |
| E-4b | 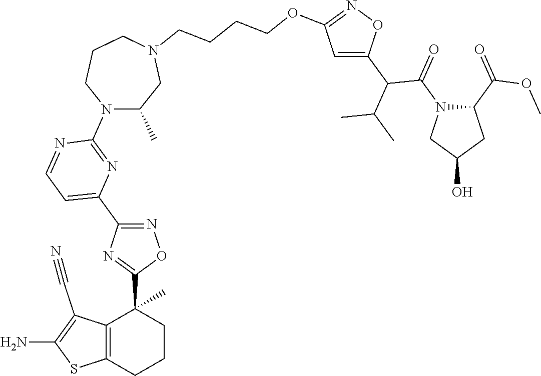 [SMILES: COC(=O)[C@@H]1C[C@@H](O)CN1C(=O)C(c1cc(OCCCCN2CCCN(c3nccc(-c4noc([C@@]5(C)CCCc6sc(N)c(C#N)c65)n4)n3)[C@@H](C)C2)no1)C(C)C] | 1.47 | 817 | A |
| E-4c | 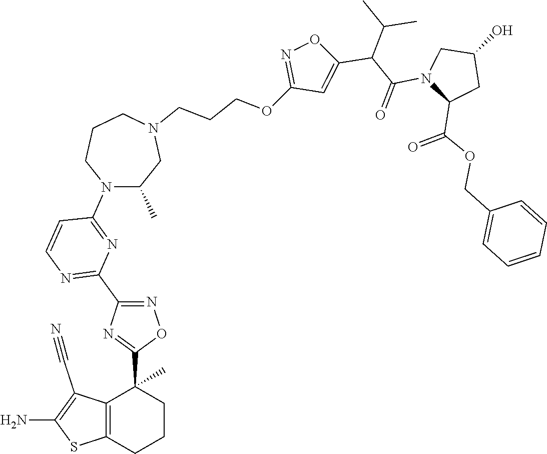 [SMILES: CC(C)C(C(=O)N1C[C@H](O)C[C@H]1C(=O)OCc1ccccc1)c1cc(OCCCN2CCCN(c3ccnc(-c4noc([C@@]5(C)CCCc6sc(N)c(C#N)c65)n4)n3)[C@@H](C)C2)no1] | 1.43 | 879 | A |
| E-4d | 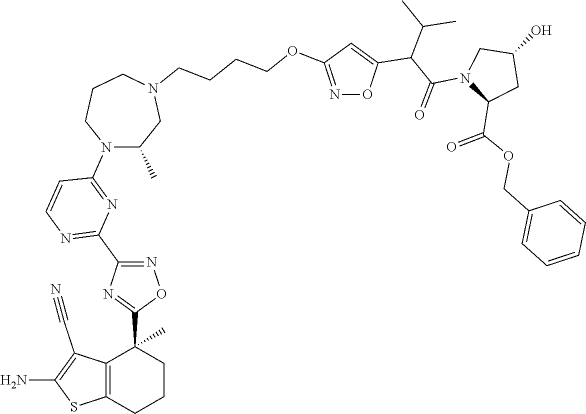 [SMILES: CC(C)C(C(=O)N1C[C@H](O)C[C@H]1C(=O)OCc1ccccc1)c1cc(OCCCCN2CCCN(c3ccnc(-c4noc([C@@]5(C)CCCc6sc(N)c(C#N)c65)n4)n3)[C@@H](C)C2)no1] | 1.49 | 893 | A |
| E-4e | 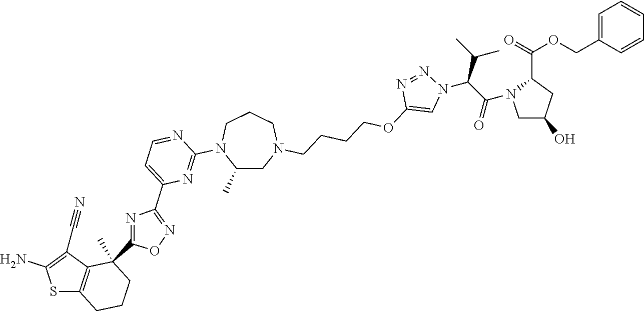 [SMILES: CC(C)[C@@H](C(=O)N1C[C@H](O)C[C@H]1C(=O)OCc1ccccc1)n1cc(OCCCCN2CCCN(c3nccc(-c4noc([C@@]5(C)CCCc6sc(N)c(C#N)c65)n4)n3)[C@@H](C)C2)nn1] | 1.56 | 893 | A |
| E-4f | 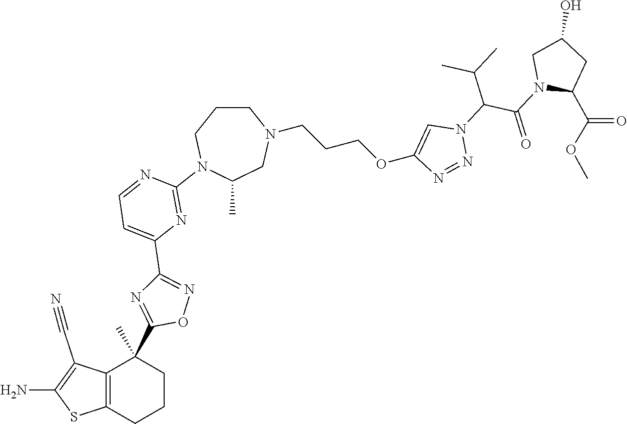 [SMILES: COC(=O)[C@@H]1C[C@@H](O)CN1C(=O)C(C(C)C)n1cc(OCCCN2CCCN(c3nccc(-c4noc([C@@]5(C)CCCc6sc(N)c(C#N)c65)n4)n3)[C@@H](C)C2)nn1] | 1.40 | 803 | A |

### Experimental Procedure for the Synthesis of E-5a

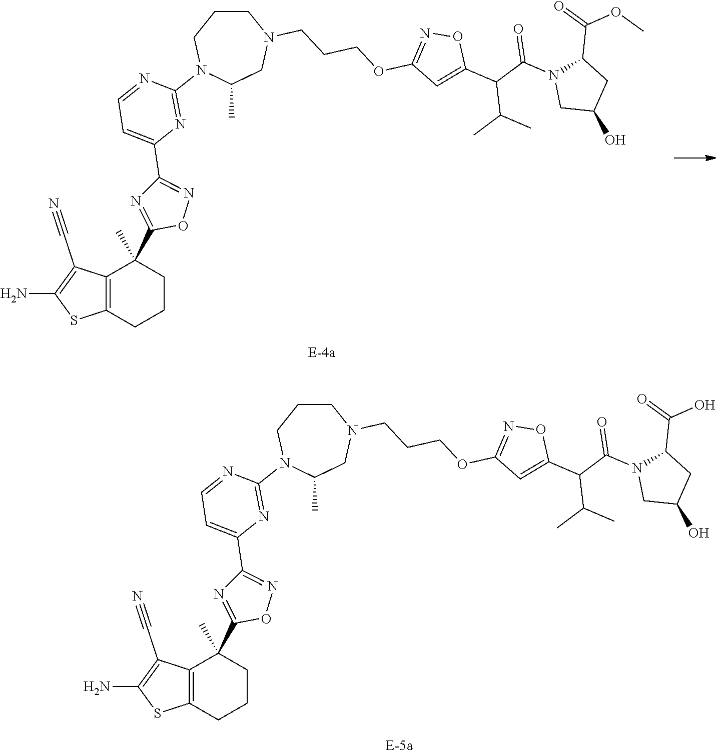 [SMILES: COC(=O)[C@@H]1C[C@@H](O)CN1C(=O)C(c1cc(OCCCN2CCCN(c3nccc(-c4noc([C@@]5(C)CCCc6sc(N)c(C#N)c65)n4)n3)[C@@H](C)C2)no1)C(C)C ; CC(C)C(C(=O)N1C[C@H](O)C[C@H]1C(=O)O)c1cc(OCCCN2CCCN(c3nccc(-c4noc([C@@]5(C)CCCc6sc(N)c(C#N)c65)n4)n3)[C@@H](C)C2)no1]

To a stirred solution of E-4a (4.47 g, 5.57 mmol, 1.0 eq.) in methanol (22.0 mL) is added sodium hydroxide solution (2 M in water, 6.00 mL, 12.00 mmol, 2.2 eq.) and the reaction mixture is stirred at 45° C. for 1 h. After complete conversion the reaction mixture is concentrated under reduced pressure. The crude product is purified by chromatography yielding E-5a.

The following intermediates E-5 (table 57) are available in an analogous manner starting from different intermediates E-4. The crude products E-5 are purified by chromatography if necessary.

TABLE 57

| | | tᵣₑₜ | | HPLC |
|---|---|---|---|---|
| # | structure | [min] | [M + H]⁺ | method |
| E-5a | 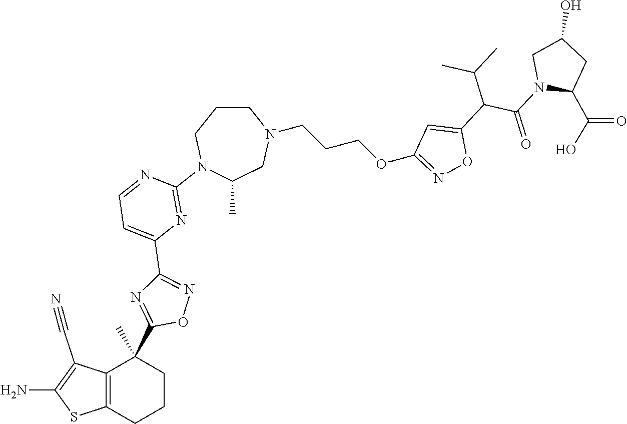 [SMILES: CC(C)C(C(=O)N1C[C@H](O)C[C@H]1C(=O)O)c1cc(OCCCN2CCCN(c3nccc(-c4noc([C@@]5(C)CCCc6sc(N)c(C#N)c65)n4)n3)[C@@H](C)C2)no1] | 1.11 | 789 | A |
| E-5b | 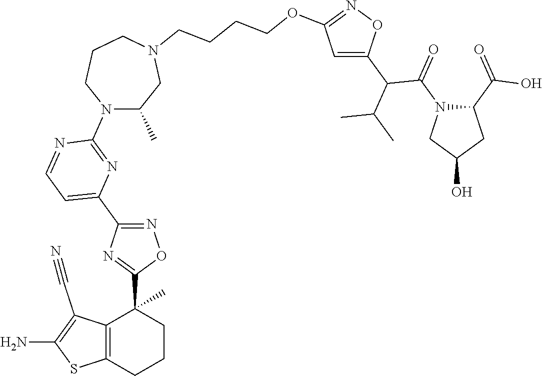 [SMILES: CC(C)C(C(=O)N1C[C@H](O)C[C@H]1C(=O)O)c1cc(OCCCCN2CCCN(c3nccc(-c4noc([C@@]5(C)CCCc6sc(N)c(C#N)c65)n4)n3)[C@@H](C)C2)no1] | 1.14 | 803 | A |
| E-5c | 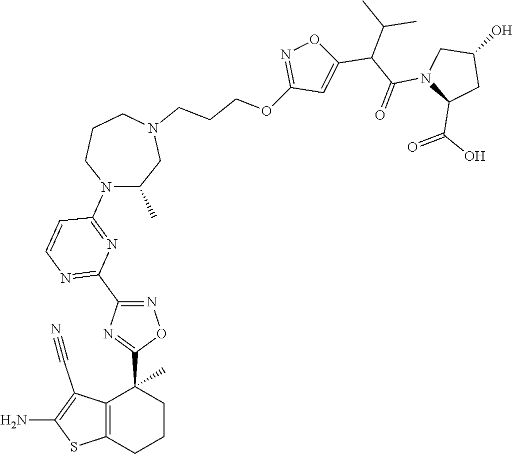 [SMILES: CC(C)C(C(=O)N1C[C@H](O)C[C@H]1C(=O)O)c1cc(OCCCN2CCCN(c3ccnc(-c4noc([C@@]5(C)CCCc6sc(N)c(C#N)c65)n4)n3)[C@@H](C)C2)no1] | 1.03 | 789 | A |
| E-5d | 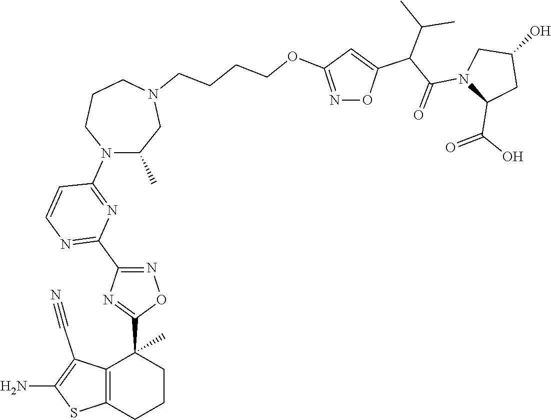 [SMILES: CC(C)C(C(=O)N1C[C@H](O)C[C@H]1C(=O)O)c1cc(OCCCCN2CCCN(c3ccnc(-c4noc([C@@]5(C)CCCc6sc(N)c(C#N)c65)n4)n3)[C@@H](C)C2)no1] | 1.06 | 803 | A |
| E-5f | 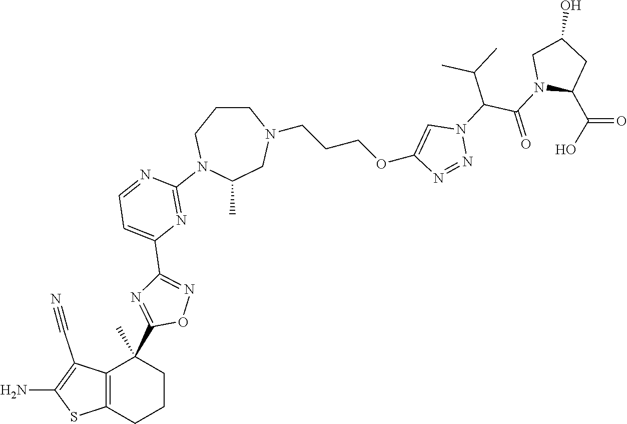 [SMILES: CC(C)C(C(=O)N1C[C@H](O)C[C@H]1C(=O)O)n1cc(OCCCN2CCCN(c3nccc(-c4noc([C@@]5(C)CCCc6sc(N)c(C#N)c65)n4)n3)[C@@H](C)C2)nn1] | 1.13 | 789 | A |

### Experimental Procedure for the Synthesis of E-5e

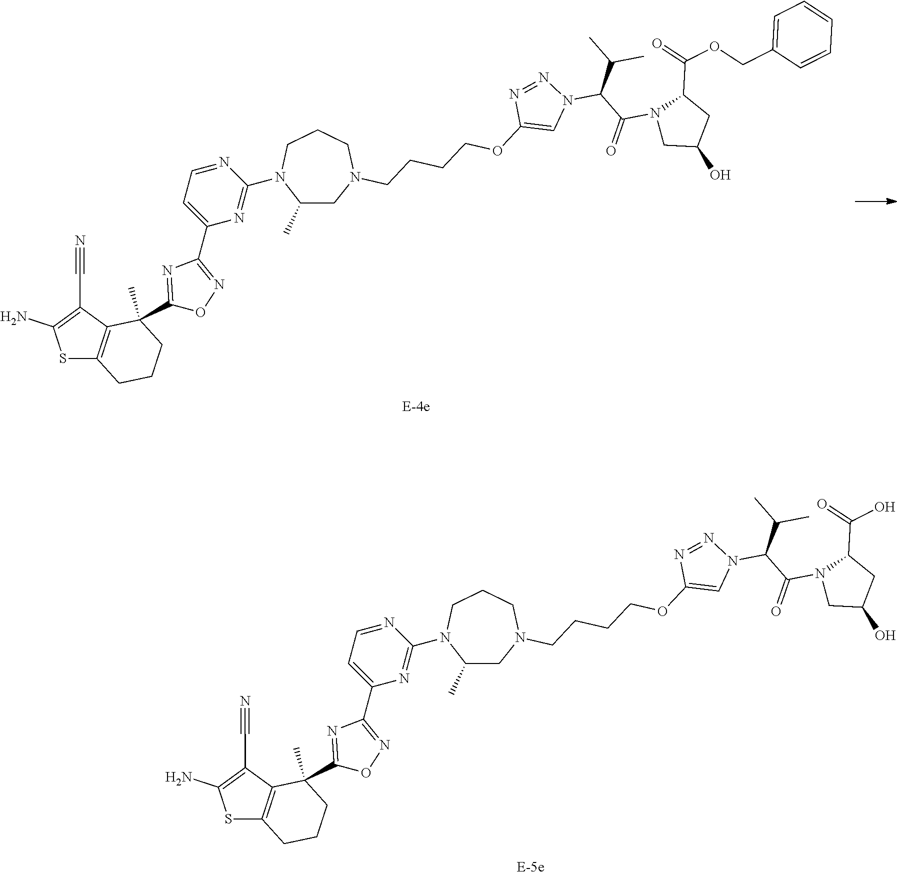 [SMILES: CC(C)[C@@H](C(=O)N1C[C@H](O)C[C@H]1C(=O)OCc1ccccc1)n1cc(OCCCCN2CCCN(c3nccc(-c4noc([C@@]5(C)CCCc6sc(N)c(C#N)c65)n4)n3)[C@@H](C)C2)nn1 ; CC(C)[C@@H](C(=O)N1C[C@H](O)C[C@H]1C(=O)O)n1cc(OCCCCN2CCCN(c3nccc(-c4noc([C@@]5(C)CCCc6sc(N)c(C#N)c65)n4)n3)[C@@H](C)C2)nn1]

To a solution of intermediate E-4e (66 mg, 0.074 mmol, 1.0 eq.) in MeOH (2 mL) in a hydrogenation reactor is added Pd/C (10%, 50.00 mg). The reaction mixture stirred under a pressure of 8 bar H₂ for 3 h. After complete conversion the reaction mixture is filtered, and the solvent is removed under reduced pressure to give intermediate E-5e (table 58).

TABLE 58

| | | tᵣₑₜ | | HPLC |
|---|---|---|---|---|
| # | structure | [min] | [M + H]⁺ | method |
| E-5e | 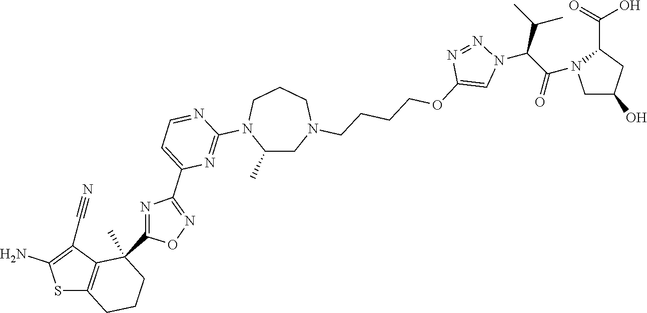 [SMILES: CC(C)[C@@H](C(=O)N1C[C@H](O)C[C@H]1C(=O)O)n1cc(OCCCCN2CCCN(c3nccc(-c4noc([C@@]5(C)CCCc6sc(N)c(C#N)c65)n4)n3)[C@@H](C)C2)nn1] | 0.53 | 803 | H |

### Experimental Procedure for the Synthesis of I-1

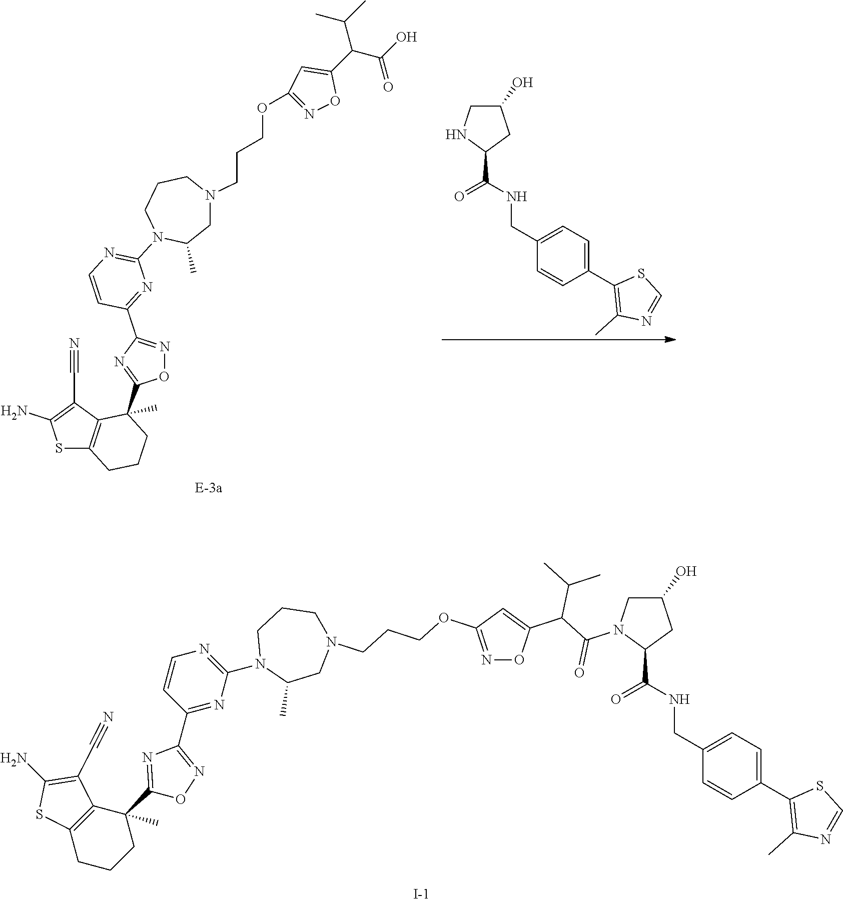 [SMILES: CC(C)C(C(=O)O)c1cc(OCCCN2CCCN(c3nccc(-c4noc([C@@]5(C)CCCc6sc(N)c(C#N)c65)n4)n3)[C@@H](C)C2)no1 ; Cc1ncsc1-c1ccc(CNC(=O)[C@@H]2C[C@@H](O)CN2)cc1 ; Cc1ncsc1-c1ccc(CNC(=O)[C@@H]2C[C@@H](O)CN2C(=O)C(c2cc(OCCCN3CCCN(c4nccc(-c5noc([C@@]6(C)CCCc7sc(N)c(C#N)c76)n5)n4)[C@@H](C)C3)no2)C(C)C)cc1 ; [I-]]

To a stirred solution of E-3a (219 mg, 0.32 mmol, 1.0 eq.), (2S,4R)-4-hydroxy-N-{[4-(4-methyl-1,3-thiazol-5-yl)phenyl]methyl}pyrrolidine-2-carboxamide (113 mg, 0.36 mmol, 1.1 eq.) and HATU (184 mg, 0.48 mmol, 1.3 eq.) in DMF (1.0 mL) is added DIPEA (0.16 mL, 0.97 mmol, 3.0 eq) and the reaction mixture is stirred at rt for 30 min. After complete conversion the reaction mixture is quenched with water, diluted with acetonitrile and purified by chromatography.

The following compounds I (table 59) are available in an analogous manner starting from different intermediates E-3 and A-4.

TABLE 59

| | | tᵣₑₜ | | HPLC |
|---|---|---|---|---|
| # | structure | [min] | [M + H]⁺ | method |
| I-1 | 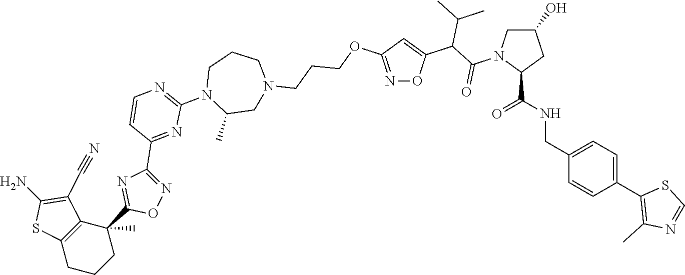 [SMILES: Cc1ncsc1-c1ccc(CNC(=O)[C@@H]2C[C@@H](O)CN2C(=O)C(c2cc(OCCCN3CCCN(c4nccc(-c5noc([C@@]6(C)CCCc7sc(N)c(C#N)c76)n5)n4)[C@@H](C)C3)no2)C(C)C)cc1] | 0.78 | 975 | H |
| I-2 | 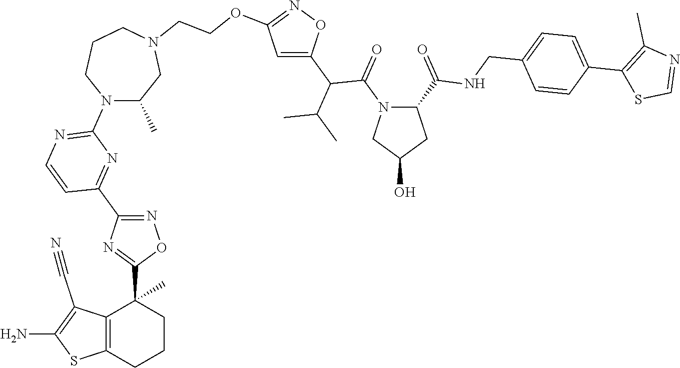 [SMILES: Cc1ncsc1-c1ccc(CNC(=O)[C@@H]2C[C@@H](O)CN2C(=O)C(c2cc(OCCN3CCCN(c4nccc(-c5noc([C@@]6(C)CCCc7sc(N)c(C#N)c76)n5)n4)[C@@H](C)C3)no2)C(C)C)cc1] | 1.41 | 961 | A |
| I-3 | 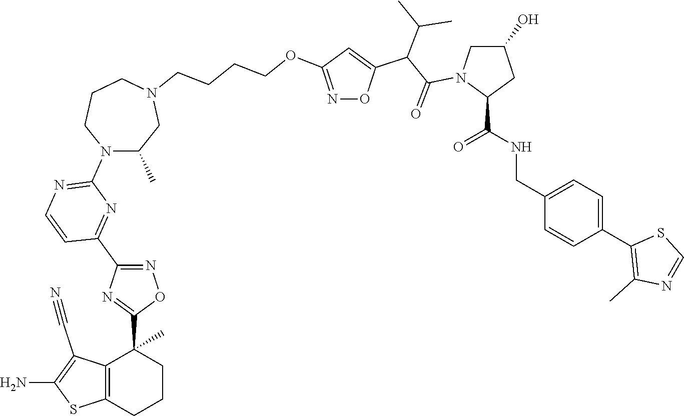 [SMILES: Cc1ncsc1-c1ccc(CNC(=O)[C@@H]2C[C@@H](O)CN2C(=O)C(c2cc(OCCCCN3CCCN(c4nccc(-c5noc([C@@]6(C)CCCc7sc(N)c(C#N)c76)n5)n4)[C@@H](C)C3)no2)C(C)C)cc1] | 1.47 | 989 | A |
| I-4 | 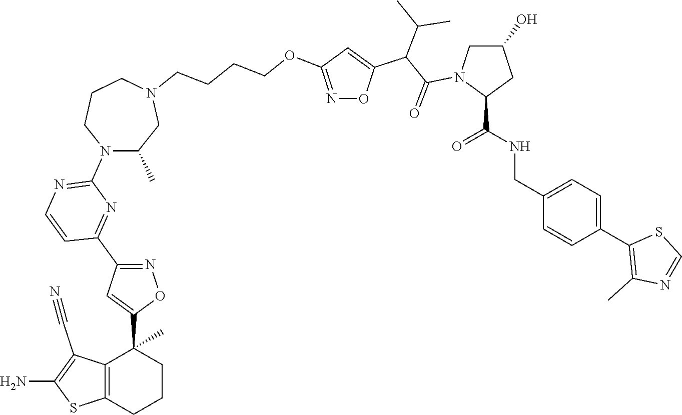 [SMILES: Cc1ncsc1-c1ccc(CNC(=O)[C@@H]2C[C@@H](O)CN2C(=O)C(c2cc(OCCCCN3CCCN(c4nccc(-c5cc([C@@]6(C)CCCc7sc(N)c(C#N)c76)on5)n4)[C@@H](C)C3)no2)C(C)C)cc1] | 0.83 | 986 [M − H]⁺ | B |
| I-5 | 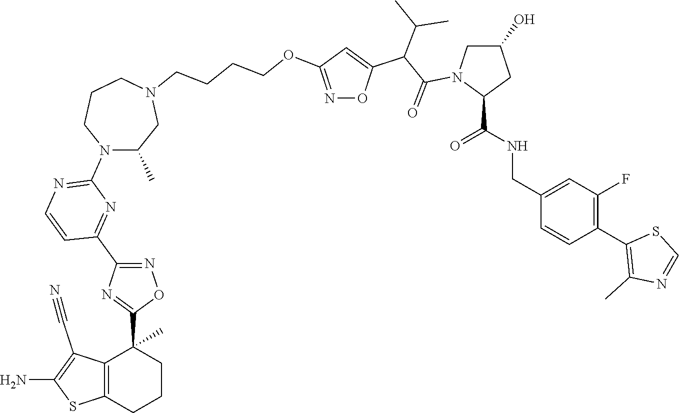 [SMILES: Cc1ncsc1-c1ccc(CNC(=O)[C@@H]2C[C@@H](O)CN2C(=O)C(c2cc(OCCCCN3CCCN(c4nccc(-c5noc([C@@]6(C)CCCc7sc(N)c(C#N)c76)n5)n4)[C@@H](C)C3)no2)C(C)C)cc1F] | 0.75 | 1007 | H |
| I-6 | 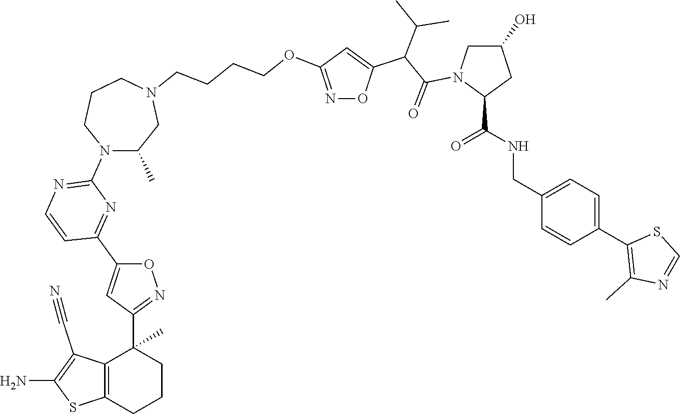 [SMILES: Cc1ncsc1-c1ccc(CNC(=O)[C@@H]2C[C@@H](O)CN2C(=O)C(c2cc(OCCCCN3CCCN(c4nccc(-c5cc([C@@]6(C)CCCc7sc(N)c(C#N)c76)no5)n4)[C@@H](C)C3)no2)C(C)C)cc1] | 0.82 | 986 [M − H]⁺ | B |
| I-7 | 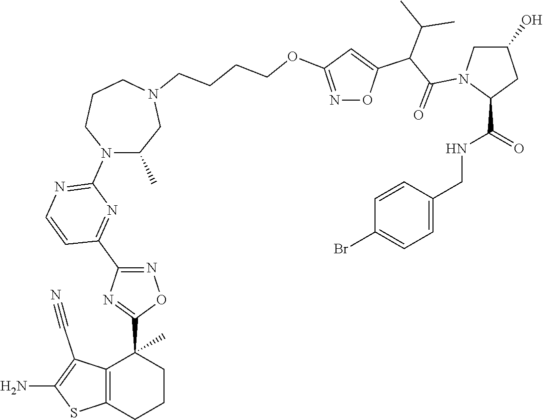 [SMILES: CC(C)C(C(=O)N1C[C@H](O)C[C@H]1C(=O)NCc1ccc(Br)cc1)c1cc(OCCCCN2CCCN(c3nccc(-c4noc([C@@]5(C)CCCc6sc(N)c(C#N)c65)n4)n3)[C@@H](C)C2)no1] | 0.88 | 970 | H |
| I-8 | 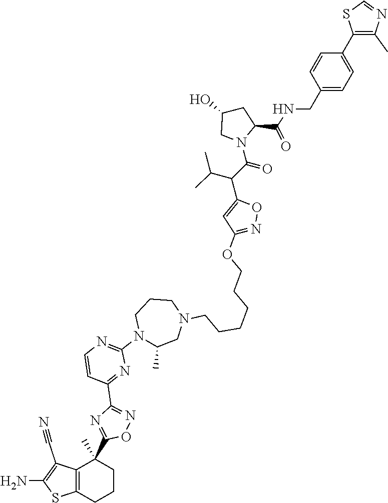 [SMILES: Cc1ncsc1-c1ccc(CNC(=O)[C@@H]2C[C@@H](O)CN2C(=O)C(c2cc(OCCCCCCN3CCCN(c4nccc(-c5noc([C@@]6(C)CCCc7sc(N)c(C#N)c76)n5)n4)[C@@H](C)C3)no2)C(C)C)cc1] | 1.54 | 1017 | A |
| I-10 | 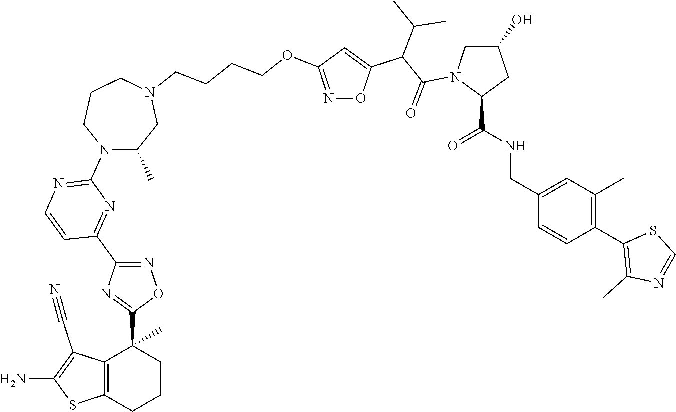 [SMILES: Cc1cc(CNC(=O)[C@@H]2C[C@@H](O)CN2C(=O)C(c2cc(OCCCCN3CCCN(c4nccc(-c5noc([C@@]6(C)CCCc7sc(N)c(C#N)c76)n5)n4)[C@@H](C)C3)no2)C(C)C)ccc1-c1scnc1C] | 0.76 | 1003 | H |
| I-11 | 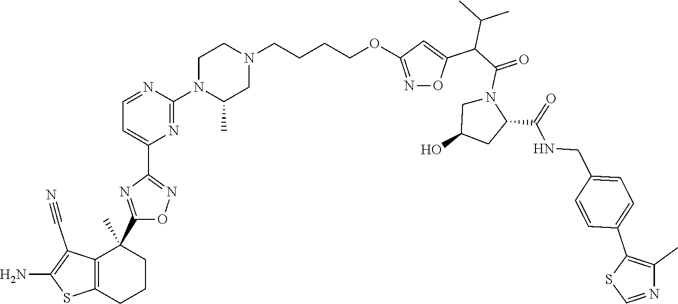 [SMILES: Cc1ncsc1-c1ccc(CNC(=O)[C@@H]2C[C@@H](O)CN2C(=O)C(c2cc(OCCCCN3CCN(c4nccc(-c5noc([C@@]6(C)CCCc7sc(N)c(C#N)c76)n5)n4)[C@@H](C)C3)no2)C(C)C)cc1] | 0.82 | 975 | H |
| I-12 | 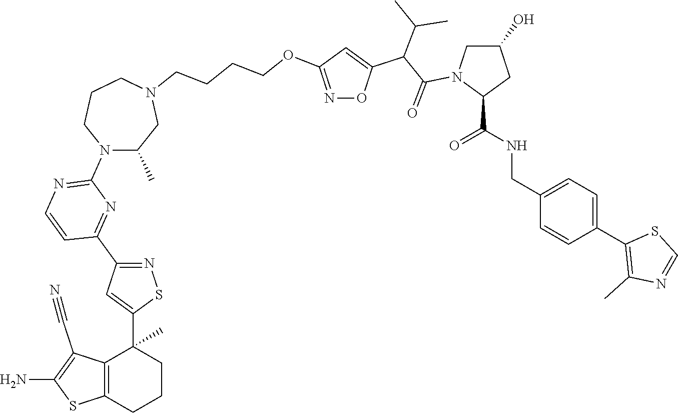 [SMILES: Cc1ncsc1-c1ccc(CNC(=O)[C@@H]2C[C@@H](O)CN2C(=O)C(c2cc(OCCCCN3CCCN(c4nccc(-c5cc([C@@]6(C)CCCc7sc(N)c(C#N)c76)sn5)n4)[C@@H](C)C3)no2)C(C)C)cc1] | 0.83 | 1004 | H |
| I-13 | 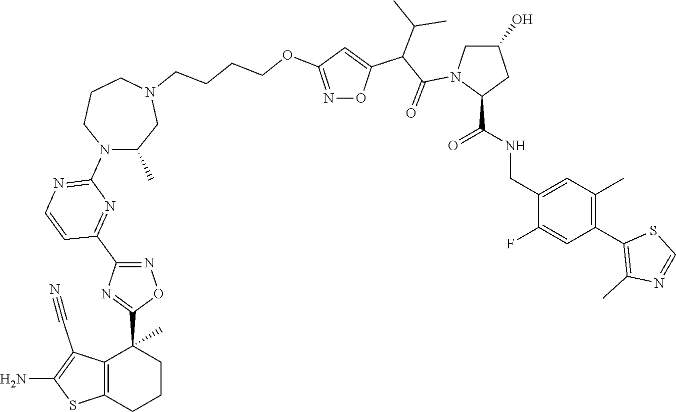 [SMILES: Cc1cc(CNC(=O)[C@@H]2C[C@@H](O)CN2C(=O)C(c2cc(OCCCCN3CCCN(c4nccc(-c5noc([C@@]6(C)CCCc7sc(N)c(C#N)c76)n5)n4)[C@@H](C)C3)no2)C(C)C)c(F)cc1-c1scnc1C] | 0.78 | 1021 | H |
| I-14 | 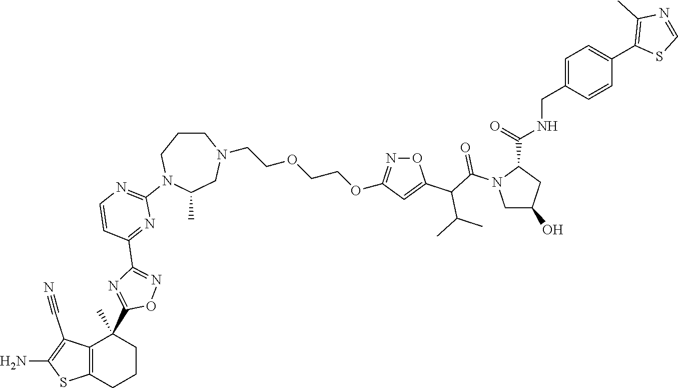 [SMILES: Cc1ncsc1-c1ccc(CNC(=O)[C@@H]2C[C@@H](O)CN2C(=O)C(c2cc(OCCOCCN3CCCN(c4nccc(-c5noc([C@@]6(C)CCCc7sc(N)c(C#N)c76)n5)n4)[C@@H](C)C3)no2)C(C)C)cc1] | 1.41 | 1005 | A |
| I-15 | 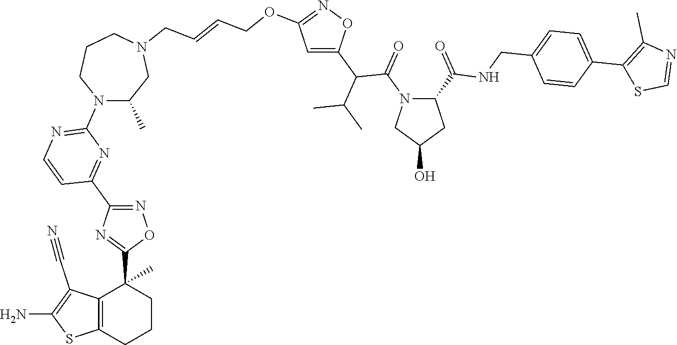 [SMILES: Cc1ncsc1-c1ccc(CNC(=O)[C@@H]2C[C@@H](O)CN2C(=O)C(c2cc(OC/C=C/CN3CCCN(c4nccc(-c5noc([C@@]6(C)CCCc7sc(N)c(C#N)c76)n5)n4)[C@@H](C)C3)no2)C(C)C)cc1] | 1.46 | 987 | A |
| I-28 | 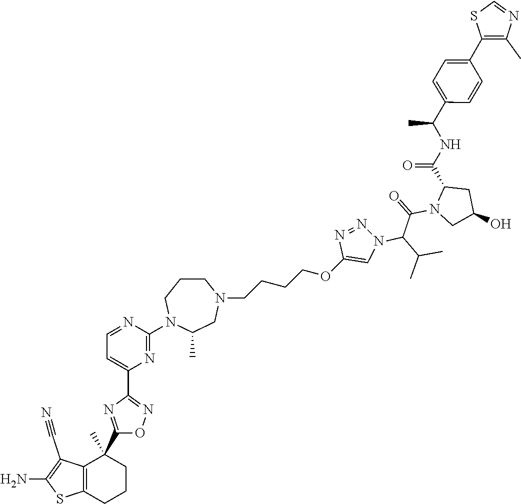 [SMILES: Cc1ncsc1-c1ccc([C@H](C)NC(=O)[C@@H]2C[C@@H](O)CN2C(=O)C(C(C)C)n2cc(OCCCCN3CCCN(c4nccc(-c5noc([C@@]6(C)CCCc7sc(N)c(C#N)c76)n5)n4)[C@@H](C)C3)nn2)cc1] | 1.47 | 1003 | A |
| I-29 | 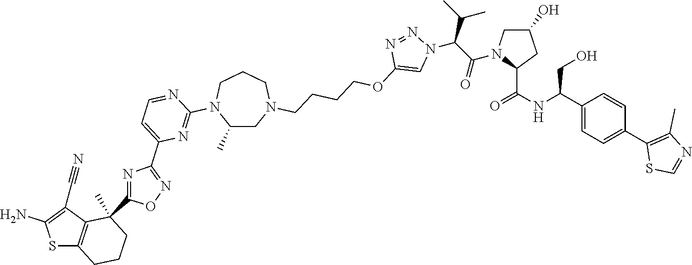 [SMILES: Cc1ncsc1-c1ccc([C@H](CO)NC(=O)[C@@H]2C[C@@H](O)CN2C(=O)[C@H](C(C)C)n2cc(OCCCCN3CCCN(c4nccc(-c5noc([C@@]6(C)CCCc7sc(N)c(C#N)c76)n5)n4)[C@@H](C)C3)nn2)cc1] | 1.38 | 1019 | A |
| I-30 | 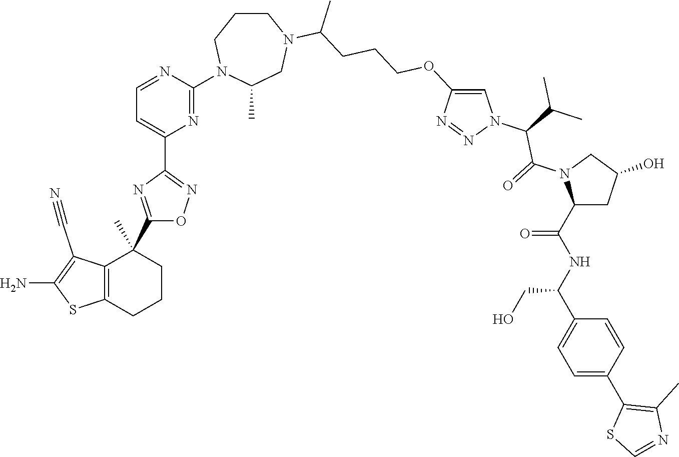 [SMILES: Cc1ncsc1-c1ccc([C@H](CO)NC(=O)[C@@H]2C[C@@H](O)CN2C(=O)[C@H](C(C)C)n2cc(OCCCC(C)N3CCCN(c4nccc(-c5noc([C@@]6(C)CCCc7sc(N)c(C#N)c76)n5)n4)[C@@H](C)C3)nn2)cc1] | 1.46 | 1033 | A |
| I-31 | 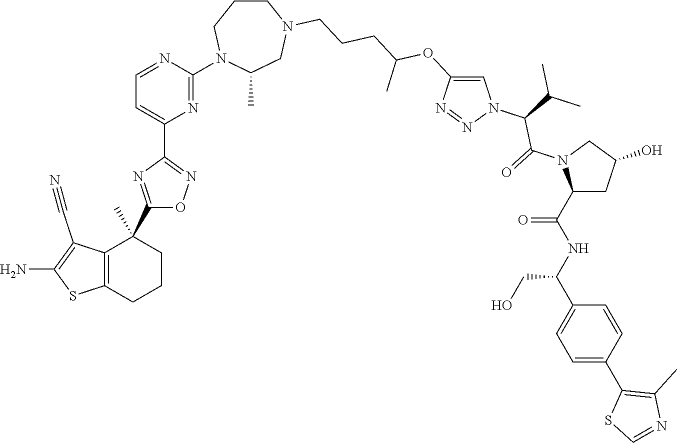 [SMILES: Cc1ncsc1-c1ccc([C@H](CO)NC(=O)[C@@H]2C[C@@H](O)CN2C(=O)[C@H](C(C)C)n2cc(OC(C)CCCN3CCCN(c4nccc(-c5noc([C@@]6(C)CCCc7sc(N)c(C#N)c76)n5)n4)[C@@H](C)C3)nn2)cc1] | 1.41 | 1033 | A |
| I-32 | 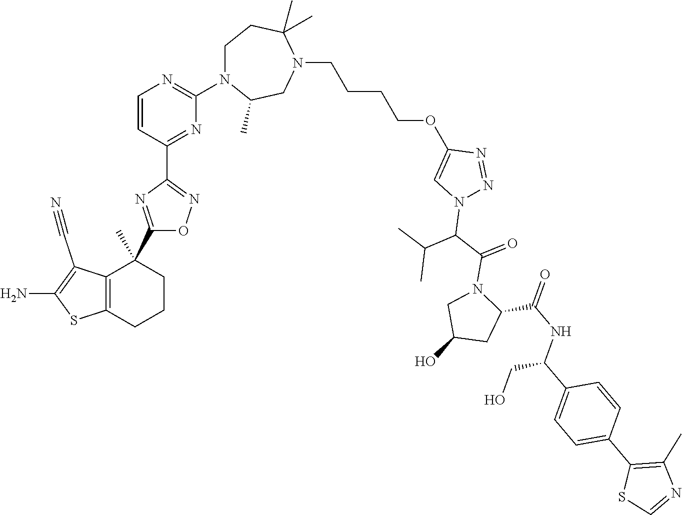 [SMILES: Cc1ncsc1-c1ccc([C@H](CO)NC(=O)[C@@H]2C[C@@H](O)CN2C(=O)C(C(C)C)n2cc(OCCCCN3C[C@H](C)N(c4nccc(-c5noc([C@@]6(C)CCCc7sc(N)c(C#N)c76)n5)n4)CCC3(C)C)nn2)cc1] | 1.49 | 1047 | A |
| I-33 | 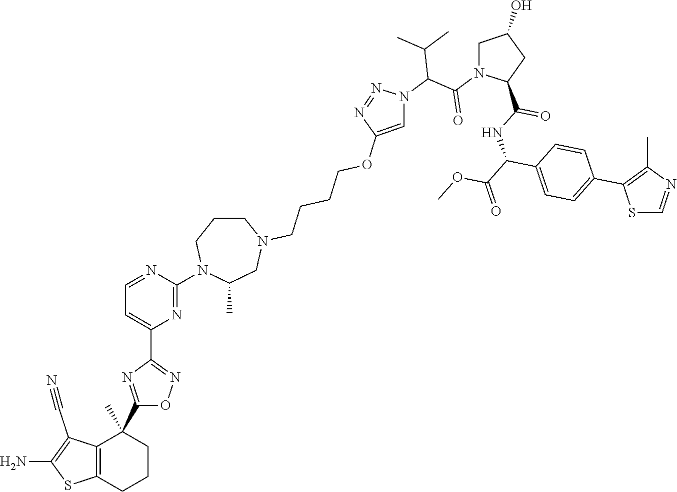 [SMILES: COC(=O)[C@H](NC(=O)[C@@H]1C[C@@H](O)CN1C(=O)C(C(C)C)n1cc(OCCCCN2CCCN(c3nccc(-c4noc([C@@]5(C)CCCc6sc(N)c(C#N)c65)n4)n3)[C@@H](C)C2)nn1)c1ccc(-c2scnc2C)cc1] | 1.46 | 1047 | A |
| I-34 | 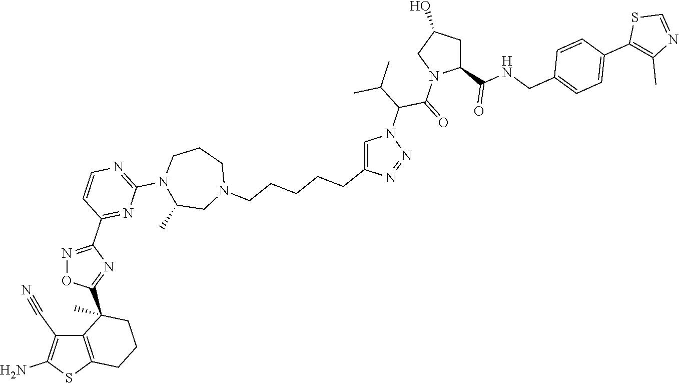 [SMILES: Cc1ncsc1-c1ccc(CNC(=O)[C@@H]2C[C@@H](O)CN2C(=O)C(C(C)C)n2cc(CCCCCN3CCCN(c4nccc(-c5noc([C@@]6(C)CCCc7sc(N)c(C#N)c76)n5)n4)[C@@H](C)C3)nn2)cc1] | 1.44 | 987 | A |

### Experimental Procedure for the Synthesis of I-17

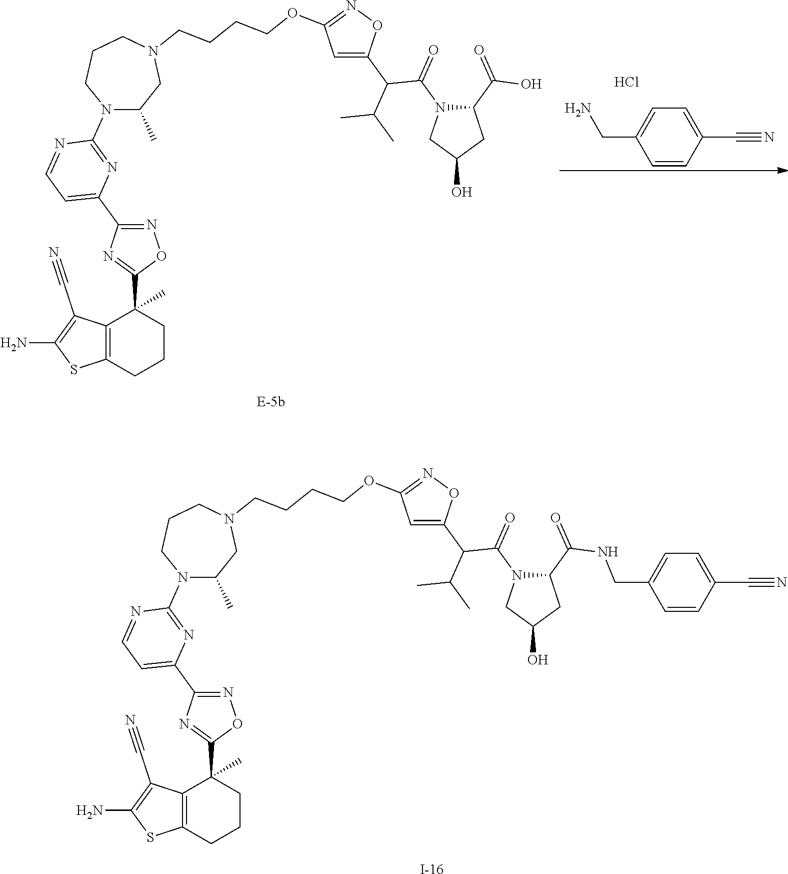

To a stirred solution of E-5b (25 mg, 0.032 mmol, 1.0 eq.), 4-(aminomethyl)benzonitrile hydrochloride (10 mg, 0.059 mmol, 1.8 eq.) and HATU (35 mg, 0.091 mmol, 2.8 eq.) in DMSO (1.0 mL) is added TEA (26 μL, 0.187 mmol, 5.8 eq.) and the reaction mixture is stirred at rt for 20 min. After complete conversion the reaction mixture is diluted with ACN and purified by chromatograph yielding I-16.

The following compounds I (table 60) are available in an analogous manner starting from different intermediates E-5 and A-2.

TABLE 60

| | | tᵣₑₜ | | HPLC |
|---|---|---|---|---|
| # | structure | [min] | [M + H]⁺ | method |
| I-16 | 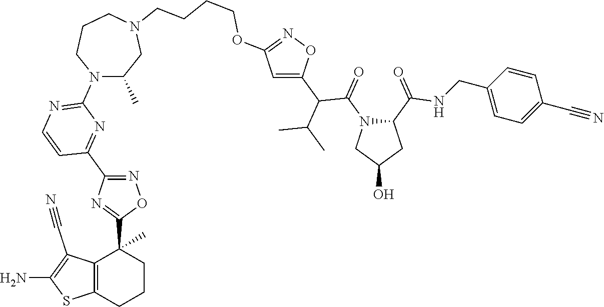 [SMILES: CC(C)C(C(=O)N1C[C@H](O)C[C@H]1C(=O)NCc1ccc(C#N)cc1)c1cc(OCCCCN2CCCN(c3nccc(-c4noc([C@@]5(C)CCCc6sc(N)c(C#N)c65)n4)n3)[C@@H](C)C2)no1] | 1.49 | 917 | A |
| I-9 | 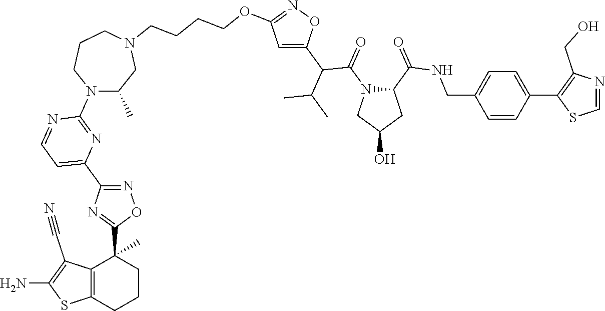 [SMILES: CC(C)C(C(=O)N1C[C@H](O)C[C@H]1C(=O)NCc1ccc(-c2scnc2CO)cc1)c1cc(OCCCCN2CCCN(c3nccc(-c4noc([C@@]5(C)CCCc6sc(N)c(C#N)c65)n4)n3)[C@@H](C)C2)no1] | 1.38 | 1005 | A |
| I-17 | 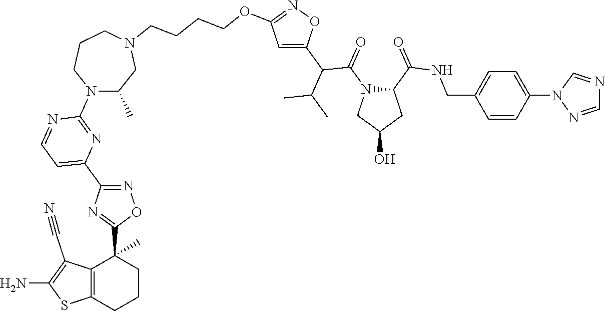 [SMILES: CC(C)C(C(=O)N1C[C@H](O)C[C@H]1C(=O)NCc1ccc(-n2cncn2)cc1)c1cc(OCCCCN2CCCN(c3nccc(-c4noc([C@@]5(C)CCCc6sc(N)c(C#N)c65)n4)n3)[C@@H](C)C2)no1] | 1.38 | 959 | A |
| I-18 | 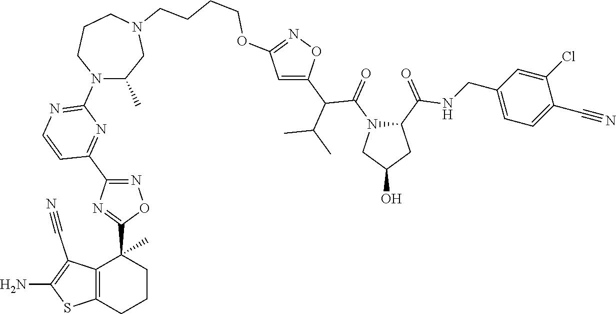 [SMILES: CC(C)C(C(=O)N1C[C@H](O)C[C@H]1C(=O)NCc1ccc(C#N)c(Cl)c1)c1cc(OCCCCN2CCCN(c3nccc(-c4noc([C@@]5(C)CCCc6sc(N)c(C#N)c65)n4)n3)[C@@H](C)C2)no1] | 1.51 | 951 | A |
| I-19 | 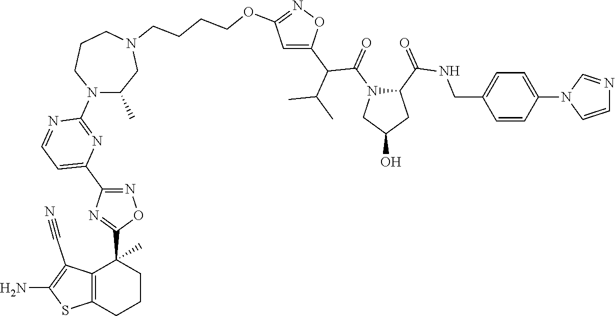 [SMILES: CC(C)C(C(=O)N1C[C@H](O)C[C@H]1C(=O)NCc1ccc(-n2ccnc2)cc1)c1cc(OCCCCN2CCCN(c3nccc(-c4noc([C@@]5(C)CCCc6sc(N)c(C#N)c65)n4)n3)[C@@H](C)C2)no1] | 1.38 | 958 | A |
| I-20 | 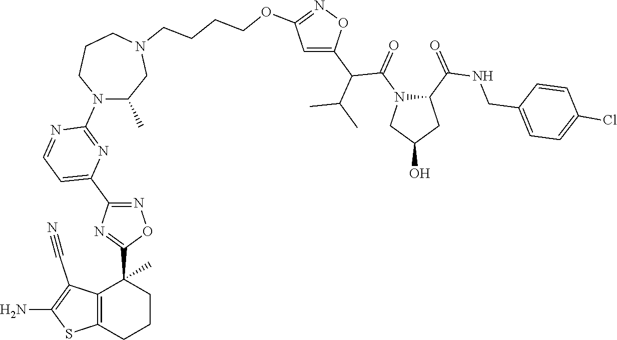 [SMILES: CC(C)C(C(=O)N1C[C@H](O)C[C@H]1C(=O)NCc1ccc(Cl)cc1)c1cc(OCCCCN2CCCN(c3nccc(-c4noc([C@@]5(C)CCCc6sc(N)c(C#N)c65)n4)n3)[C@@H](C)C2)no1] | 0.87 | 926 | H |
| I-21 | 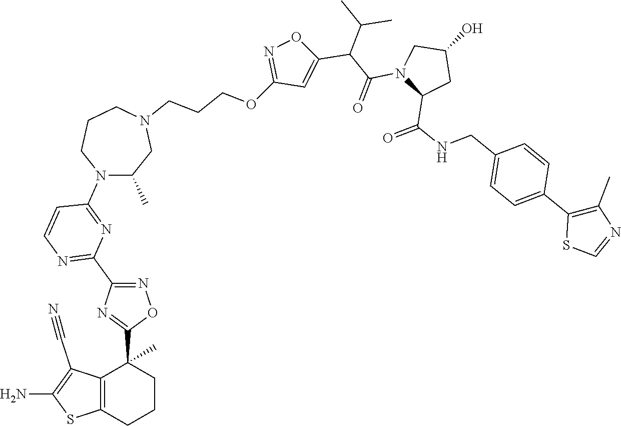 [SMILES: Cc1ncsc1-c1ccc(CNC(=O)[C@@H]2C[C@@H](O)CN2C(=O)C(c2cc(OCCCN3CCCN(c4ccnc(-c5noc([C@@]6(C)CCCc7sc(N)c(C#N)c76)n5)n4)[C@@H](C)C3)no2)C(C)C)cc1] | 1.32 | 975 | A |
| I-22 | 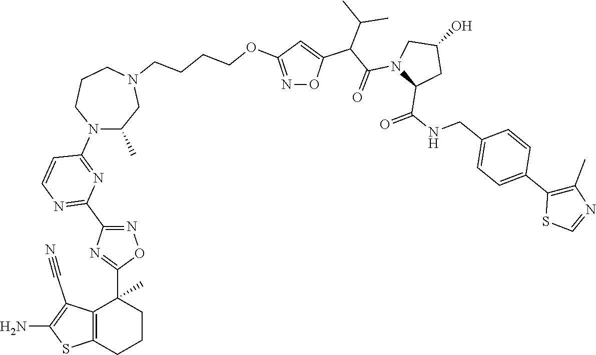 [SMILES: Cc1ncsc1-c1ccc(CNC(=O)[C@@H]2C[C@@H](O)CN2C(=O)C(c2cc(OCCCCN3CCCN(c4ccnc(-c5noc([C@@]6(C)CCCc7sc(N)c(C#N)c76)n5)n4)[C@@H](C)C3)no2)C(C)C)cc1] | 1.35 | 989 | A |
| I-23 | 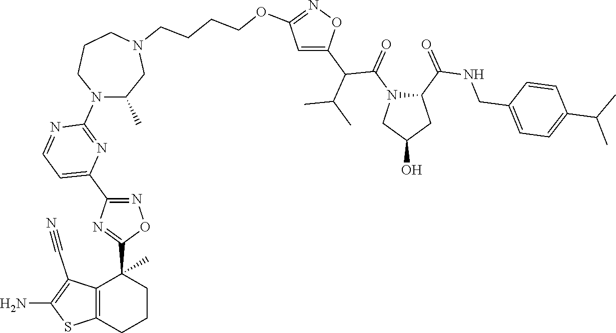 [SMILES: CC(C)c1ccc(CNC(=O)[C@@H]2C[C@@H](O)CN2C(=O)C(c2cc(OCCCCN3CCCN(c4nccc(-c5noc([C@@]6(C)CCCc7sc(N)c(C#N)c76)n5)n4)[C@@H](C)C3)no2)C(C)C)cc1] | 0.92 | 934 | H |
| I-35 | 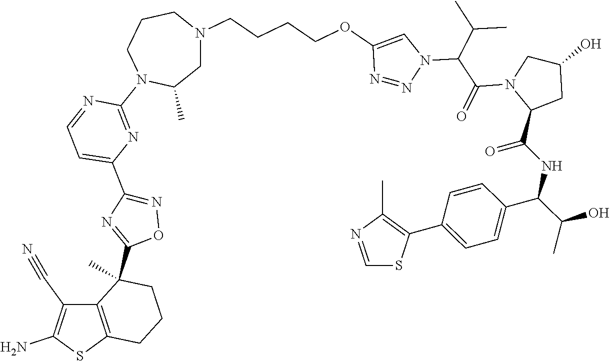 [SMILES: Cc1ncsc1-c1ccc([C@@H](NC(=O)[C@@H]2C[C@@H](O)CN2C(=O)C(C(C)C)n2cc(OCCCCN3CCCN(c4nccc(-c5noc([C@@]6(C)CCCc7sc(N)c(C#N)c76)n5)n4)[C@@H](C)C3)nn2)[C@H](C)O)cc1] | 1.40 | 1033 | A |
| I-36 | 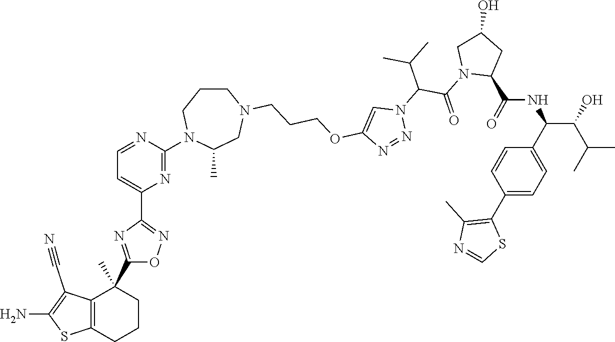 [SMILES: Cc1ncsc1-c1ccc([C@@H](NC(=O)[C@@H]2C[C@@H](O)CN2C(=O)C(C(C)C)n2cc(OCCCN3CCCN(c4nccc(-c5noc([C@@]6(C)CCCc7sc(N)c(C#N)c76)n5)n4)[C@@H](C)C3)nn2)[C@H](O)C(C)C)cc1] | 1.48 | 1047 | A |
| I-37 | 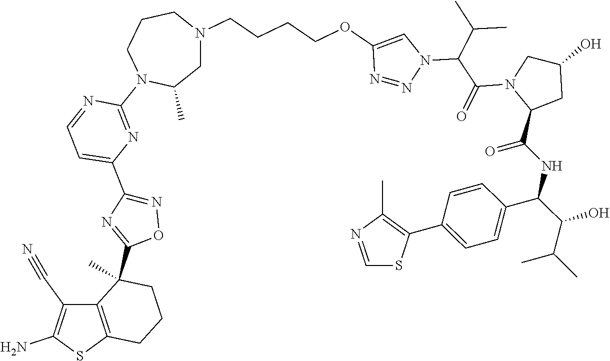 [SMILES: Cc1ncsc1-c1ccc([C@@H](NC(=O)[C@@H]2C[C@@H](O)CN2C(=O)C(C(C)C)n2cc(OCCCCN3CCCN(c4nccc(-c5noc([C@@]6(C)CCCc7sc(N)c(C#N)c76)n5)n4)[C@@H](C)C3)nn2)[C@H](O)C(C)C)cc1] | 1.49/1.51 | 1061 | A |
| I-38 | 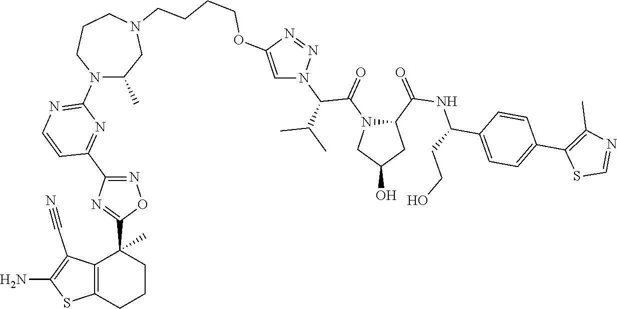 [SMILES: Cc1ncsc1-c1ccc([C@H](CCO)NC(=O)[C@@H]2C[C@@H](O)CN2C(=O)[C@H](C(C)C)n2cc(OCCCCN3CCCN(c4nccc(-c5noc([C@@]6(C)CCCc7sc(N)c(C#N)c76)n5)n4)[C@@H](C)C3)nn2)cc1] | 1.39 | 1033 | A |
| I-39 | 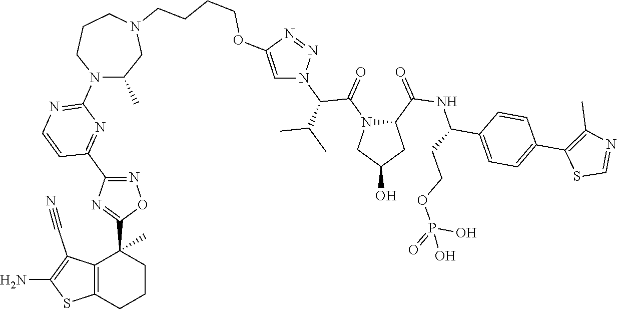 [SMILES: Cc1ncsc1-c1ccc([C@H](CCOP(=O)(O)O)NC(=O)[C@@H]2C[C@@H](O)CN2C(=O)[C@H](C(C)C)n2cc(OCCCCN3CCCN(c4nccc(-c5noc([C@@]6(C)CCCc7sc(N)c(C#N)c76)n5)n4)[C@@H](C)C3)nn2)cc1] | 1.14 | 1113 | A |

### Chiral Separation Via Chiral Column Chromatography of Compounds I:

If compounds I are obtained as mixtures of diastereomers they can be separated to single stereoisomers by chiral chromatography, e.g. as shown for I-1 which was separated to obtain I-24 and I-25, I-3 which was separated into I-26 and I-27, I-35 which was separated to obtain I-40 and I-41, I-36 which was separated to obtain I-42 and I-43, as well as I-37 which was separated to obtain I-44 and I-45 (Table 61).

TABLE 61

| | | tᵣₑₜ | | HPLC |
|---|---|---|---|---|
| # | structure | [min] | [M + H]⁺ | method |
| I-24 | 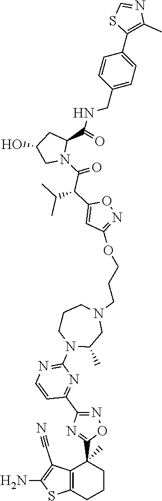 [SMILES: Cc1ncsc1-c1ccc(CNC(=O)[C@@H]2C[C@@H](O)CN2C(=O)[C@H](c2cc(OCCCN3CCCN(c4nccc(-c5noc([C@@]6(C)CCCc7sc(N)c(C#N)c76)n5)n4)[C@@H](C)C3)no2)C(C)C)cc1] | 0.78 | 975 | H |
| | | 2.20 | — | I |
| I-25 | 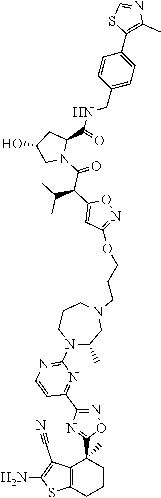 [SMILES: Cc1ncsc1-c1ccc(CNC(=O)[C@@H]2C[C@@H](O)CN2C(=O)[C@@H](c2cc(OCCCN3CCCN(c4nccc(-c5noc([C@@]6(C)CCCc7sc(N)c(C#N)c76)n5)n4)[C@@H](C)C3)no2)C(C)C)cc1] | 0.78 | 975 | H |
| | | 2.99 | — | I |
| I-26 | 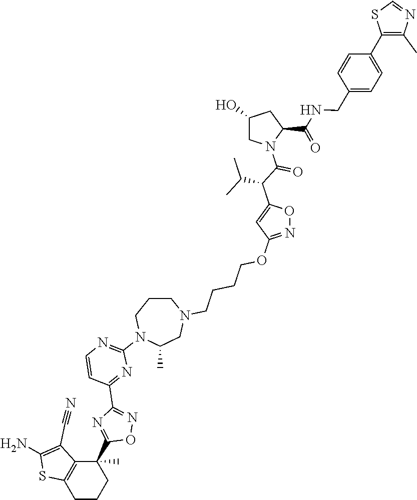 [SMILES: Cc1ncsc1-c1ccc(CNC(=O)[C@@H]2C[C@@H](O)CN2C(=O)[C@H](c2cc(OCCCCN3CCCN(c4nccc(-c5noc([C@@]6(C)CCCc7sc(N)c(C#N)c76)n5)n4)[C@@H](C)C3)no2)C(C)C)cc1] | 1.47 | 989 | A |
| | | 2.36 | — | I |
| I-27 | 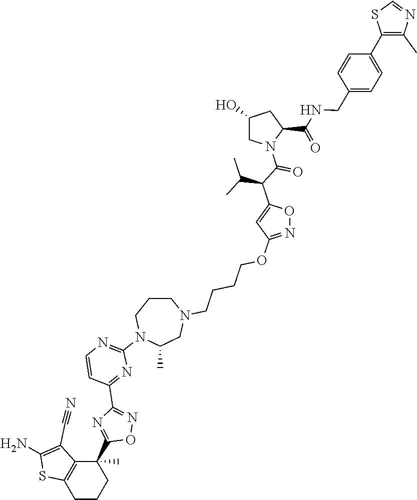 [SMILES: Cc1ncsc1-c1ccc(CNC(=O)[C@@H]2C[C@@H](O)CN2C(=O)[C@@H](c2cc(OCCCCN3CCCN(c4nccc(-c5noc([C@@]6(C)CCCc7sc(N)c(C#N)c76)n5)n4)[C@@H](C)C3)no2)C(C)C)cc1] | 1.47 | 989 | A |
| | | 3.22 | — | I |
| I-40 | 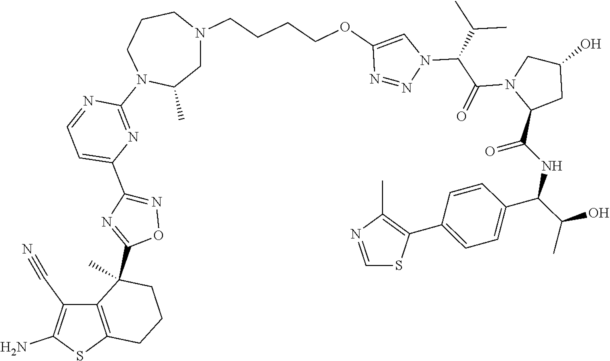 [SMILES: Cc1ncsc1-c1ccc([C@@H](NC(=O)[C@@H]2C[C@@H](O)CN2C(=O)[C@@H](C(C)C)n2cc(OCCCCN3CCCN(c4nccc(-c5noc([C@@]6(C)CCCc7sc(N)c(C#N)c76)n5)n4)[C@@H](C)C3)nn2)[C@H](C)O)cc1] | 1.39 | 1033 | A |
| | | 2.35 | — | I |
| I-41 | 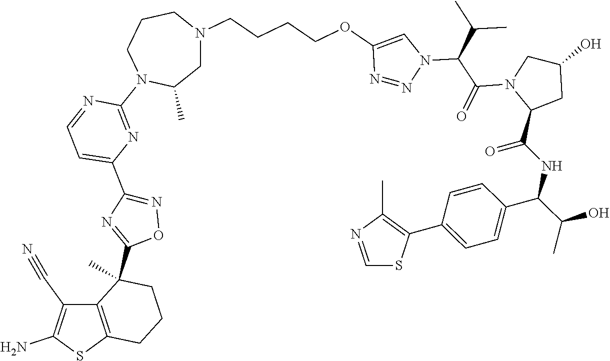 [SMILES: Cc1ncsc1-c1ccc([C@@H](NC(=O)[C@@H]2C[C@@H](O)CN2C(=O)[C@H](C(C)C)n2cc(OCCCCN3CCCN(c4nccc(-c5noc([C@@]6(C)CCCc7sc(N)c(C#N)c76)n5)n4)[C@@H](C)C3)nn2)[C@H](C)O)cc1] | 1.40 | 1033 | A |
| | | 3.09 | — | I |
| I-42 | 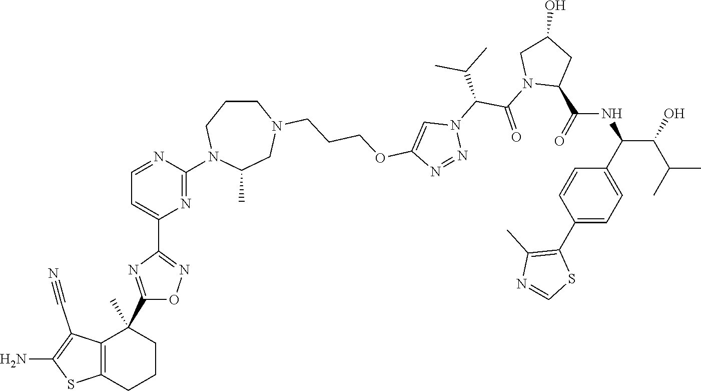 [SMILES: Cc1ncsc1-c1ccc([C@@H](NC(=O)[C@@H]2C[C@@H](O)CN2C(=O)[C@@H](C(C)C)n2cc(OCCCN3CCCN(c4nccc(-c5noc([C@@]6(C)CCCc7sc(N)c(C#N)c76)n5)n4)[C@@H](C)C3)nn2)[C@H](O)C(C)C)cc1] | 1.47 | 1047 | A |
| | | 1.98 | — | I |
| I-43 | 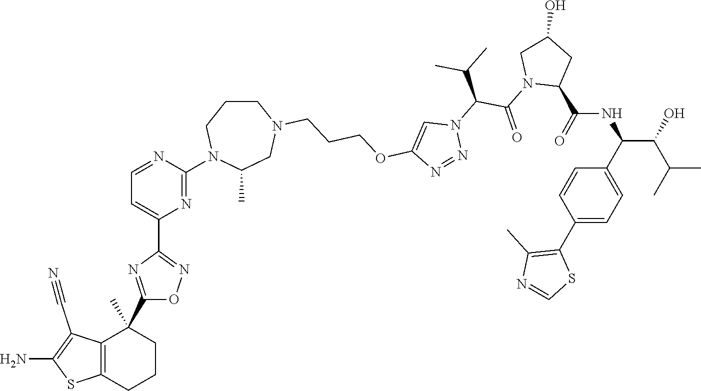 [SMILES: Cc1ncsc1-c1ccc([C@@H](NC(=O)[C@@H]2C[C@@H](O)CN2C(=O)[C@H](C(C)C)n2cc(OCCCN3CCCN(c4nccc(-c5noc([C@@]6(C)CCCc7sc(N)c(C#N)c76)n5)n4)[C@@H](C)C3)nn2)[C@H](O)C(C)C)cc1] | 1.49 | 1047 | A |
| | | 2.80 | — | I |
| I-44 | 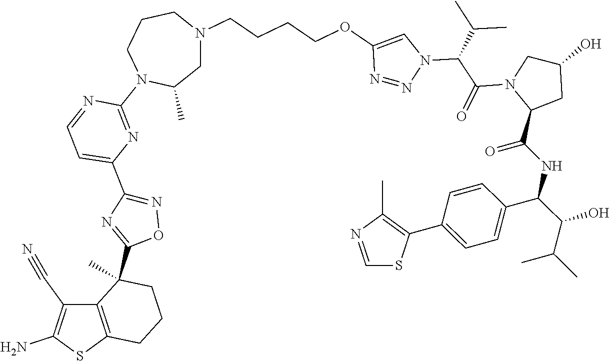 [SMILES: Cc1ncsc1-c1ccc([C@@H](NC(=O)[C@@H]2C[C@@H](O)CN2C(=O)[C@@H](C(C)C)n2cc(OCCCCN3CCCN(c4nccc(-c5noc([C@@]6(C)CCCc7sc(N)c(C#N)c76)n5)n4)[C@@H](C)C3)nn2)[C@H](O)C(C)C)cc1] | 1.48 | 1061 | A |
| | | 1.93 | — | I |
| I-45 | 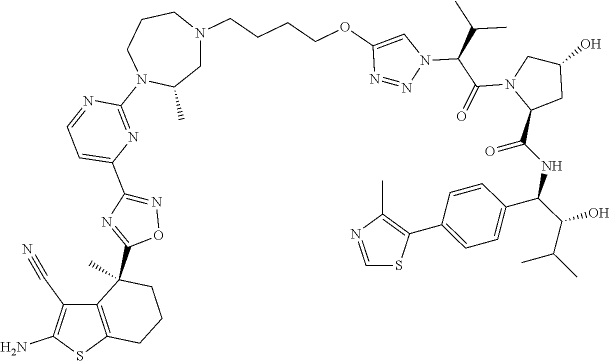 [SMILES: Cc1ncsc1-c1ccc([C@@H](NC(=O)[C@@H]2C[C@@H](O)CN2C(=O)[C@H](C(C)C)n2cc(OCCCCN3CCCN(c4nccc(-c5noc([C@@]6(C)CCCc7sc(N)c(C#N)c76)n5)n4)[C@@H](C)C3)nn2)[C@H](O)C(C)C)cc1] | 1.51 | 1061 | A |
| | | 2.95 | — | I |

### Experimental Procedure for the Synthesis of I-46

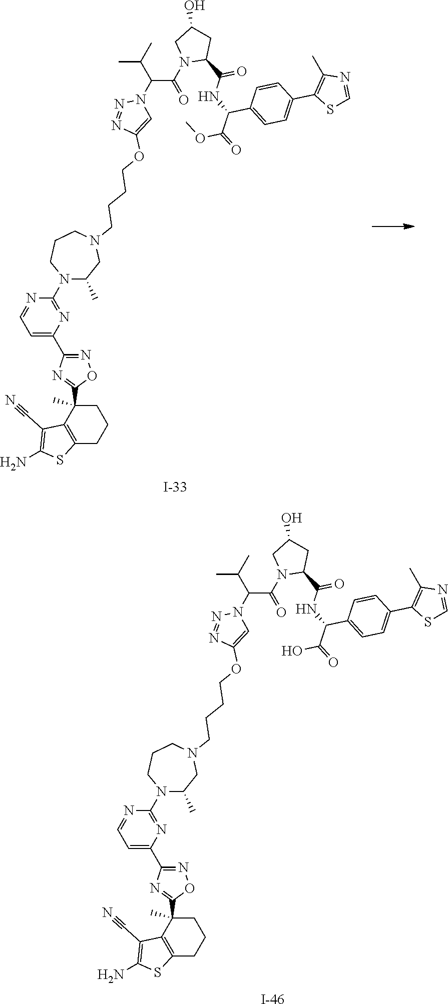

To a stirred solution of I-33 (25 mg, 0.024 mmol, 1.0 eq.) in THF (1 mL) is added LiOH solution (18 μL, 0.036, 1.5 eq., 2 N in H₂O) and the reaction mixture is stirred for 48 h. The reaction mixture is acidified with 2 N HCl and purified by chromatography to obtain I-46 (table 62).

TABLE 62

| | | tᵣₑₜ | | HPLC |
|---|---|---|---|---|
| # | Structure | [min] | [M + H]⁺ | method |
| I-46 | 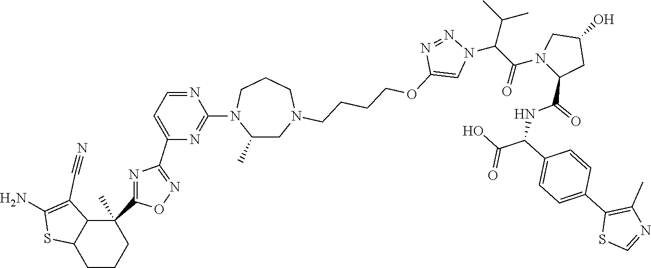 [SMILES: Cc1ncsc1-c1ccc([C@@H](NC(=O)[C@@H]2C[C@@H](O)CN2C(=O)C(C(C)C)n2cc(OCCCCN3CCCN(c4nccc(-c5noc([C@@]6(C)CCCC7SC(N)=C(C#N)C76)n5)n4)[C@@H](C)C3)nn2)C(=O)O)cc1] | 1.19 | 1033 | A |

### Experimental Procedure for the Synthesis of I-47

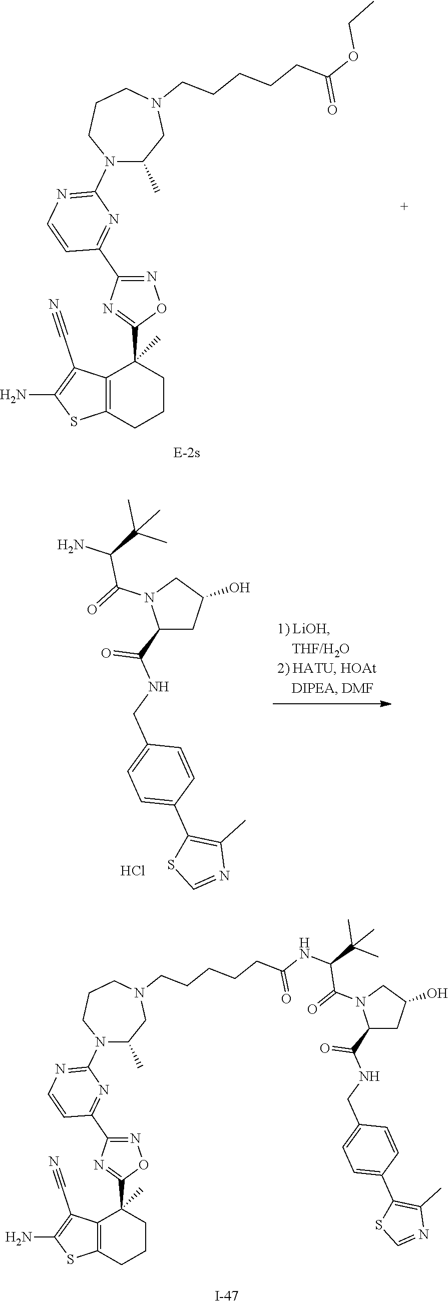

To a solution of E-2s (43 mg, 0.073 mmol, 1.0 eq.) in THF (2.0 mL) and water (2 mL) is added LiOH H₂O (15 mg, 0.36 mmol) and the reaction mixture is stirred at room temperature overnight. The reaction mixture is neutralized using 4M HCl in water, and concentrated under reduced pressure. The crude acid intermediate is taken up in DMF (2.0 mL). HATU (50 mg, 0.13 mmol, 1.8 eq.), HOAt (18 mg, 0.13 mmol, 1.8 qe.), (2S,4R)-1-[(2S)-2-amino-3,3-dimethylbutanoyl]-4-hydroxy-N-{[4-(4-methyl-1,3-thiazol-5-yl)phenyl]methyl}pyrrolidine-2-carboxamide hydrochloride (43 mg, 0.073 mmol, 1.8 eq.) and DIPEA (35 μL, 0.21 mmol, 2.9 eq.) are added. The reaction mixture is stirred at room temperature overnight. The reaction mixture is concentrated under reduced pressure. The residue is purified by chromatography to give I-47

The following compounds I (table 63) are available in an analogous manner starting from different intermediates E-2.

TABLE 63

| | | tᵣₑₜ | | HPLC |
|---|---|---|---|---|
| # | structure | [min] | [M + H]⁺ | method |
| I-47 | 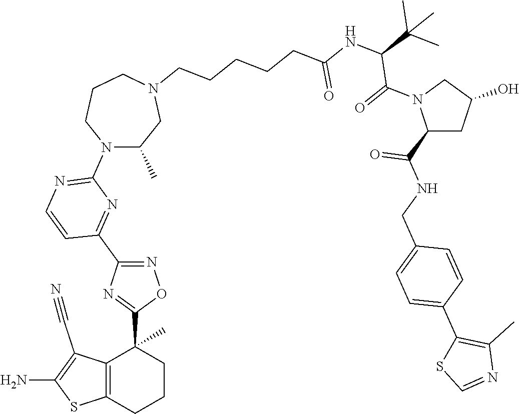 [SMILES: Cc1ncsc1-c1ccc(CNC(=O)[C@@H]2C[C@@H](O)CN2C(=O)[C@@H](NC(=O)CCCCCN2CCCN(c3nccc(-c4noc([C@@]5(C)CCCc6sc(N)c(C#N)c65)n4)n3)[C@@H](C)C2)C(C)(C)C)cc1] | 1.40 | 977 | M |
| I-48 | 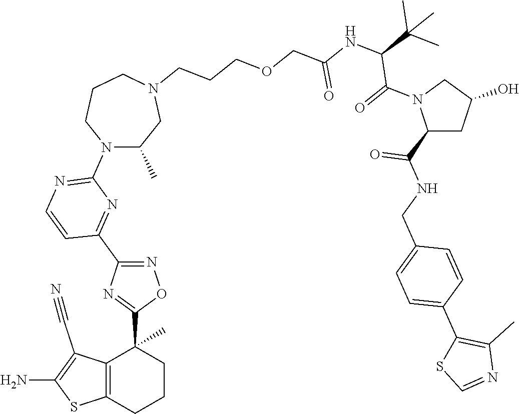 [SMILES: Cc1ncsc1-c1ccc(CNC(=O)[C@@H]2C[C@@H](O)CN2C(=O)[C@@H](NC(=O)COCCCN2CCCN(c3nccc(-c4noc([C@@]5(C)CCCc6sc(N)c(C#N)c65)n4)n3)[C@@H](C)C2)C(C)(C)C)cc1] | 4.0 | 979 | N |

Compounds I-49, I-50 and I-51 according to the invention can also be obtained analogously following the procedures as described herein and using the corresponding building blocks:

The following Examples describe the biological activity of the compounds according to the invention, without restricting the invention to these Examples.

### BIOLOGICAL EXAMPLES

### HiBit Degradation Assay

A HiBit protein detection tag (amino acid sequence VSGWRLFKKIS, Seq ID No 1) was introduced immediately downstream of the initiating Methionine codon of the endogenous KRAS locus (Ensembl gene ID ENSG00000133703.7) of GP5d cells (ECACC Cat. No. 95090715) by CRISPR-based genome engineering using a KRAS(G12D) mutant donor construct encoding the HiBit tag. This resulted in the heterozygous introduction of an N-terminal HiBit tagged version of KRAS(G12D) into the KRAS(WT) allele. Correct modification of the KRAS locus was assessed by PCR-based genotyping and Sanger sequencing of the isolated PCR products. The resulting cell line is referred to as GP5d-HiBit-KRAS(G12D).

To assess PROTAC-mediated degradation of KRAS(G12D), GP5d-HiBit-KRAS(G12D) cells were seeded at 25000 cells per well in 100 μL Dulbecco's Modified Eagle medium (Sigma cat. no. D6429) supplemented with 10% fetal calf serum into white bottom opaque 96 well plates (Perkin Elmer cat no. 5680). Plates were incubated at 37° C., 5% CO₂ in a humidified incubator over night to allow the cells to adhere. Test compounds (10 mM stock in DMSO) were added at logarithmic dose series using the HP Digital Dispenser D300 (Tecan), normalizing for added DMSO. Plates were further incubated at 37° C. for 18 hours. Following incubation, 100 μL per well Promega Nano-Glo HiBit lytic detection reagent mix (Promega Nano-Glo HiBit Lytic Detection System #N3050), prepared according to the manufacturer's instructions in the kit, were added. To allow for adequate cell lysis, plates were incubated on an orbital shaker for 15 min and further incubated 30 min at room temperature. Upon completion of cell lysis, luminescence was measured using an Envision plate reader using the Ultrasensitive Luminescence Protocol for 96 well plates. Luminescence levels were normalized by the values obtained with DMSO-treated samples and plotted as percent of DMSO control. DC₅₀ values were computed using a four parametric logistic model. Dmax values represent the maximal extent of degradation observed and is stated as percent of control (% Ctrl.) treatments. DC₅₀ and Dmax are reported in Table 64 for representative compounds of the invention.

TABLE 64

| | DC₅₀ | Dmax |
|---|---|---|
| | 18 h | 18 h |
| # | (nM) | (% Ctrl) |
| I-1 | 6.4 | 94 |
| I-2 | 262.6 | 84 |
| I-3 | 2.7 | 91 |
| I-4 | 4.3 | 94 |
| I-5 | 6.5 | 92 |
| I-6 | 8.9 | 92 |
| I-7 | 11.0 | 89 |
| I-8 | 11.4 | 86 |
| I-9 | 12.9 | 92 |
| I-10 | 15.4 | 91 |
| I-11 | 35.1 | 57 |
| I-12 | 42.1 | 86 |
| I-13 | 45.2 | 87 |
| I-14 | 76.0 | 80 |
| I-15 | 9.8 | 90 |
| I-16 | 3.9 | 91 |
| I-17 | 3.3 | 93 |
| I-18 | 4.1 | 89 |
| I-19 | 6.7 | 93 |
| I-20 | 8.6 | 89 |
| I-21 | 41.1 | 90 |
| I-22 | 48.8 | 92 |
| I-23 | 76.7 | 82 |
| I-24 | 200.2 | 81 |
| I-25 | 2.3 | 94 |
| I-26 | 100.5 | 77 |
| I-27 | 2.9 | 93 |
| I-28 | 1.5 | 93 |
| I-29 | 2.0 | 93 |
| I-30 | 1.4 | 94 |
| I-31 | 4.4 | 93 |
| I-32 | 7.6 | 83 |
| I-33 | 13.1 | 91 |
| I-34 | 10.6 | 91 |
| I-38 | 3.3 | 93 |
| I-39 | 7.7 | 87 |
| I-40 | 970.2 | 61 |
| I-41 | 1.6 | 94 |
| I-42 | 462.2 | 74 |
| I-43 | 1.2 | 93 |
| I-44 | 49.7 | 88 |
| I-45 | 1.4 | 93 |
| I-46 | 739.2 | 83 |
| I-47 | 47.7 | 94 |
| I-48 | 522.6 | 85 |

### HiBit KRAS4B Mutant Spectrum Degradation Assay

The therapeutically relevant mutant KRAS constructs (WT, G12C, G12D, G12V, G13D) were obtained by site directed mutagenesis using a KRAS4B WT cDNA construct as a template. GP5d cells (ECACC Cat. No. 95090715) were transduced with lentiviral vectors expressing mutant KRAS4B cDNA under control of a CMV promoter. Stably transduced cells were selected using a neomycin selectable marker encoded on the construct.

To assess PROTAC-mediated degradation of various KRAS mutants, GP5d cells stably transduced with KRAS4B constructs as described above were seeded at 25000 cells per well in 100 μL Dulbecco's Modified Eagle medium (Sigma cat. no. D6429) supplemented with 10% fetal calf serum into white bottom opaque 96 well plates (Perkin Elmer cat no. 5680). Plates were incubated at 37° C., 5% CO₂ in a humidified incubator over night to allow the cells to adhere. Test compounds (10 mM stock in DMSO) were added at logarithmic dose series using the HP Digital Dispenser D300 (Tecan), normalizing for added DMSO. Plates were further incubated at 37° C. for 18 hours. Following incubation, 100 μL per well Promega Nano-Glo HiBit lytic detection reagent mix (Promega Nano-Glo HiBit Lytic Detection System #N3050), prepared according to the manufacturer's instructions in the kit, were added. To allow for adequate cell lysis, plates were incubated on an orbital shaker for 15 min and further incubated 30 min at room temperature. Upon completion of cell lysis, luminescence was measured using an Envision plate reader using the Ultrasensitive Luminescence Protocol for 96 well plates. Luminescence levels were normalized by the values obtained with DMSO-treated samples and plotted as percent of DMSO control. DC₅₀ values were computed using a four parametric logistic model and are reported in nM in Table 65 for representative example (Ex.) compounds of the invention. Dmax values represent the maximal extent of degradation observed and is stated as percent of control (% Ctrl.) treatments in Table 66 for representative example (Ex.) compounds of the invention.

TABLE 65
DC₅₀ at 18 h (nM)

| KRAS | Ex. | Ex. | Ex. | Ex. | Ex. | Ex. | Ex. | Ex. |
|---|---|---|---|---|---|---|---|---|
| mutation | I-25 | I-27 | I-29 | I-30 | I-31 | I-41 | I-43 | I-47 |
| G12C | 1.9 | 3.7 | 1.4 | 1.2 | 3.6 | 1.2 | 1.1 | n.a. |
| G12D | 1.3 | 1.1 | 1.5 | 0.8 | 3.3 | 1.3 | 1.3 | 48.0 |
| G12V | 3.8 | 2.7 | 3.5 | 2.2 | 8.7 | 2.5 | 2.6 | 98.8 |
| G12WT | 0.8 | 0.7 | 0.8 | 0.4 | 1.7 | 0.5 | 0.5 | 36.2 |
| G13D | 2.5 | 1.6 | 1.2 | 1.0 | 2.7 | 0.8 | 1.1 | 44.9 |

TABLE 66
Dmax at 18 h (% Ctrl.)

| KRAS | Ex. | Ex. | Ex. | Ex. | Ex. | Ex. | Ex. | Ex. |
|---|---|---|---|---|---|---|---|---|
| mutation | I-25 | I-27 | I-29 | I-30 | I-31 | I-41 | I-43 | I-47 |
| G12C | 92.5 | 83 | 95.6 | 95.6 | 94.7 | 96.2 | 95.6 | n.a. |
| G12D | 96 | 95 | 96.0 | 96.1 | 95.8 | 96.1 | 95.8 | 94 |
| G12V | 90.5 | 86 | 90.5 | 91.0 | 89.2 | 90.8 | 89.4 | 90 |
| G12WT | 96 | 95.5 | 97.2 | 97.8 | 97.5 | 98.0 | 98.1 | 95.4 |
| G13D | 94 | 93 | 89.7 | 91.9 | 89.7 | 89.7 | 90.4 | 90 |

Additional Proliferation Assays with Mutant Cancer Cell Lines

### NCI-H358 CTG Proliferation Assay (120 h) (NSCLC, G12C)

NCI-H358 cells (ATCC No. CRL-5807) are dispensed into white bottom opaque 96 well plates (Perkin Elmer cat no. 5680) at a density of 2000 cells per well in 100 μL RPMI-1640 ATCC-Formulation (Gibco #A10491)+10% FCS (fetal calf serum) (assay 1) or into black 384-well plates, flat and clear bottom (Greiner, PNr. 781091) at a density of 200 cells per well in 60 μl RPMI-1640 ATCC-Formulation (Gibco #A10491)+10% FCS (fetal calf serum) (assay 2). Cells are incubated overnight at 37° C. in a humidified tissue culture incubator at 5% CO₂. Compounds (10 mM stock in DMSO) are added at logarithmic dose series using the HP Digital Dispenser D300 (Tecan) (assay 1) or the ECHO acoustic liquid handler system (Beckman Coulter) (assay 2), normalizing for added DMSO and including DMSO controls. For the TO time point measurement, untreated cells are analyzed at the time of compound addition. Plates are incubated for 120 h, and cell viability is measured using CellTiter-Glo luminescent cell viability reagent (Promega product code G7570). Viability (stated as percent of control) is defined as relative luminescence units RLU of each well divided by the RLU of cells in DMSO controls. IC₅₀ values are determined from viability measurements by non-linear regression using a four parameter model.

### AsPC-1 CTG Proliferation Assay (120 h) (Pancreatic Cancer, G12D)

The CTG assay is designed to measure quantitatively the proliferation of AsPC-1 cells (ATCC CRL-5985), using the CellTiter Glow Assay Kit (Promega G7571). Cells are grown in RPMI medium (ATCC) supplemented with Fetal Calf Serum (Life Technologies, Gibco BRL, Cat. No. 10270-106). On “day 0” 2000 AsPC-1 cells are seeded in 60 μL RPMI ATCC+10% FCS+ Penstrep in a 384-well plate, flat and clear bottom (Greiner, PNr. 781091). Cells are then incubated in the plates at 37° C. in a CO₂ incubator overnight. On day 1, compounds (10 mM stock in DMSO) are added with the ECHO acoustic liquid handler system (Beckman Coulter), including DMSO controls. Plates are incubated for 120 h, and cell viability is measured using CellTiter-Glo luminescent cell viability reagent (Promega product code G7570). Viability (stated as percent of control) is defined as relative luminescence units RLU of each well divided by the RLU of cells in DMSO controls. IC₅₀ values are determined from viability measurements by non-linear regression using a four-parameter model.

### GP2D Proliferation Assay (120 h) (Colorectal Cancer, G12D)

GP2D cells (ATCC No. CRL-5807) are dispensed into white 384-well plates, flat and white bottom (Perkin Elmer, 6007680) at a density of 500 cells per well in 40 μl DMEM (Sigma, D6429)+1× GlutaMAX (Gibco, 35050038)+10% FCS (fetal calf serum). Cells are incubated overnight at 37° C. in a humidified tissue culture incubator at 5% CO₂. Compounds (10 mM stock in DMSO) are added at logarithmic dose series using the HP Digital Dispenser D300 (Tecan), including DMSO controls and normalizing for added DMSO. For the TO time point measurement, untreated cells are analyzed at the time of compound addition. Plates are incubated for 120 h, and cell viability is measured using CellTiter-Glo luminescent cell viability reagent (Promega product code G7570). Viability (stated as percent of control) is defined as relative luminescence units RLU of each well divided by the RLU of cells in DMSO controls. IC₅₀ values are determined from viability measurements by non-linear regression using a four parameter model.

### SAS CTG Proliferation Assay (120 h) (HNSCC, Wt Amplified)

SAS cells (JCRB0260) are dispensed into 384-well plates, flat and clear bottom (Greiner, PNr. 781091) at a density of 300 cells per well in 60 μL DMEM:F12 (Gibco 31330-038)+10% Fetal Calf Serum (HyClone, PNr.: SH30084.03) and incubated at 37° C. in a CO₂ incubator overnight. The next day, compounds (10 mM stock in DMSO) are added with the ECHO acoustic liquid handler system (Beckman Coulter), including DMSO controls. Plates are incubated for 120 h, and cell viability is measured using CellTiter-Glo luminescent cell viability reagent (Promega product code G7570). Viability (stated as percent of control) is defined as relative luminescence units RLU of each well divided by the RLU of cells in DMSO controls. IC₅₀ values are determined from viability measurements by non-linear regression using a four-parameter model.

### SK—CO-1 CTG Proliferation Assay (120 h) (CRC, G12V)

SK—CO-1 cells (ATCC HTB-39) are dispensed into 384-well plates, flat and clear bottom (Greiner, PNr. 781091) at a density of 500 cells per well in 60 μL EMEM (Sigma M5650)+10% Fetal Calf Serum (HyClone, PNr.: SH30084.03) and incubated at 37° C. in a CO₂ incubator overnight. The next day, compounds (10 mM stock in DMSO) are added with the ECHO acoustic liquid handler system (Beckman Coulter), including DMSO controls. Plates are incubated for 120 h, and cell viability is measured using CellTiter-Glo luminescent cell viability reagent (Promega product code G7570). Viability (stated as percent of control) is defined as relative luminescence units RLU of each well divided by the RLU of cells in DMSO controls. IC₅₀ values are determined from viability measurements by non-linear regression using a four-parameter model.

### LOVO CTG Proliferation Assay (120 h) (CRC, G13D)

LOVO cells (ATCC CCL-229) are dispensed into 384-well plates, flat and clear bottom (Greiner, PNr. 781091) at a density of 1000 cells per well in 60 μL DMEM (Sigma D6429)+10% Fetal Calf Serum (HyClone, PNr.: SH30084.03) and incubated at 37° C. in a CO₂ incubator overnight. The next day, compounds (10 mM stock in DMSO) are added with the ECHO acoustic liquid handler system (Beckman Coulter), including DMSO controls. Plates are incubated for 120 h, and cell viability is measured using CellTiter-Glo luminescent cell viability reagent (Promega product code G7570). Viability (stated as percent of control) is defined as relative luminescence units RLU of each well divided by the RLU of cells in DMSO controls. IC₅₀ values are determined from viability measurements by non-linear regression using a four-parameter model.

### GP5d Cell Proliferation Assay (Colorectal Cancer, G12D)

GP5d cells (ECACC 95090715) are dispensed into white bottom opaque 96 well plates at a density of 5000 cells per well in 100 μL DMEM (BioWhittaker, Cat #BE12-605F) supplemented with 10% FCS. Cells are incubated overnight at 37° C. in a humidified tissue culture incubator at 5% CO₂. Compounds (10 mM stock in DMSO) are added at logarithmic dose series using the HP Digital Dispenser D300 (Tecan), normalizing for added DMSO. For the TO time point measurement, untreated cells are analysed at the time of compound addition. Plates are incubated for 5 days, and cell viability is measured using CellTiter-Glo luminescent cell viability reagent (Promega, Cat. No G7570). Viability (stated as percent of control) is defined as relative luminescence units RLU of each well divided by the RLU of cells in DMSO controls. IC₅₀ values were computed from relative viability values using a four parametric logistic model.

### SW620 Cell Proliferation Assay (Colorectal Cancer, G12V)

SW620 cells (ATCC CCL-227) are dispensed into white bottom opaque 96 well plates (Perkin Elmer cat no. 5680) at a density of 2000 cells per well in 100 μL DMEM (BioWhittaker, Cat #BE12-605F) supplemented with 10% FCS. Cells are incubated overnight at 37° C. in a humidified tissue culture incubator at 5% CO₂. Compounds (10 mM stock in DMSO) are added at logarithmic dose series using the HP Digital Dispenser D300 (Tecan), normalizing for added DMSO. For the TO time point measurement, untreated cells are analysed at the time of compound addition. Plates are incubated for 5 days, and cell viability is measured using CellTiter-Glo luminescent cell viability reagent (Promega, Cat. No G7570). Viability (stated as percent of control) is defined as relative luminescence units RLU of each well divided by the RLU of cells in DMSO controls. IC₅₀ values were computed from relative viability values using a four parametric logistic model.

### A375 CTG Proliferation Assay (120 h) (Melanoma, Wt, B-Raf Mutant, Negative Control)

A375 cells (ATCC CRL-1619) are dispensed into 384-well plates, flat and clear bottom (Greiner, PNr. 781091) at a density of 300 cells per well in 60 μL DMEM (Sigma D6429)+10% Fetal Calf Serum (HyClone, PNr.: SH30084.03) and incubated at 37° C. in a CO₂ incubator overnight. The next day, compounds (10 mM stock in DMSO) are added at logarithmic dose series using the HP Digital Dispenser D300 (Tecan), including DMSO controls. Plates are incubated for 120 h, and cell viability is measured using CellTiter-Glo luminescent cell viability reagent (Promega product code G7570). Viability (stated as percent of control) is defined as relative luminescence units RLU of each well divided by the RLU of cells in DMSO controls. IC₅₀ values are determined from viability measurements by non-linear regression using a four-parameter model.

IC₅₀ values of representative compounds according to the invention measured with these assays in the indicated cell lines are presented in table 67.

TABLE 67

| | H358 | H358 | AsPC-1 | GP2D | SAS | SK-CO-1 | LOVO | GP5D | SW620 | A375 |
|---|---|---|---|---|---|---|---|---|---|---|
| | (assay 1) | (assay 2) | IC₅₀ | IC₅₀ | IC₅₀ | IC₅₀ | IC₅₀ | IC₅₀ | IC₅₀ | IC₅₀ |
| # | IC₅₀ (nM) | IC₅₀ (nM) | (nM) | (nM) | (nM) | (nM) | (nM) | (nM) | (nM) | (nM) |
| I-1 | 150.9 | 96.5 | | | | | | 22.239 | 27.340 | |
| I-2 | 2928.1 | | | | | | | 9000.000 | 598.134 | |
| I-3 | 66.0 | 60.7 | | | | | | 6.140 | 13.128 | |
| I-4 | 444.8 | 195.5 | 372.1 | | 627.9 | 207.4 | 425.1 | 36.061 | 53.396 | |
| I-5 | 117.1 | 58.4 | | | | | | 13.756 | 47.900 | |
| I-6 | | 253.4 | 645.2 | | 17013.2 | 1211.3 | | | | |
| I-7 | 480.2 | 179.8 | | | | | | 23.396 | 55.926 | |
| I-8 | 724.8 | 135.8 | | | | | | 90.228 | 50.409 | |
| I-9 | | 288.2 | 470.2 | | 16379.3 | 216.4 | 59.4 | | | |
| I-10 | 264.7 | 203.2 | | | | | | 39.307 | 114.785 | |
| I-11 | 711.7 | 349.1 | | | | | | 9000.000 | 9000.000 | |
| I-12 | 407.4 | 118.1 | | | | | | 106.421 | 108.720 | |
| I-13 | 468.4 | 335.5 | | | | | | 130.904 | 205.904 | |
| I-14 | | 843.3 | | | | | | | | |
| I-15 | | 121.6 | | | | | | | | |
| I-16 | 230.9 | 85.5 | 168.8 | 33.3 | 29.7 | 183.8 | 1820.1 | | 37.117 | 3000.0 |
| I-17 | | 89.5 | | | | | | | | |
| I-18 | | 168.7 | 435.2 | | 3378.8 | 123.9 | 278.2 | | | |
| I-19 | 346.2 | | | | | | | 23.503 | 28.994 | |
| I-20 | 439.3 | 376.9 | | | | | | 92.400 | 83.929 | |
| I-21 | | 81.3 | | | | | | | | |
| I-22 | | 97.5 | | | | | | | | |
| I-23 | 1151.9 | 526.6 | | | | | | 856.981 | 266.693 | |
| I-24 | 1074.5 | 1437.2 | 1800.0 | | 1310.8 | 2224.4 | 1952.8 | 766.361 | 802.396 | |
| I-25 | 45.6 | 63.9 | 93.4 | | 17.8 | 57.3 | 53.5 | 3.968 | 9.882 | |
| I-26 | 485.0 | 434.9 | | | | | | 132.985 | 264.668 | |
| I-27 | 60.5 | 35.2 | 144.2 | 5.1 | 94.6 | 51.7 | 40.9 | 3.997 | 14.867 | 3000.0 |
| I-28 | 32.0 | 81.4 | 72.0 | 2.9 | 58.3 | 45.0 | 648.8 | | 13.187 | 1090.8 |
| I-29 | 28.8 | | 97.2 | 8.5 | 548.0 | 24.3 | 44.0 | | 26.432 | 3000.0 |
| I-30 | 29.3 | | 69.0 | 6.4 | 15.2 | 29.7 | 86.4 | | 18.004 | 3000.0 |
| I-31 | 158.7 | | 182.9 | 26.1 | 90.0 | 131.6 | 343.5 | | 62.091 | 3000.0 |
| I-32 | | | 8333.3 | | 9444.0 | 507.1 | 929.0 | | | |
| I-33 | | | 323.2 | | 84.5 | 1953.1 | 50.0 | | | |
| I-34 | 159.5 | 43.8 | | | | | | 21.712 | 51.318 | |
| I-38 | 98.8 | | 127.7 | 12.6 | 58.1 | 116.6 | 596.9 | | 28.444 | 3000.0 |
| I-39 | | 57.0 | | | | | | | | |
| I-40 | | 2715.1 | | | | | | | | |
| I-41 | 59.6 | 14.9 | 42.0 | 7.7 | 30.3 | 22.6 | 29.3 | | 10.487 | 3000.0 |
| I-42 | | 1919.7 | | | | | | | | |
| I-43 | 50.8 | 8.1 | 52.1 | 6.6 | 410.9 | 26.2 | 22.2 | | 9.398 | 3000.0 |
| I-44 | | 603.6 | | | | | | | | |
| I-45 | 98.6 | 13.4 | 51.4 | 9.4 | 1805.2 | 33.3 | 43.0 | | 14.549 | 3000.0 |
| I-47 | 2501.0 | 168.3 | | | | | | 141.028 | 280.299 | |
| I-48 | 3552.8 | | | | | | | 2632.225 | 802.540 | |

### Formulation

The formulation examples which follow illustrate the present invention without restricting its scope:

### Examples of Pharmaceutical Formulations

A)

| Tablets | per tablet |
|---|---|
| active substance according to formula (I) | 100 mg |
| lactose | 140 mg |
| corn starch | 240 mg |
| polyvinylpyrrolidone | 15 mg |
| magnesium stearate | 5 mg |
| | 500 mg |

The finely ground active substance, lactose and some of the corn starch are mixed together. The mixture is screened, then moistened with a solution of polyvinylpyrrolidone in water, kneaded, wet-granulated and dried. The granules, the remaining corn starch and the magnesium stearate are screened and mixed together. The mixture is compressed to produce tablets of suitable shape and size.

B)

| Tablets | per tablet |
|---|---|
| active substance according to formula (I) | 80 mg |
| lactose | 55 mg |
| corn starch | 190 mg |
| microcrystalline cellulose | 35 mg |
| polyvinylpyrrolidone | 15 mg |
| sodiumcarboxymethyl starch | 23 mg |
| magnesium stearate | 2 mg |
| | 400 mg |

The finely ground active substance, some of the corn starch, lactose, microcrystalline cellulose and polyvinylpyrrolidone are mixed together, the mixture is screened and worked with the remaining corn starch and water to form a granulate which is dried and screened. The sodiumcarboxymethyl starch and the magnesium stearate are added and mixed in and the mixture is compressed to form tablets of a suitable size.

C)

| Tablets | per tablet |
|---|---|
| active substance according to formula (I) | 25 mg |
| lactose | 50 mg |
| microcrystalline cellulose | 24 mg |
| magnesium stearate | 1 mg |
| | 100 mg |

The active substance, lactose and cellulose are mixed together. The mixture is screened, then either moistened with water, kneaded, wet-granulated and dried or dry-granulated or directely final blend with the magnesium stearate and compressed to tablets of suitable shape and size. When wet-granulated, additional lactose or cellulose and magnesium stearate is added and the mixture is compressed to produce tablets of suitable shape and size.

D)

| Ampoule solution | | |
|---|---|---|
| active substance according to formulae (I) | 50 | mg |
| sodium chloride | 50 | mg |
| water for inj. | 5 | mL |

The active substance is dissolved in water at its own pH or optionally at pH 5.5 to 6.5 and sodium chloride is added to make it isotonic. The solution obtained is filtered free from pyrogens and the filtrate is transferred under aseptic conditions into ampoules which are then sterilised and sealed by fusion. The ampoules contain 5 mg, 25 mg and 50 mg of active substance.

1. A compound of formula (I): 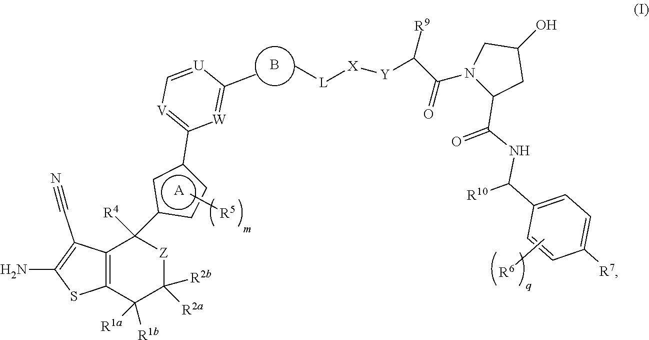
wherein:
R^{1a} and R^{1b} are both independently selected from the group consisting of hydrogen, C₁₋₄alkyl, C₁₋₄haloalkyl, C₁₋₄alkoxy, C₁₋₄haloalkoxy, halogen, —NH₂, —NH(C₁₋₄alkyl), —N(C₁₋₄alkyl)₂, C₃₋₅cycloalkyl and 3-5 membered heterocycloalkyl;
R^{2a} and R^{2b} are both independently selected from the group consisting of hydrogen, C₁₋₄alkyl, C₁₋₄haloalkyl, C₁₋₄alkoxy, C₁₋₄haloalkoxy, halogen, —NH₂, —NH(C₁₋₄alkyl), —N(C₁₋₄alkyl)₂, C₃₋₅cycloalkyl and 3-5 membered heterocycloalkyl;
and/or, optionally, one of R^{1a} or R^{1b} and one of R^{2a} or R^{2b} together with the carbon atoms they are attached to form a cyclopropane ring;
Z is —(CR^{3a}R^{3b})ₙ—;
each R^{3a} and R^{3b} is independently selected from the group consisting of hydrogen, C₁₋₄alkyl, C₁₋₄haloalkyl, C₁₋₄alkoxy, C₁₋₄haloalkoxy, halogen, —NH₂, —NH(C₁₋₄alkyl), —N(C₁₋₄alkyl)₂, C₃₋₅cycloalkyl and 3-5 membered heterocycloalkyl;
or R^{3a} and R^{3b} together with the carbon atom they are attached to form a cyclopropane ring;
n is selected from the group consisting of 0, 1 and 2;
R⁴ is selected from the group consisting of hydrogen, C₁₋₆-alkyl, C₁₋₆-haloalkyl, C₁₋₆-alkoxy, C₁₋₆-haloalkoxy, cyano-C₁₋₆-alkyl, halogen, —OH, —NH₂, —NH(C₁₋₄alkyl), —N(C₁₋₄alkyl)₂, —CN, C₃₋₅cycloalkyl and 3-5 membered heterocycloalkyl;
ring A is a 5 membered heteroarylene;
each R⁵, if present, is independently selected from the group consisting of C₁₋₆alkyl, C₁₋₆haloalkyl, C₁₋₆alkoxy, C₁₋₆haloalkoxy, cyano-C₁₋₆alkyl, halogen, —OH, —NH₂, —NH(C₁₋₄alkyl), —N(C₁₋₄alkyl)₂, —CN, C₃₋₅cycloalkyl and 3-5 membered heterocycloalkyl;
m is selected from the group consisting of 0, 1, 2 and 3;
W is nitrogen (—N═) or —CH═;
V is nitrogen (—N═) or —CH═;
U is nitrogen (—N═) or —C(R¹¹)═;
R¹¹ is selected from hydrogen, halogen and C₁₋₄alkoxy;
ring B is a 3-11 membered heterocycloalkylene optionally substituted with one or more identical or different C₁₋₆alkyl, C₁₋₆alkoxy or a 5-6 membered heterocycloalkyl, wherein the C₁₋₆alkyl is optionally substituted with cyclopropyl;
L is selected from the group consisting of a bond, C₁₋₈alkylene, C₂₋₈alkenylene, C₂₋₈alkynylene and C₁₋₈alkoxylene;
X is —(CH₂)—or —O—;
Y is a 5 membered heteroarylene or —C(O)(NR¹²)—, wherein said 5 membered heteroarylene comprises at least one nitrogen atom and wherein said —C(O)(NR¹²)— is linked to X via the C atom;
R⁹ is C₁₋₄ alkyl;
R¹⁰ is selected from the group consisting of hydrogen, C₁₋₆alkyl, C₁₋₆alkoxy, —C(O)R¹² and —C(O)OR¹², wherein said C₁₋₆alkyl is optionally substituted by —OH or —OP(O)(OH)₂;
each R¹² is independently hydrogen or C₁₋₄alkyl;
q is selected from the group consisting of 0, 1 and 2;
each R⁶, if present, is independently at each occurrence halogen or C₁₋₃alkyl;
R⁷ is selected from the group consisting of halogen, C₁₋₃alkyl, —CN and 5 membered heteroaryl, wherein said 5 membered heteroaryl comprises at least one nitrogen atom and is optionally substituted with R⁸;
R⁸ is C₁₋₃alkyl or C₁₋₃hydroxyalkyl;
or a salt thereof.

2. The compound or salt according to claim 1, wherein m is 0.

3. The compound or salt according to claim 1 or 2 of formula (I*), (I**), (I***) or (I****): 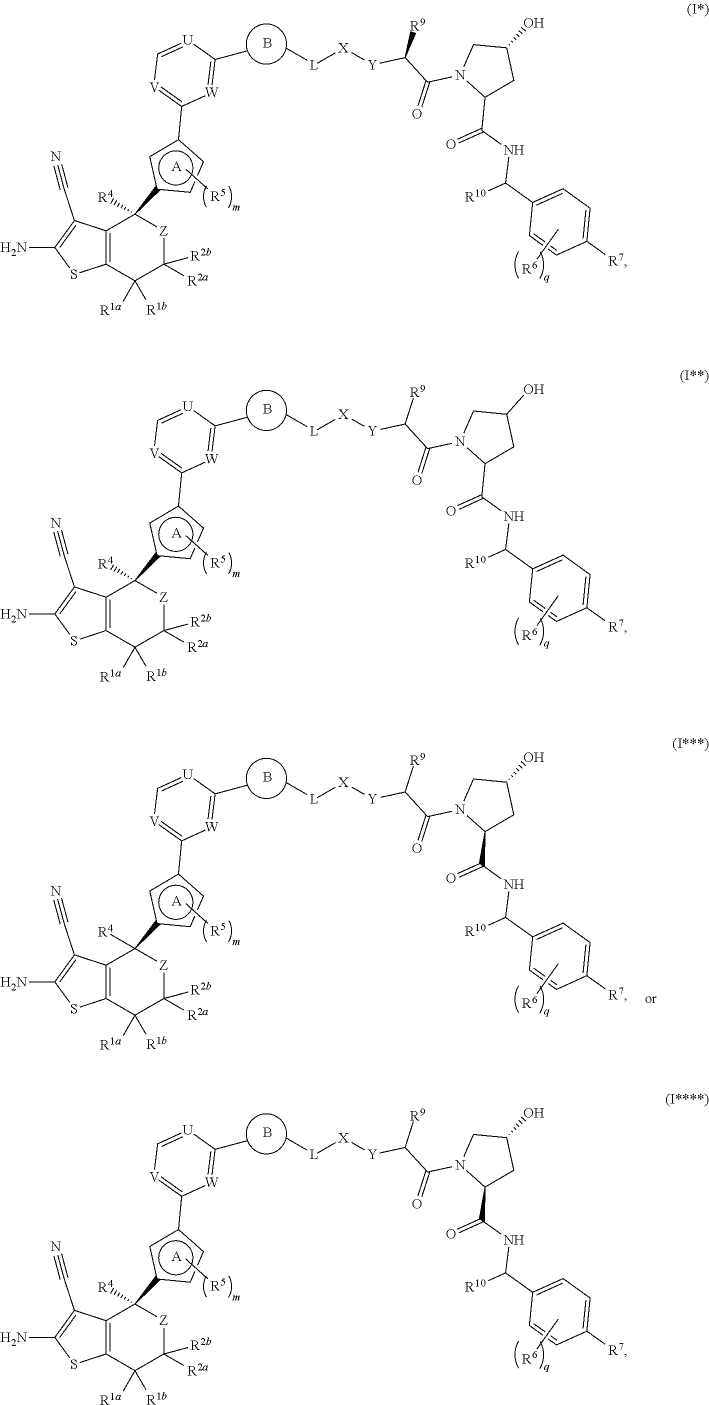
wherein R^{1a}, R^{1b}, R^{2a}, R^{2b}, Z, R⁴, ring A, R⁵, m, U, V, W, ring B, L, X, Y, R⁹, R¹⁰, q, R⁵ and R⁷ are as defined in claim 1 or 2.

4. The compound or salt according to any one of claims 1 to 3, wherein ring A is selected from the group consisting of pyrrole, furan, thiophene, imidazole, pyrazole, oxazole, isoxazole, thiazole, isothiazole, oxadiazole, thiadiazole and triazole.

5. The compound or salt according to any one of claims 1 to 4, wherein ring A is selected from the group consisting of isoxazole, isothiazole and oxadiazole.

6. The compound or salt according to any one of previous claims of formula (Ia), (Ib), (Ic), (Id) or (Ie): 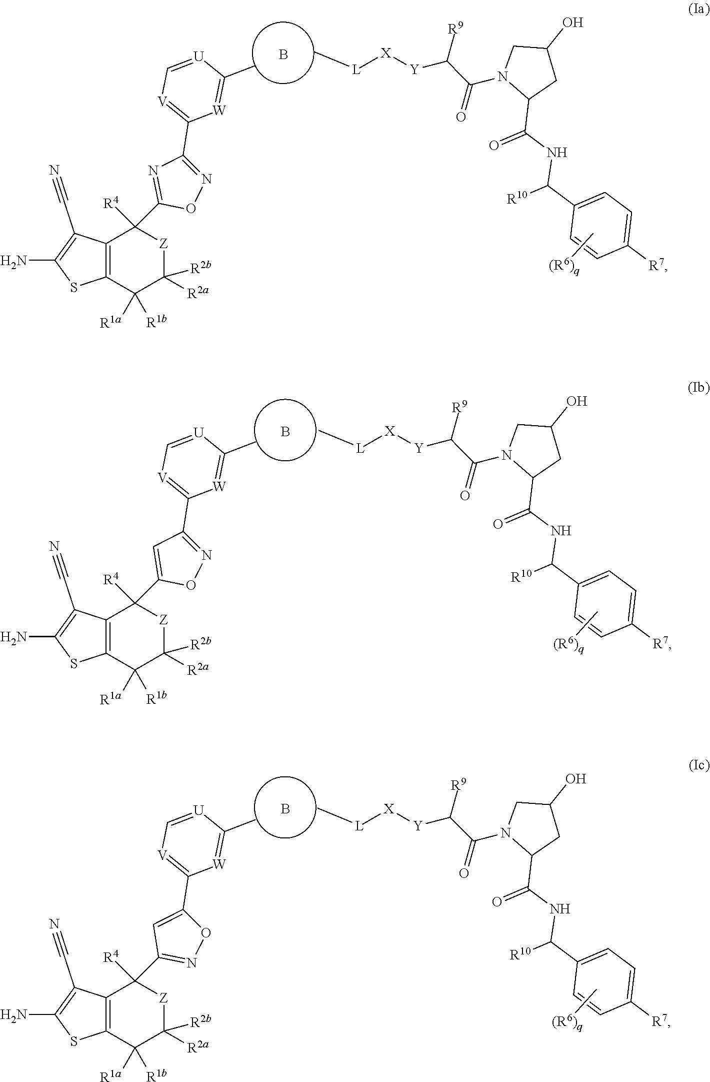 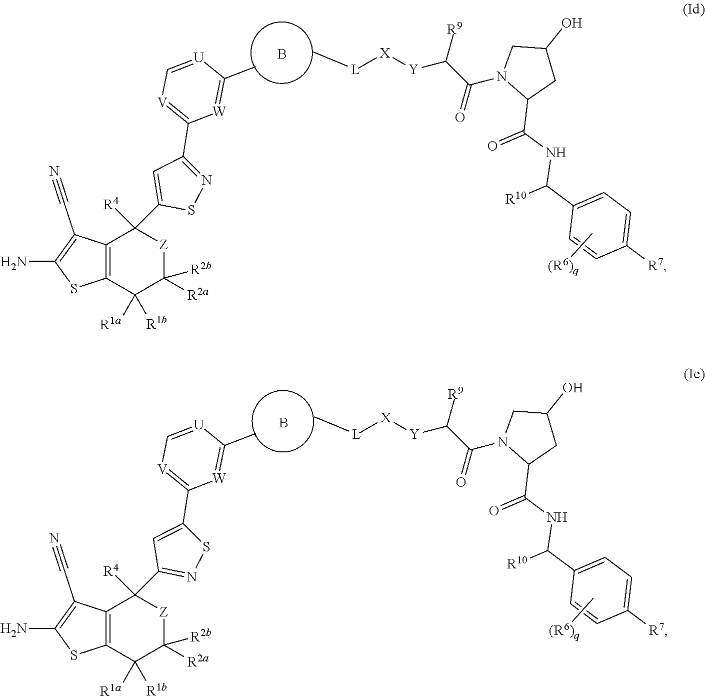
wherein R^{1a}, R^{1b}, R^{2a}, R^{2b}, Z, R⁴, U, V, W, ring B, L, X, Y, R⁹, R¹⁰, q, R⁵, R⁷ and the stereochemistry are defined in anyone of claims 1 to 4.

7. The compound or salt according to any one of claims 1 to 6, wherein:
X is —(CH₂)— and Y is a 5 membered heteroarylene or —C(O)(NR¹²)—, wherein said 5 membered heteroarylene comprises at least one nitrogen atom and wherein said —C(O)(NR¹²)— is linked to X via the C atom, or
X is —O— and Y is a 5 membered heteroarylene comprising at least one nitrogen atom.

8. The compound or salt according to any one of claims 1 to 7, wherein Y is isoxazole or triazole.

9. The compound or salt according to any one of claims 1 to 8 of formula (If): 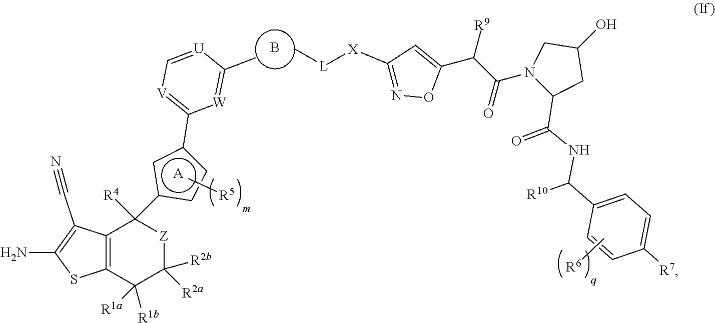
wherein R^{1a}, R^{1b}, R^{2a}, R^{2b}, Z, R⁴, ring A, R⁵, m, U, V, W, ring B, L, X, R⁹, R¹⁰, q, R⁵, R⁷ and the stereochemistry are as defined in any one of claims 1 to 8.

10. The compound or salt according to any one of claims 1 to 8 of formula (Ig): 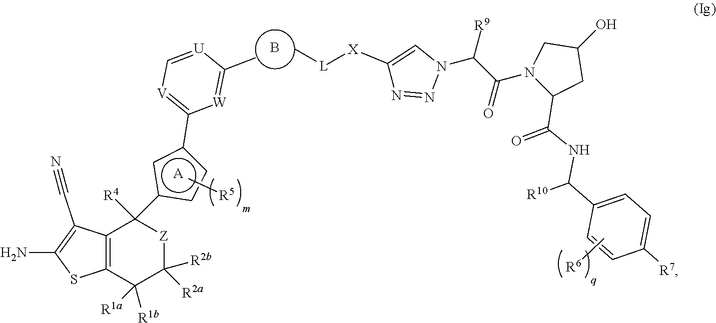
wherein R^{1a}, R^{1b}, R^{2a}, R^{2b}, Z, R⁴, ring A, R⁵, m, U, V, W, ring B, L, X, R⁹, R¹⁰, q, R⁶, R⁷ and the stereochemistry are as defined in any one of claims 1 to 8.

11. The compound or salt according to any one of claims 1 to 7 of formula (Ih): 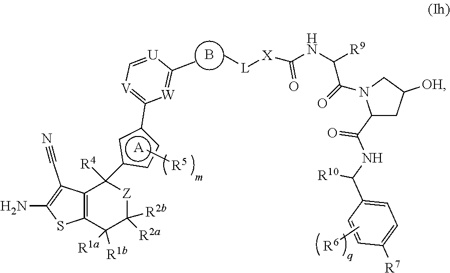
wherein R^{1a}, R^{1b}, R^{2a}, R^{2b}, Z, R⁴, ring A, R⁵, m, U, V, W, ring B, L, X, R⁹, R¹⁰, q, R⁶, R⁷ and the stereochemistry are as defined in any one of claims 1 to 7.

12. The compound or salt according to claim 9 or 10, wherein X is —(CH₂)—.

13. The compound or salt according to claim 9 or 10, wherein X is —O—.

14. The compound or salt according to any one of claims 1 to 13, wherein R^{1a}, R^{1b}, R^{2a} and R^{2b} are hydrogen and Z is —CH₂—.

15. The compound or salt according to any one of claims 1 to 14, wherein R⁴ is methyl.

16. The compound or salt according to any one of claims 1 to 15, wherein at least one of V or W is ═N—.

17. The compound or salt according to any one of claim 1 to 16, wherein ring B is 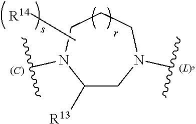
wherein:
r is 0, 1 or 2,
s is 0, 1, or 2,
R¹³ is C₁₋₃alkyl,
each R¹⁴ is independently at each occurrence the same or different C₁₋₃alkyl,
(C) and (L) indicate the atom or substituent of formula (I) to which ring B is attached.

18. The compound or salt according to any one of claim 1 to 17, wherein L is selected from the group consisting of C₁₋₆alkylene, C₂₋₆alkenylene and C₁₋₆alkoxylene.

19. The compound or salt according to any one of claim 1 to 18, wherein R⁹ is iso-propyl and/or the carbon atom to which R⁹ is attached is in the (S) configuration.

20. The compound or salt according to any one of claim 1 to 19, wherein R¹⁰ is selected from the group consisting of hydrogen, C₁₋₆alkyl, and —C(O)OR¹², wherein said C₁₋₆alkyl is optionally substituted by —OH or —OP(O)(OH)₂.

21. The compound or salt according to any one of claim 1 to 20, wherein R¹⁰ is hydrogen.

22. The compound or salt according to any one of claims 1 to 21, wherein R⁷ is selected from the group consisting of: chlorine, bromine, iso-propyl, —CN,

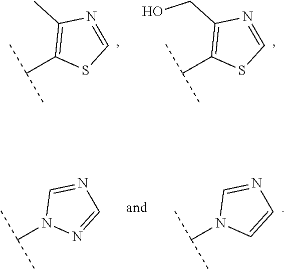

23. The compound or salt according to any one of claims 1 to 22, being selected from the group consisting of: 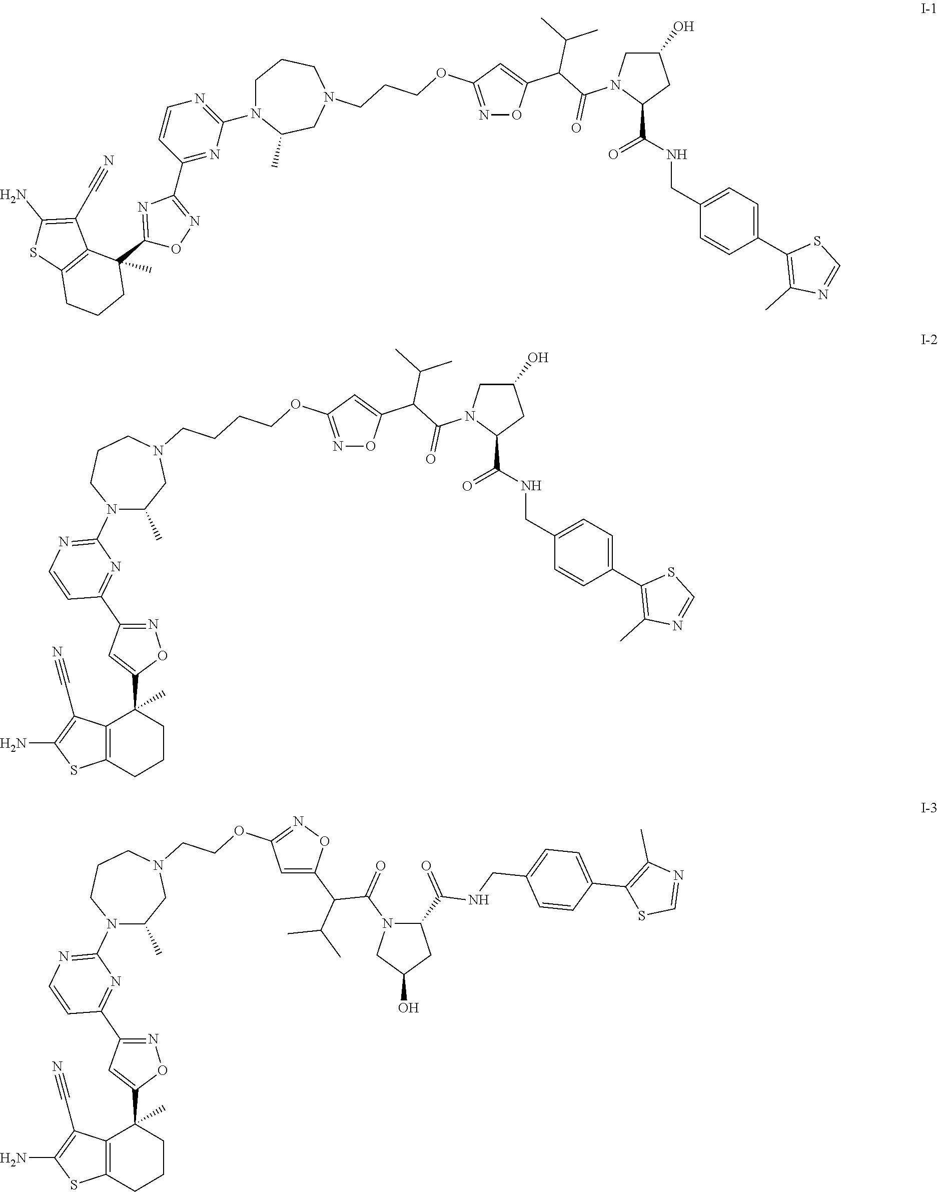 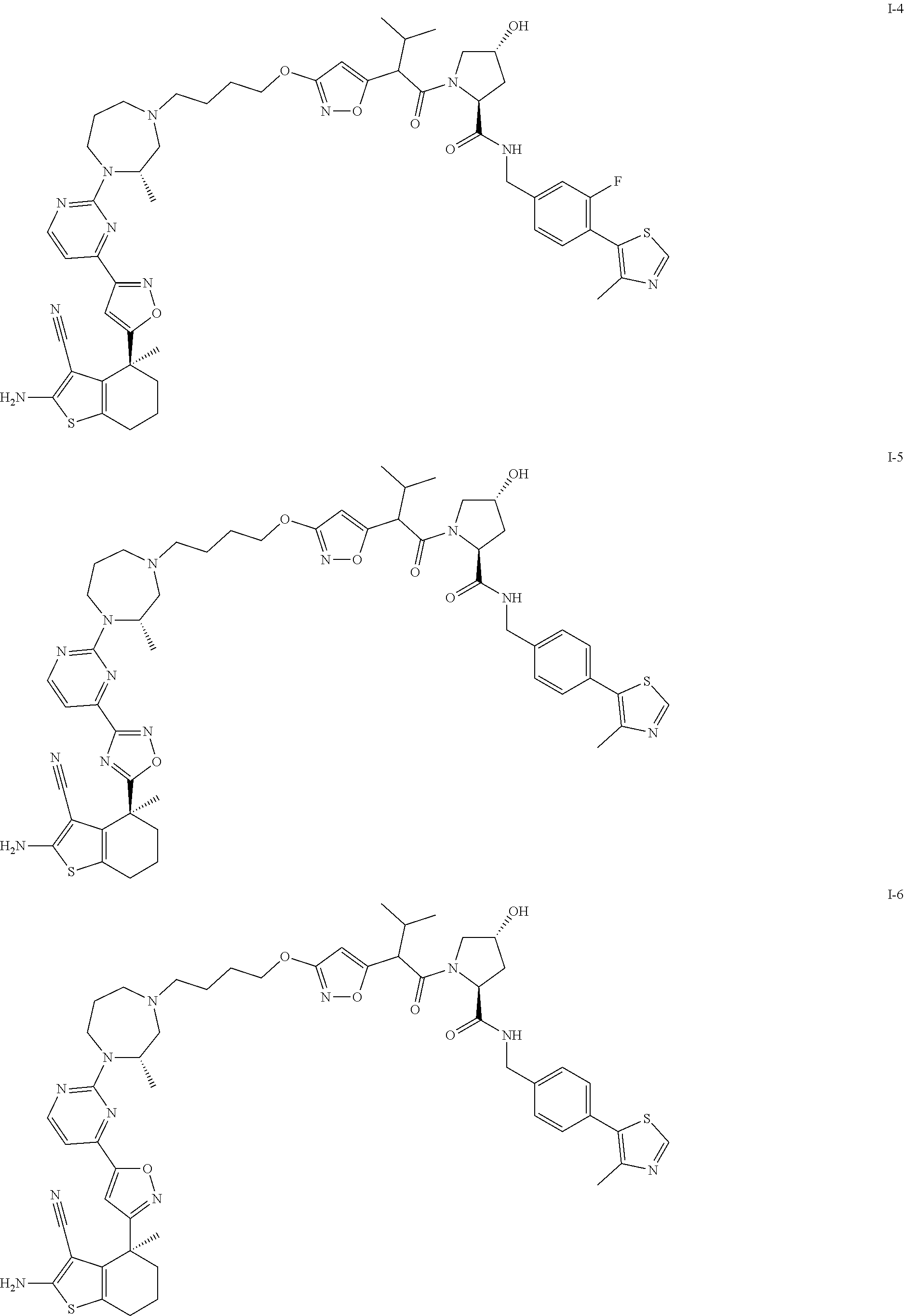 [SMILES: Cc1ncsc1-c1ccc(CNC(=O)[C@@H]2C[C@@H](O)CN2C(=O)C(c2cc(OCCCCN3CCCN(c4nccc(-c5cc([C@@]6(C)CCCc7sc(N)c(C#N)c76)on5)n4)[C@@H](C)C3)no2)C(C)C)cc1F ; Cc1ncsc1-c1ccc(CNC(=O)[C@@H]2C[C@@H](O)CN2C(=O)C(c2cc(OCCCCN3CCCN(c4nccc(-c5noc([C@@]6(C)CCCc7sc(N)c(C#N)c76)n5)n4)[C@@H](C)C3)no2)C(C)C)cc1 ; Cc1ncsc1-c1ccc(CNC(=O)[C@@H]2C[C@@H](O)CN2C(=O)C(c2cc(OCCCCN3CCCN(c4nccc(-c5cc([C@@]6(C)CCCc7sc(N)c(C#N)c76)no5)n4)[C@@H](C)C3)no2)C(C)C)cc1] 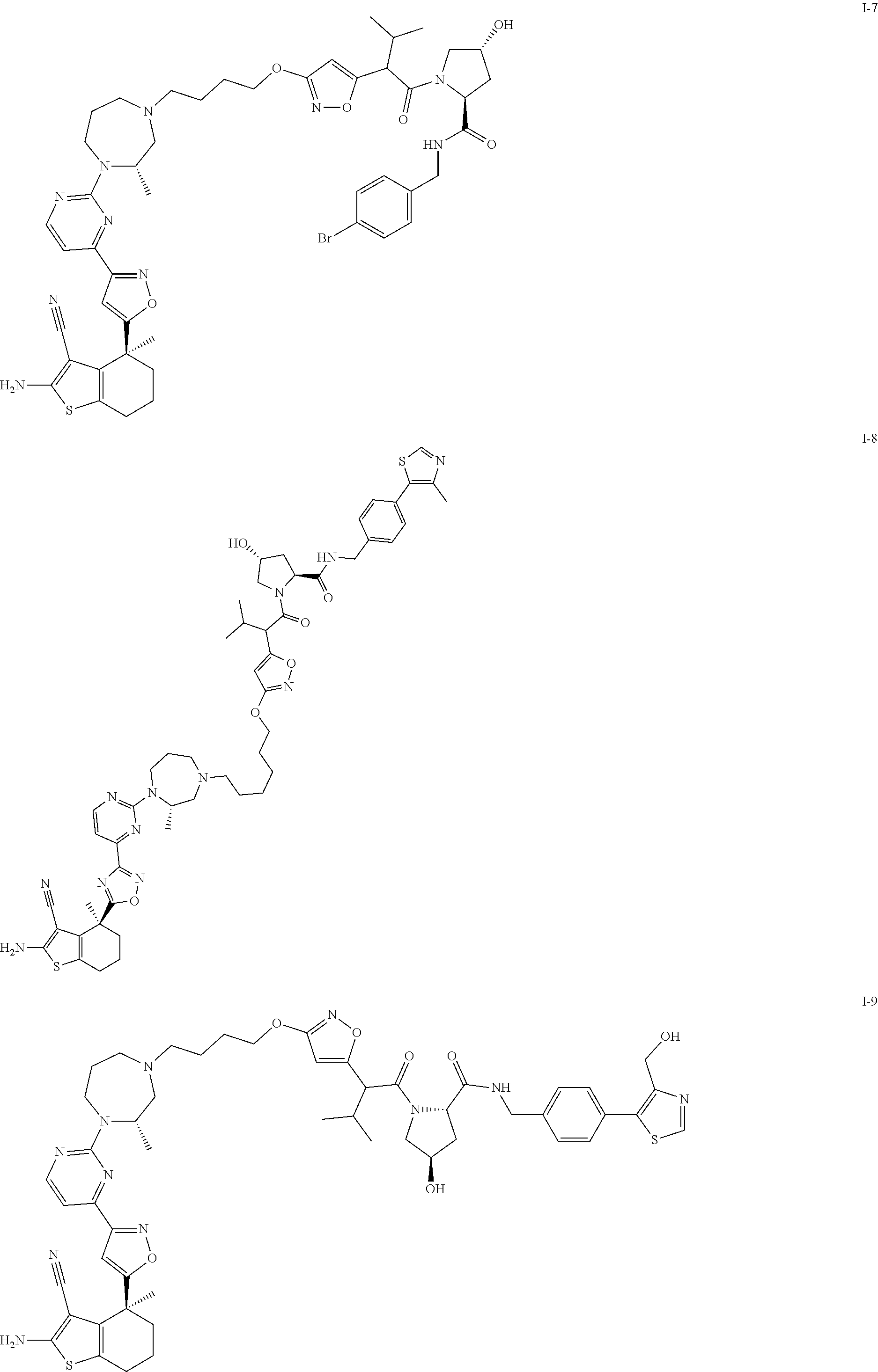 [SMILES: CC(C)C(C(=O)N1C[C@H](O)C[C@H]1C(=O)NCc1ccc(Br)cc1)c1cc(OCCCCN2CCCN(c3nccc(-c4cc([C@@]5(C)CCCc6sc(N)c(C#N)c65)on4)n3)[C@@H](C)C2)no1 ; Cc1ncsc1-c1ccc(CNC(=O)[C@@H]2C[C@@H](O)CN2C(=O)C(c2cc(OCCCCCCN3CCCN(c4nccc(-c5noc([C@@]6(C)CCCc7sc(N)c(C#N)c76)n5)n4)[C@@H](C)C3)no2)C(C)C)cc1 ; CC(C)C(C(=O)N1C[C@H](O)C[C@H]1C(=O)NCc1ccc(-c2scnc2CO)cc1)c1cc(OCCCCN2CCCN(c3nccc(-c4cc([C@@]5(C)CCCc6sc(N)c(C#N)c65)on4)n3)[C@@H](C)C2)no1] 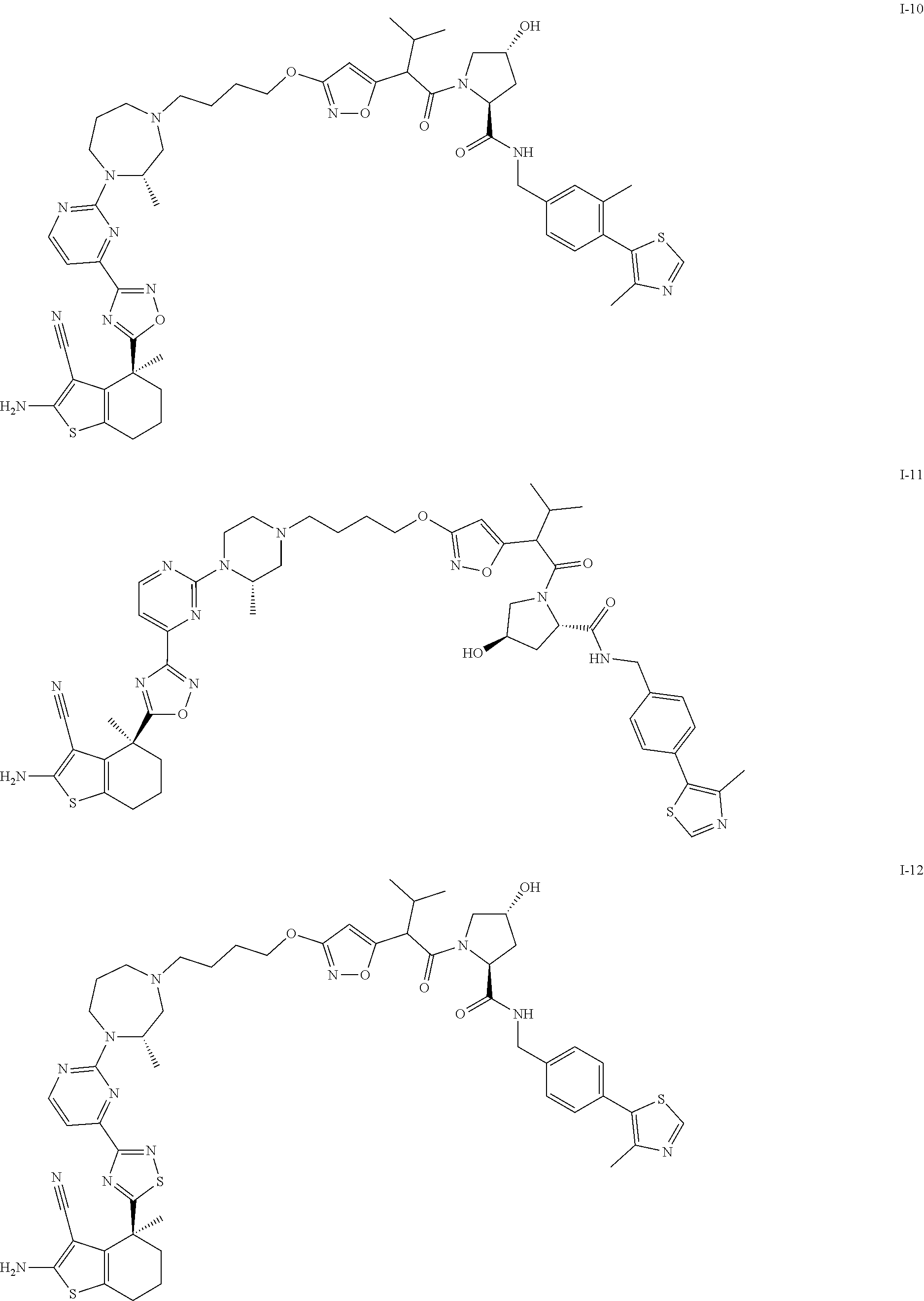 [SMILES: Cc1cc(CNC(=O)[C@@H]2C[C@@H](O)CN2C(=O)C(c2cc(OCCCCN3CCCN(c4nccc(-c5noc([C@@]6(C)CCCc7sc(N)c(C#N)c76)n5)n4)[C@@H](C)C3)no2)C(C)C)ccc1-c1scnc1C ; Cc1ncsc1-c1ccc(CNC(=O)[C@@H]2C[C@@H](O)CN2C(=O)C(c2cc(OCCCCN3CCN(c4nccc(-c5noc([C@@]6(C)CCCc7sc(N)c(C#N)c76)n5)n4)[C@@H](C)C3)no2)C(C)C)cc1 ; Cc1ncsc1-c1ccc(CNC(=O)[C@@H]2C[C@@H](O)CN2C(=O)C(c2cc(OCCCCN3CCCN(c4nccc(-c5nsc([C@@]6(C)CCCc7sc(N)c(C#N)c76)n5)n4)[C@@H](C)C3)no2)C(C)C)cc1] 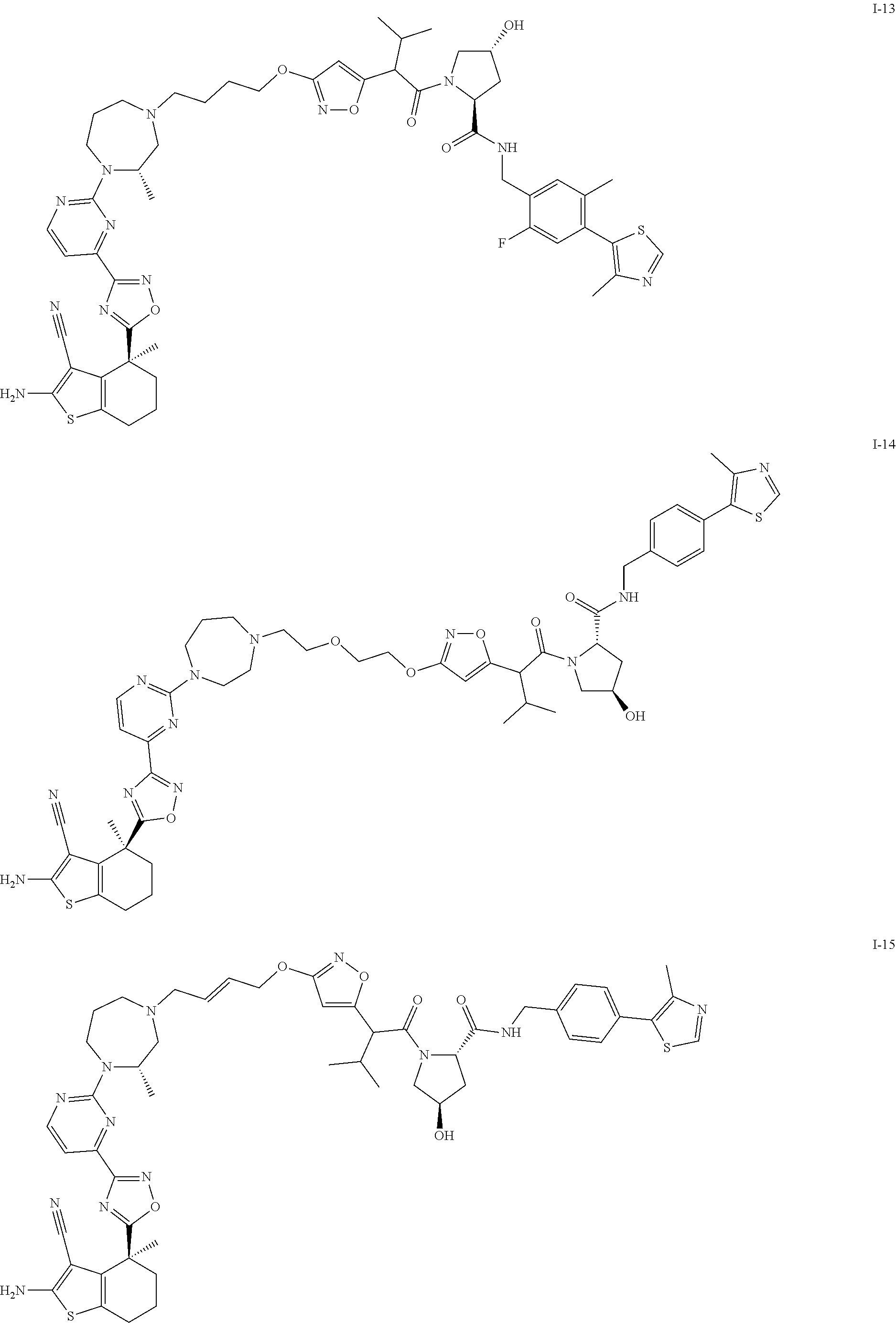 [SMILES: Cc1cc(CNC(=O)[C@@H]2C[C@@H](O)CN2C(=O)C(c2cc(OCCCCN3CCCN(c4nccc(-c5noc([C@@]6(C)CCCc7sc(N)c(C#N)c76)n5)n4)[C@@H](C)C3)no2)C(C)C)c(F)cc1-c1scnc1C ; Cc1ncsc1-c1ccc(CNC(=O)[C@@H]2C[C@@H](O)CN2C(=O)C(c2cc(OCCOCCN3CCCN(c4nccc(-c5noc([C@@]6(C)CCCc7sc(N)c(C#N)c76)n5)n4)CC3)no2)C(C)C)cc1 ; Cc1ncsc1-c1ccc(CNC(=O)[C@@H]2C[C@@H](O)CN2C(=O)C(c2cc(OC/C=C/CN3CCCN(c4nccc(-c5noc([C@@]6(C)CCCc7sc(N)c(C#N)c76)n5)n4)[C@@H](C)C3)no2)C(C)C)cc1] 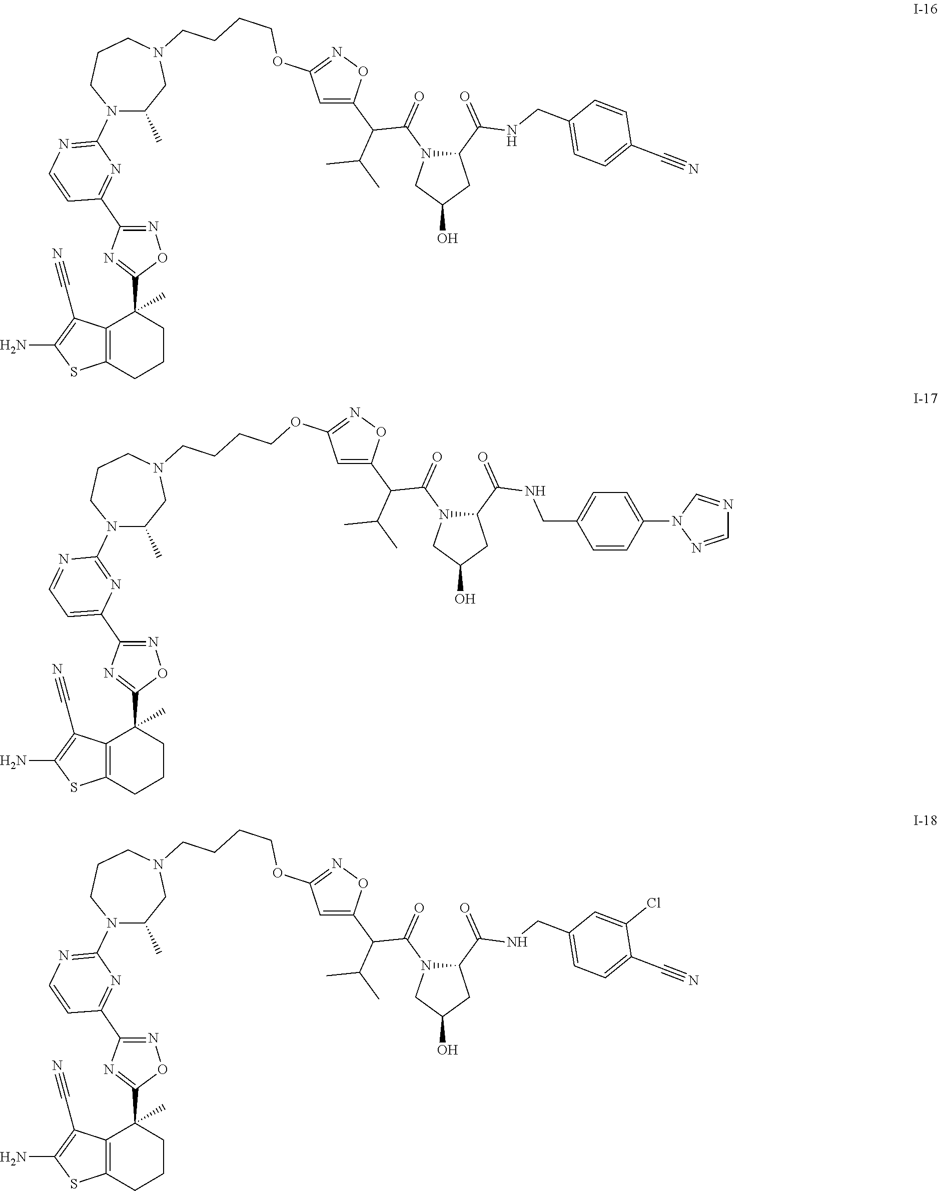 [SMILES: CC(C)C(C(=O)N1C[C@H](O)C[C@H]1C(=O)NCc1ccc(C#N)cc1)c1cc(OCCCCN2CCCN(c3nccc(-c4noc([C@@]5(C)CCCc6sc(N)c(C#N)c65)n4)n3)[C@@H](C)C2)no1 ; CC(C)C(C(=O)N1C[C@H](O)C[C@H]1C(=O)NCc1ccc(-n2cncn2)cc1)c1cc(OCCCCN2CCCN(c3nccc(-c4noc([C@@]5(C)CCCc6sc(N)c(C#N)c65)n4)n3)[C@@H](C)C2)no1 ; CC(C)C(C(=O)N1C[C@H](O)C[C@H]1C(=O)NCc1ccc(C#N)c(Cl)c1)c1cc(OCCCCN2CCCN(c3nccc(-c4noc([C@@]5(C)CCCc6sc(N)c(C#N)c65)n4)n3)[C@@H](C)C2)no1] 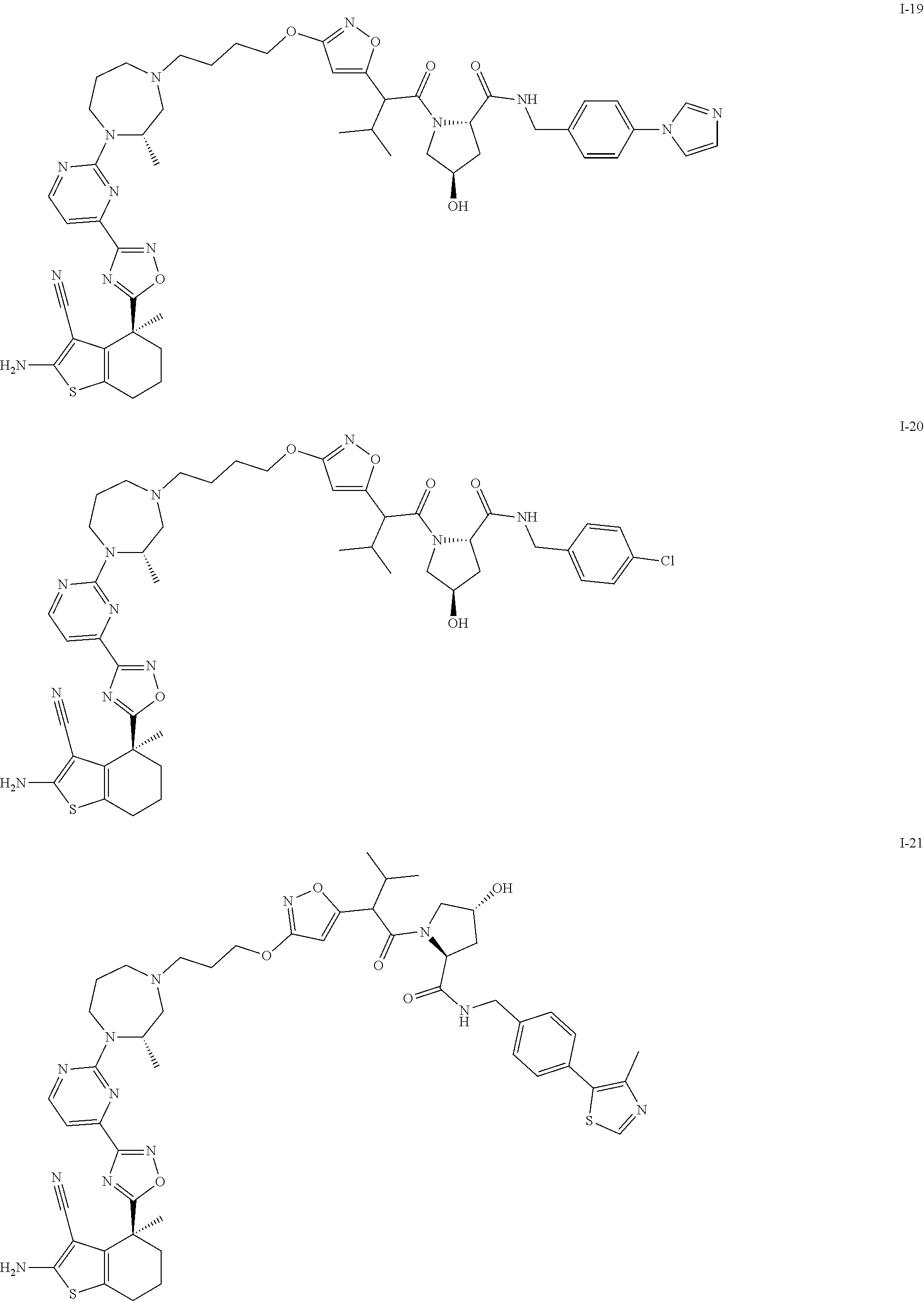 [SMILES: CC(C)C(C(=O)N1C[C@H](O)C[C@H]1C(=O)NCc1ccc(-n2ccnc2)cc1)c1cc(OCCCCN2CCCN(c3nccc(-c4noc([C@@]5(C)CCCc6sc(N)c(C#N)c65)n4)n3)[C@@H](C)C2)no1 ; CC(C)C(C(=O)N1C[C@H](O)C[C@H]1C(=O)NCc1ccc(Cl)cc1)c1cc(OCCCCN2CCCN(c3nccc(-c4noc([C@@]5(C)CCCc6sc(N)c(C#N)c65)n4)n3)[C@@H](C)C2)no1 ; Cc1ncsc1-c1ccc(CNC(=O)[C@@H]2C[C@@H](O)CN2C(=O)C(c2cc(OCCCN3CCCN(c4nccc(-c5noc([C@@]6(C)CCCc7sc(N)c(C#N)c76)n5)n4)[C@@H](C)C3)no2)C(C)C)cc1] 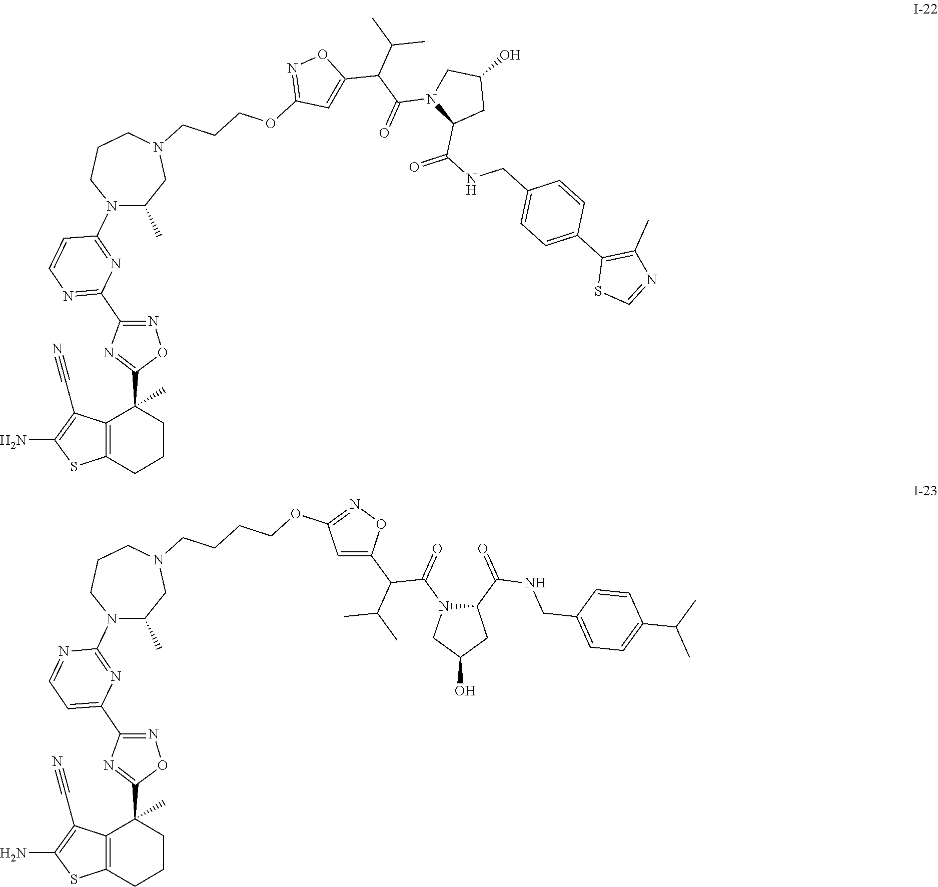 [SMILES: Cc1ncsc1-c1ccc(CNC(=O)[C@@H]2C[C@@H](O)CN2C(=O)C(c2cc(OCCCN3CCCN(c4ccnc(-c5noc([C@@]6(C)CCCc7sc(N)c(C#N)c76)n5)n4)[C@@H](C)C3)no2)C(C)C)cc1 ; CC(C)c1ccc(CNC(=O)[C@@H]2C[C@@H](O)CN2C(=O)C(c2cc(OCCCCN3CCCN(c4nccc(-c5noc([C@@]6(C)CCCc7sc(N)c(C#N)c76)n5)n4)[C@@H](C)C3)no2)C(C)C)cc1] 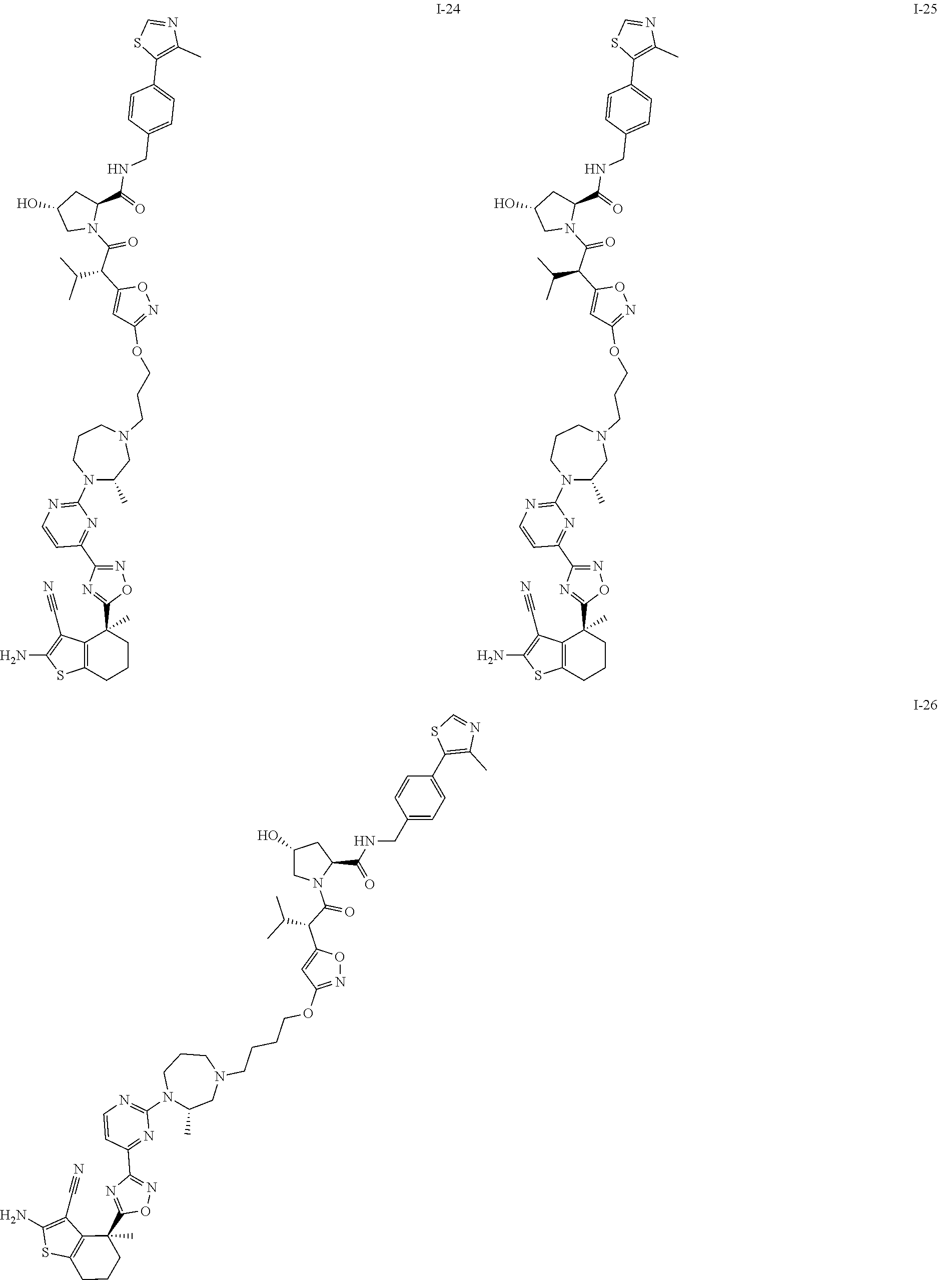 [SMILES: Cc1ncsc1-c1ccc(CNC(=O)[C@@H]2C[C@@H](O)CN2C(=O)[C@H](c2cc(OCCCN3CCCN(c4nccc(-c5noc([C@@]6(C)CCCc7sc(N)c(C#N)c76)n5)n4)[C@@H](C)C3)no2)C(C)C)cc1 ; Cc1ncsc1-c1ccc(CNC(=O)[C@@H]2C[C@@H](O)CN2C(=O)[C@@H](c2cc(OCCCN3CCCN(c4nccc(-c5noc([C@@]6(C)CCCc7sc(N)c(C#N)c76)n5)n4)[C@@H](C)C3)no2)C(C)C)cc1 ; Cc1ncsc1-c1ccc(CNC(=O)[C@@H]2C[C@@H](O)CN2C(=O)[C@H](c2cc(OCCCCN3CCCN(c4nccc(-c5noc([C@@]6(C)CCCc7sc(N)c(C#N)c76)n5)n4)[C@@H](C)C3)no2)C(C)C)cc1] 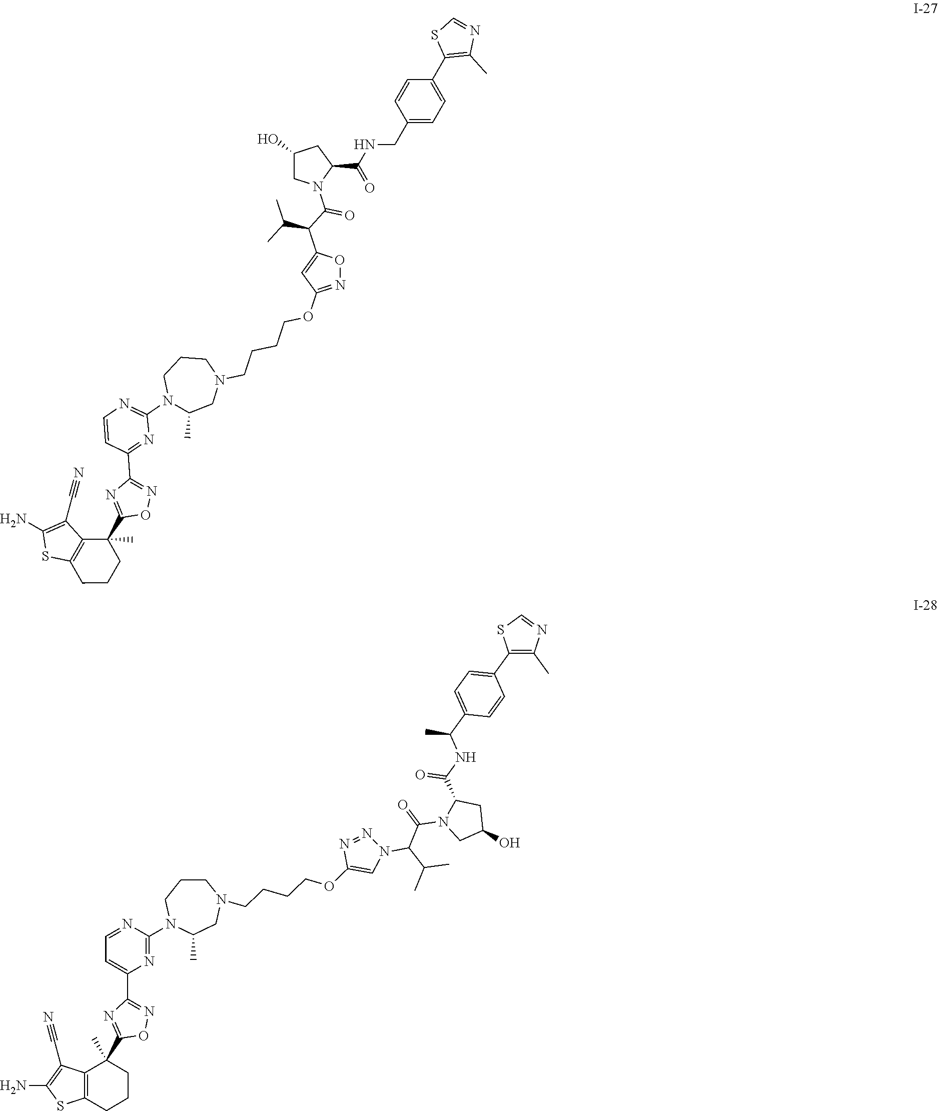 [SMILES: Cc1ncsc1-c1ccc(CNC(=O)[C@@H]2C[C@@H](O)CN2C(=O)[C@@H](c2cc(OCCCCN3CCCN(c4nccc(-c5noc([C@@]6(C)CCCc7sc(N)c(C#N)c76)n5)n4)[C@@H](C)C3)no2)C(C)C)cc1 ; Cc1ncsc1-c1ccc([C@H](C)NC(=O)[C@@H]2C[C@@H](O)CN2C(=O)C(C(C)C)n2cc(OCCCCN3CCCN(c4nccc(-c5noc([C@@]6(C)CCCc7sc(N)c(C#N)c76)n5)n4)[C@@H](C)C3)nn2)cc1] 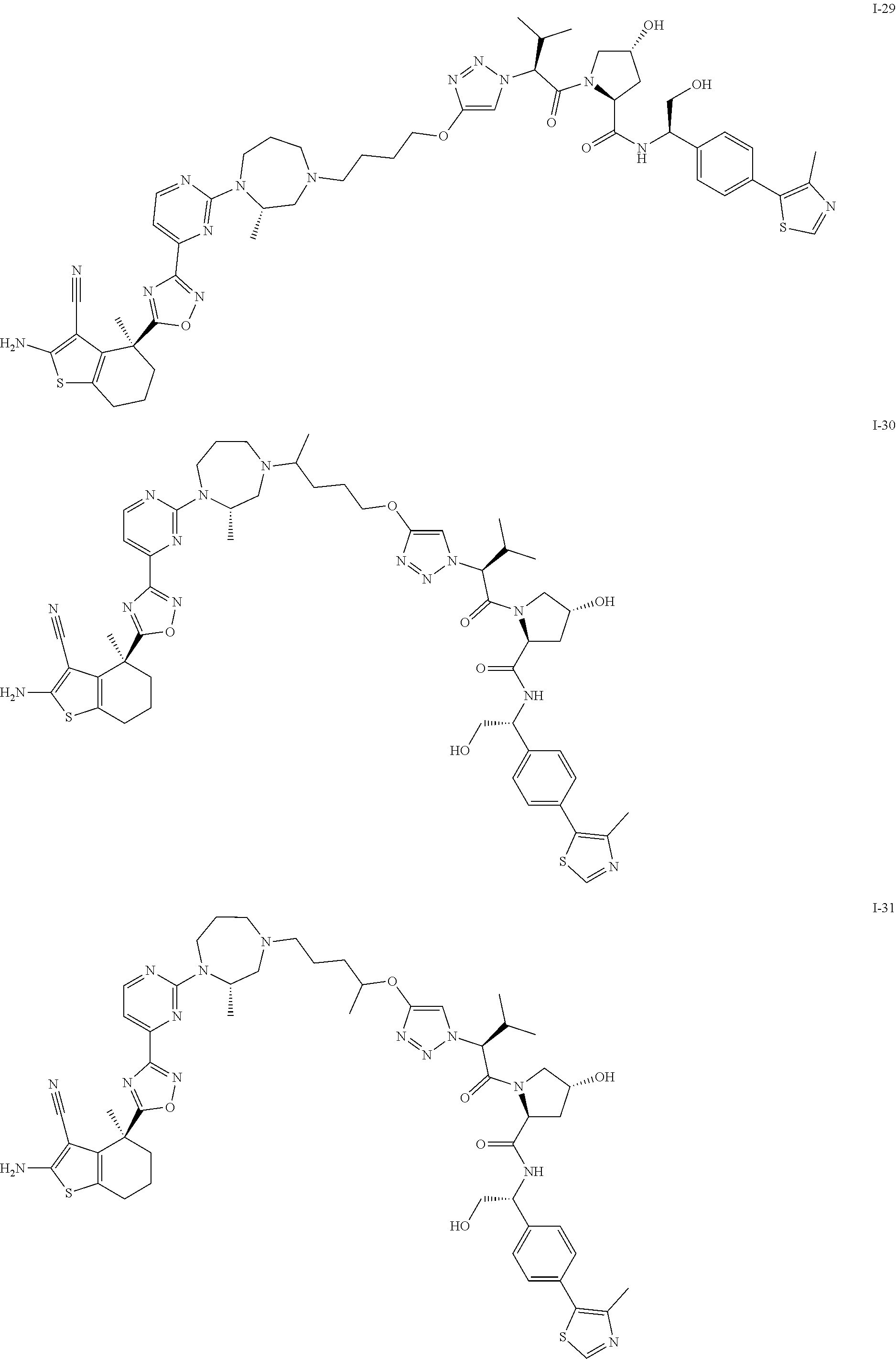 [SMILES: Cc1ncsc1-c1ccc([C@H](CO)NC(=O)[C@@H]2C[C@@H](O)CN2C(=O)[C@H](C(C)C)n2cc(OCCCCN3CCCN(c4nccc(-c5noc([C@@]6(C)CCCc7sc(N)c(C#N)c76)n5)n4)[C@@H](C)C3)nn2)cc1 ; Cc1ncsc1-c1ccc([C@H](CO)NC(=O)[C@@H]2C[C@@H](O)CN2C(=O)[C@H](C(C)C)n2cc(OCCCC(C)N3CCCN(c4nccc(-c5noc([C@@]6(C)CCCc7sc(N)c(C#N)c76)n5)n4)[C@@H](C)C3)nn2)cc1 ; Cc1ncsc1-c1ccc([C@H](CO)NC(=O)[C@@H]2C[C@@H](O)CN2C(=O)[C@H](C(C)C)n2cc(OC(C)CCCN3CCCN(c4nccc(-c5noc([C@@]6(C)CCCc7sc(N)c(C#N)c76)n5)n4)[C@@H](C)C3)nn2)cc1] 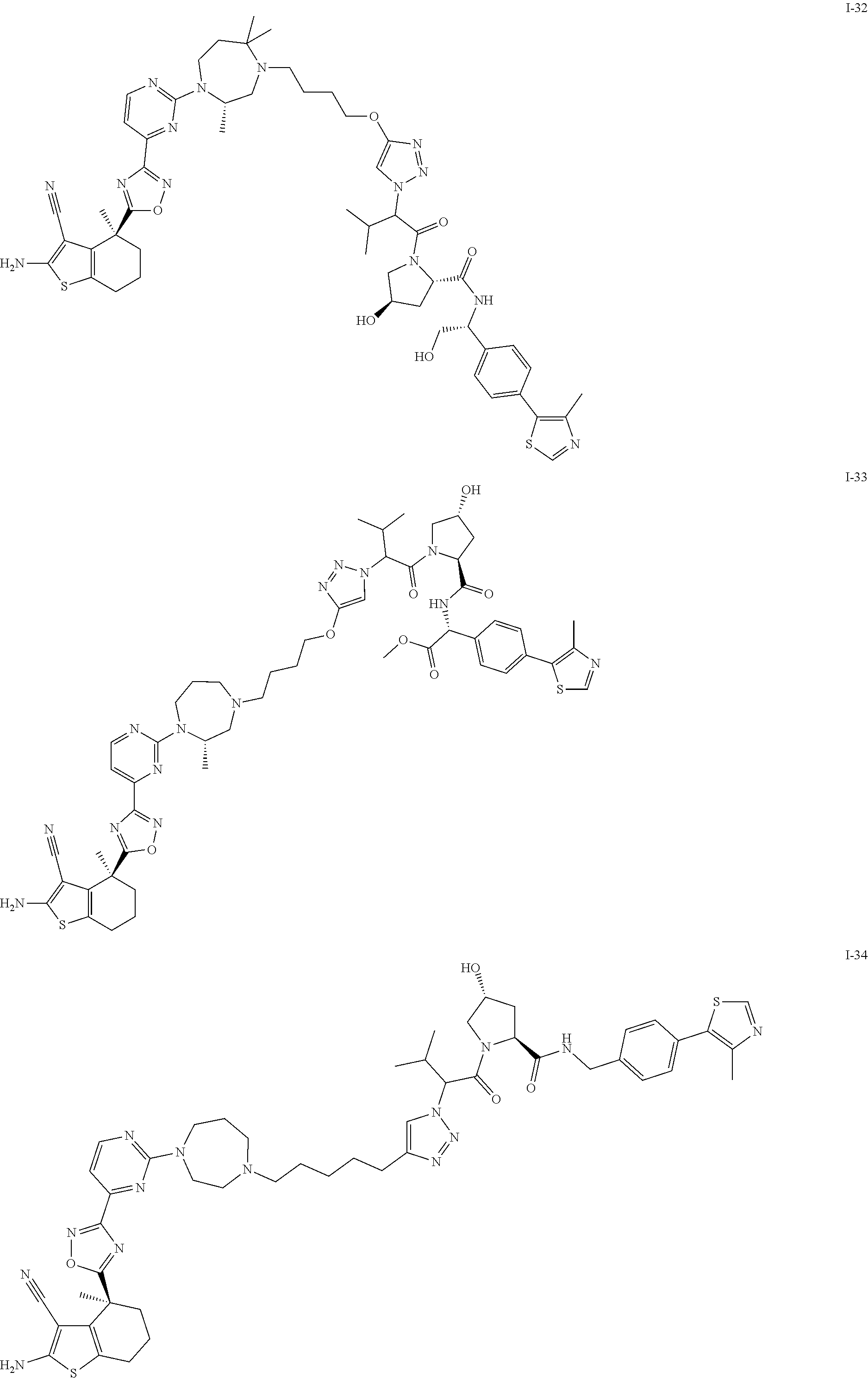 [SMILES: Cc1ncsc1-c1ccc([C@H](CO)NC(=O)[C@@H]2C[C@@H](O)CN2C(=O)C(C(C)C)n2cc(OCCCCN3C[C@H](C)N(c4nccc(-c5noc([C@@]6(C)CCCc7sc(N)c(C#N)c76)n5)n4)CCC3(C)C)nn2)cc1 ; COC(=O)[C@H](NC(=O)[C@@H]1C[C@@H](O)CN1C(=O)C(C(C)C)n1cc(OCCCCN2CCCN(c3nccc(-c4noc([C@@]5(C)CCCc6sc(N)c(C#N)c65)n4)n3)[C@@H](C)C2)nn1)c1ccc(-c2scnc2C)cc1 ; Cc1ncsc1-c1ccc(CNC(=O)[C@@H]2C[C@@H](O)CN2C(=O)C(C(C)C)n2cc(CCCCCN3CCCN(c4nccc(-c5noc([C@@]6(C)CCCc7sc(N)c(C#N)c76)n5)n4)CC3)nn2)cc1] 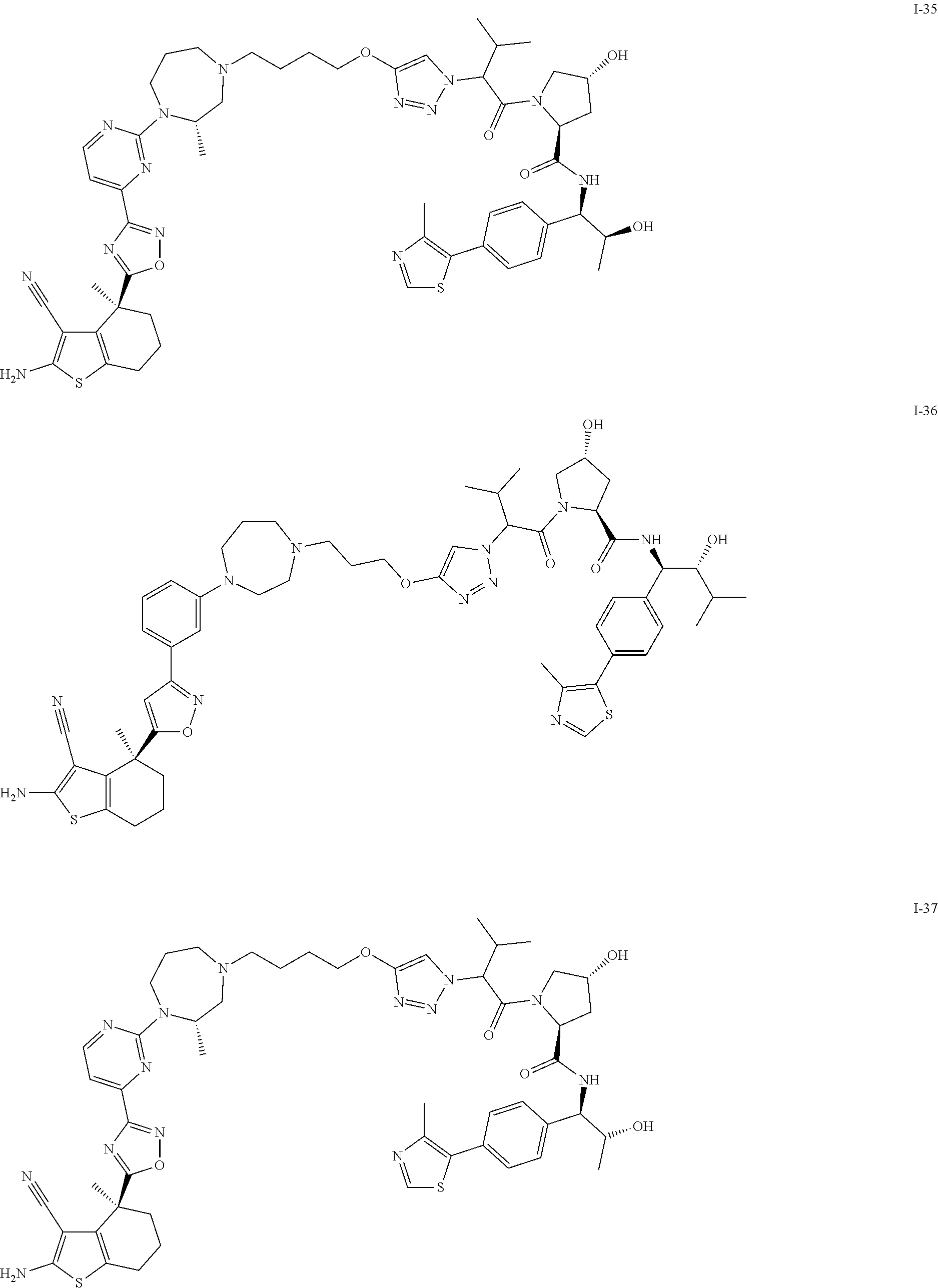 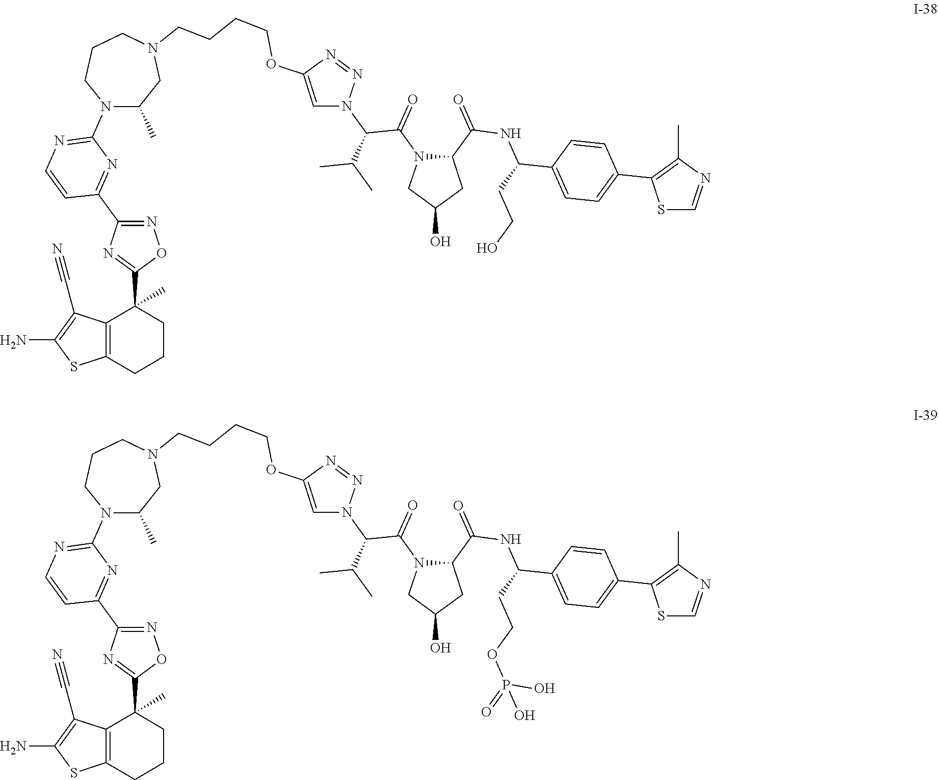 [SMILES: Cc1ncsc1-c1ccc([C@H](CCO)NC(=O)[C@@H]2C[C@@H](O)CN2C(=O)[C@H](C(C)C)n2cc(OCCCCN3CCCN(c4nccc(-c5noc([C@@]6(C)CCCc7sc(N)c(C#N)c76)n5)n4)[C@@H](C)C3)nn2)cc1 ; Cc1ncsc1-c1ccc([C@H](CCOP(=O)(O)O)NC(=O)[C@@H]2C[C@@H](O)CN2C(=O)[C@H](C(C)C)n2cc(OCCCCN3CCCN(c4nccc(-c5noc([C@@]6(C)CCCc7sc(N)c(C#N)c76)n5)n4)[C@@H](C)C3)nn2)cc1] 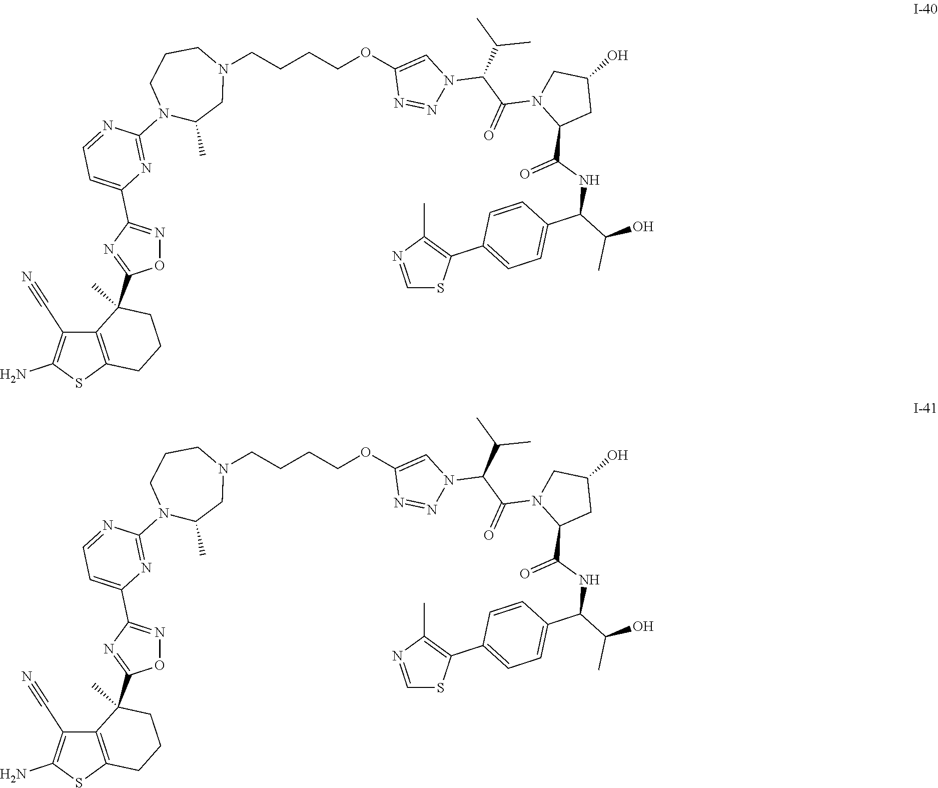 [SMILES: Cc1ncsc1-c1ccc([C@@H](NC(=O)[C@@H]2C[C@@H](O)CN2C(=O)[C@@H](C(C)C)n2cc(OCCCCN3CCCN(c4nccc(-c5noc([C@@]6(C)CCCc7sc(N)c(C#N)c76)n5)n4)[C@@H](C)C3)nn2)[C@H](C)O)cc1 ; Cc1ncsc1-c1ccc([C@@H](NC(=O)[C@@H]2C[C@@H](O)CN2C(=O)[C@H](C(C)C)n2cc(OCCCCN3CCCN(c4nccc(-c5noc([C@@]6(C)CCCc7sc(N)c(C#N)c76)n5)n4)[C@@H](C)C3)nn2)[C@H](C)O)cc1] 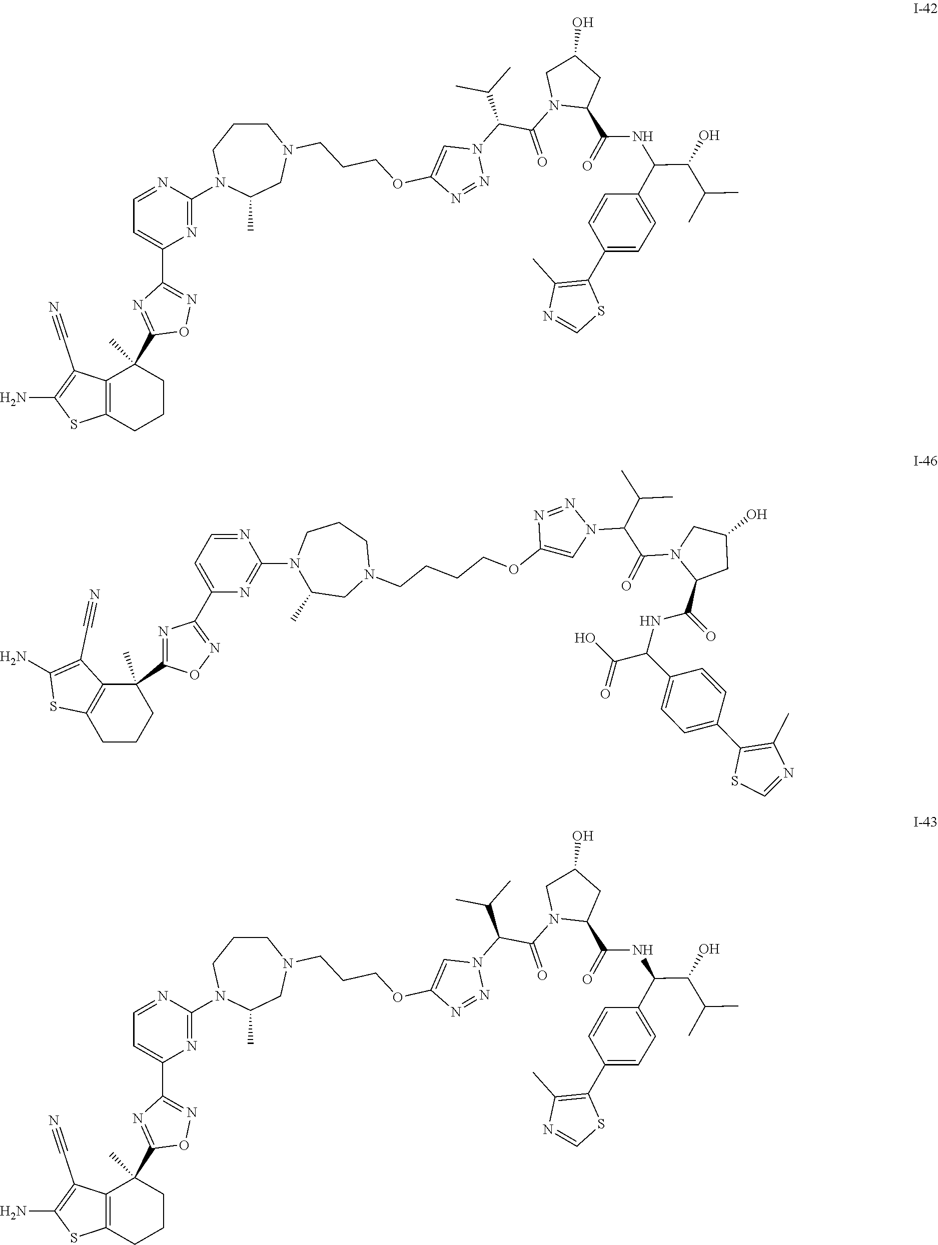 [SMILES: Cc1ncsc1-c1ccc(C(NC(=O)[C@@H]2C[C@@H](O)CN2C(=O)[C@@H](C(C)C)n2cc(OCCCN3CCCN(c4nccc(-c5noc([C@@]6(C)CCCc7sc(N)c(C#N)c76)n5)n4)[C@@H](C)C3)nn2)[C@H](O)C(C)C)cc1 ; Cc1ncsc1-c1ccc(C(NC(=O)[C@@H]2C[C@@H](O)CN2C(=O)C(C(C)C)n2cc(OCCCCN3CCCN(c4nccc(-c5noc([C@@]6(C)CCCc7sc(N)c(C#N)c76)n5)n4)[C@@H](C)C3)nn2)C(=O)O)cc1 ; Cc1ncsc1-c1ccc([C@@H](NC(=O)[C@@H]2C[C@@H](O)CN2C(=O)[C@H](C(C)C)n2cc(OCCCN3CCCN(c4nccc(-c5noc([C@@]6(C)CCCc7sc(N)c(C#N)c76)n5)n4)[C@@H](C)C3)nn2)[C@H](O)C(C)C)cc1] 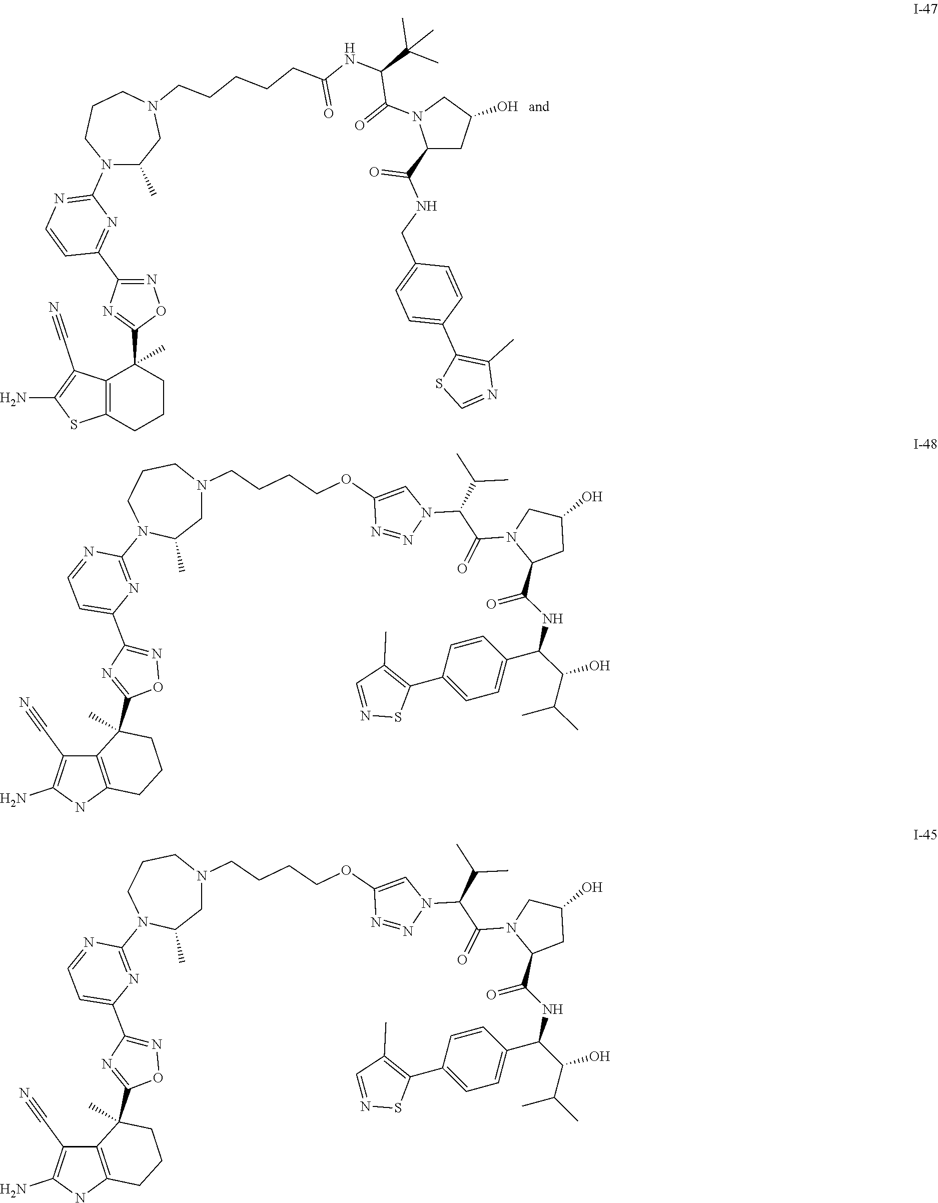 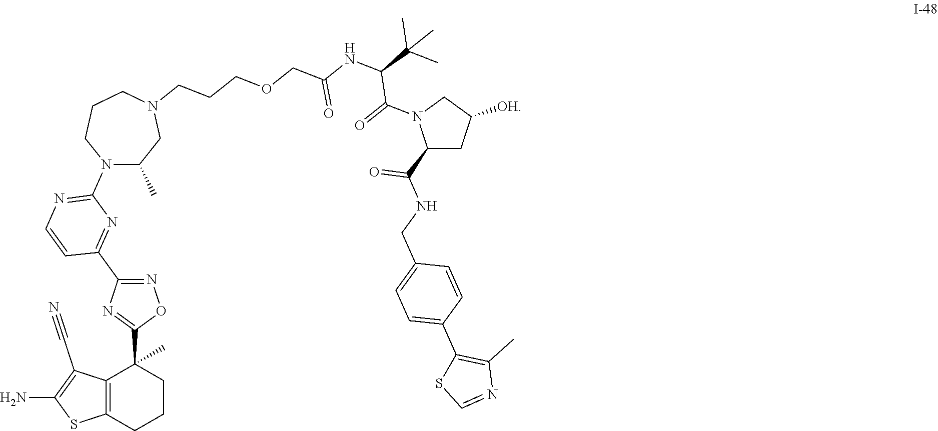 [SMILES: Cc1ncsc1-c1ccc(CNC(=O)[C@@H]2C[C@@H](O)CN2C(=O)[C@@H](NC(=O)COCCCN2CCCN(c3nccc(-c4noc([C@@]5(C)CCCc6sc(N)c(C#N)c65)n4)n3)[C@@H](C)C2)C(C)(C)C)cc1]
or a pharmaceutically acceptable salt thereof.

24. A compound selected from the group consisting of: 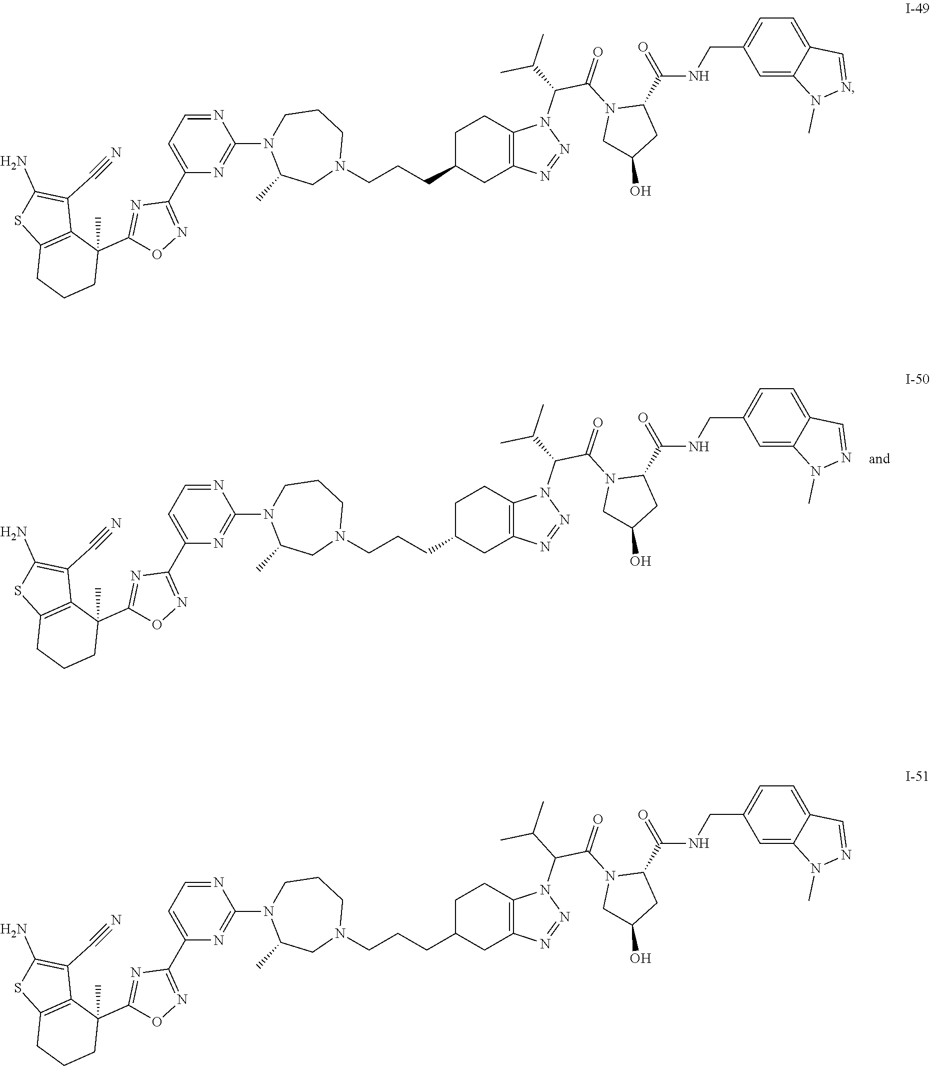 [SMILES: CC(C)[C@H](C(=O)N1C[C@H](O)C[C@H]1C(=O)NCc1ccc2cnn(C)c2c1)n1nnc2c1CC[C@H](CCCN1CCCN(c3nccc(-c4noc([C@@]5(C)CCCc6sc(N)c(C#N)c65)n4)n3)[C@@H](C)C1)C2 ; CC(C)[C@H](C(=O)N1C[C@H](O)C[C@H]1C(=O)NCc1ccc2cnn(C)c2c1)n1nnc2c1CC[C@@H](CCCN1CCCN(c3nccc(-c4noc([C@@]5(C)CCCc6sc(N)c(C#N)c65)n4)n3)[C@@H](C)C1)C2 ; CC(C)C(C(=O)N1C[C@H](O)C[C@H]1C(=O)NCc1ccc2cnn(C)c2c1)n1nnc2c1CCC(CCCN1CCCN(c3nccc(-c4noc([C@@]5(C)CCCc6sc(N)c(C#N)c65)n4)n3)[C@@H](C)C1)C2]
or a pharmaceutically acceptable salt thereof.

25. A compound as defined in any one of claims 1 to 24—or a pharmaceutically acceptable salt thereof—for use as a medicament.

26. A compound as defined in any one of claims 1 to 24—or a pharmaceutically acceptable salt thereof—for use in the treatment and/or prevention of cancer.

27. The compound—or a pharmaceutically acceptable salt thereof—for use according to claim 26, wherein said compound or salt is administered in combination with one or more other pharmacologically active substance(s).

28. The compound—or the pharmaceutically acceptable salt thereof—for use according to claim 26 or 27, wherein the cancer is selected from the group consisting of pancreatic cancer, lung cancer, colorectal cancer, cholangiocarcinoma, appendiceal cancer, multiple myeloma, melanoma, uterine cancer, endometrial cancer, thyroid cancer, acute myeloid leukaemia, bladder cancer, urothelial cancer, gastric cancer, cervical cancer, head and neck squamous cell carcinoma, diffuse large B cell lymphoma, oesophageal cancer, gastroesophageal cancer, chronic lymphocytic leukaemia, hepatocellular cancer, breast cancer, ovarian cancer, prostate cancer, glioblastoma, renal cancer and sarcoma.

29. The compound—or the pharmaceutically acceptable salt thereof—for use according to any one of claims 26 to 28, wherein the cancer comprises tumor cells harbouring a KRAS mutation or an amplification of KRAS wildtype.

30. The compound—or the pharmaceutically acceptable salt thereof—for use according to claim 29, wherein the KRAS mutation is selected from the group consisting of: KRAS G12C, KRAS G12D, KRAS G12V and KRAS G13D.

31. A pharmaceutical composition comprising a compound as defined in any one of claims 1 to 24—or a pharmaceutically acceptable salt thereof— and one or more pharmaceutically acceptable excipient(s).
